(19) United States
(12) Patent Application Publication
(10) Pub. No.: US 2023/0128402 A1
(43) Pub. Date: Apr. 27, 2023

(54) HETEROCYCLIC COMPOUNDS AND USES THEREOF

(71) Applicants: The Trustees of Princeton University, Princeton, NJ (US); KayoThera Inc., Princeton, NJ (US)

(72) Inventors: Mark ESPOSITO, Cazenovia, NY (US); John PROUDFOOT, Newtown, CT (US); Yibin KANG, Princeton, NJ (US)

(21) Appl. No.: 17/795,047

(22) PCT Filed: Jan. 25, 2021
(86) PCT No.: PCT/US2021/014883

§ 371 Date: Jul. 25, 2022

Related U.S. Application Data
(60) Provisional application No. 62/965,371, filed on Jan. 24, 2020.
(60) Provisional application No. 63/094,741, filed on Oct. 21, 2020.

(51) Int. Cl. C07D 401/12; C07D 471/04; C07D 487/04; C07D 413/12; C07D 498/04; A61P 35/00

(52) U.S. Cl. CPC C07D 401/12; C07D 471/04; C07D 487/04; C07D 413/12; C07D 498/04; A61P 35/00

(57) ABSTRACT

Provided herein are novel heterocyclic compounds, for example, compounds having Formula I, I-P, II, II-P, or III. Also provided herein are pharmaceutical compositions comprising the compounds and methods of using the same, for example, in inhibiting aldehyde dehydrogenases and/or for treating various cancers, cancer metastasis, type 2 diabetes, pulmonary arterial hypertension (PAH) or neointimal hyperplasia (NIH).

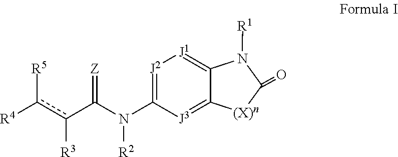

### CROSS REFERENCE TO RELATED APPLICATIONS

This application claims the benefit of United States Provisional Application Nos. 62/965,371, filed Jan. 24, 2020, and 63/094,741, filed Oct. 21, 2020, the content of each of which is incorporated herein by reference in its entirety.

In various embodiments, the present disclosure generally relates to novel heterocyclic compounds, pharmaceutical compositions, and methods of using the same, such as for inhibiting aldehyde dehydrogenases, treating various cancers, cancer metastasis, metabolic diseases such as type 2 diabetes, pulmonary arterial hypertension (PAH) or neointimal hyperplasia (NIH).

### BACKGROUND

Aldehyde dehydrogenases (ALDHs) belong to a superfamily of NAD(P+)-dependent enzymes that play a role in the metabolism of aldehydes by irreversibly catalyzing the oxidation of both endogenously and exogenously produced aldehydes to their respective carboxylic acids. ALDHs have a broad spectrum of biological activities, including biosynthesis of retinoic acid (RA), oxidation of lipid peroxides, and alcohol metabolism, among others.

The ALDH family of enzymes contains 19 members with diverse functions. Enzymes within this family irreversibly catalyze the oxidation of an aldehyde into the corresponding carboxylic acid while reducing NAD+/NADP+ to NADH/NADPH. These enzymes are found in several cellular compartments, however, most are localized to the cytosol or the mitochondria.

### BRIEF SUMMARY

Some ALDH enzymes participate in global metabolism via expression in the liver where they function to detoxify acetylaldehyde formed from alcohol dehydrogenases, biosynthesize vitamin A from retinal stereoisomers, or detoxify other reactive aldehydes. In contrast, most ALDH enzymes are expressed in a cell- or disease-specific manner and modulate cellular biochemistry, often with unknown mechanisms of action.

The present disclosure is based, in part, on the discovery that aldehyde dehydrogenase (Aldh, ALDH), and particularly ALDH isoform 1a3 (ALDH1a3), is implicated in various diseases or disorders such as proliferative diseases or disorders, metabolic diseases or disorders, endothelial cell or smooth muscle cell diseases or disorders, cancer and metastasis, etc. The present disclosure further shows that inhibition of the ALDH enzymes such as ALDH1a3 can be useful in treating or preventing various cancers, cancer metastasis, and other ALDH1a3-mediated diseases and disorders, metabolic diseases, such as such as type 2 diabetes, pulmonary arterial hypertension (PAH) and neointimal hyperplasia (NIH). See also, PCT/US2019/044278, which has an international filing date of Jul. 31, 2019, the content of which is incorporated by reference in its entirety.

Accordingly, in various embodiments, the present disclosure provides novel compounds and pharmaceutical compositions, which are useful in inhibiting aldehyde dehydrogenase (Aldh, ALDH), and particularly ALDH isoform 1a3 (ALDH1a3). In some embodiments, the present disclosure also provides methods of using the novel compounds and pharmaceutical compositions herein for treating various diseases or disorders, such as various cancers, cancer metastasis, metabolic diseases such as type 2 diabetes, pulmonary arterial hypertension (PAH) and neointimal hyperplasia (NIH).

Some embodiments of the present disclosure are directed to a compound of Formula I, I-P, II, II-P, or III, or a pharmaceutically acceptable salt thereof:

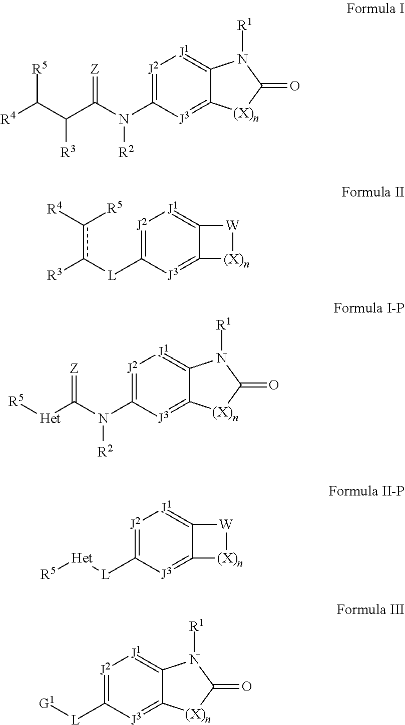

wherein the variables are defined herein. In some embodiments, the compound of Formula I can be characterized as having a subformula of Formula I as defined herein, such as Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C. In some embodiments, the compound of Formula II can be characterized as having a subformula of Formula II as defined herein, such as Formula II-1, II-2, II-3, or II-4. In some embodiments, the compound of Formula III can be characterized as having a subformula of Formula III as defined herein, such as Formula III-1, III-2. In some embodiments, the present disclosure also provides specific compounds, Compound Nos. 1-138, or a pharmaceutically acceptable salt thereof.

Certain embodiments of the present disclosure are directed to a pharmaceutical composition comprising one or more of the compounds of the present disclosure (e.g., a compound of Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), Formula I-P, Formula II (e.g., Formula II-1, II-2, II-3, or II-4,), Formula II-P, Formula III (e.g., Formula III-1 or III-2), or any of Compound Nos. 1-138, or a pharmaceutically acceptable salt thereof) and optionally a pharmaceutically acceptable excipient. The pharmaceutical composition described herein can be formulated for different routes of administration, such as oral administration, parenteral administration, or inhalation etc.

Some embodiments of the present disclosure are directed to a method of inhibiting an aldehyde dehydrogenase, in particular, ALDH1a3, in a subject in need thereof.

In some embodiments, the present disclosure provides a method of treating or preventing a disease or disorder associated with aldehyde dehydrogenase, preferably, a disease or disorder associated with aldehyde dehydrogenase isoform 1a3 (ALDH1a3) in a subject in need thereof. In some embodiments, the disease or disorder is a proliferative disease such as cancer (e.g., as described herein) associated with aldehyde dehydrogenase isoform 1a3 (ALDH1a3). In some embodiments, the disease or disorder is a metabolic disease such as type 2 diabetes associated with aldehyde dehydrogenase isoform 1a3 (ALDH1a3). In some embodiments, the disease or disorder is an endothelial cell or smooth muscle cell disease or disorder, such as pulmonary arterial hypertension or neointimal hyperplasia, associated with aldehyde dehydrogenase isoform 1a3 (ALDH1a3).

In some embodiments, the present disclosure provides a method of treating cancer in a subject in need thereof. In some embodiments, the cancer is associated with ALDH1a3 activites, such as having cancer cells with higher expression level compared to a control, and/or having cancer cells with ALDH1a3 activities, e.g., positive in Aldefluor™ assay, which can be reduced with an ALDH1a3 inhibitor or genetic knockout or knockdown. In some embodiments, the cancer is a solid cancer. In some embodiments, the cancer is metastatic cancer or chemoresistant cancer. In some embodiments, the cancer can be a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, and/or sarcoma.

In some embodiments, the present disclosure provides a method of treating or preventing metastasis of a cancer in a subject in need thereof. In some embodiments, the cancer has established metastasis. In some embodiments, the cancer has not metastasized prior to treatment with the methods herein, and the method delays or prevents metastasis of the cancer. In some embodiments, the cancer is associated with ALDH1a3 activites.

In some embodiments, the present disclosure provides a method of treating a metabolic disease, such as type 2 diabetes in a subject in need thereof. In some embodiments, the present disclosure further provides a method of treating an endothelial cell or smooth muscle cell disease or disorder, such as pulmonary arterial hypertension or neointimal hyperplasia, in a subject in need thereof.

The method described herein typically comprises administering to the subject an effective amount of a compound of the present disclosure (e.g., a compound of Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), Formula I-P, Formula II (e.g., Formula II-1, II-2, II-3, or II-4,), Formula II-P, Formula III (e.g., Formula III-1 or III-2), or any of Compound Nos. 1-138, or a pharmaceutically acceptable salt thereof) or an effective amount of a pharmaceutical composition described herein. The administering is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally. In some embodiments, compounds of the present disclosure can be administered as the only active ingredient(s). In some embodiments, compounds of the present disclosure can be used in combination with an additional therapy, such as conventional surgery or radiotherapy, immunotherapy, cell therapy, therapeutic antibodies, or chemotherapy.

It is to be understood that both the foregoing summary and the following detailed description are exemplary and explanatory only, and are not restrictive of the invention herein.

### BRIEF DESCRIPTION OF THE DRAWINGS/FIGURES

FIG. 1A is flow cytometry spectra, and shows that genetic knockout of ALDH1a3 (middle and rightmost spectra, two distinct ALDH1a3-targeting CRISPR gRNAs) in MDA-MB-468 breast cancer cells substantially decreases ALDEFLUOR™ activity compared to control MDA-MB-468 cells (leftmost spectra).

FIG. 1B is a line graph of tumor volume (mm³) versus time (days), and shows that genetic knockout of ALDH1a3 (KO #1 and KO #2) in MDA-MB-468 breast cancer cells slows primary tumor growth and sensitizes tumors to paclitaxel (ptx) compared to control cells (Vec).

FIG. 1C is a bar graph of tumor mass (g) versus ALDH1a3 genetic knockout (KO #1 and KO #2), and shows that genetic knockout of ALDH1a3 in MDA-MB-468 breast cancer cells slows primary tumor growth compared to control (Vec) and sensitizes tumors to paclitaxel (ptx).

FIG. 2A is flow cytometry spectra, and shows that genetic knockout of ALDH1a3 in Sum159-M1a breast cancer cells nearly abolishes ALDEFLUOR™ activity in the cells, and that ALDEFLUOR™ activity can be rescued by transducing the cells with a rescue vector encoding ALDH1a3 compared to empty vector.

FIG. 2B is a line graph of bone metastasis, as measured by bioluminescence (ph/s), versus time (days), and shows that knockout of ALDH1a3 in Sum159-M1a breast cancer cells slows bone metastasis growth.

FIG. 2C is a Kaplan-Meier plot of bone metastasis-free survival over time, and shows that knockout of ALDH1a3 in Sum159-M1a breast cancer cells significantly increases survival time. Statistics by Cox's proportional hazards model.

FIG. 3A is a line graph of bioluminescence (ph/s) versus time (days), and shows the development of lung metastasis in mice injected with SUM 159-M1b cells transduced with vectors encoding three ALDH enzymes, ALDH1a1, ALDH1a3 and ALDH3a1 compared to empty vector (vector).

FIG. 3B is a plot of lung nodes counted ex vivo at the endpoint of the experiment described in FIG. 3A. Student's t-test, two-tailed, assuming unequal variance.

FIG. 3C shows sample images of bioluminescence at Day1 (left) and endpoint (right) from the experiment described in FIG. 3A and FIG. 3B.

FIG. 4A is a patient survival curve stratified by high (red) and low (black) Aldh1a3 expression based on the data analysis tool hosted at kmplot.com, and shows the distant metastasis-free survival for breast cancer patients as a function of ALDH1a3 expression level.

FIG. 4B is a patient survival curve stratified by high (red) and low (black) Aldh1a3 expression based on the data analysis tool hosted at kmplot.com, and shows the overall survival for renal clear cell cancer patients as a function of ALDH1a3 expression level.

FIG. 4C is a patient survival curve stratified by high (red) and low (black) Aldh1a3 expression based on the data analysis tool hosted at kmplot.com, and shows the overall survival for gastric cancer patients as a function of ALDH1a3 expression level.

FIG. 4D is a patient survival curve stratified by high (red) and low (black) Aldh1a3 expression based on the data analysis tool hosted at kmplot.com, and shows the overall survival for bladder cancer patients as a function of ALDH1a3 expression level.

FIG. 4E is a patient survival curve stratified by high (red) and low (black) Aldh1a3 expression based on the data analysis tool hosted at kmplot.com, and shows the overall survival for ovarian cancer patients as a function of ALDH1a3 expression level.

FIG. 4F is a patient survival curve stratified by high (red) and low (black) Aldh 1a3 expression based on the data analysis tool hosted at kmplot.com, and shows the overall survival for lung squamous cancer patients as a function of ALDH1a3 expression level.

FIG. 4G is a patient survival curve stratified by high (red) and low (blue) Aldh1a3 expression based on survival time series data and patient-level RNA expression data from The Cancer Genome Atlas, and shows the overall survival for colorectal cancer patients as a function of ALDH1a3 expression level.

FIG. 4H is a patient survival curve stratified by high (red) and low (blue) Aldh1a3 expression based on survival time series data and patient-level RNA expression data from The Cancer Genome Atlas, and shows the overall survival for low-grade glioma patients as a function of ALDH1a3 expression level.

FIG. 5A is graph of mRNA expression of Aldh1a3 from the METABRIC clinical breast cancer dataset, and shows expression of Aldh1a3 by breast cancer subtype and history of chemotherapy. Statistics by Student's t-test, two sided.

FIG. 5B is a set of survival curves based on the Erasmus Medical Center-Memorial Sloan-Kettering (EMC-MSK) dataset, and shows the survival time of breast cancer patients by subtype and stratification by median ALDH1a3 expression level. Statistics by Cox's proportional hazards model.

FIG. 6A is a bar graph of percentage of ALDEFLUOR™-positive cells in the presence of various compounds described herein, and shows the percentage of SUM159-M1a-Aldh 1a3 cells that are above background fluorescence levels, as detected by flow cytometry after incubation using the standard ALDEFLUOR™ protocol described herein with compounds at a concentration of 100 nanomolar. Gating for background fluorescence was performed using 1 millimolar N,N-diethylaminobenzaldehyde (DEAB) as a negative control.

FIG. 6B is a line graph of percentage of ALDEFLUOR™-positive cells in the presence of varying concentrations of MBE1 or MBE1.5, and shows the percentage of SUM 159-M1a-Aldh 1a3 cells that are above background fluorescence levels, as detected by flow cytometry after incubation according to the standard ALDEFLUOR™ protocol described herein combined with a dose titration of MBE1 or MBE1-5. The [inh-min]threshold was set at the lower bound of two standard deviations of control samples, while the IC₅₀ threshold was set at 50% of the average of control samples.

FIG. 6C is a graph of ALDEFLUOR™ activity in SUM 159-M1a-Aldh1a3 cells versus concentration of various inhibitors described herein, and shows the ALDEFLUOR™ inhibitory activity of several compounds described herein at concentrations of 10 nM and 100 nM.

FIG. 7A is a Western blot, and shows the expression of various ALDH isoforms, including 1a1, 1a2, 1a3 and 3a1, in MCF7 and SUM 159 cells.

FIG. 7B is a line graph of percentage of ALDEFLUOR™-positive MCF7 cells expressing the indicated ALDH isoform versus the log of MBE 1.5 concentration, and shows that MBE 1.5 specifically inhibits ALDH1a3 at concentrations below 10 μM.

FIG. 7C is a line graph of percentage of ALDEFLUOR™-positive SUM 159 cells expressing the indicated ALDH isoform versus the log of MBE 1.5 concentration, and shows that MBE 1.5 specifically inhibits ALDH1a3 at concentrations below 10 μM.

FIG. 8 is a bar graph of ALDEFLUOR™ activity in a variety of cancer types in the presence of 1 mM DEAB (a pan-ALDH inhibitor) or 100 nM MBE1.5 (a specific ALDH1a3 inhibitor described herein), and shows that the majority of human cancer cell lines show Aldh1a3 activity.

FIG. 9A is a diagram of the dosing strategy used to administer MBE1 and paclitaxel to mice injected with M1a-Aldh1a3 cells via intravenous tail-vein injection, and shows the design of an in vivo experiment designed to test the efficacy of MBE1 in treating metastatic cancer.

FIG. 9B is a line graph of lung metastasis, as measured using bioluminescence imaging (BLI), versus time (days), and compares lung metastasis in the presence and absence of MBE1 in the mice from the experiment outlined in FIG. 9A. Student's t-test, two-tailed, assuming unequal variance.

FIG. 10A is a diagram of the dosing strategy used to administer MBE1 and paclitaxel to mice injected with M1a-Aldh1a3 cells via intracardiac injection, and shows the design of an in vivo experiment designed to test the efficacy of MBE1 in treating metastatic cancer.

FIG. 10B is a line graph of bone metastasis, as measured using BLI, versus time (days), and compares bone metastasis in the presence and absence of MBE1 in the mice from the experiment outlined in FIG. 10A. Student's t-test, one-tailed, assuming unequal variance.

FIG. 11A is a line graph of lung metastasis, as measured by bioluminescent imaging (BLI), versus time (days), and shows that three doses of 50 mg/kg MBE1.5 in combination with 25 mg/kg paclitaxel, administered on days 17, 19 and 21 caused regression of established metastatic disease in a mouse xenograft model. Student's t-test, two-tailed, assuming unequal variance.

FIG. 11B shows images of all mice are shown with equal exposure settings from the experiment described in FIG. 11A.

FIG. 12A is a line graph of body mass (g) versus time (days), and shows that there was no gross toxicity associated with MBE 1.5 treatment in this experiment.

FIG. 12B is a line graph of tumor volume (mm³) versus time (days), and shows that 12-day treatment with MBE1.5 compared to vehicle caused regression of MDA-MB-468 primary breast tumors in combination with 4 doses of paclitaxel administered to both groups. Statistics by Student's t-test.

FIG. 12C shows images of primary tumors at endpoint of the experiment described in FIG. 12B. Images of two tumors in the MBE1.5 group missing as these were fully eliminated.

FIG. 13A is a line graph of lung metastasis bioluminescence versus time (days), and shows the progression of lung metastasis before and after treatment with MBE1.5 or vehicle. Statistics by Student's t-test.

FIG. 13B is a Kaplan-Meier plot of mouse survival over time as a function of treatment group, and shows that 12-day treatment with MBE1.5 extended survival in mice with late-stage established breast cancer lung metastasis. Statistics by Cox's proportional hazards model.

FIG. 13C shows sample bioluminescent images of each treatment group before and after treatment.

FIG. 14 is a line graph of colorectal metastasis bioluminescence versus time (days), and shows the progression of colorectal metastasis after treatment with MBE1.5 or vehicle. FIG. 14 shows that combination treatment of MBE1.5 and paclitaxel slows colorectal cancer metastasis. Statistics by Student's t-test. *p<0.05.

FIG. 15A is a line graph of the pharmacokinetics of compounds MBE1 that shows that oral gavage (PO) and intravenous (IV) administration of compound MBE1 leads to plasma concentrations that exceed 5-fold the IC50 for >10 hours. Data points are the average of biological replicates, n=3 mice per group.

FIG. 15B is a line graph of the pharmacokinetics of compounds MBE1.5 that shows that oral gavage (PO) and intravenous (IV) administration of compound MBE1.5 leads to plasma concentrations that exceed 5-fold the IC50 for >10 hours. Data points are the average of biological replicates, n=3 mice per group.

FIG. 16A is a bar graph showing the LC-MS quantification of the medium chain fatty aldehyde adipate semialdehyde in HEK293T cells treated with vehicle control or compound MBE1.5 (10 μM) for 1 hour showing the inhibition of Aldh1a3 leads to accumulation of medium chain fatty aldehydes implicated in Type II Diabetes pathogenesis and endothelial proliferation associated with PAH. n=3 cells per group

FIG. 16B is a bar graph showing the LC-MS quantification of reduced NADH in HEK293T cells treated with vehicle control or compound MBE1.5 (10 μM) for 1 hour and shows that inhibition of Aldh1a3 leads to a reduction in NADH in cells. n=3 cells per group

FIG. 17 is a line graph of ELISA quantification of plasma insulin levels in mice that had received MBE1 once daily for 14 days and were challenged with a standard fasting and refeeding assay to measure insulin secretion. n=10 mice per group.

FIG. 18 is a bar graph of pancreatic islet cells extracted from diet-induced diabetic or healthy C57BL6 mice that were isolated into a single cell suspension and assessed via the ALDEFLUOR™ assay in the presence of DMSO (vehicle), 1 mM DEAB, 10 μM MBE1.5 (n=2 biological replicates per group) and demonstrates that only diabetic mouse pancreatic islet cells express Aldh1a3 that is inhibited by compound MBE1.5.

### DETAILED DESCRIPTION OF THE INVENTION

As explained in more detail in the Examples section, Aldh1a3 was found to be an essential driver of tumor metastasis and resistance to chemotherapy. Data herein demonstrated that genetic ablation of Aldh1a3 in the triple negative breast cancer models Sum159-M1a and MDA-MB-468 sensitizes orthotopic tumors to paclitaxel treatment. Aldh1a3 was found to be a critical determinant of metastasis initiation and growth both as a single genetic element and when combined with chemotherapy. Genetic experiments demonstrate that Aldh1a3 is necessary for lung and bone metastasis in triple negative breast cancer metastasis. Further, clinical analysis of multiple cancer types supports Aldh1a3 as the differentiated Aldh isoform predicting worse outcome across multiple solid tumor indications. For example, high Aldh1a3 expression predicts worse overall survival in the more metastatic and aggressive estrogen receptor negative (ER−) breast cancer patients, and this prognosis is further worsened if those patients had received neoadjuvant chemotherapy (Table 1).

Also shown herein, genetic knockout of ALDH1a3 or inhibition of ALDH1a3 with representative ALDH1a3 inhibitors can slow primary tumor growth, sensitize tumors to chemotherapy, slow metastasis, and enhance survival time. As detailed in Biological Example 6, in mouse xenograph models, ALDH1a3 inhibitors (MBE1 or MBE1.5), in conjunction with a chemotherapy agent (paclitaxel), were effective in treating established metastatic diseases and can cause regression of primary tumors, slow various metastasis, and extend survival time. Research has also shown that diseases such as type 2 diabetes, pulmonary arterial hypertension (PAH) or neointimal hyperplasia (NIH) are also caused by ALDH1a3 expression and/or activities.

As also detailed herein, compounds described herein are orally available and exhibit sufficient pharmacokinetic exposure to effectively inhibit Aldh1a3 in mouse models.

In addition, Aldh1a3 was found to be an important driver of Type 2 Diabetes progression. Data herein demonstrate that ALDH1a3 is involved in the metabolism of medium chain fatty acids known to cause pathogenesis of Type 2 Diabetes and various endothelial disorders such as PAH and NIH. Data herein also demonstrated that pharmacologic inhibition of Aldh1a3 in the leptin-deficient db/db mouse strain effectively treats Type 2 Diabetes by restoring insulin secretion and subsequent blood glucose control.

Also shown herein, pancreatic islet cells isolated from obese diabetic C57/BL6 wild-type mice express active Aldh1a3 that is inhibited by compound MBE1.5 while pancreatic cells from non-obese, non-diabetic C57/BL6 mice do not express Aldh1a3.

Accordingly, in various embodiments, the present disclosure provides novel compounds and compositions, which are useful for inhibiting ALDH such as ALDH1a3, and methods of using the same, for example, for treating various cancers, cancer metastasis, metabolic diseases such as type 2 diabetes, pulmonary arterial hypertension (PAH) or neointimal hyperplasia (NIH).

### Compounds

Provided herein are a range of compounds that can be useful for inhibiting ALDH1a3. In PCT/US2019/044278, which has an international filing date of Jul. 31, 2019, it was shown that certain tetrahydroquinolinone compounds, such as Compound Nos. 1-17, can inhibit ALDH1a3, for example, in the ALDEFLUOR™ assay. Further, Compound MBE1 (Compound No. 1) was shown to shrink metastatic lesions in mice without toxicity. The present disclosure describes further compounds as ALDH inhibitors, in particular, ALDH1a3 inhibitors.

Formula I

In some embodiments, the present disclosure provides a compound of Formula I, or a pharmaceutically acceptable salt thereof:

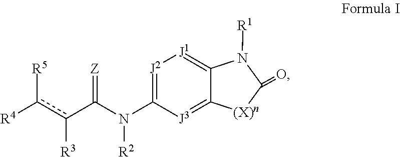

wherein:
X at each occurrence is independently selected from O, NR¹⁰, and CR²⁰R²¹, provided that at most one X is selected from O and NR¹⁰;
n is 1, 2, 3, or 4;
J¹, J², and J³ are each independently selected from CR²² or N, preferably, at least one of J¹, J², and J³ is not N;
R¹ and R² are each independently hydrogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), or a nitrogen protecting group;
R³ and R⁴ are joined to form an optionally substituted aryl, an optionally substituted heteroaryl, an optionally substituted carbocyclic (e.g., C₃₋₈ carbocyclic), or an optionally substituted heterocyclic ring (e.g., 3-8 membered heterocyclic ring);
Z is O, and R⁵ is hydrogen, —NR¹¹R¹², —CR²³R²⁴R¹⁵, or —OR³⁰;
or Z is O, and R³, R⁴ and R⁵ are joined to form an optionally substituted bicyclic or polycyclic ring system, wherein the ring system is an aryl, heteroaryl, carbocyclic, or heterocyclic ring system;
or R⁵ and Z are joined to form an optionally substituted aryl, an optionally substituted heteroaryl, an optionally substituted carbocyclic (e.g., C₃₋₈ carbocyclic), or an optionally substituted heterocyclic ring (e.g., 3-8 membered heterocyclic ring); and
“ [symbol] ” in Formula I indicates the bond is an aromatic bond, a double bond or a single bond as valance permits, and when a single bond, the two carbons forming the bond can be optionally further substituted as valance permits;
wherein:
   R¹⁰ at each occurrence is independently hydrogen, a nitrogen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, or an optionally substituted 3-8 membered heterocyclic ring;
   R²⁰ and R²¹ at each occurrence are each independently hydrogen, halogen, —OR³¹, —NR¹³R¹⁴, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl; or
   R¹⁰ and one of R²⁰ and R²¹ are joined to form a bond, an optionally substituted 4-8 membered heterocyclic ring or an optionally substituted 5 or 6 membered heteroaryl ring, wherein the other of R²⁰ and R²¹ is defined above;
   R²⁰ and R²¹ together with the carbon they are both attached to form —C(O)—, an optionally substituted C₃₋₈ carbocyclic ring, or an optionally substituted 3-8 membered heterocyclic ring; or
   one of R²⁰ and R²¹ in one CR²⁰R²¹ is joined with one of R²⁰ and R²¹ in a different CR²⁰R²¹ to form a bond, an optionally substituted C₃₋₈ carbocyclic ring, an optionally substituted 3-8 membered heterocyclic ring, wherein the others of R²⁰ and R²¹ are defined above;
   R²² at each occurrence is independently hydrogen, halogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), —CN, —S(O)-alkyl (e.g., —S(O)—C₁₋₆ alkyl), —S(O)₂-alkyl (e.g., —S(O)₂—C₁₋₆ alkyl), or —OR³¹;
one of R¹¹ and R¹² is hydrogen or a nitrogen protecting group, and the other of R¹¹ and R¹² is hydrogen, a nitrogen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl;
one of R²³, R²¹, and R²⁵ is hydrogen, halogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, an optionally substituted 5-10 membered heteroaryl, —OR³¹, or —NR¹³R¹⁴, and the other two of R²³, R²⁴, and R²⁵ are independently selected from hydrogen, fluorine, or methyl, preferably, —CR²³R²⁴R²⁵ is not —CH₃;
R³⁰ is hydrogen, an oxygen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, or an optionally substituted 3-8 membered heterocyclic ring; and
wherein:
   each of R¹³ and R¹⁴ at each occurrence is independently hydrogen, a nitrogen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl;
   or R¹³ and R¹⁴ are joined to form a 3-8 membered optionally substituted heterocyclic or a 5-10 membered optionally substituted heteroaryl; and
      R³¹ at each occurrence is hydrogen, an oxygen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl.

Typically, Z in Formula I is O and the compound can be characterized as having Formula I-O:

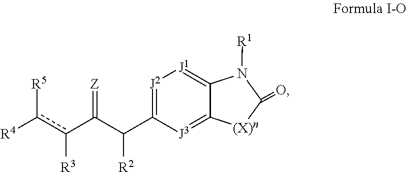

wherein R¹, R², R³, R⁴, R⁵, J¹, J², J³, X, and n are defined herein.

Typically, R³ and R⁴ in Formula I (e.g., Formula I-O) are joined to form an optionally substituted phenyl, an optionally substituted 5 or 6-membered heteroaryl, e.g., having one or two ring nitrogen atoms, an optionally substituted 04-7 cycloalkyl group (preferably cyclopentyl or cyclohexyl), or an optionally substituted 4 to 7-membered (preferably 6-membered) heterocyclic ring having one or two ring heteroatoms. To be clear, when it is said that R³ and R⁴ in Formula I are joined to form a ring system described herein, it should be understood that R³ and R⁴, together with the two intervening carbon atoms, are joined to form the ring system.

In some embodiments, R³ and R⁴ in Formula I (e.g., Formula I-O) can be joined to form an optionally substituted phenyl ring, i.e., the moiety of

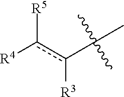

### in Formula I is

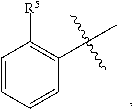

wherein R⁵ is defined herein, and wherein the phenyl can be further optionally substituted at any available position, for example, with one or two substituents independently selected from F; Cl; hydroxyl; C₁₋₄ alkyl optionally substituted with 1-3 fluorines, preferably, methyl, ethyl, n-propyl, isopropyl, or —CF₃; a C₁₋₄ alkoxy optionally substituted with 1-3 fluorines, preferably, methoxy, ethoxy, n-propoxy, isopropoxy, or —OCF₃; a C₃₋₈ cycloalkyl optionally substituted with 1-3 substituents independently selected from fluorine and methyl, preferably, cyclopropyl or cyclobutyl; and —CN. In some embodiments, R⁵ is —O—R³⁰ or —CR²³R²⁴R²⁵ as defined and preferred herein. For example, in some embodiments, R⁵ is ethyl, n-propyl, isopropyl, n-butyl, isobutyl, sec-butyl, —CH₂—CHF₂, —CH₂—CF₃, —CF₃, —CH₂-cyclopropyl, —CH₂-cyclobutyl, —CH₂—O—CH₃, —CH₂—O—C₂H₃, —CH₂—O-n-propyl, —CH₂—O-isopropyl, —C₂H₄-cyclopropyl, —C₂H₄-cyclobutyl, methoxy, ethoxy, n-propoxy, isopropoxy, n-butoxy, isobutoxy, sec-butoxy, —O—CH₂—CF₃, —O—CF₃, —O—CH₂-cyclopropyl, —O—CH₂-cyclobutyl, —O—C₂H₄-cyclopropyl, or —O—C₂H₄-cyclobutyl. In some embodiments, R⁵ is hydrogen.

In some embodiments, R³ and R⁴ in Formula I (e.g., Formula I-O) can be joined to form an optionally substituted 5 or 6-membered heteroaryl, such as those described herein. For example, in some embodiments, R³ and R⁴ in Formula I (e.g., Formula I-O) can be joined to form an optionally substituted pyrazole, imidazole, oxazole, thiazole, isoxazole, isothiazole, pyridyl, pyrimidinyl, pyridazinyl, or pyrazinyl. For example, in some embodiments, the moiety of

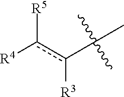

in Formula I (e.g., Formula I-O) can be selected from the following:

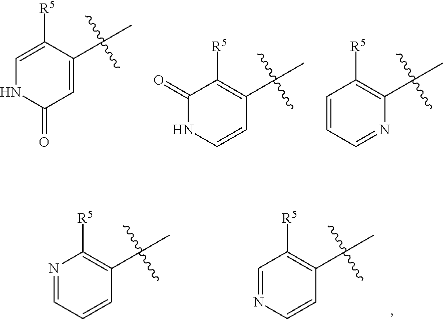

wherein R⁵ is defined herein, and wherein the pyridyl or pyridone can be further optionally substituted at any available position, including the ring nitrogen in the case of pyridone, for example, with one or two substituents (preferably one) independently selected from F; Cl; OH; C₁₋₄alkyl optionally substituted with 1-3 fluorines, preferably, methyl, ethyl, n-propyl, isopropyl, or —CF₃; a C₁₋₄ alkoxy optionally substituted with 1-3 fluorines, preferably, methoxy, ethoxy, n-propoxy, isopropoxy, or —OCF₃; a C₃₋₈ cycloalkyl optionally substituted with 1-3 substituents independently selected from fluorine and methyl, preferably, cyclopropyl or cyclobutyl; and —CN. In some embodiments, the moiety of

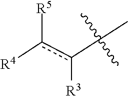

### in Formula I can be

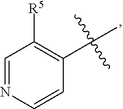

wherein R⁵ is defined herein, and wherein the pyridyl can be further optionally substituted at any available position, for example, with one or two substituents (preferably one) independently selected from F; Cl; C₁₋₄ alkyl optionally substituted with 1-3 fluorines, preferably, methyl, ethyl, n-propyl, isopropyl, or —CF₃; a C₁₋₄ alkoxy optionally substituted with 1-3 fluorines, preferably, methoxy, ethoxy, n-propoxy, isopropoxy, or —OCF₃; a C₃₋₄ cycloalkyl optionally substituted with 1-3 substituents independently selected from fluorine and methyl, preferably, cyclopropyl or cyclobutyl; and —CN. In some embodiments, R⁵ is —O—R³⁰ or —CR²³R²⁴R²⁵ as defined and preferred herein. For example, in some embodiments, R⁵ is ethyl, n-propyl, isopropyl, n-butyl, isobutyl, sec-butyl, —CH₂—CHF₂, —CH₂—CF₃, —CF₃, —CH₂-cyclopropyl, —CH₂-cyclobutyl, —CH₂—O—CH₃, —CH₂—O—C₂H₅, —CH₂—O-n-propyl, —CH₂—O-isopropyl, —C₂H₄-cyclopropyl, —C₂H₄-cyclobutyl, methoxy, ethoxy, n-propoxy, isopropoxy, n-butoxy, isobutoxy, sec-butoxy, —O—CH₂—CF₃, —O—CF₃, —O—CH₂-cyclopropyl, —O—CH₂-cyclobutyl, —O—C₂H₄-cyclopropyl, or —O—C₂H₄-cyclobutyl. In some embodiments, R⁵ can also be hydrogen.

In some embodiments, R³ and R⁴ in Formula I (e.g., Formula I-O) can be joined to form an optionally substituted 5 or 6-membered saturated ring system optionally containing one or two (preferably one) ring heteroatoms selected from O or N, such as cylopentyl, cyclohexyl, tetrahydropyranyl, piperidinyl, etc. Typically, when substituted, the 5 or 6-membered saturated ring system can be further optionally substituted by one or two substituents independently selected from F and C₁₋₄ alkyl optionally substituted with 1-3 fluorines. In some embodiments, the moiety of

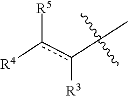

### in Formula I can be

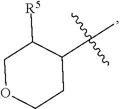

wherein R⁵ is defined herein, and wherein the tetrahydropyranyl can be further optionally substituted at any available position, for example, with one or two substituents independently selected from F and C₁₋₄ alkyl optionally substituted with 1-3 fluorines. In some embodiments, R⁵ is —O—R³⁰ or —CR²³R²⁴R²⁵ as defined and preferred herein. For example, in some embodiments, R⁵ is ethyl, n-propyl, isopropyl, n-butyl, isobutyl, sec-butyl, —CH₂—CHF₂, —CH₂—CF₃, —CF₃, —CH₂-cyclopropyl, —CH₂-cyclobutyl, —CH₂—O—CH₃, —CH₂—O—C₂H₅, —CH₂—O-n-propyl, —CH₂—O-isopropyl, —C₂H₄-cyclopropyl, —C₂H₄-cyclobutyl, methoxy, ethoxy, n-propoxy, isopropoxy, n-butoxy, isobutoxy, sec-butoxy, —O—CH₂—CF₃, —O—CF₃, —O—CH₂-cyclopropyl, —O—CH₂-cyclobutyl, —O—C₂H₄-cyclopropyl, or —O—C₂H₄-Cyclobutyl.

In some embodiments, R⁵ in Formula I (e.g., Formula I-O) can be hydrogen. However, typically, R⁵ in Formula I (e.g., Formula I-O) is —NR¹¹R¹², —CR²³R²⁴R²⁵, or —OR³⁰, more typically, —CR²³R²⁴R²⁵ or —OR³⁰, wherein R¹¹, R¹², R²³, R²⁴, R²⁵, and R³⁰ are defined herein. For example, in any of the embodiments described herein, unless specified or obviously contradictory from context, R⁵ in Formula I (e.g., Formula I-O) can be —CR²³R²⁴R²⁵, wherein
R²³ is hydrogen or fluorine;
R²⁴ is hydrogen or fluorine; and
R²⁵ is hydrogen, halogen, an optionally substituted C₁₋₄ alkyl, an optionally substituted C₃₋₆ carbocyclic ring, an optionally substituted 3-6 membered heterocyclic ring, an optionally substituted phenyl, or an optionally substituted 5 or 6 membered heteroaryl.

In some embodiments, R²⁵ can be fluorine. In some embodiments, R²⁵ can be a C₁₋₄ alkyl optionally substituted with one or more (e.g., 1, 2, or 3) substituents independently selected from fluorine, hydroxyl, fluoro-substituted C₁₋₄ alkyl (e.g., CF₃), C₁₋₄ alkoxy, fluoro-substituted C₁₋₄ alkoxy (e.g., —OCF₃), NH₂, —NH(C₁₋₄ alkyl), —N(C₁₋₄ alkyl)(C₁₋₄ alkyl), C₃₋₆ cycloalkyl, and 3-6 membered heterocyclic ring. As used herein, the two “C₁₋₄ alkyl” in —N(C₁₋₄ alkyl)(C₁₋₄ alkyl) can be the same or different. In some embodiments, Ru can be a C₃₋₆ cycloalkyl, such as cyclopropyl or cyclobutyl, which is optionally substituted with one or more (e.g., 1, 2, or 3) substituents independently selected from fluorine, C₁₋₄ alkyl, fluoro-substituted C₁₋₄ alkyl (e.g., CF₃), C₁₋₄ alkoxy, fluoro-substituted C₁₋₄ alkoxy (e.g., —OCF₃), NH₂, —NH(C₁₋₄ alkyl), and —N(C₁₋₄ alkyl)(C₁₋₄ alkyl). In some embodiments, R²⁵ can also be an optionally substituted 3-6 membered heterocyclic ring, such as an oxetanyl ring. In some embodiments, Ru can be an optionally substituted phenyl. In some embodiments, R²⁵ can be an optionally substituted 5 or 6 membered heteroaryl, e.g., those described herein.

In some embodiments, R⁵ in Formula I (e.g., Formula I-O) can be —CR²³R²⁴R²⁵, wherein
R²³ is hydrogen or fluorine;
R²⁴ is hydrogen or fluorine;
R²⁵ is hydrogen; fluorine; C₁₋₄ alkyl optionally substituted with 1-3 fluorines and/or a C₃₋₈ cycloalkyl; a C₁₋₄ alkoxy optionally substituted with 1-3 fluorines and/or a C₃₋₆ cycloalkyl; a C₃₋₆ cycloalkoxy optionally substituted with 1-3 substituents independently selected from fluorine and methyl; a C₃₋₆ cycloalkyl optionally substituted with 1-3 substituents independently selected from fluorine and methyl; or a 3-6 membered heterocyclic ring optionally substituted with 1-3 substituents independently selected from fluorine and methyl; and

preferably, at least one of R²³, R²⁴, and R²⁵ is not hydrogen. More preferably, R²⁵ is fluorine; C₁₋₄ alkyl optionally substituted with 1-3 fluorines and/or a C₃₋₆ cycloalkyl; or a C₃₋₆ cycloalkyl (e.g., cyclopropyl or cyclobutyl) optionally substituted with 1-3 substituents independently selected from fluorine and methyl. To be clear, when a C₁₋₄ alkyl is said to be optionally substituted with 1-3 fluorines and/or a C₃₋₈ cycloalkyl, it should be understood as encompassing unsubstituted C₁₋₄ alkyl, a C₁₋₄ alkyl substituted with 1-3 fluorines (e.g., CF₃), a C₁₋₄ alkyl substituted with a C₃₋₆ cycloalkyl (e.g., —CH₂-cyclopropyl), and a Cis alkyl substituted with 1-3 fluorines and a C₃₋₆ cycloalkyl (e.g., —CF₂—CH₂-cyclopropyl). Other similar expressions should be interpreted similarly.

In some embodiments, R⁵ in Formula I (e.g., Formula I-O) can be —CH₂R²⁵, wherein R²⁵ is defined herein, for example, R²⁵ can be hydrogen; fluorine; C₁₋₄ alkyl optionally substituted with 1-3 fluorines and/or a C₃₋₆cycloalkyl; a C₁₋₄ alkoxy optionally substituted with 1-3 fluorines and/or a C₃₋₆ cycloalkyl; a C₃₋₆ cycloalkoxy optionally substituted with 1-3 substituents independently selected from fluorine and methyl; a C₃₋₆ cycloalkyl optionally substituted with 1-3 substituents independently selected from fluorine and methyl; or a 3-6 membered heterocyclic ring optionally substituted with 1-3 substituents independently selected from fluorine and methyl, preferably, R²⁵ is not hydrogen. In any of the embodiments described herein, unless specified or obviously contradictory from context, R⁵ in Formula I (e.g., Formula I-O) can be —CH₂R²⁵, wherein R²⁵ is C₁₋₄ alkyl optionally substituted with 1-3 fluorines and/or a C₃₋₆ cycloalkyl, preferably, methyl, ethyl, n-propyl, isopropyl, or —CF₃; or a C₃₋₆ cycloalkyl optionally substituted with 1-3 substituents independently selected from fluorine and methyl, preferably, cyclopropyl or cyclobutyl. In any of the embodiments described herein, unless specified or obviously contradictory from context, R⁵ in Formula I (e.g., Formula I-O) can be —CH₂R²⁵, wherein R²⁵ can be methyl, ethyl, n-propyl, isopropyl, difluoromethyl, trifluoromethyl, —CH₂—CF₃, —CH₂-cyclopropyl, cyclopropyl or cyclobutyl.

In any of the embodiments described herein, unless specified or obviously contradictory from context, R⁵ in Formula I (e.g., Formula I-O) can be ethyl, n-propyl, isopropyl, n-butyl, isobutyl, sec-butyl, —CH₂—CHF₂, —CH₂—CF₃, —CF₃, —CH₂-cyclopropyl, —CH₂— cyclobutyl, —CH₂—O—CH₃, —CH₂—O—C₂H₅, —CH₂—O-n-propyl, —CH₂—O-isopropyl, —C₂H₄-cyclopropyl, —C₂H₄-cyclobutyl, methoxy, ethoxy, n-propoxy, isopropoxy, n-butoxy, isobutoxy, sec-butoxy, —O—CH₂—CF₃, —O—CF₃, —O—CH₂-cyclopropyl, —O—CH₂-cyclobutyl, —O—C₂H₄-cyclopropyl, or —O—C₂H₄-cyclobutyl.

In some embodiments, the compound of Formula I-O can be characterized in that R³, R⁴ and R⁵ are joined to form an optionally substituted bicyclic or polycyclic ring system, wherein the ring system is an aryl, heteroaryl, carbocyclic, or heterocyclic ring system. For example, in some embodiments, the moiety of

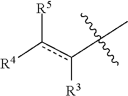

### in Formula I can be

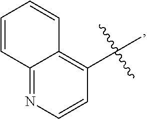

which is optionally substituted.

In some embodiments, Z in Formula I is joined with R⁵ to form an optionally substituted aryl, an optionally substituted heteroaryl, an optionally substituted carbocyclic (e.g., C₃₋₈ carbocyclic), or an optionally substituted heterocyclic ring (e.g., 3-8 membered heterocyclic ring). For example, in some embodiments, Z in Formula I is joined with R⁵ to form an optionally substituted heteroaryl. In some embodiments, the compound of Formula I can have a formula of Formula I-F:

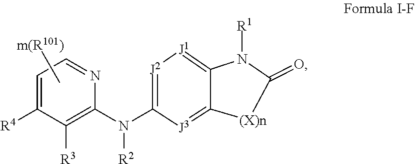

wherein R¹⁰¹ at each occurrence is independently selected from halogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), —CN, or —OR³¹; and
m is 0, 1, 2, or 3, preferably, m is 0 or 1; and
wherein R¹, R², R³, R⁴, R³¹, J¹, J², J³, X, and n are defined herein. In some embodiments, R³ and R⁴ in Formula I-F are joined to form an optionally substituted phenyl, an optionally substituted 5 or 6-membered heteroaryl, e.g., having one or two ring nitrogen atoms, an optionally substituted C₄₋₇ cycloalkyl group (e.g., cyclopentyl or cyclohexyl), or an optionally substituted 4 to 7-membered (e.g., 6-membered) heterocyclic ring having one or two ring heteroatoms. In some embodiments, R³ and R⁴ in Formula I-F can be joined to form an optionally substituted phenyl, for example, unsubstituted phenyl, or phenyl substituted with one or two substituents independently selected from F; Cl; Cu alkyl optionally substituted with 1-3 fluorines, preferably, methyl, ethyl, n-propyl, isopropyl, or —CF₃; a C₁₋₄alkoxy optionally substituted with 1-3 fluorines, preferably, methoxy, ethoxy, n-propoxy, isopropoxy, or —OCF₃; a C₃₋₆ cycloalkyl optionally substituted with 1-3 substituents independently selected from fluorine and methyl, preferably, cyclopropyl or cyclobutyl; and —CN. In some embodiments, R³ and R⁴ in Formula I F can be joined to form an optionally substituted 5 or 6-membered heteroaryl.

In some specific embodiments, the compound of Formula I can be characterized as having Formula I-1 or I-2:

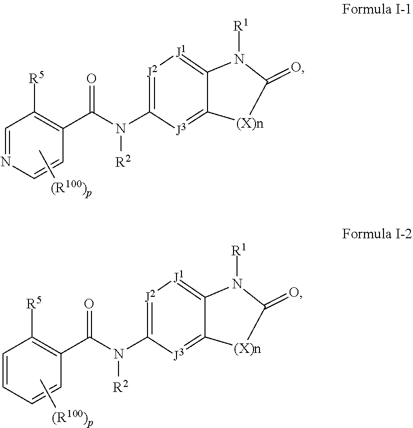

wherein:
R¹⁰⁰ at each occurrence is independently selected from halogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), —CN, or —OR³¹;
p is 0, 1, 2, or 3, preferably, p is 0 or 1; and

R¹, R², R⁵, R³¹, J¹, J², J³, X, and n are defined herein. In some embodiments, in Formula I-1 or I-2, R¹⁰⁰ at each occurrence is independently selected from F; Cl; C₁₋₄ alkyl optionally substituted with 1-3 fluorines, preferably, methyl, ethyl, n-propyl, isopropyl, or —CF₃; a C₁₋₄ alkoxy optionally substituted with 1-3 fluorines, preferably, methoxy, ethoxy, n-propoxy, isopropoxy, or —OCF₃; a C₃₋₆ cycloalkyl optionally substituted with 1-3 substituents independently selected from fluorine and methyl, preferably, cyclopropyl or cyclobutyl; and —CN. In some embodiments, in Formula I-1 or I-2, p is 0. In some embodiments, in Formula I-1 or I-2, p is 1. In some embodiments, in Formula I-1 or I-2, p is 1, and R¹⁰³ is F, Cl, methyl, ethyl, n-propyl, isopropyl, —CF₃, methoxy, ethoxy, n-propoxy, isopropoxy, —OCF₃, cyclopropyl, or —CN. In some embodiments, in Formula I-1 or I-2, p is 1, and R¹⁰⁰ is OH. In some embodiments, in Formula I-1 or I-2, p is 1, and R¹⁰⁰ is F, Cl, OH, methyl, or ethyl.

In some specific embodiments, the compound of Formula I can be characterized as having Formula I-1-A or Formula I-2-A:

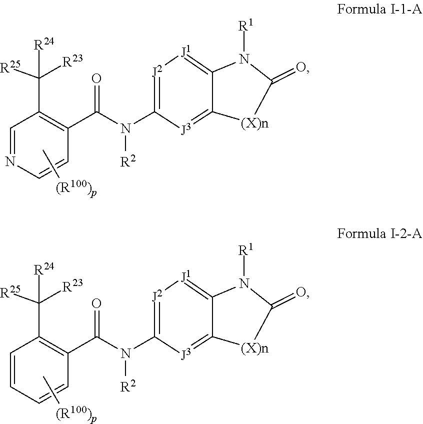

wherein R¹, R², R²³, R²⁴, R²⁵, R¹⁰⁰, J¹, J², J³, X, p, and n are defined herein. In some embodiments, in Formula I-1-A or I-2-A:
R²³ is hydrogen or fluorine;
R²⁴ is hydrogen or fluorine;
R²⁵ is hydrogen; fluorine; C₁₋₄ alkyl optionally substituted with 1-3 fluorines and/or a C₃₋₆ cycloalkyl; a C₁₋₄ alkoxy optionally substituted with 1-3 fluorines and/or a C₃₋₆ cycloalkyl; a C₃₋₆ cycloalkoxy optionally substituted with 1-3 substituents independently selected from fluorine and methyl; a C₃₋₆ cycloalkyl optionally substituted with 1-3 substituents independently selected from fluorine and methyl; or a 3-6 membered heterocyclic ring optionally substituted with 1-3 substituents independently selected from fluorine and methyl; and

preferably at least one of R²³, R²⁴, and R²³ is not hydrogen.

In some embodiments, R²³ in Formula I-1-A or I-2-A is hydrogen. In some embodiments, in Formula I-1-A or I-2-A, R²³ and R²⁴ are both hydrogen. In some embodiments, in Formula I 1-A or I-2-A, R²⁵ is a C₁₋₄ alkyl optionally substituted with 1-3 fluorines and/or a C₃₋₆ cycloalkyl, preferably, methyl, ethyl, n-propyl, isopropyl, or —CF₃; or a C₃₋₈ cycloalkyl optionally substituted with 1-3 substituents independently selected from fluorine and methyl, preferably, cyclopropyl or cyclobutyl. For example, in some embodiments, in Formula I-1-A or I-2-A, R²⁵ is methyl, ethyl, n-propyl, isopropyl, difluoromethyl, trifluoromethyl, —CH₂—CF₃, —CH₂-cyclopropyl, cyclopropyl or cyclobutyl.

In some embodiments, the compound of Formula I-1-A or I-2-A can be characterized as having Formula I-1-A1, Formula I-1-A2, Formula I-1-A3, Formula I-2-A1, Formula I-2-A2; Formula I-2-A3:

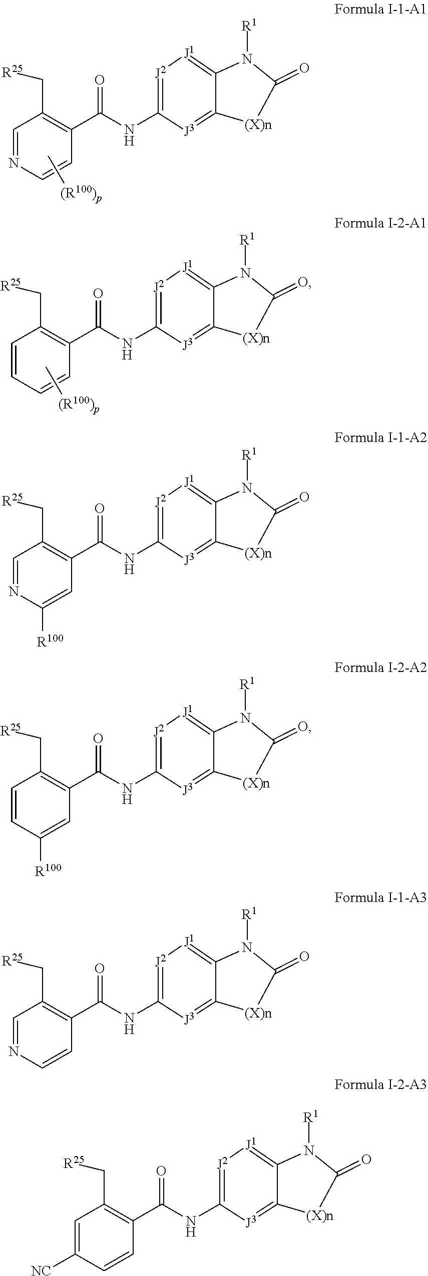

wherein R¹, R²⁵, R¹⁰⁰, J¹, J², J³, X, p, and n are defined herein. In some embodiments, in Formula I-1-A1, Formula I-1-A2, Formula I-1-A3, Formula I-2-A1, Formula I-2-A2, or Formula I-2-A3, R²⁵ is C₁₋₄ alkyl optionally substituted with 1-3 fluorines and/or a C₃₋₆ cycloalkyl, preferably, methyl, ethyl, n-propyl, isopropyl, or —CF₃; or a C₃₋₆ cycloalkyl optionally substituted with 1-3 substituents independently selected from fluorine and methyl, preferably, cyclopropyl or cyclobutyl. In some specific embodiments, R²⁵ in Formula I-1-A1, Formula I-1-A2, Formula I-1-A3, Formula I-2-A1, Formula I-2-A2, or Formula I-2-A3 can be methyl, ethyl, n-propyl, isopropyl, difluoromethyl, trifluoromethyl, —CH₂—CF₃, —CH₂-cyclopropyl, cyclopropyl or cyclobutyl.

In some embodiments, in Formula I-1-A1, Formula I-1-A2, Formula I-2-A1, or Formula I-2-A2, R¹⁰⁰ at each occurrence is independently selected from F; Cl; C₁₋₄ alkyl optionally substituted with 1-3 fluorines, preferably, methyl, ethyl, n-propyl, isopropyl, or —CF₃; a C₁₋₄ alkoxy optionally substituted with 1-3 fluorines, preferably, methoxy, ethoxy, n-propoxy, isopropoxy, or —OCF₃; a C₃₋₆ cycloalkyl optionally substituted with 1-3 substituents independently selected from fluorine and methyl, preferably, cyclopropyl or cyclobutyl; and —CN. In some embodiments, in Formula I-1-A1, Formula I-1-A2, Formula I-2-A1, or Formula I 2-A2, one instance of R¹⁰⁰ can be hydroxyl. In some embodiments, in Formula I-1-A1 or I-2-A1, p is 1. In some embodiments, in Formula I-1-A1 or I-2-A1, p is 2. In some embodiments, in Formula I-1-A1 or I-2-A1, p is 1, and R¹⁰⁰ is F, Cl, methyl, ethyl, n-propyl, isopropyl, —CF₃, methoxy, ethoxy, n-propoxy, isopropoxy, —OCF₃, cyclopropyl, or —CN. In some embodiments, in Formula I-1-A1 or I-2-A1, p is 1, and R¹⁰⁰ is F, Cl, or methyl. In some embodiments, in Formula I-1-A2 or Formula I-2-A2, R¹⁰⁰ is F, Cl, methyl, ethyl, n-propyl, isopropyl, —CF₃, methoxy, ethoxy, n-propoxy, isopropoxy, —OCF₃, cyclopropyl, or —CN. In some embodiments, in Formula I-1-A2 or Formula I-2-A2, R¹⁰⁰ is F, Cl, or methyl.

In some embodiments, the compound of Formula I-1 or I-2 can be characterized as having Formula I-1-B, I-1-C, I-2-B, or I-2-C:

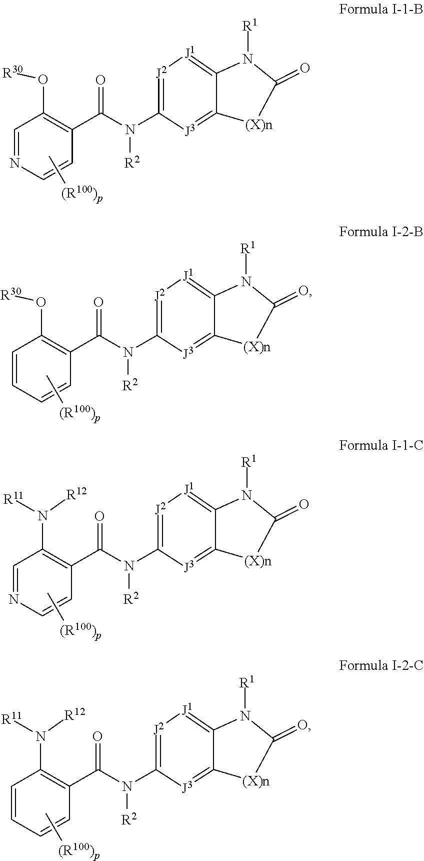

wherein R¹, R², R³⁰, R¹¹, R¹², R¹⁰⁰, J¹, J², J³, X, p, and n are defined herein. In some embodiments, in Formula I-1-B or I-2-B, R³⁰ can be hydrogen; C₁₋₄ alkyl optionally substituted with 1-3 fluorines and/or a C₃₋₈ cycloalkyl, preferably, methyl, ethyl, n-propyl, isopropyl, difluoromethyl, trifluoromethyl, —CH₂—CF₃, or —CH₂-cyclopropyl; a C₃₋₆ cycloalkyl optionally substituted with 1-3 substituents independently selected from fluorine and methyl, preferably, cyclopropyl or cyclobutyl; or a 3-6 membered heterocyclic ring optionally substituted with 1-3 substituents independently selected from fluorine and methyl, preferably,

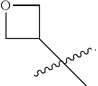 [SMILES: *C1COC1]

In some embodiments, R³⁰ can be methyl, ethyl, n-propyl, isopropyl, difluoromethyl, trifluoromethyl, —CH₂—CF₃, or —CH₂-cyclopropyl. In some embodiments, R³⁰ can be cyclopropyl, cyclobutyl; or

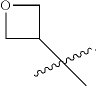 [SMILES: *C1COC1]

In some embodiments, in Formula I-1-C or I-2-C, one of R¹¹ and R¹² is hydrogen or a nitrogen protecting group, and the other of R¹¹ and R¹² is hydrogen, a nitrogen protecting group, C₁₋₄ alkyl optionally substituted with 1-3 fluorines or a C₃₋₆ cycloalkyl, preferably, methyl, ethyl, n-propyl, isopropyl, difluoromethyl, trifluoromethyl, —CH₂—CF₃, or —CH₂-cyclopropyl; a C₃₋₆ cycloalkyl optionally substituted with 1-3 substituents independently selected from fluorine and methyl, preferably, cyclopropyl or cyclobutyl; or a 3-6 membered heterocyclic ring optionally substituted with 1-3 substituents independently selected from fluorine and methyl, preferably,

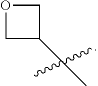 [SMILES: *C1COC1]

In some embodiments, in Formula I-1-B, I-1-C, I-2-B, or I-2-C, R¹⁰⁰ at each occurrence is independently selected from F; Cl; C₁₋₄ alkyl optionally substituted with 1-3 fluorines, preferably, methyl, ethyl, n-propyl, isopropyl, or —CF₃; a C₁₋₄ alkoxy optionally substituted with 1-3 fluorines, preferably, methoxy, ethoxy, n-propoxy, isopropoxy, or —OCF₃; a C₃₋₈ cycloalkyl optionally substituted with 1-3 substituents independently selected from fluorine and methyl, preferably, cyclopropyl or cyclobutyl; and —CN. In some embodiments, in Formula I-1-B, I-1-C, I-2-B, or I-2-C, p is 0. In some embodiments, in Formula I-1-B, I-1-C, I-2-B, or I-2-C, p is 1. In some embodiments, in Formula I-1-B, I-1-C, I-2-B, or I-2-C, p is 1 and R¹⁰⁰ is F, Cl, methyl, ethyl, n-propyl, isopropyl, —CF₃, methoxy, ethoxy, n-propoxy, isopropoxy, —OCF₃, cyclopropyl, or —CN.

In some embodiments, the compound of Formula I-1 or I-2 can be characterized as having Formula I-1-B1, Formula I-1-B2, Formula I-2-B1, Formula I-2-B2:

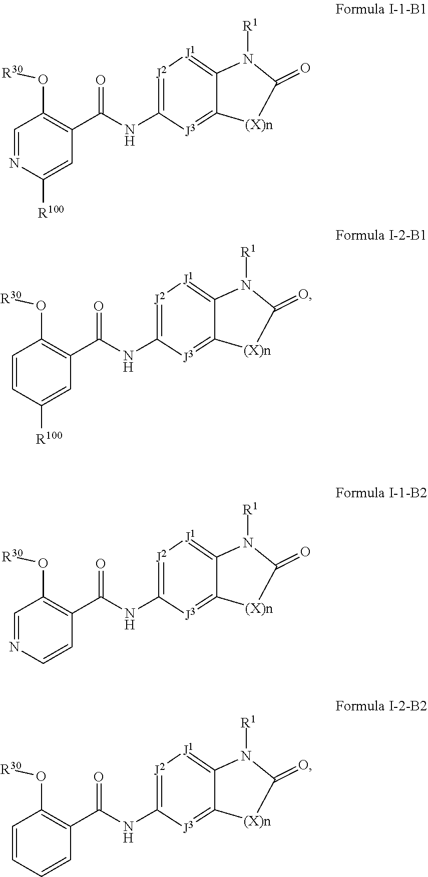

wherein R¹, R³⁰, R¹⁰⁰, J¹, J², J³, X, p, and n are defined herein. In some embodiments, R³⁰ can be hydrogen; C₁₋₄ alkyl optionally substituted with 1-3 fluorines and/or a C₃₋₆ cycloalkyl, preferably, methyl, ethyl, n-propyl, isopropyl, difluoromethyl, trifluoromethyl, —CH₂—CF₃, or —CH₂-cyclopropyl; a C₃₋₆ cycloalkyl optionally substituted with 1-3 substituents independently selected from fluorine and methyl, preferably, cyclopropyl or cyclobutyl; or a 3-6 membered heterocyclic ring optionally substituted with 1-3 substituents independently selected from fluorine and methyl, preferably,

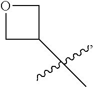 [SMILES: *C1COC1]

In some embodiments, R³⁰ can be hydrogen, methyl, ethyl, n-propyl, isopropyl, difluoromethyl, trifluoromethyl, —CH₂—CF₃, —CH₂-cyclopropyl, cyclopropyl or cyclobutyl. In some embodiments, R^{IG)} in Formula I-1-B1 or I-2-B1 can be F, Cl, methyl, ethyl, n-propyl, isopropyl, —CF₃, methoxy, ethoxy, n-propoxy, isopropoxy, —OCF₃, cyclopropyl, or —CN.

In some specific embodiments, the moiety of

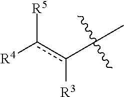

in Formula I (e.g., any of the applicable subformulae) can have a structure according to one of the following:

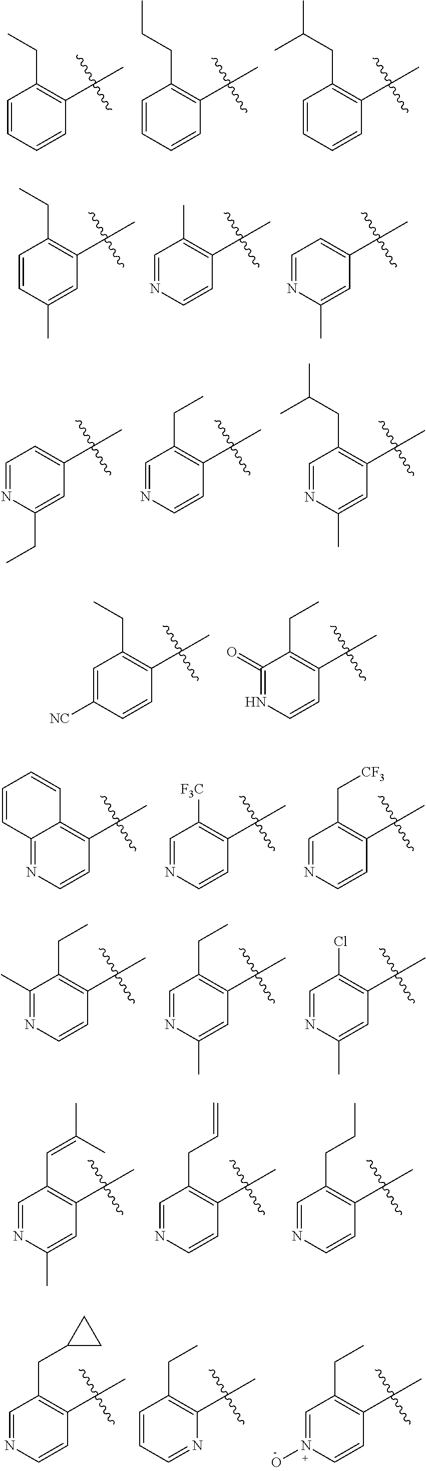

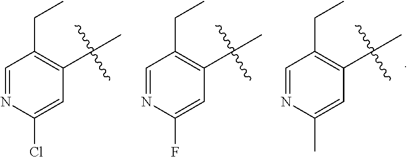

In some specific embodiments, the moiety of

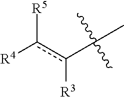

in Formula I (e.g., any of the applicable subformulae) can have a structure according to one of the following:

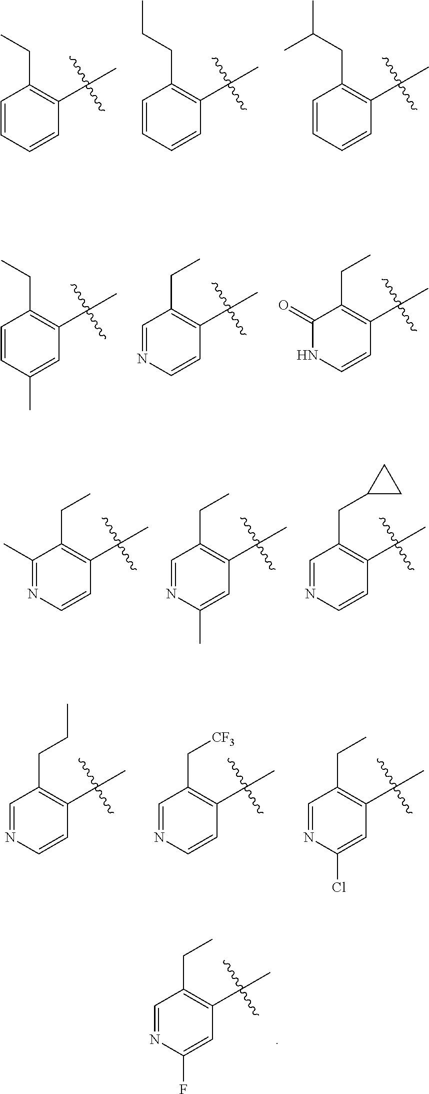

In some embodiments, the moiety of

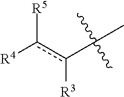

in Formula I (e.g., any of the applicable subformulae) can have a structure of any of the corresponding moieties in Compound Nos. 1-138 as disclosed herein, as applicable In some embodiments, the moiety of

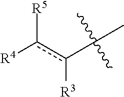

in Formula I (e.g., any of the applicable subformulae) can have a structure of any of the corresponding moieties in the specific compounds disclosed herein, as applicable, that have an activity level of A or B shown in Table 3 of the present disclosure in inhibiting hALDH 1a3.

Typically, R¹ and R² in Formula I are both hydrogen. For example, in some embodiments, R¹ and R² in any of the sub-formulae of Formula I, such as Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C, can be both hydrogen.

Typically, J¹ in Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C) is CH. In some embodiments, J¹ in Formula I (including any of the subformulae of Formula I) can also be N.

Typically, J² in Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C) is CR²², wherein R²² is defined herein. In some embodiments, R²² is hydrogen, F, Cl, CN, or methyl. In some embodiments, J² in Formula I (including any of the subformulae of Formula I) can also be N.

Typically, J³ in Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C) is CH. In some embodiments, J³ in Formula I (including any of the subformulae of Formula I) can also be N.

Typically, in Formula I (e.g., Formula I-O, I-F, I-1, I-2, I-1-A, I-2-A, I-1-A1, I-1 A2, I-1-A3, I-2-A1, I-2-A2, I-2-A3, I-1-B, I-2-B, I-1-C, or I-2-C) at least one of J¹, J², and J³ is not N. In some embodiments, none of J¹, J², and J³ is N, for example, J¹ can be CH, J² can be CR²², and J³ can be CH, wherein R²² is hydrogen, F, Cl, CN, or methyl.

Typically, in Formula I, n is 1, 2, or 3. Preferably, n is 2.

In Formula I, each instance of X can be O, NR¹⁰, or CR²⁰R²¹, provided that at most one X is selected from O and NR¹⁰. In some embodiments, at least one instance of X is CR²⁰R²¹, wherein R²⁰ and R²¹ are defined herein.

In some embodiments, n is 1 and X is O. In some embodiments, n is 1 and X is NR¹⁰, wherein R¹⁰ is defined herein, for example, hydrogen or C₁₋₄ alkyl. In some embodiments, n is 1 and X is CR²⁰R²¹, wherein R²⁰ and R²¹ are defined herein. In some embodiments, in the CR²⁰R²¹ unit,

R²⁰ and R²¹ are both methyl;

one of R²⁰ and R²¹ is methyl, and the other of R²⁰ and R²¹ is ethyl or methoxy, or

R²⁰ and R²¹, together with the carbon they are both attached to, form a C₃₋₆ cycloalkyl (preferably cyclopropyl, cyclobutyl, or cyclopentyl), or an oxetanyl ring. In some embodiments, in the CR²⁰R²¹ unit, one of R²⁰ and R²¹ is methyl, and the other of R²⁰ and R²¹ is hydrogen. In some embodiments, in the CR²⁰R²¹ unit, R²⁰ and R²¹ are both hydrogen. In some embodiments, in the CR²⁰R²¹ unit, R²⁰ and R²¹ are both fluorine.

In some embodiments, n is 2, one instance of X is O, and one instance of X is CR²⁰R²¹, wherein R²⁰ and R²¹ are defined herein. In some embodiments, n is 2, one instance of X is NR¹⁰, and one instance of X is CR²⁰R²¹, wherein R¹⁰, R²⁰ and R²¹ are defined herein. In some embodiments, n is 2, and both instances of X are CR²⁰R²¹ as defined herein. In some embodiments, R²⁰ and R²¹ are independently hydrogen or C₁₋₄ alkyl, or R²⁰ and R²¹, together with the carbon they are both attached to, form a C₃₋₆ cycloalkyl (preferably cyclopropyl, cyclobutyl, or cyclopentyl), or an oxetanyl ring. In some embodiments, R¹⁰ is hydrogen or C₁₋₄alkyl. In some embodiments, the compound includes at least one CR²⁰R²¹ unit, wherein:

R²⁰ and R²¹ are both methyl;

one of R²⁰ and R²¹ is methyl, and the other of R²⁰ and R²¹ is ethyl or methoxy; or

R²⁰ and R²¹, together with the carbon they are both attached to, form a cyclopropyl, cyclobutyl, or an oxetanyl ring. In some embodiments, in the at least one CR²⁰R²¹ unit, one of R²⁰ and R²¹ is methyl, and the other of R²⁰ and R²¹ is hydrogen. In some embodiments, in the CR²⁰R²¹ unit, R²⁰ and R²¹ are both hydrogen.

In some embodiments, n is 3, one instance of X is O, and two instances of X are independently selected CR²⁰R²¹, wherein R²⁰ and R²¹ are defined herein. In some embodiments, n is 3, one instance of X is NR¹⁰, and two instances of X are independently selected CR²⁰R²¹, wherein R¹⁰, R²⁰ and R²¹ are defined herein. In some embodiments, n is 3, and all instances of X are CR²⁰R²¹ as defined herein. In some embodiments, R²⁰ and R²¹ are independently hydrogen or C₁₋₄ alkyl, or R²⁰ and R²¹, together with the carbon they are both attached to, form a C₃₋₆ cycloalkyl (preferably cyclopropyl, cyclobutyl, or cyclopentyl), or an oxetanyl ring. In some embodiments, R¹⁰ is hydrogen or C₁₋₄ alkyl. In some embodiments, the compound includes at least one CR²⁰R²¹ unit, wherein:

R²⁰ and R²¹ are both methyl;

one of R²⁰ and R²¹ is methyl, and the other of R²⁰ and R²¹ is ethyl or methoxy; or

R²⁰ and R²¹, together with the carbon they are both attached to, form a cyclopropyl, cyclobutyl, or an oxetanyl ring. In some embodiments, in at least one CR²⁰R²¹ unit, one of R²⁰ and R²¹ is methyl, and the other of R²⁰ and R²¹ is hydrogen. In some embodiments, in at least one CR²⁰R²¹ unit, R²⁰ and R²¹ are both hydrogen.

In some embodiments, in Formula I (e.g., Formula I-O, I-F, I-1, I-2, I-1-A, I-2-A, I-1-A1, I-1-A2, I-1-A3, I-2-A 1, I-2-A2, I-2-A3, I-1-B, I-2-B, I-1-C, or I-2-C), the

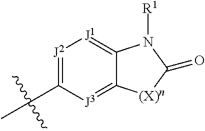

can be selected from the following:

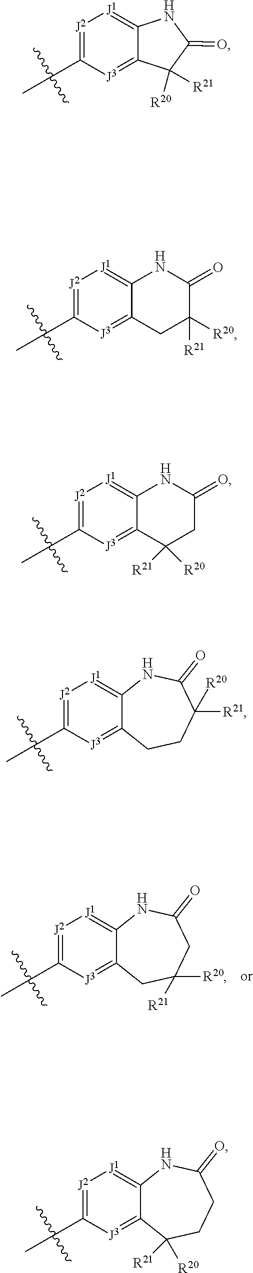

wherein J¹, J², J³, R²⁰ and R²¹ are defined herein. In some embodiments, J¹ is CH. In some embodiments, J² is N or CR²², wherein R²² is defined herein, for example, hydrogen, F, Cl, CN, or methyl. In some embodiments, J³ is CH. In some embodiments, R²⁰ and R²¹ are independently hydrogen or C₁₋₄ alkyl (e.g., methyl, ethyl, etc.), or R²⁰ and R²¹, together with the carbon they are both attached to, form a cyclopropyl, cyclobutyl, cyclopentyl, or an oxetanyl ring. In some embodiments, in the CR²⁰R²¹ unit:

R²⁰ and R²¹ are both methyl;

one of R²⁰ and R²¹ is methyl, and the other of R²⁰ and R²¹ is ethyl or methoxy; or

R²⁰ and R²¹, together with the carbon they are both attached to, form a cyclopropyl, cyclobutyl, or an oxetanyl ring. In some embodiments, in the CR²⁰R²¹ unit, one of R²⁰ and R²¹ is methyl, and the other of R²⁰ and R²¹ is hydrogen. In some embodiments, in the CR²⁰R²¹ unit, R²⁰ and R²¹ are both hydrogen.

In some embodiments, in Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), the

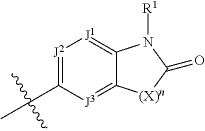

can be selected from the following:

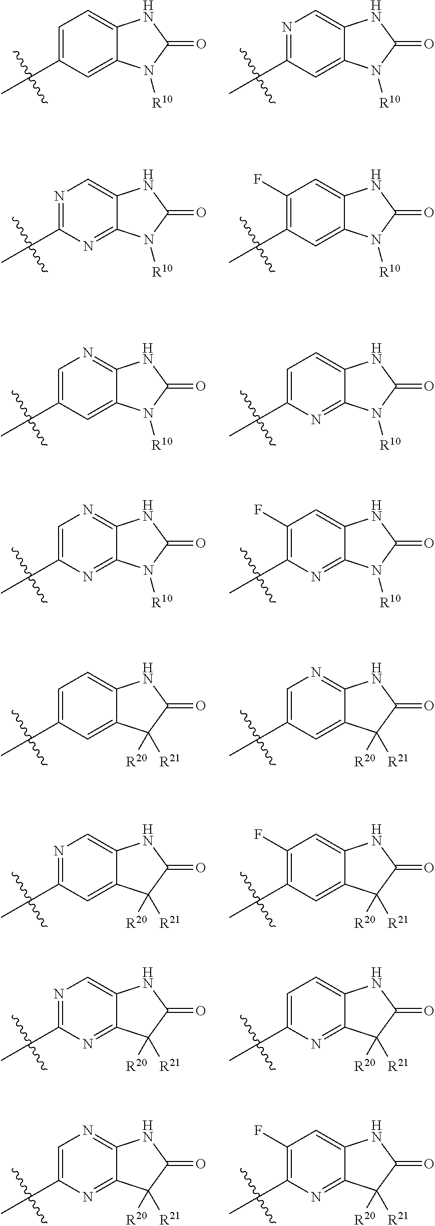

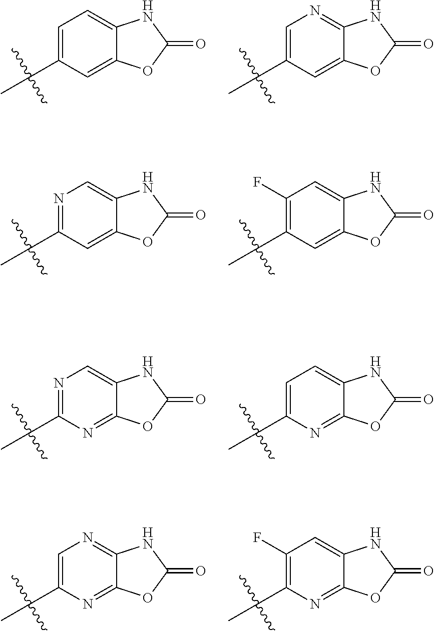

wherein:
R¹⁰ is independently hydrogen or C₁₋₄ alkyl (e.g., methyl, ethyl, etc.);
R²⁰ and R²¹ are independently hydrogen or C₁₋₄ alkyl (e.g., methyl, ethyl, etc.), or R²⁰ and R²¹, together with the carbon they are both attached to, form a cyclopropyl, cyclobutyl, cyclopentyl, or an oxetanyl ring. In some embodiments, one of R²⁰ and R²¹ is methyl, and the other of R²⁰ and R²¹ is hydrogen. In some embodiments, R²⁰ and R²¹ are both hydrogen.

In some embodiments, in Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), the

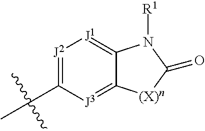

can be selected from the following:

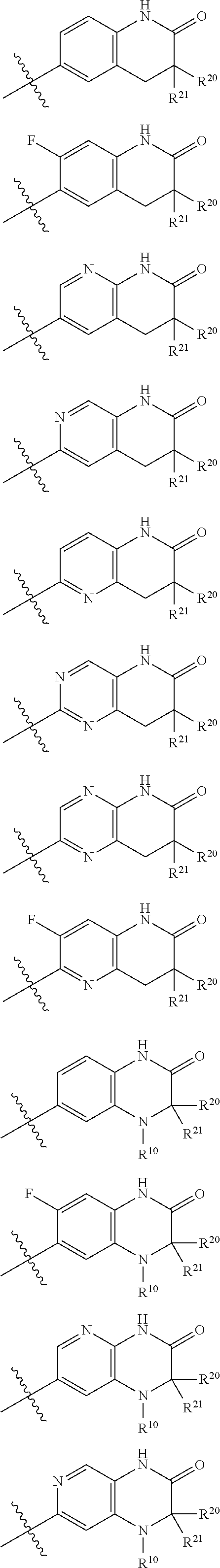

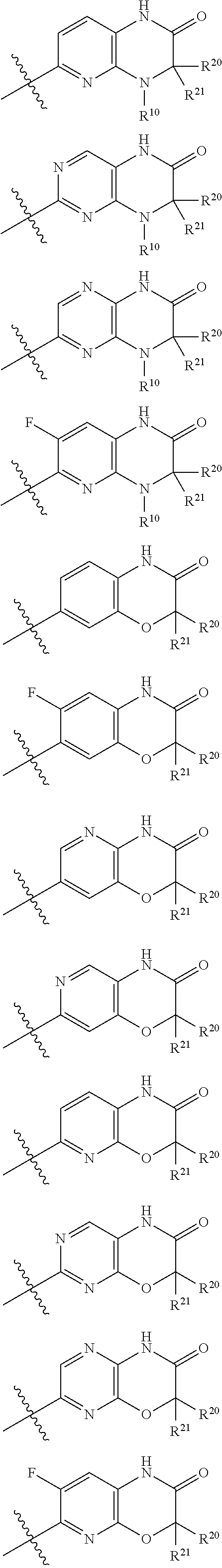

wherein R¹⁰, R²⁰, and R²¹ are defined herein. In some embodiments, R¹⁰ is independently hydrogen or C₁₋₄ alkyl (e.g., methyl, ethyl, etc.). In some embodiments, R²⁰ and R²¹ are independently hydrogen or C₁₋₃ alkyl (e.g., methyl, ethyl, etc.), or R²⁰ and R²¹, together with the carbon they are both attached to, form a cyclopropyl, cyclobutyl, cyclopentyl, or an oxetanyl ring. In some embodiments, in the CR²⁰R²¹ unit:

R²⁰ and R²¹ are both methyl;

one of R²⁰ and R²¹ is methyl, and the other of R²⁰ and R²¹ is ethyl or methoxy; or R²⁰ and R²¹, together with the carbon they are both attached to, form a cyclopropyl, cyclobutyl, or an oxetanyl ring. In some embodiments, one of R²⁰ and R²¹ is methyl, and the other of R²⁰ and R²¹ is hydrogen. In some embodiments, R²⁰ and R²¹ are both hydrogen.
In some embodiments, in Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), the

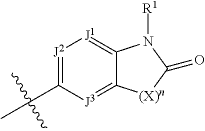

can be selected from the following:

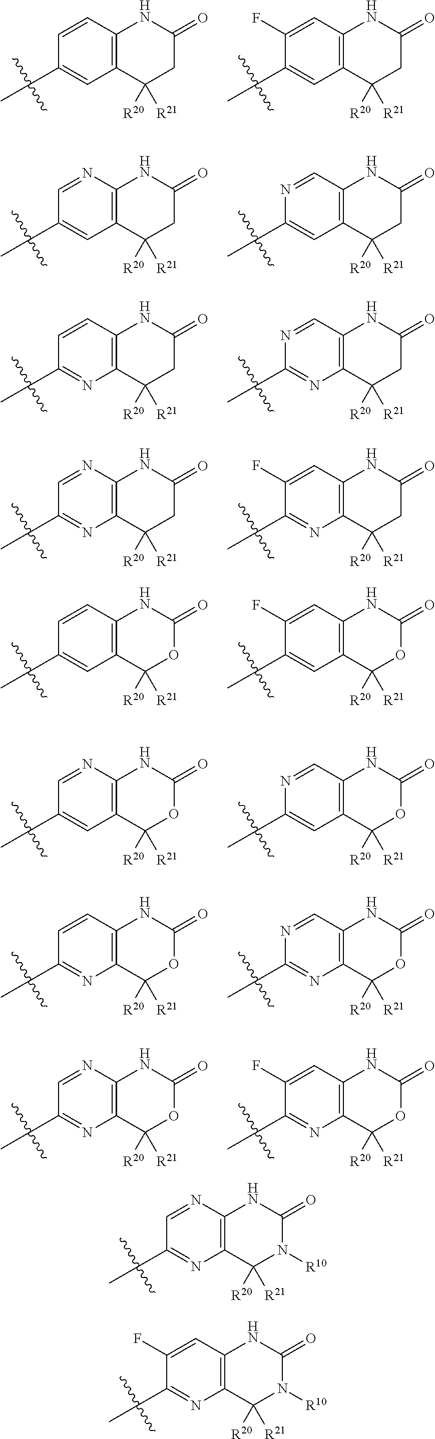

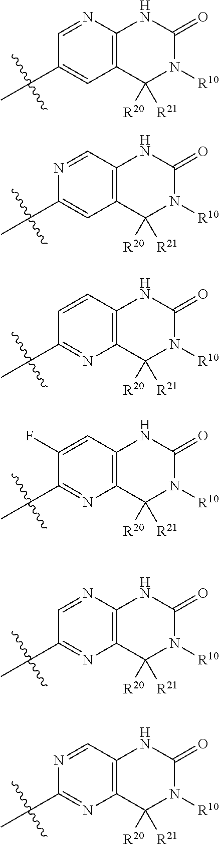

wherein R¹⁰, R²⁰, and R²¹ are defined herein. In some embodiments, R¹⁰ is independently hydrogen or C₁₋₄ alkyl (e.g., methyl, ethyl, etc.). In some embodiments, R²⁰ and R²¹ are independently hydrogen or C₁₋₄ alkyl (e.g., methyl, ethyl, etc.), or R²⁰ and R²¹, together with the carbon they are both attached to, form a cyclopropyl, cyclobutyl, cyclopentyl, or an oxetanyl ring. In some embodiments, in the CR²⁰R²¹ unit:

R²⁰ and R²¹ are both methyl;

one of R²⁰ and R²¹ is methyl, and the other of R²⁰ and R²¹ is ethyl or methoxy; or

R²⁰ and R²¹, together with the carbon they are both attached to, form a cyclopropyl, cyclobutyl, or an oxetanyl ring. In some embodiments, one of R²⁰ and R²¹ is methyl, and the other of R²⁰ and R²¹ is hydrogen. In some embodiments, R²⁰ and R²¹ are both hydrogen.

In some embodiments, in Formula I (e.g., Formula I-O, I-F, I-1, I-2, I-1-A, I-2-A, I-1-A1, I-1-A2, I-1-A3, I-2-A 1, I-2-A2, I-2-A3, I-1-B, I-2-B, I-1-C, or I-2-C), the

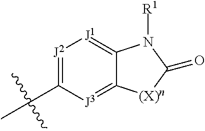

can be selected from the following:

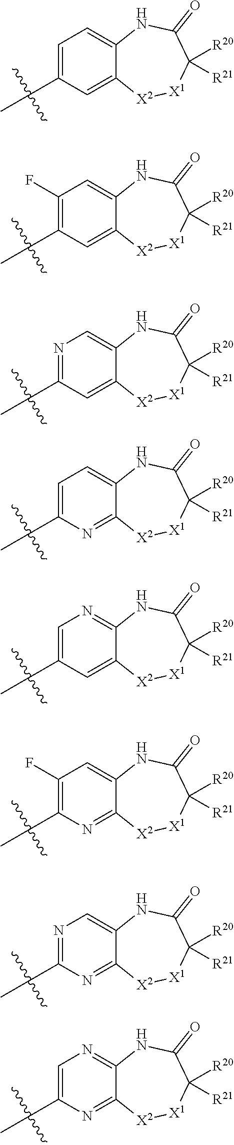

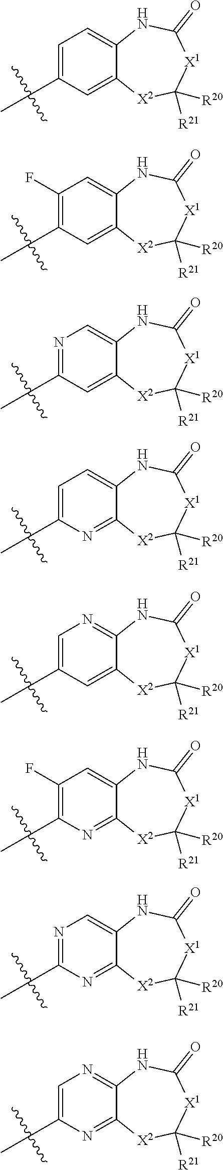

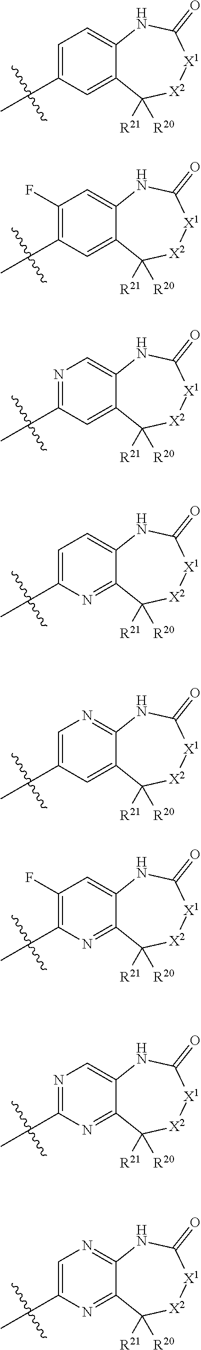

wherein X¹ and X² are independently O, NR¹⁰, or CH₂, provided that at least one of X¹ and X² is CH₂; and R¹⁰, R²⁰, and R²¹ are defined herein. In some embodiments, both X¹ and X² are CH₂. In some embodiments, one of X¹ and X² is NR¹⁰. In some embodiments, R¹⁰ is independently hydrogen or C₁₋₄ alkyl (e.g., methyl, ethyl, etc.). In some embodiments, R²⁰ and R²¹ are independently hydrogen or C₁₋₄ alkyl (e.g., methyl, ethyl, etc.), or R²⁰ and R²¹, together with the carbon they are both attached to, form a cyclopropyl, cyclobutyl, cyclopentyl, or an oxetanyl ring. In some embodiments, in the CR²⁰R²¹ unit:

R²⁰ and R²¹ are both methyl;

one of R²⁰ and R²¹ is methyl, and the other of R²⁰ and R²¹ is ethyl or methoxy; or

R²⁰ and R²¹, together with the carbon they are both attached to, form a cyclopropyl, cyclobutyl, or an oxetanyl ring. In some embodiments, in the CR²⁰R²¹ unit, one of R²⁰ and R²¹ is methyl, and the other of R²⁰ and R²¹ is hydrogen. In some embodiments, in the CR²⁰R²¹ unit, R²⁰ and R²¹ are both hydrogen.

In some embodiments, in Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), the

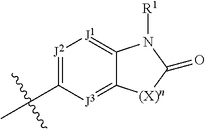

can be selected from the following:

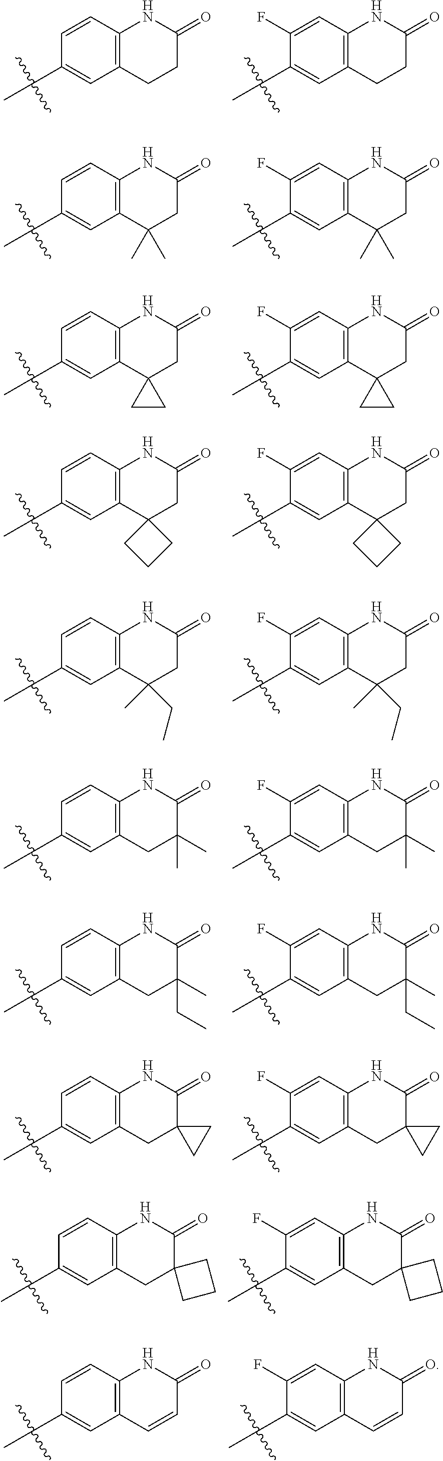

In some embodiments, in Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), the

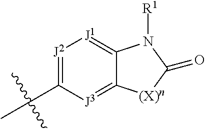

can be selected from the following:

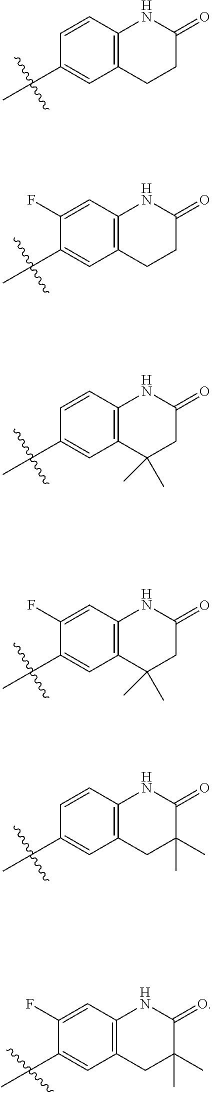 [SMILES: *c1ccc2c(c1)CCC(=O)N2 ; *c1cc2c(cc1F)NC(=O)CC2 ; *c1ccc2c(c1)C(C)(C)CC(=O)N2 ; *c1cc2c(cc1F)NC(=O)CC2(C)C ; *c1ccc2c(c1)CC(C)(C)C(=O)N2 ; *c1cc2c(cc1F)NC(=O)C(C)(C)C2]

In some embodiments, in Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), the

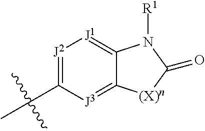

can be selected from the following:

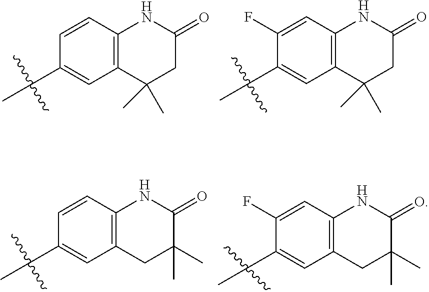

In some embodiments, in Formula I (e.g., Formula I-O, I-F, I-1, 1-2, I-1-A, I-2-A, I-1-A1, I-1-A2, I-1-A3, I-2-A1, I-2-A2, I-2-A3, I-1-B, I-2-B, I-1-C, or I-2-C), the

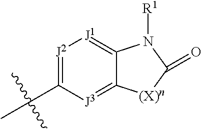

can be selected from the following:

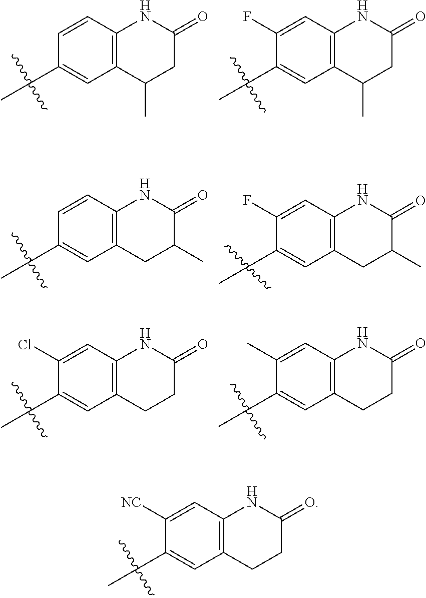

In some embodiments, in Formula I (e.g., Formula I-O, I-F, I-1, I-2, I-1-A, I-2-A, I-1-A1, I-1-A2, I-1-A3, I-2-A 1, I-2-A2, I-2-A3, I-1-B, I-2-B, I-1-C, or I-2-C), the

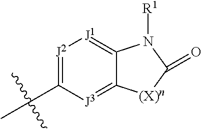

can be selected from the following:

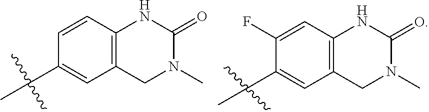

In some embodiments, in Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), the

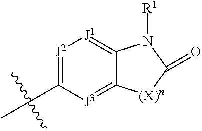

be selected from the following:

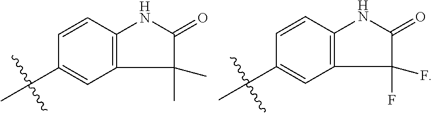

In some embodiments, in Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), the

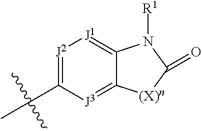

can be any of the corresponding moieties shown in Compound Nos. 1-138 disclosed herein, as applicable.

In some embodiments, the present disclosure also provides a compound of Formula I-P, or a pharmaceutically acceptable salt thereof:

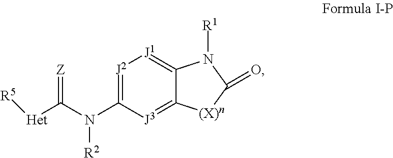

wherein Het represents an optionally substituted heterocyclic or heteroaryl ring structure, preferably, 5 or 6 membered heterocyclic ring or 5 or 6 membered heteroaryl ring, wherein R¹, R², R⁵, J¹, J², J³, X, and n can be any of those defined herein for Formula I (including its subformulae). Preferably, when Z is O, Het is a 5 or 6 membered heteroaryl, and in Formula I-P, R⁵ is attached to the Het at an ortho position of —C(═Z)—. It will also be understood that in Formula I-P, R⁵ can be attached to a ring nitrogen as valance permits.

In some embodiments, in Formula I-P, Z is O, R² is hydrogen or methyl,

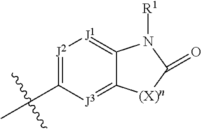

and R⁵ can be any of those described for Formula I (including its subformulae), and Het is an optionally substituted 5 or 6 membered heteroaryl described herein, for example, Het is a 5 or 6 membered heteroaryl, preferably, a pyrazole, imidazole, oxazole, thiazole, isoxazole, isothiazole, pyridyl, pyrimidinyl, pyridazinyl, or pyrazinyl, which is optionally substituted with one or two (preferably one) substituents independently selected from F; Cl; C₁₋₄ alkyl optionally substituted with 1-3 fluorines, preferably, methyl, ethyl, n-propyl, isopropyl, or —CF₃; a C₁₋₄ alkoxy optionally substituted with 1-3 fluorines, preferably, methoxy, ethoxy, n-propoxy, isopropoxy, or —OCF₃; a C₃₋₆ cycloalkoy optionally substituted with 1-3 substituents independently selected from fluorine and methyl; a C₃₋₆ cycloalkyl optionally substituted with 1-3 substituents independently selected from fluorine and methyl, preferably, cyclopropyl or cyclobutyl; and —CN. In some embodiments,

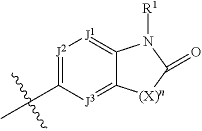

in Formula I-P can be selected from the following:

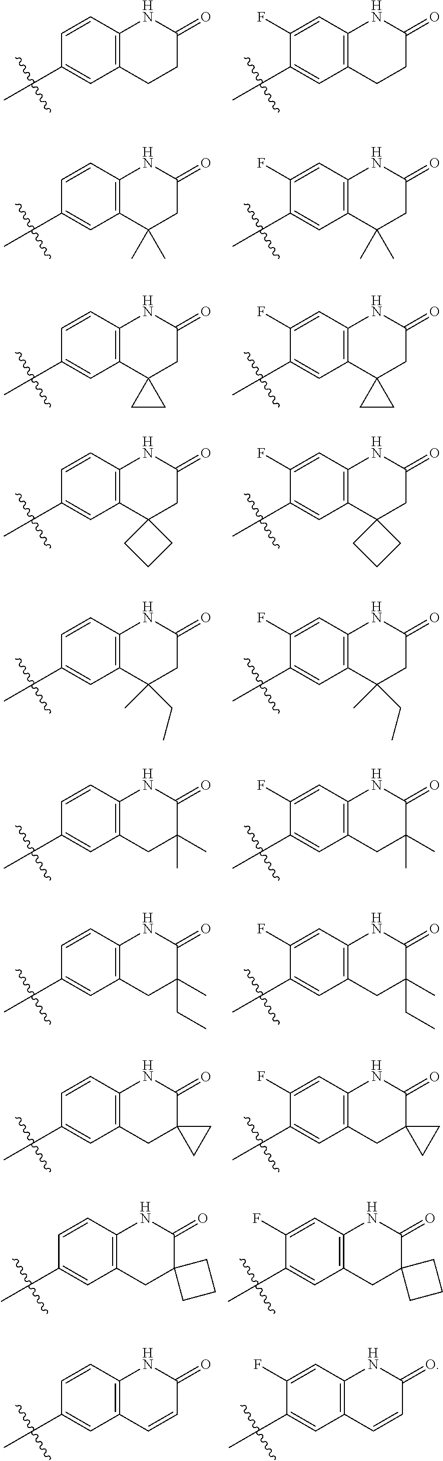

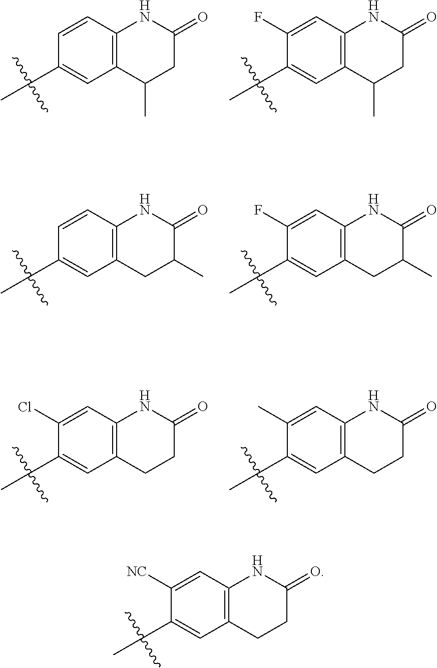

In some embodiments, R⁵ in Formula I-P is —O—R³⁰ or —CR²³R²⁴R²⁵ as defined and preferred herein. In some embodiments, R⁵ in Formula I-P is ethyl, n-propyl, isopropyl, n-butyl, isobutyl, sec-butyl, —CH₂—CHF₂, —CH₂—CF₃, —CF₃, —CH₂-cyclopropyl, —CH₂-cyclobutyl, —CH₂—O—CH₃, —CH₂—O—C₂H₅, —CH₂—O-n-propyl, —CH₂—O-isopropyl, —C₂H₄-cyclopropyl, —C₂H₄-cyclobutyl, methoxy, ethoxy, n-propoxy, isopropoxy, n-butoxy, isobutoxy, sec-butoxy, —O—CH₂—CF₃, —O—CF₃, —O—CH₂-cyclopropyl, —O—CH₂-cyclobutyl, —O—C₂H₄-cyclopropyl, or —O—C₂H₄-cyclobutyl.

Formula II

Some embodiments of the present disclosure are directed to compounds of Formula II, or a pharmaceutically acceptable salt thereof:

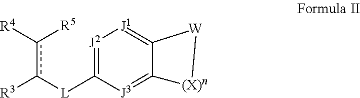

wherein:
W is —N(R¹)—C(O)—, —N(R¹)—S(O)—, or —N(R¹)—S(O)₂—;
L is —(CR^{A1}R^{B1})ₜ₁-Q¹-Q²-Q³-(CR^{A2}R^{B2})ₜ₂—, wherein:
   Q¹ and Q³ are independently null, O or NR²;
   Q² is null, —C(O)—, —C(═Z)—, —S(O)—, or —S(O)₂—;
   t1 is 0, 1, 2, or 3;
   t2 is 0, 1, 2, or 3; and
   R^{A1}, R^{B1}, R^{A2}, and R^{B2} at each occurrence are independently hydrogen, C₁₋₄ alkyl (e.g., methyl), or fluorine, or
   two adjacent CR^{A1}R^{B1} or two adjacent CR^{A2}R^{B2} can form —C(R^{A1})═C(R^{B1}), —C(R^{A2})═C(R^{B2})—, or

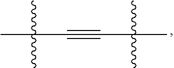

wherein R^{A1}, R^{B1}, R^{A2}, and R^{B2} at each occurrence are independently hydrogen, C₁₋₄ alkyl (e.g., methyl), or fluorine;
X at each occurrence is independently selected from O, NR¹⁰, and CR²⁰R²¹, provided that at most one X is selected from O and NR¹⁰;
n is 1, 2, 3, or 4;
J¹, J, and J³ are each independently selected from CR²² or N, preferably, at least one of J¹, J², and J³ is not N;
R¹ and R² at each occurrence are each independently hydrogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), or a nitrogen protecting group;
R³ and R⁴ are joined to form an optionally substituted aryl, an optionally substituted heteroaryl, an optionally substituted carbocyclic (e.g., C₃₋₈ carbocyclic), or an optionally substituted heterocyclic ring (e.g., 3-8 membered heterocyclic ring);
R⁵ is hydrogen, —NR¹¹R¹², —CR²³R²⁴R²⁵, or —OR³⁰;
R³, R⁴ and R⁵ are joined to form an optionally substituted bicyclic or polycyclic ring system, wherein the ring system is an aryl, heteroaryl, carbocyclic, or heterocyclic ring system;
or when Q² is —C(═Z)—, R⁵ and Z are joined to form an optionally substituted aryl, an optionally substituted heteroaryl, an optionally substituted carbocyclic (e.g., C₃₋₈ carbocyclic), or an optionally substituted heterocyclic ring (e.g., 3-8 membered heterocyclic ring);
“ [symbol] ” in Formula II indicates the bond is an aromatic bond, a double bond or a single bond as valance permits, and when a single bond, the two carbons forming the bond can be optionally further substituted as valance permits;
wherein:
   R¹⁰ at each occurrence is independently hydrogen, a nitrogen protecting group, an optionally substituted alkyl (e.g., optionally substituted C₁₋₆alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, or an optionally substituted 3-8 membered heterocyclic ring;
   R²⁰ and R²¹ at each occurrence are each independently hydrogen, halogen, —OR³¹, —NR¹³R¹⁴, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl; or
   R¹⁰ and one of R²⁰ and R²¹ are joined to form a bond, an optionally substituted 4-8 membered heterocyclic ring or an optionally substituted 5 or 6 membered heteroaryl ring, wherein the other of R²⁰ and R²¹ is defined above;
   R²⁰ and R²¹ together with the carbon they are both attached to form —C(O)—, an optionally substituted C₃₋₈ carbocyclic ring, or an optionally substituted 3-8 membered heterocyclic ring; or
   one of R²⁰ and R²¹ in one CR²⁰R²¹ is joined with one of R²⁰ and R²¹ in a different CR²⁰R²¹ to form a bond, an optionally substituted C₃₋₈ carbocyclic ring, an optionally substituted 3-8 membered heterocyclic ring, wherein the others of R²⁰ and R²¹ are defined above;
   R²² at each occurrence is independently hydrogen, halogen, an optionally substituted alkyl (e.g., optionally substituted C₁₋₄ alkyl), an optionally substituted alkenyl (e.g., optionally substituted C₂₋₆ alkenyl), an optionally substituted alkynyl (e.g., optionally substituted C₂₋₆ alkynyl), —CN, —S(O)-alkyl, —S(O)₂-alkyl, or —OR³¹;
   one of R¹¹ and R¹² is hydrogen or a nitrogen protecting group, and the other of R¹¹ and R¹² is hydrogen, a nitrogen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl;
   one of R²³, R²⁴, and R²⁵ is hydrogen, halogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₄ carbocyclic ring, an optionally substituted 3-8 membered heterocyclic ring, an optionally substituted phenyl, an optionally substituted 5-10 membered heteroaryl, —OR³¹, or —NR¹³R¹⁴, and the other two of R²³, R²⁴, and R²⁵ are independently selected from hydrogen, fluorine, or methyl, preferably, —CR²³R²⁴R²³ is not —CH₃;
   R³⁰ is hydrogen, an oxygen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, or an optionally substituted 3-8 membered heterocyclic ring; and
   wherein:
      each of R¹³ and eat each occurrence is independently hydrogen, a nitrogen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl; or R¹³ and R¹⁴ are joined to form a 3-8 membered optionally substituted heterocyclic or a 5-10 membered optionally substituted heteroaryl; and
      R³¹ at each occurrence is hydrogen, an oxygen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl.

Typically, in Formula II, the variables R³, R⁴, R⁵, J¹, J², J³, X, and n can be any of those described hereinabove in connection with Formula I and its subformulae. For example, in some embodiments, R³ and R⁴ in Formula II are joined to form an optionally substituted phenyl, an optionally substituted 5 or 6-membered heteroaryl, e.g., having one or two ring nitrogen atoms, an optionally substituted C7 cycloalkyl group (preferably cyclopentyl or cyclohexyl), or an optionally substituted 4 to 7-membered (preferably 6-membered) heterocyclic ring having one or two ring heteroatoms. In some embodiments, the moiety of

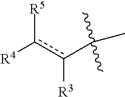

### in Formula II is

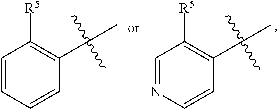

wherein R⁵ is defined herein, and wherein the phenyl or pyridyl can be further optionally substituted at any available position, for example, with one or two substituents independently selected from F; Cl; C₁₋₄ alkyl optionally substituted with 1-3 fluorines, preferably, methyl, ethyl, n-propyl, isopropyl, or —CF₃; a C₁₋₄ alkoxy optionally substituted with 1-3 fluorines, preferably, methoxy, ethoxy, n-propoxy, isopropoxy, or —OCF₃; a C₃₋₆ cycloalkyl optionally substituted with 1-3 substituents independently selected from fluorine and methyl, preferably, cyclopropyl or cyclobutyl; and —CN. In some embodiments, R⁵ is —O—R³⁰ or —CR²³R²⁴R²⁵ as defined and preferred herein. In some embodiments, R⁵ is ethyl, n-propyl, isopropyl, n-butyl, isobutyl, sec-butyl, —CH₂—CHF₂, —CH₂—CF₃, —CF₃, —CH₂-cyclopropyl, —CH₂-cyclobutyl, —CH₂—O—CH₃, —CH₂—O—C₂H₅, —CH₂—O-n-propyl, —CH₂—O-isopropyl, —C₂H₄-cyclopropyl, —C₂H₄-cyclobutyl, methoxy, ethoxy, n-propoxy, isopropoxy, n-butoxy, isobutoxy, sec-butoxy, —O—CH₂—CF₃, —O—CF₃, —O—CH₂-cyclopropyl, —O—CH₂-cyclobutyl, —O—C₂H₄-cyclopropyl, or —O—C₂H₄-cyclobutyl. In some embodiments, n is 2. In some embodiments, J¹ is CH. In some embodiments, J² is N or CR²², wherein R²² is defined herein. In some embodiments, R²² is hydrogen, F, Cl, CN, or methyl. In some embodiments, J³ is CH. In some embodiments, each instance of X in Formula II is independently selected CR²⁰R²¹, and R²⁰ and R²¹ are independently hydrogen or C₁₋₄ alkyl (e.g., methyl, ethyl, etc.), or R²⁰ and R²¹, together with the carbon they are both attached to, form a cyclopropyl, cyclobutyl, cyclopentyl, or an oxetanyl ring. In some embodiments, in the CR²⁰R²¹ unit:

R²⁰ and R²¹ are both methyl;

one of R²⁰ and R²¹ is methyl, and the other of R²⁰ and R²¹ is ethyl or methoxy; or

R²⁰ and R²¹, together with the carbon they are both attached to, form a cyclopropyl, cyclobutyl, or an oxetanyl ring. In some embodiments, in the CR²⁰R²¹ unit, one of R²⁰ and R²¹ is methyl, and the other of R²⁰ and R²¹ is hydrogen. In some embodiments, in the CR²⁰R²¹ unit, R²⁰ and R²¹ are both hydrogen.

W in Formula II is typically N(R¹)—C(O)— or —N(R′)—S(O)₂—, wherein either the nitrogen atom or the C(O)— or S(O)₂— can be directly attached to an X, in other words, the expression is bi-directional. Typically, R¹ is hydrogen or a C₁₋₄ alkyl. For example, in some embodiments, the compound of Formula II can have a Formula II-1, II-2, II-3, or II-4:

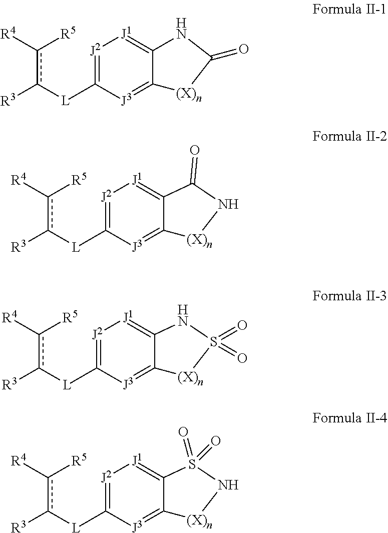

wherein R³, R⁴, R⁵, L, J¹, J², J³, X, and n are defined herein.

L in Formula II (e.g., Formula II-1, II-2, II-3, or II-4) is typically —(CR^{A1}R^{B1})ₜ₁-Q¹-Q²-Q³-(CR^{A2}R^{B2})ₜ₂—, wherein:
(1) Q² is —C(O)—, one of Q¹ and Q³ is null, the other of Q¹ and Q³ is NR² as defined herein, t1 is 0 or 1, and t2 is 0 or 1, preferably, both t1 and t2 are 0, R² is hydrogen or methyl;
(2) Q¹, Q² and Q³ are null, t1 is 0, t2 is 2, and two adjacent CR^{A2}R^{B2} form —C(R^{A1})═C(R^{B2})— as defined herein, preferably, R^{A2} and R^{B2} are both hydrogen; or
(3) Q² is null, and one of Q¹ and Q³ is null, the other of Q¹ and Q³ is NR² as defined herein, t1 is 0 or 1, and t2 is 0 or 1, preferably, R² is hydrogen or methyl, and t1 and t2 are not both 0.
   It should also be noted that the bivalent linker L, —(CR^{A1}R^{B1})ₜ₁-Q¹-Q²-Q³-(CR^{A2}R^{B2})ₜ₂—, in Formula II can link the remaining structures in either direction. For example, the

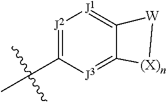

unit can be directly attached to the —(CR^{A1}R^{B1})ₜ₁ end of the linker or the (CR^{A2}R^{B2})ₜ₂— end of the linker. In some preferred embodiments, a NR² is directly linked to the

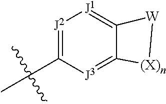

unit.

In some embodiments, the

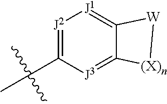

unit in Formula II (e.g., Formula II-1, II-2, II-3, or II-4) can be selected from any of those described as suitable

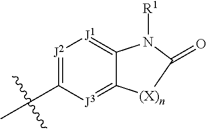

in connection with Formula I herein, for example,

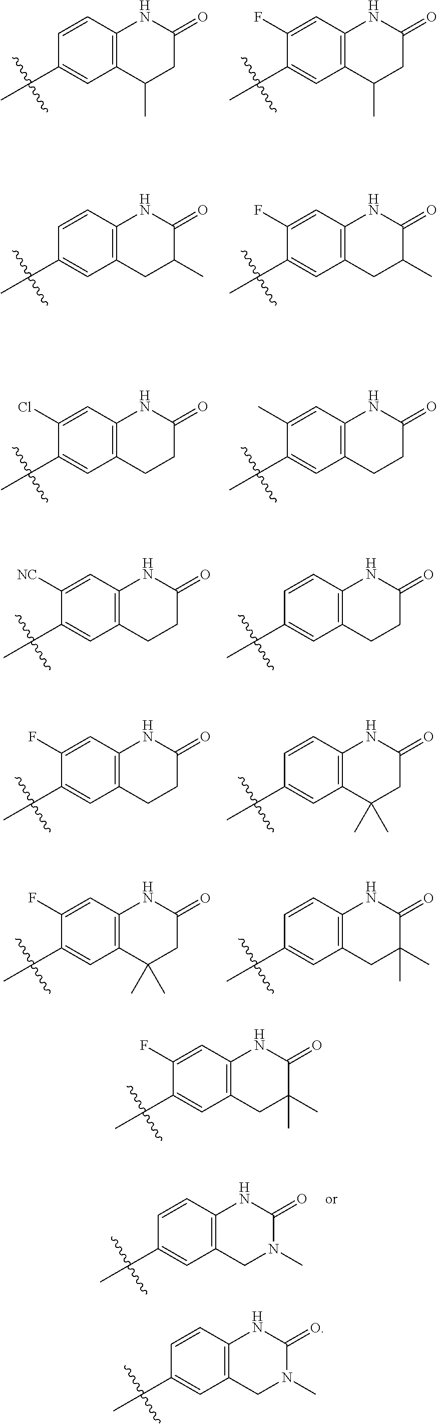

In some embodiments, the present disclosure also provides a compound of Formula II-P, or a pharmaceutically acceptable salt thereof:

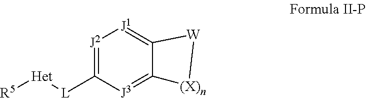

wherein Het represents an optionally substituted heterocyclic or heteroaryl ring structure, preferably, 5 or 6 membered heterocyclic ring or 5 or 6 membered heteroaryl ring, wherein R⁵, J¹, J², J³, L, W, X, and n can be any of those defined herein for Formula II (including its subformulae). Preferably, Het is a 5 or 6 membered heteroaryl, and in Formula II-P, R⁵ is attached to the Het at an ortho position of the linker L. It will also be understood that in Formula II-P, R⁵ can be attached to a ring nitrogen as valance permits.

In some embodiments, in Formula II-P, Het is an optionally substituted 5 or 6 membered heteroaryl described herein, for example, Het is a 5 or 6 membered heteroaryl, preferably, a pyrazole, imidazole, oxazole, thiazole, isoxazole, isothiazole, pyridyl, pyrimidinyl, pyridazinyl, or pyrazinyl, which is optionally substituted with one or two (preferably one) substituents independently selected from F; Cl; C₁₋₄ alkyl optionally substituted with 1-3 fluorines, preferably, methyl, ethyl, n-propyl, isopropyl, or —CF₃; a C₁₋₄ alkoxy optionally substituted with 1-3 fluorines, preferably, methoxy, ethoxy, n-propoxy, isopropoxy, or —OCF₃; a C₃₋₆ cycloalkoy optionally substituted with 1-3 substituents independently selected from fluorine and methyl; a C₃₋₆ cycloalkyl optionally substituted with 1-3 substituents independently selected from fluorine and methyl, preferably, cyclopropyl or cyclobutyl; and —CN. In some embodiments, R⁵ is —O—R³⁰ or —CR²³R²⁴R²⁵ as defined and preferred herein. In some embodiments, R⁵ is ethyl, n-propyl, isopropyl, n-butyl, isobutyl, sec-butyl, —CH₂—CHF₂, —CH₂—CF₃, —CF₃, —CH₂-cyclopropyl, —CH₂-cyclobutyl, —CH₂—O—CH₃, —CH₂—O—C₂H₅, —CH₂—O-n-propyl, —CH₂—O-isopropyl, —C₂H₄-cyclopropyl, —C₂₁₁₄-cyclobutyl, methoxy, ethoxy, n-propoxy, isopropoxy, n-butoxy, isobutoxy, sec-butoxy, —O—CH₂—CF₃, —O—CF₃, —O—CH₂-cyclopropyl, —O—CH₂-cyclobutyl, —O—C₂H₄-cyclopropyl, or —O—C₂H₄-cyclobutyl.

Formula III

In some embodiments, the present disclosure provides a compound of Formula III, or a pharmaceutically acceptable salt thereof:

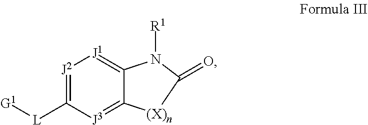

wherein:
X at each occurrence is independently selected from O, NR¹⁰, and CR²⁰R²¹, provided that at most one X is selected from O and NR¹⁰;
n is 1, 2, 3, or 4;
J¹, J², and J³ are each independently selected from CR²² or N, preferably, at least one of J¹, J², and J³ is not N;
R¹ is hydrogen, an optionally substituted alkyl (e.g., optionally substituted C₁₋₄alkyl), an optionally substituted alkenyl (e.g., optionally substituted C₂₋₆ alkenyl), an optionally substituted alkynyl (e.g., optionally substituted C₂₋₆ alkynyl), or a nitrogen protecting group;
L is NH, O, or selected from:

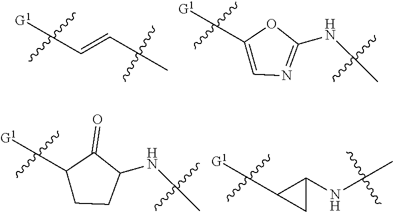

G¹ is an optionally substituted phenyl, optionally substituted heteroaryl (e.g., 5- or 6-membered heteroaryl, or 8-10 membered bicyclic heteroaryl), or an optionally substituted heterocyclyl,
wherein:
   R¹⁰ at each occurrence is independently hydrogen, a nitrogen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, or an optionally substituted 3-8 membered heterocyclic ring;
   R²⁰ and R²¹ at each occurrence are each independently hydrogen, halogen, —OR³¹, —NR¹³R¹⁴, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl; or
   R¹⁰ and one of R²⁰ and R²¹ are joined to form a bond, an optionally substituted 4-8 membered heterocyclic ring or an optionally substituted 5 or 6 membered heteroaryl ring, wherein the other of R²⁰ and R²¹ is defined above;
   R²⁰ and R²¹ together with the carbon they are both attached to form —C(O)—, an optionally substituted C₃₋₈ carbocyclic ring, or an optionally substituted 3-8 membered heterocyclic ring; or
   one of R²⁰ and R²¹ in one CR²⁰R²¹ is joined with one of R²⁰ and R²¹ in a different CR²⁰R²¹ to form a bond, an optionally substituted C₃₋₈ carbocyclic ring, an optionally substituted 3-8 membered heterocyclic ring, wherein the others of R²⁰ and R²¹ are defined above;
   R²² at each occurrence is independently hydrogen, halogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂. alkynyl), —CN, —S(O)-alkyl (e.g., —S(O)—C₁₋₆ alkyl), —S(O)₂-alkyl (e.g., —S(O)₂—C₁₋₆ alkyl), or —OR³¹;
   wherein:
      each of R¹³ and R¹⁴ at each occurrence is independently hydrogen, a nitrogen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl; or R¹³ and R¹⁴ are joined to form a 3-8 membered optionally substituted heterocyclic or a 5-10 membered optionally substituted heteroaryl; and
R³¹ at each occurrence is hydrogen, an oxygen protecting group, an optionally substituted alkyl (e.g., optionally substituted C₁₋₄ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl.

In some embodiments, the compound of Formula III can have a Formula III-1 or III-2:

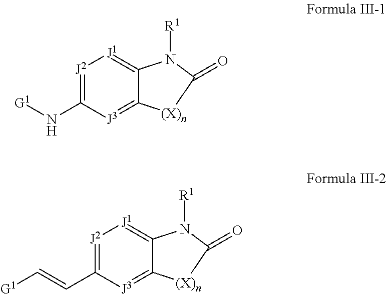

wherein R¹, G¹, J¹, J², J³, X, and n are defined herein.

In some embodiments,

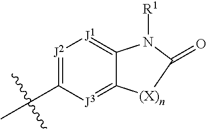

in Formula III (e.g., III-1 or III-2) can be any of those described for Formula I (including its subformulae). For example, in some embodiments,

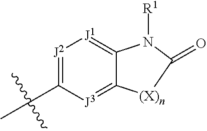

in Formula III (e.g., III-1 or III-2) can be selected from the following:

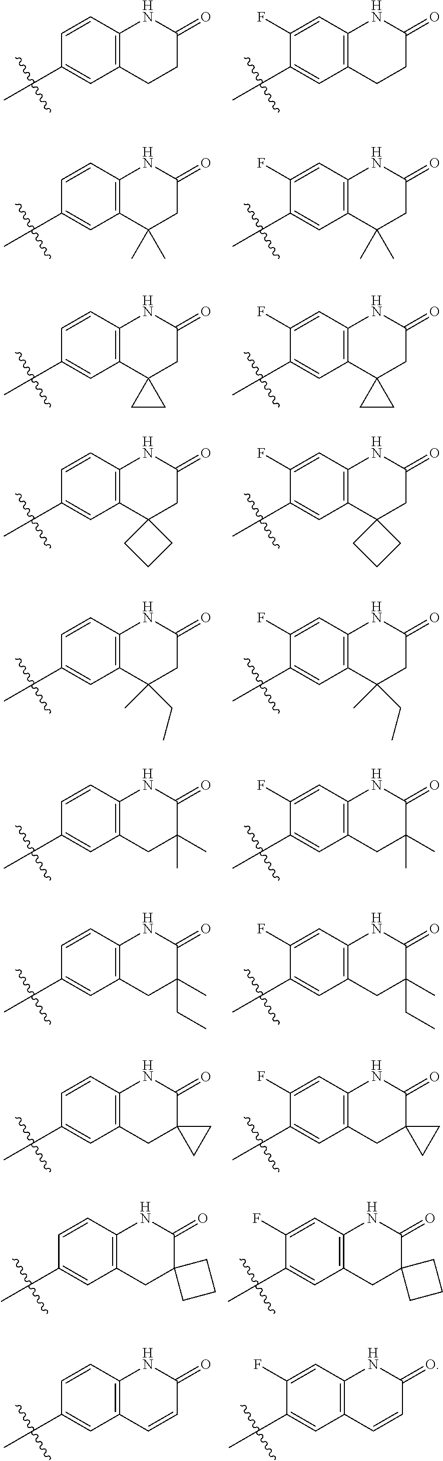

In some embodiments,

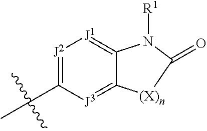

in Formula III (e.g., III-1 or III-2) can be selected from the following:

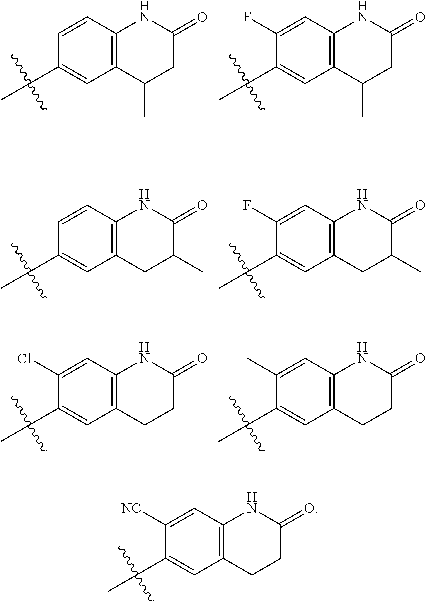

In some embodiments,

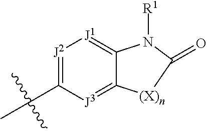

in Formula III (e.g., III-1 or III-2) can be selected from the following:

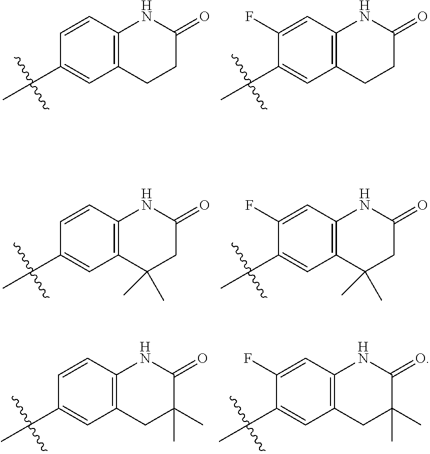

In some embodiments, in Formula III (e.g., Formula III-1 or III-2), the

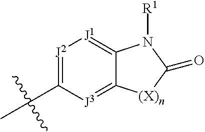

can be any of the corresponding moieties shown in Compound Nos. 1-138 disclosed herein, as applicable.

G¹ in Formula III is typically an optionally substituted phenyl or optionally substituted heteroaryl, which includes any of those described herein.

In some embodiments, the compound of Formula III is characterized as having a formula of III-1, wherein G¹ is an optionally substituted 5- or 6-membered heteroaryl or an optionally substituted 8-10 membered bicyclic heteroaryl. In some embodiments, the compound of Formula III is characterized as having a formula of III-1, wherein G¹ is selected from the following:

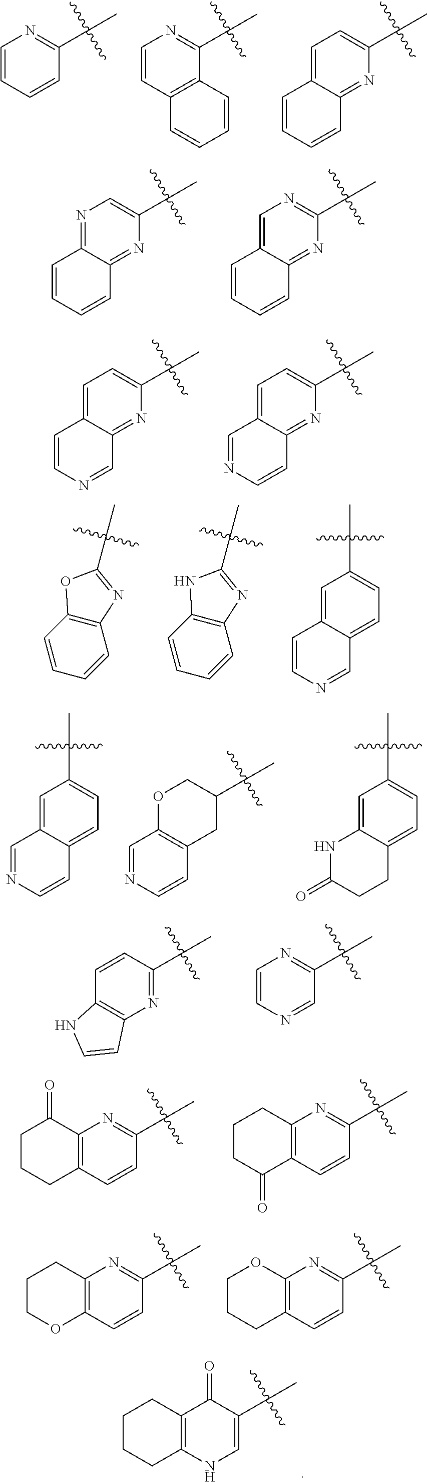

wherein each of the groups is optionally further substituted, for example, with one or two substituents each independently halogen (e.g., Cl), C₁₋₄ alkyl, CN, hydroxyl, COOH, C(O)—O—(C₁₋₄ alkyl), etc. In some embodiments, the compound of Formula III is characterized as having a formula of III-1, wherein G¹ is

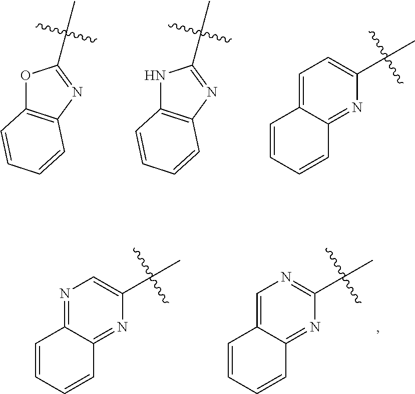 [SMILES: *c1nc2ccccc2o1 ; *c1nc2ccccc2[nH]1 ; *c1ccc2ccccc2n1 ; *c1cnc2ccccc2n1 ; *c1ncc2ccccc2n1]

wherein the bicyclic heteroaryl is unsubstituted or further substituted with one or two (preferably one) substituents. When substituted, the substituents can be preferably independently selected from Cl, methyl, and hydroxyl. Representative heteroaryls suitable as G¹ for Formula III-1 are shown in the exemplified compounds herein.

In some embodiments, the compound of Formula III is characterized as having a formula of III-2, wherein G¹ can be any of those described herein as suitable as the moiety of

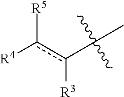

in Formula I (e.g., any of the applicable subformulae). For example, in some embodiments, the compound of Formula III is characterized as having a formula of III-2, wherein G¹ can be selected from any of the following:

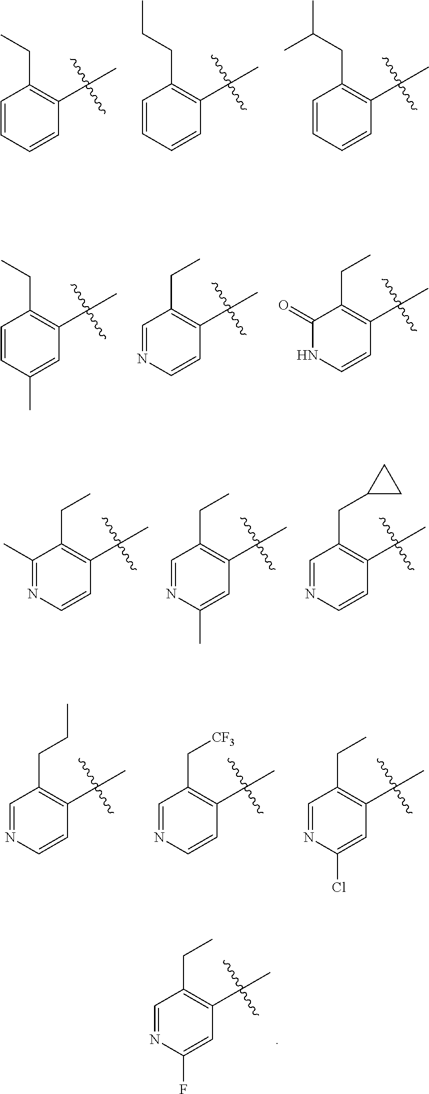

In some embodiments, the present disclosure also provides a compound selected from compound Nos. 1-138, or a pharmaceutically acceptable salt thereof:

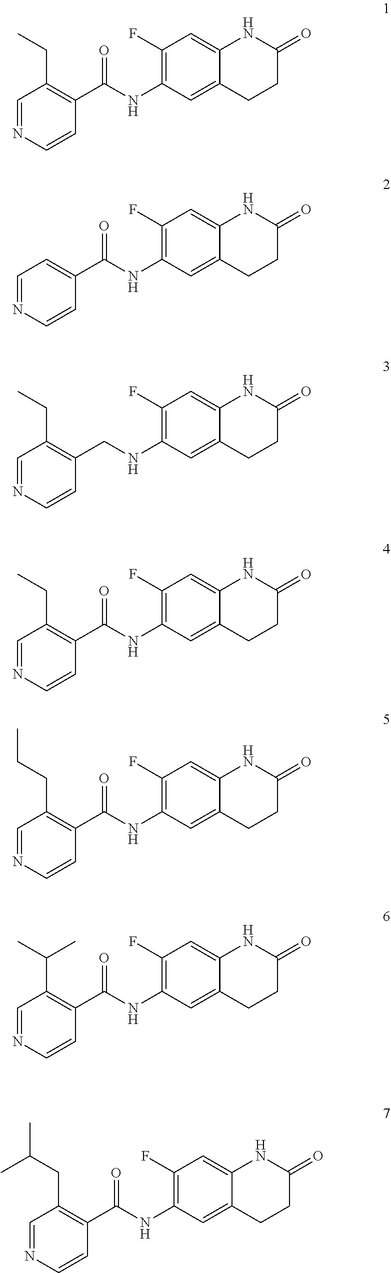

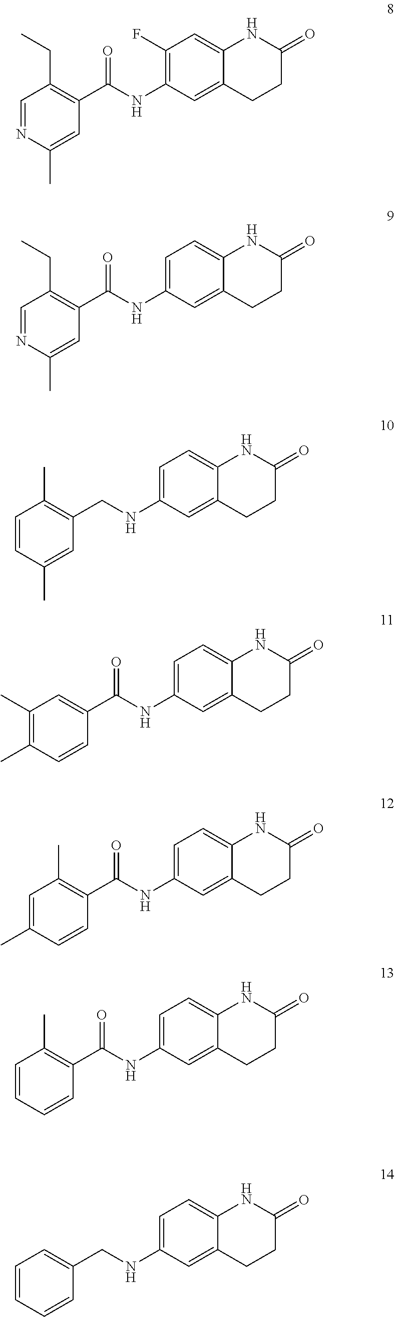

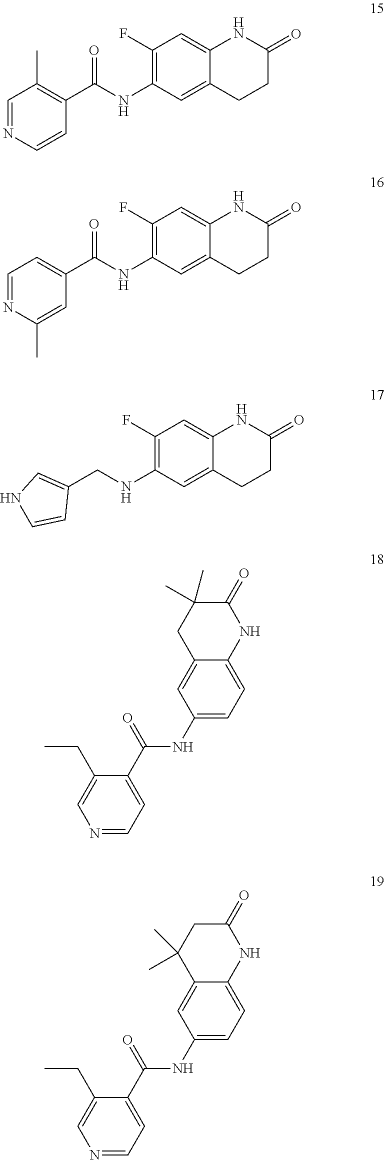 [SMILES: Cc1cnccc1C(=O)Nc1cc2c(cc1F)NC(=O)CC2 ; Cc1cc(C(=O)Nc2cc3c(cc2F)NC(=O)CC3)ccn1 ; O=C1CCc2cc(NCc3cc[nH]c3)c(F)cc2N1 ; CCc1cnccc1C(=O)Nc1ccc2c(c1)CC(C)(C)C(=O)N2 ; CCc1cnccc1C(=O)Nc1ccc2c(c1)C(C)(C)CC(=O)N2]

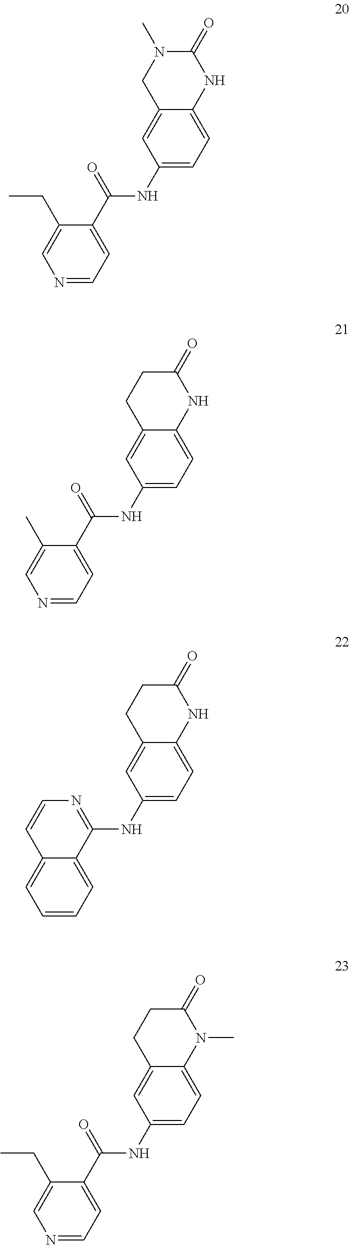 [SMILES: CCc1cnccc1C(=O)Nc1ccc2c(c1)CN(C)C(=O)N2 ; Cc1cnccc1C(=O)Nc1ccc2c(c1)CCC(=O)N2 ; O=C1CCc2cc(Nc3nccc4ccccc34)ccc2N1 ; CCc1cnccc1C(=O)Nc1ccc2c(c1)CCC(=O)N2C]

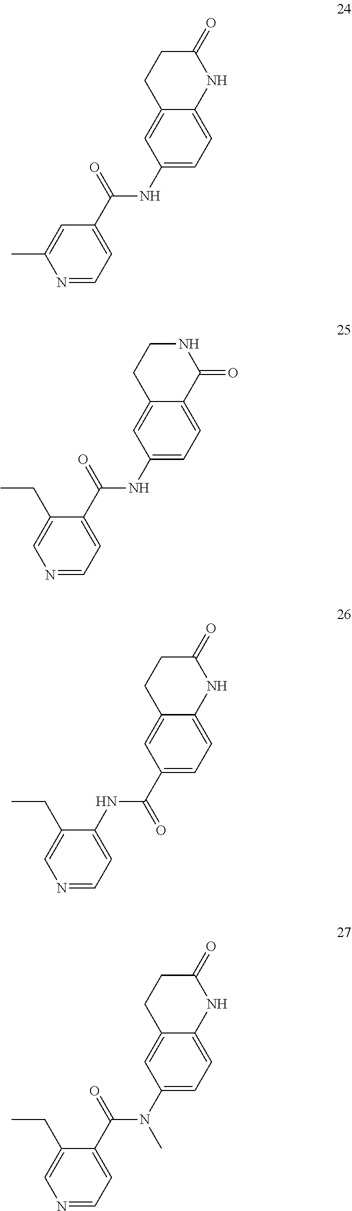 [SMILES: Cc1cc(C(=O)Nc2ccc3c(c2)CCC(=O)N3)ccn1 ; CCc1cnccc1C(=O)Nc1ccc2c(c1)CCNC2=O ; CCc1cnccc1NC(=O)c1ccc2c(c1)CCC(=O)N2 ; CCc1cnccc1C(=O)N(C)c1ccc2c(c1)CCC(=O)N2]

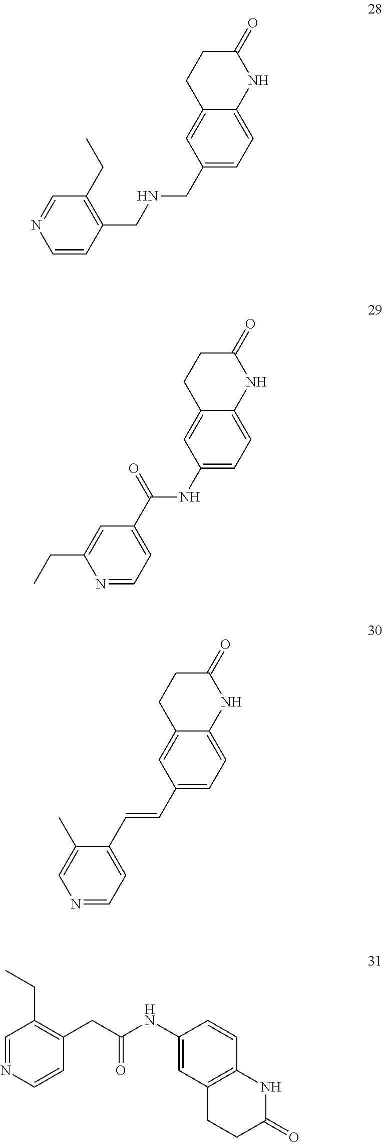 [SMILES: CCc1cnccc1CNCc1ccc2c(c1)CCC(=O)N2 ; CCc1cc(C(=O)Nc2ccc3c(c2)CCC(=O)N3)ccn1 ; Cc1cnccc1/C=C/c1ccc2c(c1)CCC(=O)N2 ; CCc1cnccc1CC(=O)Nc1ccc2c(c1)CCC(=O)N2]

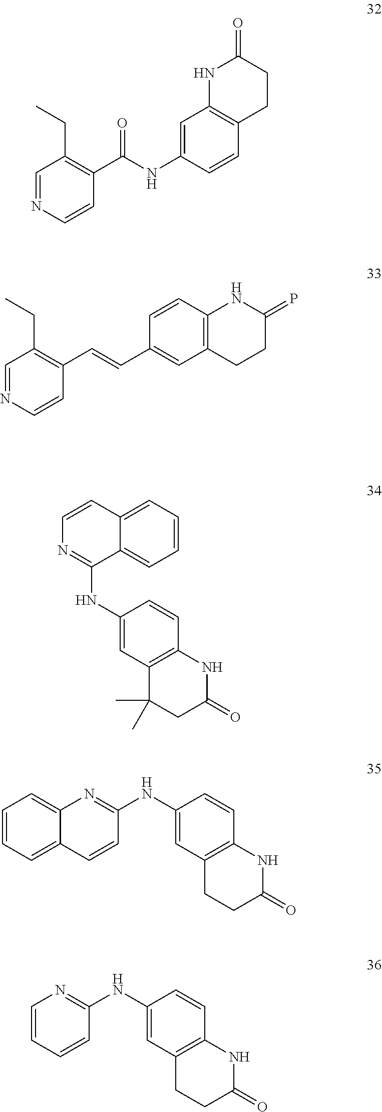

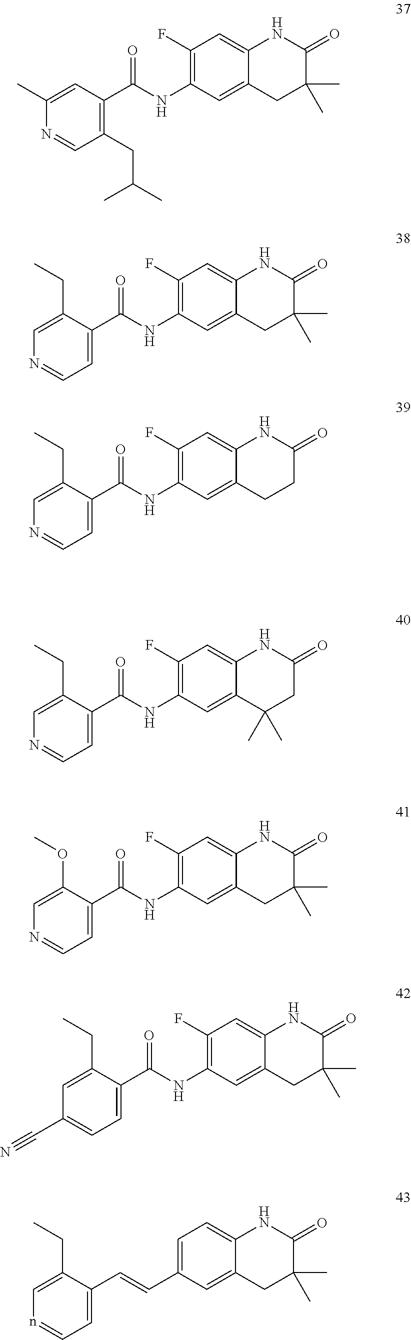

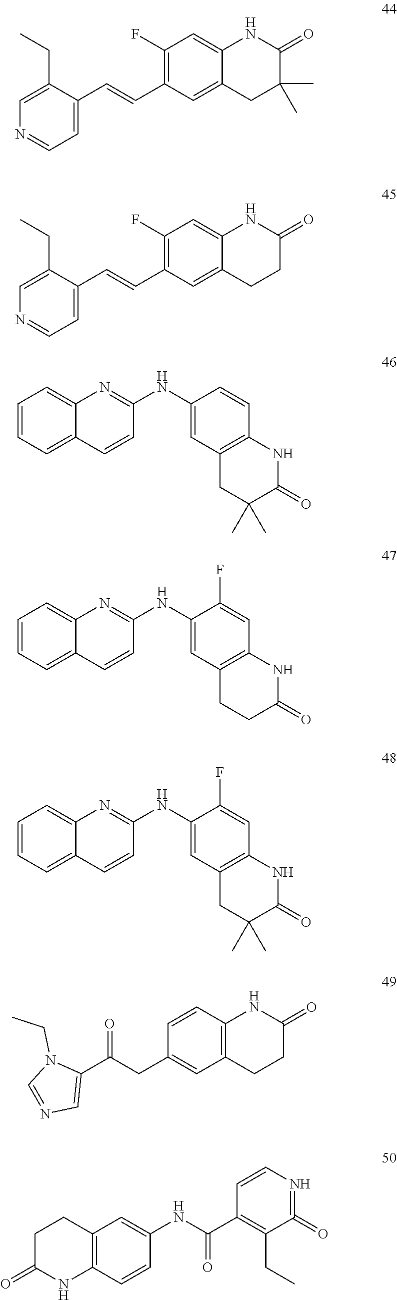

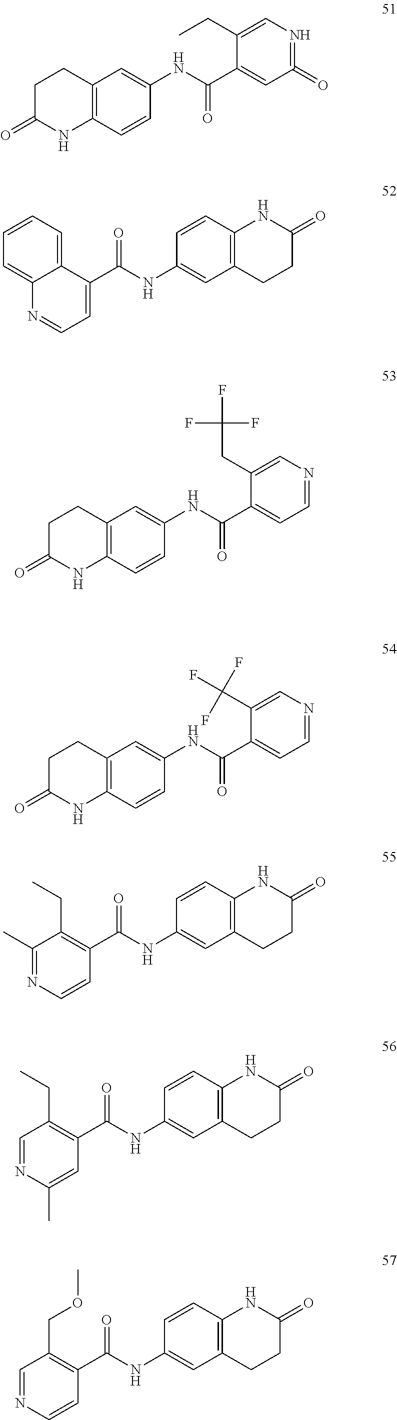

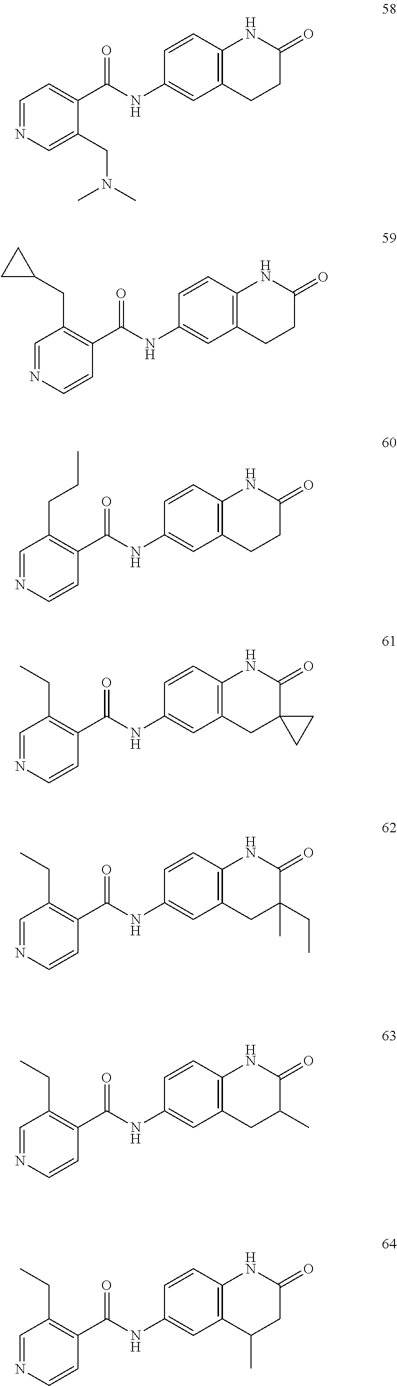

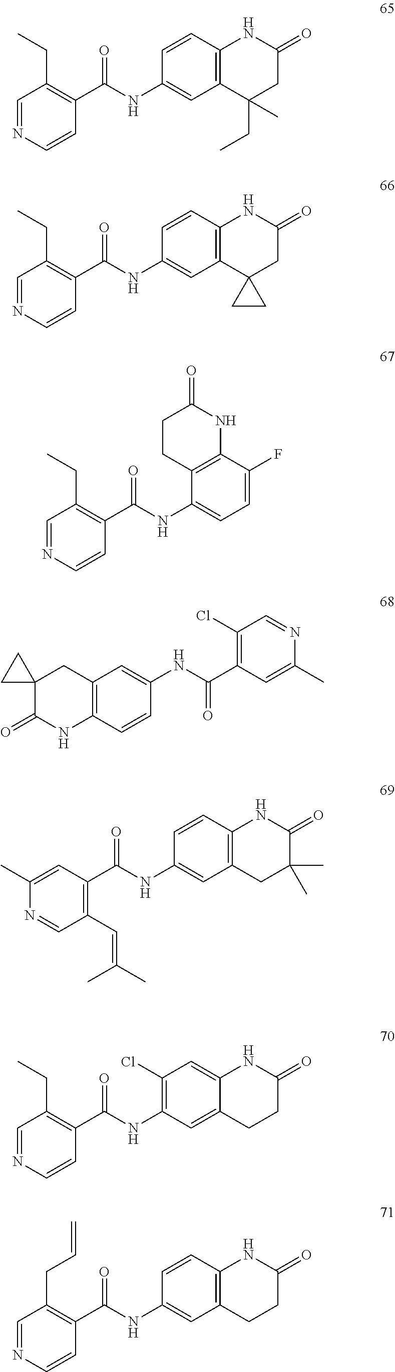

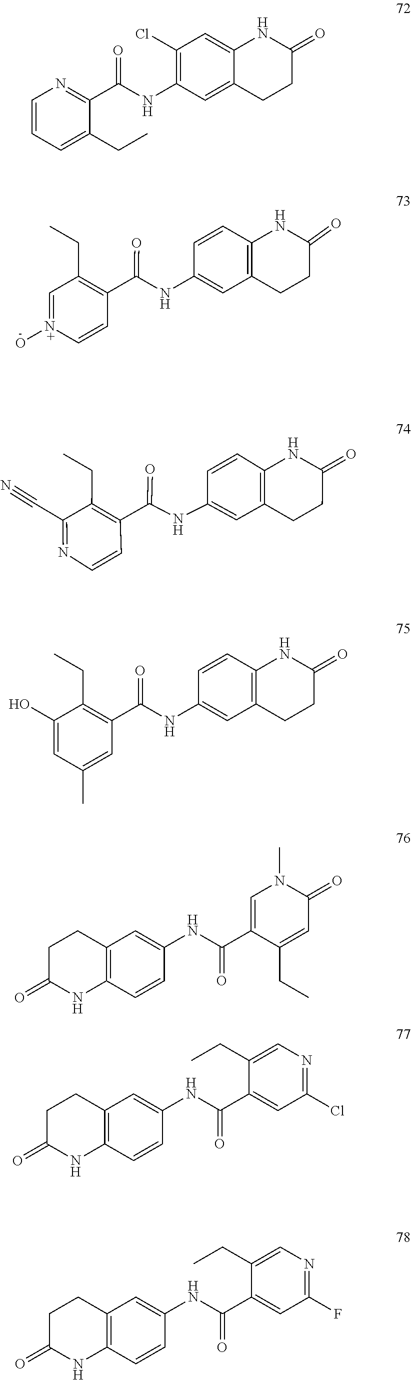

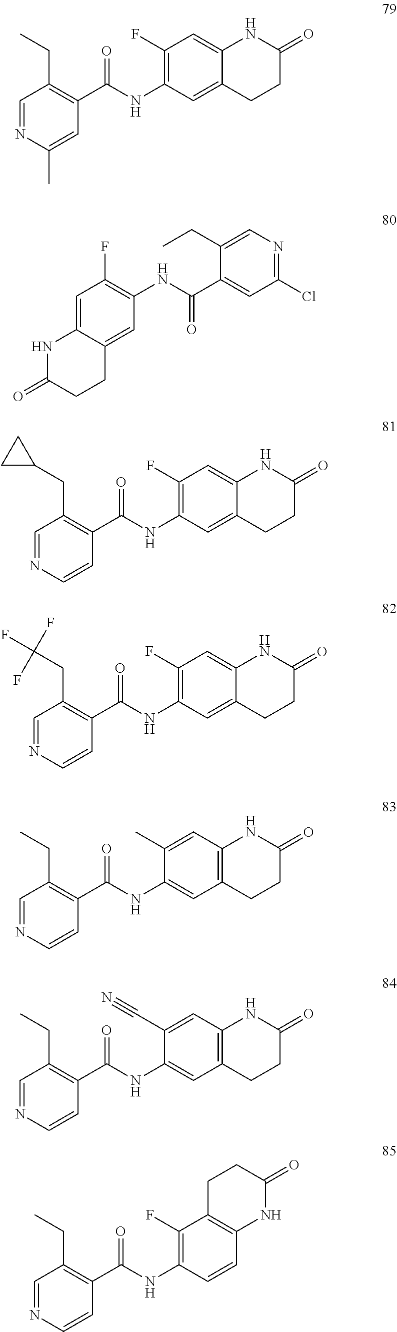

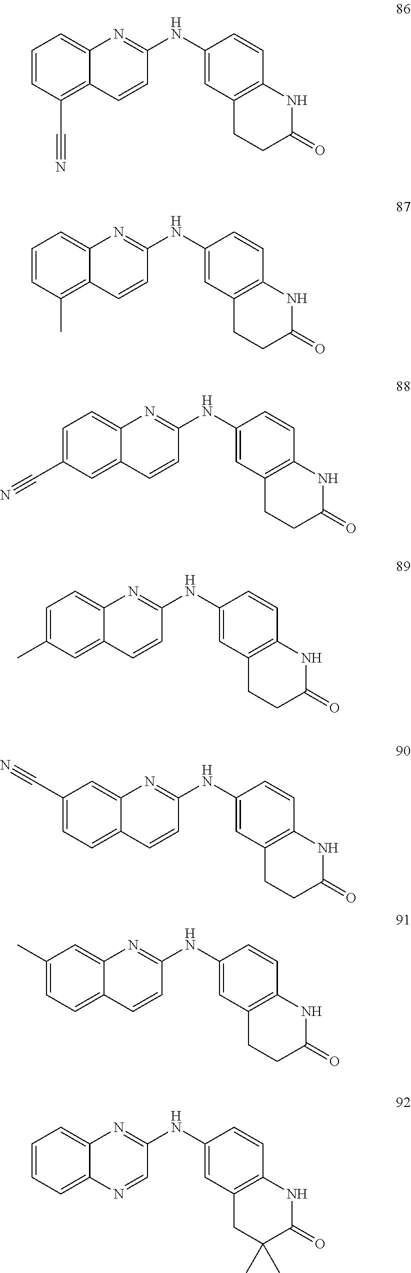

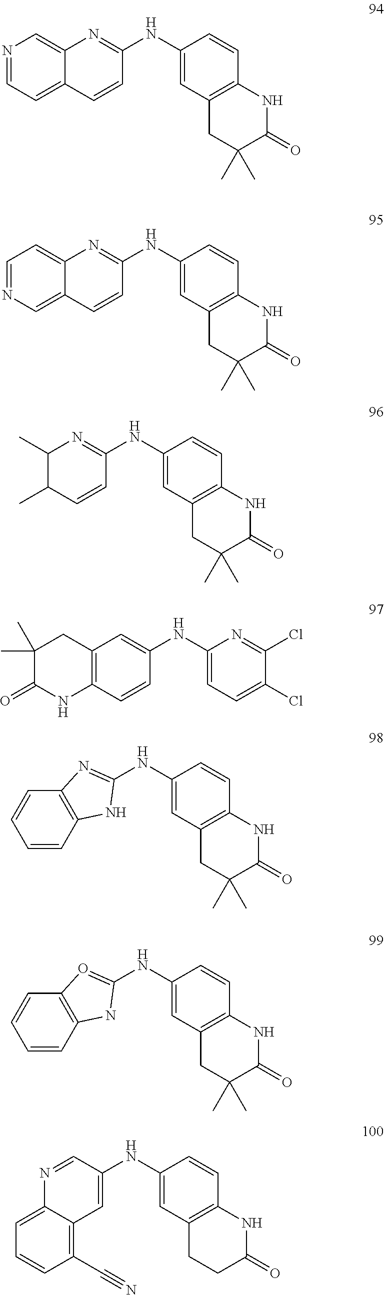

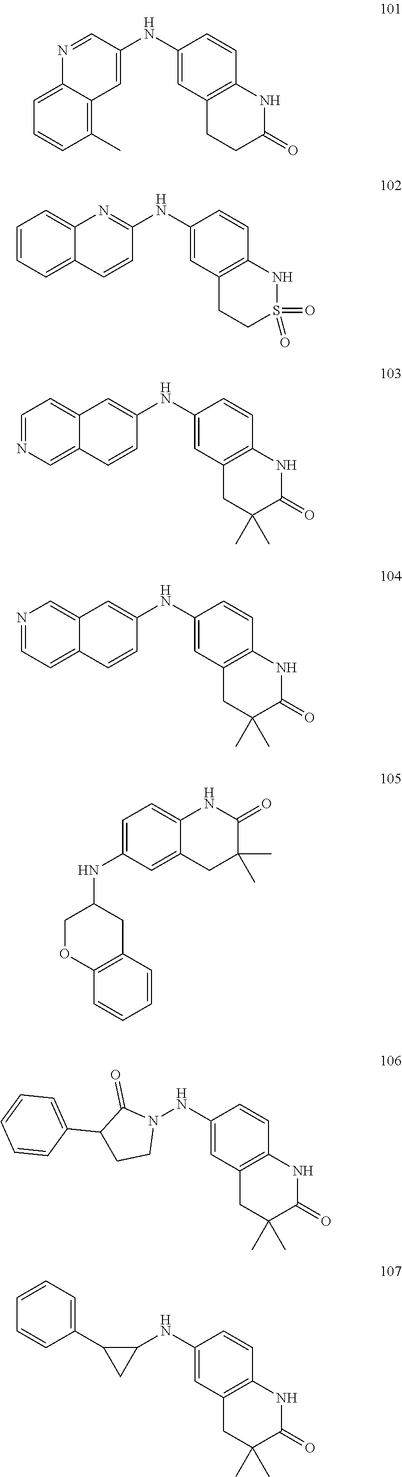

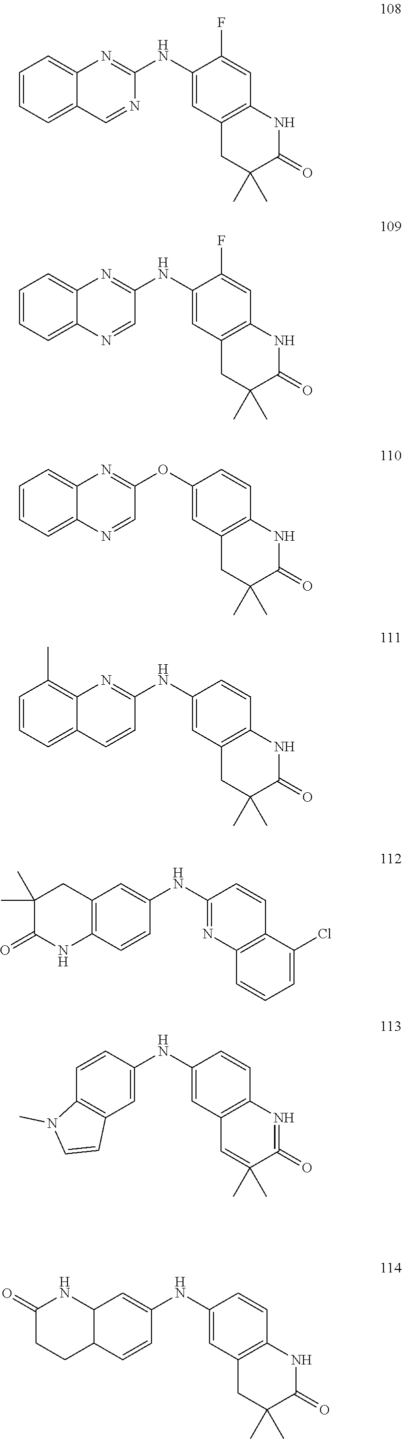

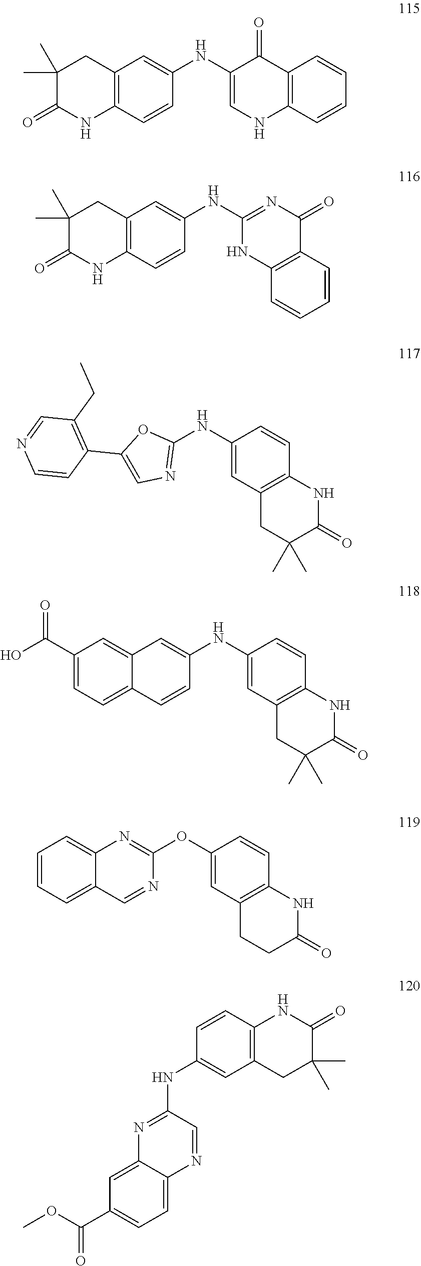

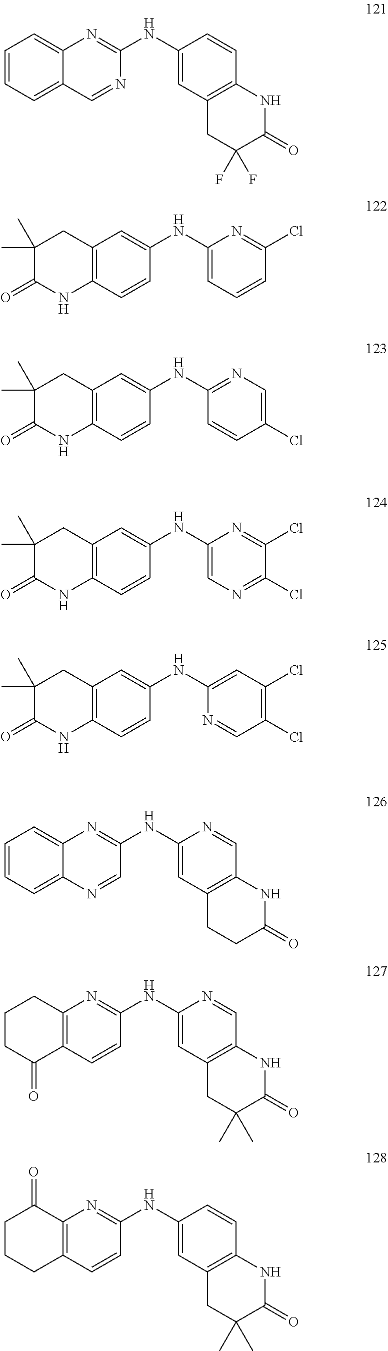

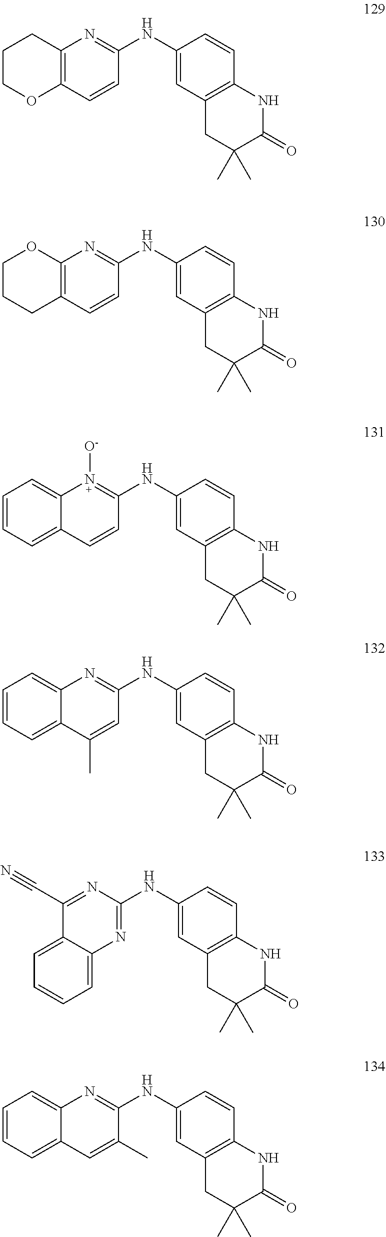

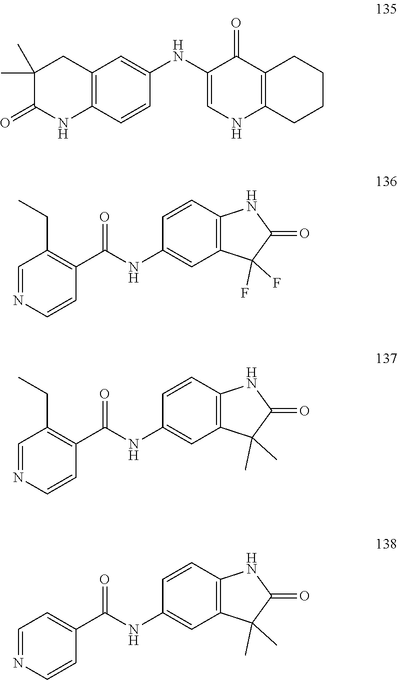 [SMILES: CC1(C)Cc2cc(Nc3c[nH]c4c(c3=O)CCCC4)ccc2NC1=O ; CCc1cnccc1C(=O)Nc1ccc2c(c1)C(F)(F)C(=O)N2 ; CCc1cnccc1C(=O)Nc1ccc2c(c1)C(C)(C)C(=O)N2 ; CC1(C)C(=O)Nc2ccc(NC(=O)c3ccncc3)cc21]

In some embodiments, to the extent applicable, the genus of compounds described herein also excludes any specifically known single compound(s) prior to this disclosure. In some embodiments, to the extent applicable, any sub-genus of compounds prior to this disclosure that are entirely within a genus of compounds described herein can also be excluded from such genus herein.

### Method of Synthesis

Compounds of the present disclosure can be readily synthesized by those skilled in the art in view of the present disclosure. Exemplified synthesis are also shown in the Examples section.

The synthesis of compounds of Formula I-1 as shown in Scheme 1 is a representative method for the preparation of compounds herein.

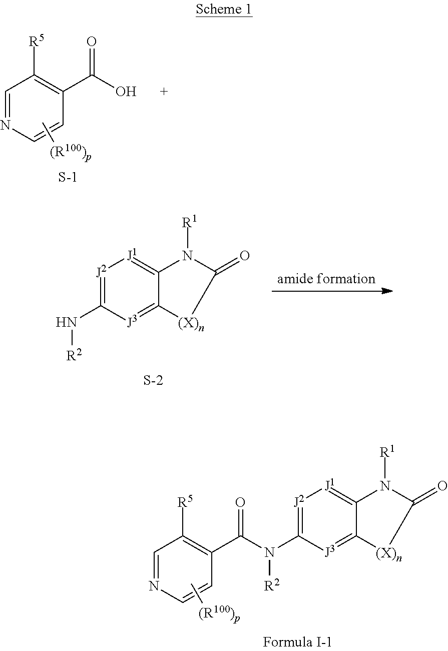

As shown in Scheme 1, compounds of Formula I-1 can be typically prepared by an amide coupling reaction between suitable coupling partners, S-1 and S-2. Amide coupling reaction conditions are generally known by those skilled in the art and also exemplified in the Examples section herein. Typically, the acid S-1 can be converted into an activated form, such as acyl chloride, anhydride, active esters, etc., which can then react with the amine S-2 to form the compound of Formula I-1. For example, the Examples section describe a representative EDCI (1-Ethyl-3-(3-dimethylaminopropyl)carbodiimide) mediated amide coupling reaction. The acid S-1 and amine S-2 can be readily available or be prepared by those skilled in the art in view of the present disclosure. The variables of R¹, R², R⁵, R¹⁰⁰, J¹, J², J³, X, p, and n are defined herein in connection with Formula I-1. Typically, R² in S-2 is hydrogen. Other compounds of Formula I, I-P, II, or II-P with an amide linkage can be prepared similary.

Compounds of Formula I, I-P, II, II-P or III that are not connected with an amide linkage can be typically prepared by other cross coupling reactions known to those skilled in the art, such as various palladium catalyzed cross-coupling reactions including Hartwig-Buchwald amination, Heck reaction, Suzuki reaction, etc. Exemplified procedures are described in the Examples section herein.

As will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4^{th} ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wis., USA), Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7^{th} Edition), and Larock's Comprehensive Organic Transformations (Wiley-VCH, 1999), and any of available updates as of this filing.

### Pharmaceutical Compositions

Certain embodiments are directed to a pharmaceutical composition comprising one or more compounds of the present disclosure.

The pharmaceutical composition can optionally contain a pharmaceutically acceptable excipient. In some embodiments, the pharmaceutical composition comprises a compound of the present disclosure (e.g., a compound of Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), Formula I-P, Formula II (e.g., Formula II-1, II-2, II-3, or II-4,), Formula II-P, Formula III (e.g., Formula III-1 or III-2), or any of Compound Nos. 1-138, or a pharmaceutically acceptable salt thereof) and a pharmaceutically acceptable excipient. Pharmaceutically acceptable excipients are known in the art. Non-limiting suitable excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The pharmaceutical composition can include any one or more of the compounds of the present disclosure. For example, in some embodiments, the pharmaceutical composition comprises a compound of Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), Formula I-P, Formula II (e.g., Formula II-1, II-2, II-3, or II-4,), Formula II-P, Formula III (e.g., Formula III-1 or III-2), or any of Compound Nos. 1-138, or a pharmaceutically acceptable salt thereof, e.g., in a therapeutically effective amount. In any of the embodiments described herein, the pharmaceutical composition can comprise a therapeutically effective amount of a compound selected from Compound Nos. 1-138 (e.g., any of the compounds having an activity level of A or B shown in Table 3 of the present disclosure), or a pharmaceutically acceptable salt thereof. In some preferred embodiments, the pharmaceutical composition can comprise a therapeutically effective amount of any compound of the present disclosure having an efficacy in ALDH1a3 inhibition comparable to Compound 1 or better, e.g., measured by any of the methods described herein. In some preferred embodiments, the pharmaceutical composition can comprise a therapeutically effective amount of any compound of the present disclosure having an IC50 value of less than 250 nM (preferably, less than 100 nM, such as about 1-100 nM, about 10-100 nM, about 10-50 nM, about 20-100 nM, about 20-50 nM, etc.) in inhibiting hALDH1a3 when measured by the method described herein according to Biological Example 5B.

The pharmaceutical composition can also be formulated for delivery via any of the known mutes of delivery, which include but are not limited to oral, parenteral, inhalation, etc. For example, in some embodiments, the pharmaceutical composition can be formulated for administering to a subject orally, nasally, transdermally, pulmonary, inhalationally, buccally, sublingually, intraperintoneally, subcutaneously, intramuscularly, intravenously, rectally, intrapleurally, intrathecally or parenterally.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

In some embodiments, the pharmaceutical composition is formulated for parenteral administration (such as intravenous injection or infusion, subcutaneous or intramuscular injection). The parenteral formulations can be, for example, an aqueous solution, a suspension, or an emulsion. Excipients for the preparation of parenteral formulations are known in the art. Non-limiting suitable excipients include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof.

In some embodiments, the pharmaceutical composition is formulated for inhalation. The inhalable formulations can be, for example, formulated as a nasal spray, dry powder, or an aerosol administrable through a metered-dose inhaler. Excipients for preparing formulations for inhalation are known in the art. Non-limiting suitable excipients include, for example, lactose, talc, silicic acid, aluminum hydroxide, calcium silicates and polyamide powder, and mixtures of these substances. Sprays can additionally contain propellants, such as chlorofluorohydrocarbons and volatile unsubstituted hydrocarbons, such as butane and propane.

Compounds of the present disclosure can be used alone, in combination with each other, or in combination with one or more additional therapeutic agents, e.g., metformin, recombinant insulin, liraglutide, semaglutide, empagliflozin, paclitaxel, doxorubicin, 5-fluorouracil, tamoxifen, octreotide, etc. When used in combination with one or more additional therapeutic agents, compounds of the present disclosure or pharmaceutical compositions herein can be administered to the subject either concurrently or sequentially in any order with such additional therapeutic agents. In some embodiments, the pharmaceutical composition can comprise one or more compounds of the present disclosure and the one or more additional therapeutic agents in a single composition. In some embodiments, the pharmaceutical composition comprising one or more compounds of the present disclosure can be included in a kit which also comprises a separate pharmaceutical composition comprising the one or more additional therapeutic agents.

As discussed herein, compounds of the present disclosure can sensitize the cancer for chemotherapy treatment. In some embodiments, compounds of the present disclosure can be used in combination with a chemotherapeutic agent, for example, for treating cancer. Any of the known chemotherapeutic agents can be used in combination with one or more compounds of the present disclosure. Non-limiting useful examples of chemotherapeutic agents include antineoplastic agents and combinations thereof, such as DNA alkylating agents (for example cisplatin, oxaliplatin, carboplatin, cyclophosphamide, nitrogen mustards like ifosfamide, bendamustine, melphalan, chlorambucil, busulphan, temozolamide and nitrosoureas like carmustine); antimetabolites (for example gemcitabine and antifolates such as fluoropyrimidines like 5-fluorouracil and tegafur, raltitrexed, methotrexate, cytosine arabinoside, and hydroxyurea); anti-tumour antibiotics (for example anthracyclines like adriamycin, bleomycin, doxorubicin, liposomal doxorubicin, pirarubicin, daunomycin, valrubicin, epirubicin, idarubicin, mitomycin-C, dactinomycin, amrubicin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, irinotecan, topotecan and camptothecin); inhibitors of DNA repair mechanisms such as CHK kinase; DNA-dependent protein kinase inhibitors; inhibitors of poly (ADP-ribose) polymerase (PARP inhibitors, including olaparib); and Hsp90 inhibitors such as tanespimycin and retaspimycin, inhibitors of ATR kinase (such as AZD6738); and inhibitors of WEE1 kinase (such as AZD1775/MK-1775).

In some embodiments, compounds of the present disclosure can also be used for treating type 2 diabetes in combination with one or more additional therapeutic agents useful for treating type 2 diabetes, e.g., metformin, recombinant insulin, liraglutide, semaglutide, empagliflozin etc.

The pharmaceutical composition can include various amounts of the compounds of the present disclosure, depending on various factors such as the intended use and potency and selectivity of the compounds. In some embodiments, the pharmaceutical composition comprises a therapeutically effective amount of a compound of the present disclosure. In some embodiments, the pharmaceutical composition comprises a therapeutically effective amount of the compound of the present disclosure and a pharmaceutically acceptable excipient. As used herein, a therapeutically effective amount of a compound of the present disclosure is an amount effective to treat a disease or disorder as described herein, which can depend on the recipient of the treatment, the disease or disorder being treated and the severity thereof, the composition containing the compound, the time of administration, the route of administration, the duration of treatment, the compound potency, its rate of clearance and whether or not another drug is co-administered.

### Method of Treatment

Compounds of the present disclosure have various utilities. For example, compounds of the present disclosure can be used as therapeutic active substances for the treatment and/or prophylaxis of diseases or disorders that are associated with aldehyde dehydrogenase, preferably, a disease or disorder associated with aldehyde dehydrogenase isoform 1a3 (ALDH1a3), such as proliferative diseases or disorders, metabolic diseases or disorders, endothelial cell or smooth muscle cell diseases or disorders, metastasis, etc. Accordingly, some embodiments of the present disclosure are also directed to methods of using one or more compounds of the present disclosure for inhibiting ALDH enzymes such as ALDH1a3, and methods of treating or preventing various cancers, cancer metastasis, and/or other ALDH1a3-mediated diseases and disorders, such as type 2 diabetes, pulmonary arterial hypertension (PAH) and neointimal hyperplasia (NIH).

Aldehyde dehydrogenase isoform 1a3 (ALDH1a3) is an isoform/isozyme of the ALDH1a subfamily that is crucial in the biosynthesis of RA and the regulation of RA signaling, and is cell- and disease-specific. ALDH1a3 was known as ALDH6 prior to 2000, and as Raldh3 from 2000-2007 in developmental studies. In normal conditions, ALDH1a3 is only required during embryonic development and is dispensable to healthy adult mice. In adult physiology, humans with homozygous inactivating mutations in Aldh 1a3 have been described with incompletely penetrant anopthalmia and no other described pathologies. In contrast to its minor role in normal physiology, ALDH1a3 has recently been shown to be the major determinant of ALDEFLUOR™ reactivity across most cancer types and in de differentiated pancreatic islet cells. ALDEFLUOR™ activity has long been used as a marker to differentiate aggressive cancer cells from the bulk tumor despite an overlying ignorance regarding if/how ALDEFLUOR™ activity affects tumor progression.

It has been discovered that ALDEFLUOR™ activity driven by ALDH1a3 is a functional driver of cancer aggressiveness, and is critical for tumor progression, metastasis, and resistance to chemotherapy. Thus, human ALDH1a3 (UniProtKB Accession No.: P47895) is a functional driver of chemoresistant and metastatic phenotypes in cancer, including breast cancer. Accordingly, ALDH1a3 represents a potential therapeutic target in multiple pathologies, and targeting ALDH1a3 may overcome the current barrier in treating Stage 3/4 patients whose tumors are resistant to conventional forms of therapy.

### ALDH1a3 in Development and Adult Physiology; Mechanism of Action

While certain enzymes within the ALDH family have well-characterized substrate preferences, regulation, and function, most members of this family are either poorly studied or the main mechanism of action is not understood. For example, it has been shown how ALDH1L1 and ALDH1L2 function in folate metabolism by oxidizing 10-formyl-THF. Another key example of a well-characterized Aldh enzyme is ALDH3a1, which constitutes 50% of soluble corneal protein and functions to protect against UV-induced oxidative damage of the retina and cornea by oxidizing 4-hydroxynonenal. Perhaps the most studied ALDH enzyme is ALDH2, the key catalyzer of acetylaldehyde oxidation to acetic acid in liver mitochondria. ALDH2 is inhibited by ANTABUSE® (disulfiram), a therapy given to alcoholics to prevent substance abuse. On the other hand, the ALDH1a subfamily has shown broad significance across developmental biology and various pathologies, yet its mechanism of action and key regulators remain to be elucidated.

The ALDH1a sub-family is the most disease relevant group among the ALDH family, and has recently become the focus of considerable research given its importance to developmental biology and the notable ability of the ALDEFLUOR™ assay (Stem Cell Technology) to identify stem-like cells, particularly in cancer. As described herein, the ALDEFLUOR™ assay predominantly measures activity from ALDH1a3 such as in pancreatic cells from diabetic mice.

Total knockout of ALDH1a3 results in postnatal mouse death due to nasal closure defects. Importantly, this phenotype can be rescued by all-trans retinoic acid supplementation during a short window of pregnancy, resulting in normal adults. In humans, homozygous mutations in ALDH1a3 are associated with small-eye disease, but this phenotype is not fully penetrant and additional pathologies were not mentioned. Further supporting the idea that ALDH1a3 is developmentally restricted, recent studies have shown that ALDH1a3 is specifically repressed in certain developmental tissues to prevent vitamin A signaling. Additional analyses have shown ALDH1a3 is not needed for the developing ovary, and it is expressed only in the prostate and salivary gland of adults. Among the colon, liver, lung, bladder, prostate and ovary, only the ovary has a significant ALDEFLUOR™-positive population, and this small population is only partially inhibited by an ALDH1a3 inhibitor.

In metabolic disease, ALDH1a3 is a marker of failing pancreatic islet cells. Evidence shows the pancreatic beta cells do not die during the progression of Type 2 Diabetes, but rather de-differentiate into non-exocrine cells that are no longer capable of controlling blood glucose via insulin secretion. The FOXO1 transcription factor suppresses Aldh1a3 in normal pancreatic cells, this suppression is lost during progression to Type 2 Diabetes. Studies on pancreatic islets extracted from clinical patients with Type 2 Diabetes have validated that dedifferentiation is observed and these cells are marked by Aldh1a3. Interventions to reduce Type 2 Diabetes progression, such as pair feeding, similarly reduce Aldh 1a3 expression. Additional research indicates that ALDH1a3 expression directly reduces insulin secretion by pancreatic islet cell clones while increasing glucagon secretion. Treatment of diabetic Otsuka Long-Evans Tokushima Fatty rats with disulfiram, a broad specificity inhibitor of Aldh1a enzymes, reduces plasma glucose and triglyceride levels while increasing insulin secretion. This suggests that ALDH1a3 can also drive the pathology of metabolic diseases such as Type 2 Diabetes.

ALDH1a3 has also been implicated in endothelial cell or smooth muscle cell diseases or disorders, for example, pulmonary arterial hypertension (PAH) and neointimal hyperplasia (NIH), see e.g., Rabinovitch, M. et al. NIH Project No. 2R01HL074186-14; Li, D. et al. Circulation 138(Supp. 1):abstract 17192 (2018); and Xie, X. et al. iScience 19:872-882 (2019).

Taken together, these results suggest that targeted inhibition of ALDH1a3 would be effective in treating various diseases described herein such as various cancer, metastasis, metabolic syndromes such as Type 2 Diabetes. These studies also suggest that Aldh1a3 can potentially be ablated without significant on-target toxicity. Data indicate that ALDH1a3 is dispensable to adult mammals. Inhibition of ALDH1a3 during pregnancy would likely be contraindicated due to loss of retinoic acid signaling.

Published research has predominantly claimed two mechanisms for the cancer-promoting effect of ALDH1a3. These are split between the detoxification of reactive oxygen species or the oxidation of retinal into bioactive retinoic acid. Multiple reasons exist for the discrepancy between the number of papers detailing the functional consequence of ALDH1a3 expression compared to its mechanism of action. Primarily, reactive oxygen species are transient and difficult to detect. Current methods use fluorescent reporters, such as DCFDA and DHE, both of which are insensitive. Furthermore, oxidative stress in in vitro conditions is not reflective of the in vivo condition. We have made attempts to induce oxidative stress in vitro with paclitaxel and detect via DCFDA, however, the detection range for this assay is smaller than the deviations inherent within the data. However, additional literature evidence does support a ROS-related mechanism: ALDH1a3 was found to detoxify 4-HNE in stallion sperm samples, which led to improved motility. In Type 2 Diabetes, disease progression occurs in some instances as a result of lipotoxicity from high-fat diets. Given the pathologic mechanism of Aldh1a3 in Type 2 Diabetes, it is possible that Aldh 1a3 is a key mediator of pancreatic beta cell dedifferentiation. ALDH1a3 was also induced by radiotherapy in head and neck squamous cell carcinoma (SCC), indicating it may respond to cellular damage.

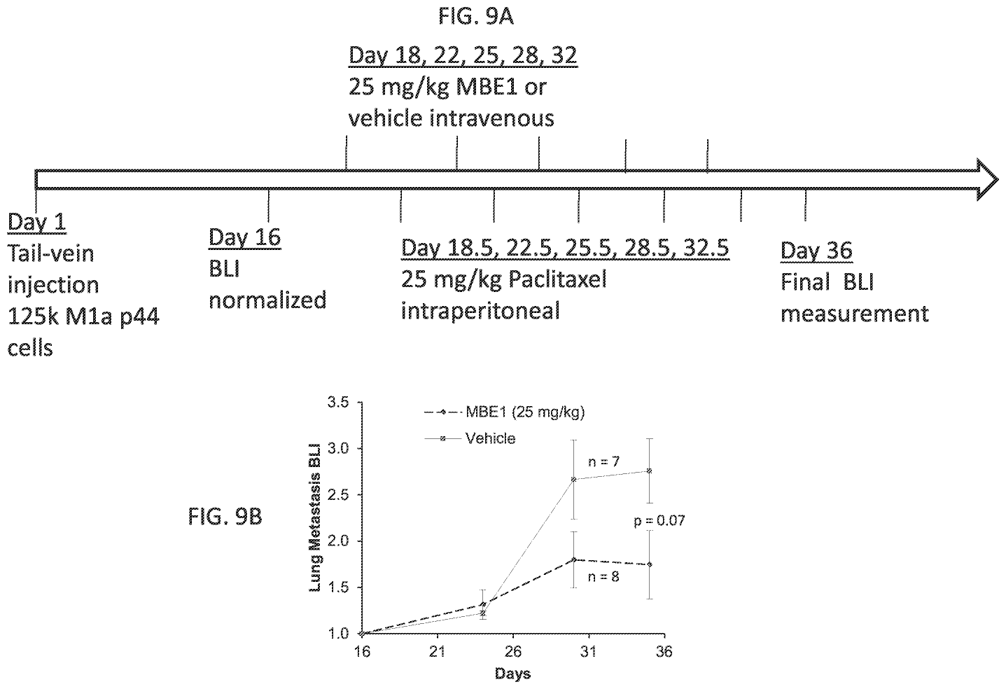

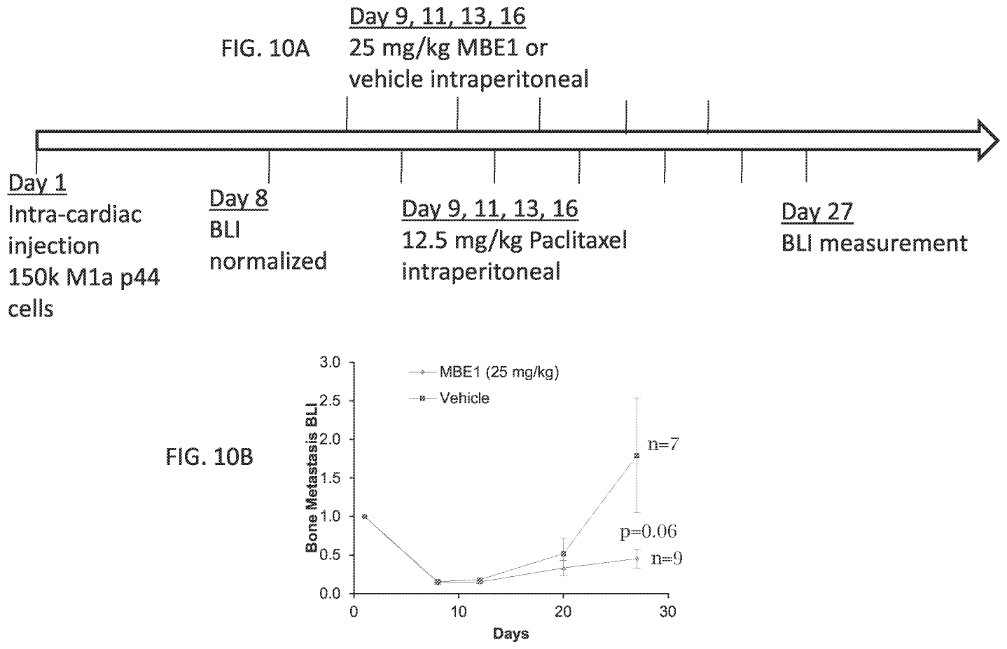

Interestingly, high profile work in melanoma has demonstrated that oxidative stress is the major determinant of metastatic dissemination. In this work, it was shown that oxidative stress is not present in a primary tumor, whereas it was dramatically induced and affected the fitness of metastatic cells. Systemic antioxidant delivery could then facilitate lung metastasis in normally non-metastatic cells. Manipulation of ALDH1a3 expression does not strongly affect primary tumor growth until challenged with an oxidative stressor, such as chemotherapy. On the other hand, it has been observed that the greatest effect of ALDH1a3 inhibition is on lung or bone metastasis, sites with high levels of oxidative stress (FIGS. 9A-10B).

Studies of retinoic acid (RA) signaling dependent on ALDH1a3 are equally difficult, as they require exogenous supplementation with retinal in tissue culture conditions and are far removed from the microenvironmental conditions of the tumor. Data showing gene correlations between each of the ALDH1a enzymes and each component of the RA signaling pathway in tumors from breast cancer patients has been developed, and demonstrates that of ALDH1a1, ALDH1a2 and ALDH1a3, ALDH1a3 shows the least correlation with components of the RA signaling pathway in breast tumors.

The functional importance of ALDH1a3 to therapeutic resistance, tumor progression and metastasis across most solid tumor types makes it an appealing target for drug discovery. Coupled with the low chance for on-target toxicity, systemic pharmacological inhibition of ALDH1a3 alone or in combination with other therapeutics (e.g., approved therapeutics) is expected to be useful for treating primary cancers, as well as indolent and overt metastatic disease.

In some embodiments, the present disclosure provides a method of inhibiting an aldehyde dehydrogenase, in particular, ALDH1a3, in a subject in need thereof. In some embodiments, the method comprises administering an effective amount of a compound of the present disclosure (e.g., a compound of Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), Formula I-P, Formula II (e.g., Formula II-1, II-2, II-3, or II-4,), Formula II-P, Formula III (e.g., Formula III-1 or III-2), or any of Compound Nos. 1-138, or a pharmaceutically acceptable salt thereof) or an effective amount of a pharmaceutical composition described herein. In some embodiments, the subject suffers from a disease or disorder associated with aldehyde dehydrogenase, preferably, a disease or disorder associated with aldehyde dehydrogenase isoform 1a3 (ALDH1a3) in a subject in need thereof. For example, in some embodiments, the subject suffers from a proliferative disease such as cancer (e.g., as described herein). In some embodiments, the subject suffers from a metabolic disease such as type 2 diabetes. In some embodiments, the subject suffers from an endothelial cell or smooth muscle cell disease or disorder, such as pulmonary arterial hypertension or neointimal hyperplasia.

In some embodiments, the present disclosure also provides a method of treating a disease or disorder associated with aldehyde dehydrogenase, preferably, a disease or disorder associated with aldehyde dehydrogenase isoform 1a3 (ALDH1a3) in a subject in need thereof. In some embodiments, the method comprises administering an effective amount of a compound of the present disclosure (e.g., a compound of Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), Formula I-P, Formula II (e.g., Formula II-1, II-2, II-3, or II-4,), Formula II-P, Formula III (e.g., Formula III-1 or III-2), or any of Compound Nos. 1-138, or a pharmaceutically acceptable salt thereof) or an effective amount of a pharmaceutical composition described herein. In some embodiments, the disease or disorder is associated with aldehyde dehydrogenase isoform 1a3 (ALDH1a3) in the subject. For example, in some embodiments, the disease or disorder is a proliferative disease such as cancer (e.g., as described herein) associated with aldehyde dehydrogenase isoform 1a3 (ALDH1a3). In some embodiments, the disease or disorder is a metabolic disease, such as type 2 diabetes, associated with aldehyde dehydrogenase isoform 1a3 (ALDH1a3). In some embodiments, the disease or disorder is an endothelial cell or smooth muscle cell disease or disorder, such as pulmonary arterial hypertension or neointimal hyperplasia, associated with aldehyde dehydrogenase isoform 1a3 (ALDH1a3).

In some embodiments, the present disclosure provides a method of treating cancer in a subject in need thereof. In some embodiments, the method comprises administering to the subject a therapeutically effective amount of a compound of the present disclosure (e.g., a compound of Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), Formula I-P, Formula II (e.g., Formula II-1, II-2, II-3, or II-4,), Formula II-P, Formula III (e.g., Formula III-1 or III-2), or any of Compound Nos. 1-138, or a pharmaceutically acceptable salt thereof) or a therapeutically effective amount of a pharmaceutical composition described herein.

The methods herein are not particularly limited to any specific cancer type. As shown in the Examples section, many cancer types were shown to have ALDH1a3 activities which can be inhibited by representative compounds of the present disclosure. In some embodiments, the cancer is a solid cancer. In some embodiments, the cancer is metastatic cancer or chemoresistant cancer. In some embodiments, the cancer can be a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, and/or sarcoma. In some embodiments, the cancer is breast caner (e.g., (e.g., ER negative breast cancer, triple negative breast cancer, basal-like breast cancers, or HER2-positive breast cancers), clear cell renal cell cancer, gastric cancer, bladder cancer, ovarian cancer, squamous cell lung cancer, colorectal cancer or glioma (e.g., low-grade glioma) cancer. In some embodiments, the cancer can also be any of those described as treatable with an ALDH1a3 inhibitor in PCT/US2019/044278, which has an international filing date of Jul. 31, 2019, the content of which is incorporated by reference in its entirety.

In some embodiments, the cancer has established metastasis. In some embodiments, the cancer has not metastasized prior to treatment with the methods herein, and the method comprises administering an effective amount of one or more compounds of the present disclosure to delay or prevent metastasis of the cancer. In any of the embodiments described herein, the cancer is associated with ALDH1a3 activites, such as having higher expression level compared to a control, and/or having cancer cells with ALDH1a3 activities, e.g., positive in Aldefluor™ assay, which can be reduced with an ALDH1a3 inhibitor or genetic knockout or knockdown. In some embodiments, the method further comprises administering to the subject an effective amount of a second anti-cancer therapy, such as a chemotherapeutic agent (e.g., described herein, such as paclitaxel) or a therapeutic antibody.

In some embodiments, the present disclosure provides a method of treating metastatic cancer in a subject in need thereof, the method comprising administering to the subject a therapeutically effective amount of a compound of the present disclosure (e.g., a compound of Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), Formula I-P, Formula II (e.g., Formula II-1, II-2, II-3, or II-4,), Formula II-P, Formula III (e.g., Formula III-1 or III-2), or any of Compound Nos. 1-138, or a pharmaceutically acceptable salt thereof) or a therapeutically effective amount of a pharmaceutical composition described herein. In some embodiments, the metastatic cancer is a solid cancer. In some embodiments, the metastatic cancer can be a metastatic breast cancer, metastatic colorectal cancer, metastatic kidney cancer, metastatic ovarian cancer, metastatic gastric cancer, metastatic thyroid cancer, metastatic testicular cancer, metastatic cervical cancer, metastatic nasopharyngeal cancer, metastatic esophageal cancer, metastatic bile duct cancer, metastatic lung cancer, metastatic pancreatic cancer, metastatic prostate cancer, metastatic bone cancer, metastatic blood cancer, metastatic brain cancer, metastatic liver cancer, metastatic mesothelioma, metastatic melanoma, and/or metastatic sarcoma. In some embodiments, the cancer is metastatic breast (e.g., ER negative breast cancer, triple negative breast cancer, basal-like breast cancers, or HER2-positive breast cancers), clear cell renal cell, gastric, bladder, ovarian, squamous cell lung, colorectal or glioma (e.g., low-grade glioma) cancer. In some embodiments, the metastatic cancer is associated with ALDH1a3 activites. In some embodiments, the metastatic cancer can be breast cancer with established lung metastasis, colorectal metastasis, and/or bone metastasis. In some embodiments, the method further comprises administering to the subject an effective amount of a second anti-cancer therapy, such as a chemotherapeutic agent (e.g., described herein, such as paclitaxel) or a therapeutic antibody.

In some embodiments, the present disclosure provides a method of treating chemoresistant cancer in a subject in need thereof, the method comprising administering to the subject a therapeutically effective amount of a compound of the present disclosure (e.g., a compound of Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), Formula I-P, Formula II (e.g., Formula II-1, II-2, II-3, or II-4,), Formula II-P, Formula III (e.g., Formula III-1 or III-2), or any of Compound Nos. 1-138, or a pharmaceutically acceptable salt thereof) or a therapeutically effective amount of a pharmaceutical composition described herein. “Chemoresistant cancer,” as used herein, refers to a cancer that does not respond to treatment with one or more chemotherapeutic agents. “Chemoresistant cancers” include those that are non-responsive to treatment with one or more therapeutic agents at the beginning of treatment, and those that become non-responsive to treatment with one or more therapeutic agents during treatment. Chemoresistant cancers that are particularly suitable for treatment using the methods described herein include, but are not limited to, cancers that are resistant to treatment with paclitaxel and/or doxorubicin. In some embodiments, the chemoresistant cancer is a solid cancer. In some embodiments, the chemoresistant cancer can be a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, and/or sarcoma. In some embodiments, the cancer can be a breast (e.g., triple negative breast), clear cell renal cell, gastric, bladder, ovarian, squamous cell lung, colorectal or glioma (e.g., low-grade glioma) cancer. In some embodiments, the chemoresistant cancer is associated with ALDH1a3 activites. In some embodiments, the method further comprises administering to the subject an effective amount of a second anti cancer therapy, such as a chemotherapeutic agent (e.g., described herein, such as paclitaxel) or a therapeutic antibody.

In some embodiments, the present disclosure provides a method of sensitizing cancer for chemotherapy in a subject in need thereof, the method comprising administering to the subject an effective amount of a compound of the present disclosure (e.g., a compound of Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), Formula I-P, Formula II (e.g., Formula II-1, II-2, II-3, or II-4,), Formula II-P, Formula III (e.g., Formula III-1 or III-2), or any of Compound Nos. 1-138, or a pharmaceutically acceptable salt thereof) or an effective amount of a pharmaceutical composition described herein. Typically, the method can cause the cancer more responsive to treatment with chemotherapeutic agent. In some embodiments, the cancer is a solid cancer. In some embodiments, the cancer can be a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, and/or sarcoma. In some embodiments, the cancer is associated with ALDH1a3 activites. In some embodiments, the method further comprises administering to the subject an effective amount of a second anti-cancer therapy, such as a chemotherapeutic agent (e.g., described herein, such as paclitaxel) or a therapeutic antibody.

In some embodiments, the present disclosure provides a method of treating or preventing metastasis of a cancer in a subject in need thereof, the method comprising administering to the subject an effective amount of a compound of the present disclosure (e.g., a compound of Formula I (e.g., Formula I-O, I-F, I-1, I-2, I-1-A, I-2-A, I-1-A 1, I-1-A2, I-1-A3, I-2-A1, I-2-A2, I-2-A3, I-1-B, I-2-B, I-1-C, or I-2-C), Formula I-P, Formula II (e.g., Formula II-1, II-2, II-3, or II-4,), Formula II-P, Formula III (e.g., Formula III-1 or III-2), or any of Compound Nos. 1-138, or a pharmaceutically acceptable salt thereof) or an effective amount of a pharmaceutical composition described herein. In some embodiments, the cancer is a solid cancer. In some embodiments, the cancer can be a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, and/or sarcoma. In some embodiments, the cancer is associated with ALDH1a3 activites. In some embodiments, the cancer has established metastasis. In some embodiments, the cancer has not metastasized prior to treatment with the methods herein, and the method delays or prevents metastasis of the cancer. In some embodiments, the method further comprises administering to the subject an effective amount of a second anti-cancer therapy, such as a chemotherapeutic agent (e.g., described herein, such as paclitaxel) or a therapeutic antibody.

In some embodiments, the present disclosure provides a method of treating or preventing a metabolic disease, such as Type 2 Diabetes, in a subject in need thereof, the method comprising administering to the subject a therapeutically effective amount of a compound of the present disclosure (e.g., a compound of Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), Formula I-P, Formula II (e.g., Formula II-1, II-2, II-3, or II-4,), Formula II-P, Formula III (e.g., Formula III-1 or III-2), or any of Compound Nos. 1-138, or a pharmaceutically acceptable salt thereof) or a therapeutically effective amount of a pharmaceutical composition described herein. As discussed herein, metabolic diseases such as type 2 diabetes are associated with a pathology driven by ALDH1a3 activities. In some embodiments, the method further comprises administering to the subject an effective amount of an additional anti-metabolic diseases agents, such as anti-type 2 diabetes agent. Suitable additional anti-metabolic diseases agents include without limitation an incretin mimic, recombinant insulin, a biguanide, SGLT2 inhibitors, a therapeutic antibody, etc. Any of the known Type 2 Diabetes treatments can be used in combination with the compounds of the present disclosure, for example, for treating Type 2 Diabetes (e.g., described herein) or treating or preventing other metabolic syndromes.

In some embodiments, the present disclosure provides a method of treating an endothelial cell or smooth muscle cell disease or disorder, such as pulmonary arterial hypertension or neointimal hyperplasia, in a subject in need thereof, the method comprising administering to the subject a therapeutically effective amount of a compound of the present disclosure (e.g., a compound of Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), Formula I-P, Formula II (e.g., Formula II-1, II-2, II-3, or II-4,), Formula II-P, Formula III (e.g., Formula III-1 or III-2), or any of Compound Nos. 1-138, or a pharmaceutically acceptable salt thereof) or a therapeutically effective amount of a pharmaceutical composition described herein. In some embodiments, the endothelial cell or smooth muscle cell disease or disorder is associated with a pathology driven by ALDH1a3 activities. In some embodiments, the endothelial cell or smooth muscle cell disease or disorder is pulmonary arterial hypertension. In some embodiments, the endothelial cell or smooth muscle cell disease or disorder is neointimal hyperplasia.

Also provided herein is a method of inhibiting the proliferation of a cancer cell (e.g., a metastatic cancer cell, a chemoresistant cancer cell). The method comprises administering to the cell (e.g., an effective amount of) one or more compounds of the present disclosure. In a particular embodiment, the cancer cell is a breast cancer cell (e.g., a basal-like breast cancer cell or a HER-2 positive breast cancer cell). The cell can be a cultured cell (e.g., cell line) or a cell in a subject. In a particular embodiment, the cell is present in a human subject (e.g., a human subject with a cancer).

In any of the embodiments described herein, the compound of the present disclosure recited in the methods herein can be any of the compounds having an activity level of A or B shown in Table 3 of the present disclosure. In some embodiments, the compound of the present disclosure recited in the methods herein can also be any compound of the present disclosure having an efficacy in ALDH1a3 inhibition comparable to Compound 1 or better, e.g., measured by any of the methods described herein. In some preferred embodiments, the compound of the present disclosure recited in the methods herein can be any compound of the present disclosure having an 1050 value of less than 250 nM (preferably, less than 100 nM, such as about 1-100 nM, about 10-100 nM, about 10-50 nM, about 20-100 nM, about 20-50 nM, etc.) in inhibiting hALDH1a3 when measured by the method described herein according to Biological Example 5B.

The administering in the methods herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the administering is orally.

As discussed herein, compounds of the present disclosure can be used as a monotherapy or in a combination therapy. In some embodiments according to the methods described herein, compounds of the present disclosure can be administered as the only active ingredient(s). In some embodiments according to the methods described herein, compounds of the present disclosure can be used in combination with conventional surgery or radiotherapy, immunotherapy, cell therapy, therapeutic antibodies, or chemotherapy. In some embodiments, compounds of the present disclosure can be used in combination with, either concurrently or sequentially in any order, a chemotherapy (e.g., paclitaxel, doxorubicin, tamoxifen, cisplatin, mitomycin, 5-fluorouracil, sorafenib, octreotide, dacarbazine (DTIC), cis-platinum, cimetidine, cyclophosphamide), radiation therapy (e.g., proton beam therapy), hormone therapy (e.g., anti-estrogen therapy, androgen deprivation therapy (ADD, luteinizing hormone-releasing hormone (LH-RH) agonists, aromatase inhibitors (Ads, such as anastrozole, exemestane, letrozole), estrogen receptor modulators (e.g., tamoxifen, raloxifene, toremifene), or biological therapy. In some embodiments according to the methods described herein, compounds of the present disclosure can be used in combination with conventional treatments, SGLT inhibitors, cell therapy, therapeutic antibodies, or incretin analogues.

In some embodiments according to the methods described herein, compounds of the present disclosure can also be co-administered with an additional pharmaceutically active compound, either concurrently or sequentially in any order, to the subject in need thereof. In some embodiments, the additional pharmaceutically active compound can be a chemotherapeutic agent, a therapeutic antibody, etc. Any of the known chemotherapeutics, immunotherapy, cell therapy, or therapeutic antibodies can be used in combination with the compounds of the present disclosure, for example, for treating cancer (e.g., described herein) or treating or preventing metastasis. Some examples of such additional pharmaceutically active compounds, such as chemotherapeutics, are exemplified herein, which in dude for example, DNA alkylating agents (for example cisplatin, oxaliplatin, carboplatin, cyclophosphamide, nitrogen mustards like ifosfamide, bendamustine, melphalan, chlorambucil, busulphan, temozolamide and nitrosoureas like carmustine); antimetabolites (for example gemcitabine and antifolates such as fluoropyrimidines like 5-fluorouracil and tegafur, raltitrexed, methotrexate, cytosine arabinoside, and hydroxyurea); anti-tumour antibiotics (for example anthracyclines like adriamycin, bleomycin, doxorubicin, liposomal doxorubicin, pirarubicin, daunomycin, valrubicin, epinibicin, idarubicin, mitomycin-C, dactinomycin, amrubicin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, irinotecan, topotecan and camptothecin); inhibitors of DNA repair mechanisms such as CHK kinase; DNA-dependent protein kinase inhibitors; inhibitors of poly (ADP-ribose) polymerase (PARP inhibitors, including olaparib); and Hsp90 inhibitors such as tanespimycin and retaspimycin, inhibitors of ATR kinase (such as AZD6738); and inhibitors of WEE1 kinase (such as AZD1775/MK-1775). In some embodiments, the additional pharmaceutically active compound can be an incretin mimic, recombinant insulin, a biguanide, a therapeutic antibody, etc. Any of the known Type 2 Diabetes treatments can be used in combination with the compounds of the present disclosure, for example, for treating Type 2 Diabetes (e.g., described herein) or treating or preventing other metabolic syndromes.

Dosing regimen including doses for the methods described herein can vary and be adjusted, which can depend on the recipient of the treatment, the disease or disorder being treated and the severity thereof, the composition containing the compound, the time of administration, the route of administration, the duration of treatment, the compound potency, its rate of clearance and whether or not another drug is co-administered.

### Definitions

It is meant to be understood that proper valences are maintained for all moieties and combinations thereof.

It is also meant to be understood that a specific embodiment of a variable moiety herein can be the same or different as another specific embodiment having the same identifier.

Suitable groups for in compounds of Formula I, II, I-P, II-P, III, or subformula thereof, as applicable, are independently selected. The described embodiments of the present disclosure can be combined. Such combination is contemplated and within the scope of the present disclosure. For example, it is contemplated that the definition(s) of any one or more of R¹, R², R³, R⁴, R⁵, J¹, J², J³, Z, X, and n of Formula I can be combined with the definition of any one or more of the other(s) of R¹, R², R³, R⁴, R⁵, J¹, J², J³, Z, X, and n of Formula I, as applicable, and the resulted compounds from the combination are within the scope of the present disclosure. Combinations of other variables for other Formulae should be understood similarly.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The disclosure additionally encompasses compounds described herein as individual isomers substantially free of other isomers, and alternatively, as mixtures of various isomers including racemic mixtures.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

As used herein, the term “compound(s) of the present disclosure” refers to any of the compounds described herein according to Formula I (e.g., Formula I-O, I-F, I-1, I-2, I-1-A, I-2-A, I-1-A1, I-1-A2, I-1-A3, I-2-A1, I-2-A2, I-2-A3, I-1-B, I-2-B, I-1-C, or I-2-C), Formula I-P, Formula II (e.g., Formula II-1, II-2, II-3, or II-4,), Formula II-P, Formula III (e.g., Formula III-1 or III-2), or any of Compound Nos. 1-138, isotopically labeled compound(s) thereof (such as a deuterated analog wherein one or more of the hydrogen atoms is/are substituted with a deuterium atom with an abundance above its natural abundance), possible stereoisomers thereof (including diastereoisomers, enantiomers, and racemic mixtures), tautomers thereof, conformational isomers thereof, and/or possible pharmaceutically acceptable salts thereof (e.g., acid addition salt such as HCl salt or base addition salt such as Na salt). Hydrates and solvates of the compounds of the present disclosure are considered compositions of the present disclosure, wherein the compound(s) is in association with water or solvent, respectively.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention.

As used herein, the phrase “administration” of a compound, “administering” a compound, or other variants thereof means providing the compound or a prodrug of the compound to the individual in need of treatment.

As used herein, the term “alkyl” as used by itself or as part of another group refers to a straight- or branched-chain aliphatic saturated hydrocarbon. In some embodiments, the alkyl which can include one to twelve carbon atoms (i.e., C₁₋₁₂ alkyl) or the number of carbon atoms designated. In one embodiment, the alkyl group is a straight chain C₁₋₁₀alkyl group. In another embodiment, the alkyl group is a branched chain C₃₋₁₀ alkyl group. In another embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. For example, a C₁₋₄ alkyl group as used herein refers to a group selected from methyl, ethyl, propyl (n-propyl), isopropyl, butyl (n-butyl), sec-butyl, tert-butyl, and iso-butyl. An optionally substituted C₁₋₄ alkyl group refers to the C₁₋₄alkyl group as defined, optionally substituted with one or more permissible substituents as described herein.

As used herein, the teen “alkenyl” as used by itself or as part of another group refers to a straight- or branched-chain aliphatic hydrocarbon containing one or more, for example, one, two or three carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl.

As used herein, the teen “alkynyl” as used by itself or as part of another group refers to a straight- or branched-chain aliphatic hydrocarbon containing one or more, for example, one to three carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups.

As used herein, the term “alkoxy” as used by itself or as part of another group refers to a radical of the formula OR^{a1}, wherein R^{a1} is an alkyl.

As used herein, the teen “cycloalkoxy” as used by itself or as part of another group refers to a radical of the formula OR^{a1}, wherein R^{a1} is a cycloalkyl.

As used herein, the term “haloalkyl” as used by itself or as part of another group refers to an alkyl substituted with one or more fluorine, chlorine, bromine and/or iodine atoms. In preferred embodiments, the haloalkyl is an alkyl group substituted with one, two, or three fluorine atoms. In one embodiment, the haloalkyl group is a C₁₋₁₀ haloalkyl group. In one embodiment, the haloalkyl group is a C₁₋₆ haloalkyl group. In one embodiment, the haloalkyl group is a C₁₋₄ haloalkyl group.

“Carbocyclyl” or “carbocyclic” as used by itself or as part of another group refers to a radical of a non-aromatic cyclic hydrocarbon group having from 3 to 10 ring carbon atoms (“C₃₋₁₀ carbocyclyl”) and zero heteroatoms in the non-aromatic ring system. The carbocyclyl group can be either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Non-limiting exemplary carbocyclyl groups include cyclopropyl, cyclobutyl, cyclopentyl, cyclohexyl, cycloheptyl, cyclooctyl, norbornyl, decalin, adamantyl, cyclopentenyl, and cyclohexenyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₈ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”).

“Heterocyclyl” or “heterocyclic” as used by itself or as part of another group refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is on the heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system.

Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” as used by itself or as part of another group refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Aralkyl” as used by itself or as part of another group refers to an alkyl substituted with one or more aryl groups, preferably, substituted with one aryl group. Examples of aralkyl include benzyl, phenethyl, etc. When an aralkyl is said to be optionally substituted, either the alkyl portion or the aryl portion of the aralkyl can be optionally substituted.

“Heteroaryl” as used by itself or as part of another group refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, and the like) the point of attachment can be on either ring, i.e., either the ring bearing a heteroatom (e.g., 2-indolyl) or the ring that does not contain a heteroatom (e.g., 5-indolyl).

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” as used by itself or as part of another group refers to an alkyl substituted with one or more heteroaryl groups, preferably, substituted with one heteroaryl group. When a heteroaralkyl is said to be optionally substituted, either the alkyl portion or the heteroaryl portion of the heteroaralkyl can be optionally substituted.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable. Two of the optional substituents can join to form an optionally substituted cycloalkyl, heterocylyl, aryl, or heteroaryl ring. Substitution can occur on any available carbon, oxygen, or nitrogen atom, and can form a spirocycle. Typically, substitution herein does not result in an O—O, O—N, S—S, S—N (except SO₂—N bond), heteroatom-halogen, or —C(O)—S bond or three or more consecutive heteroatoms, with the exception of O—SO₂—O, O—SO₂—N, and N—SO₂—N, except that some of such bonds or connections may be allowed if in a stable aromatic system.

In a broad aspect, the permissible substituents herein include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a cycloalkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aryl, or a heteroaryl, each of which can be substituted, if appropriate.

Exemplary substituents include, but not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O—C(O)-alkyl, —O—C(O)-aryl, —O—C(O)-cycloalkyl, —C(—N—CN) NH₂, —C(—NH)—NH₂, —C(—NH)—NH(alkyl), —N(Y₁)(Y₂), -alkylene-N(Y₁)(Y₂), —C(O)N(Y₁)(Y₂) and —S(O)₂N(Y₁)(Y₂), wherein Y₁ and Y₂ can be the same or different and are independently selected from the group consisting of hydrogen, alkyl, aryl, cycloalkyl, and -alkylene-aryl.

Some examples of suitable substituents include, but not limited to, (C₁-C₈)alkyl groups, (C₂-C₈)alkenyl groups, (C₂-C₈)alkynyl groups, (C₃-C₁₀)cycloalkyl groups, halogen (F, Cl, Br or I), halogenated (C₁-C₈)alkyl groups (for example but not limited to —CF₃), —O—(C₁-C₈)alkyl groups, —OH, —S—(C₁-C₈)alkyl groups, —SH, —NH(C₁-C₈)alkyl groups, —N((C₁-C₈)alkyl)₂ groups, —NH₂, —C(O)NH₂, —C(O)NH(C₁-C₈)alkyl groups, —C(O)N((C₁-C₈)alkyl)₂, NHC(O)H, —NHC(O) (C₁-C₈)alkyl groups, —NHC(O) (C₃-C₈)cycloalkyl groups, —N((C₁-C₈)alkyl)C(O)H, N((C₁-C₈)alkyl)C(O)(C₁-C₈)alkyl groups, NHC(O)NH₂, NHC(O)NH(C₁-C₈)alkyl groups, N((C₁-C₈)alkyl)C(O)NH₂ groups, —NHC(O)N((C₁-C₈)alkyl)₂ groups, N((C₁-C₈)alkyl)C(O)N((C₁-C₈)alkyl)₂ groups, N((C₁-C₈)alkyl)C(O)NH((C₁-C₈)alkyl), —C(O)H, —C(O)(C₁-C₈)alkyl groups, —CN, —NO₂, —S(O)(C₁-C₈)alkyl groups, —S(O)₂(C₁-C₈)alkyl groups, —S(O)₂N((C₁-C₈)alkyl)₂ groups, —S(O)₂NH(C₁-C₈)alkyl groups, —S(O)₂NH(C₃-C₈)cycloalkyl groups, —S(O)₂NH₂ groups, —NHS(O)₂(C₁-C₈)alkyl groups, N((C₁-C₈)alkyl)S(O)₂(C₁-C₈)alkyl groups, —(C₁-C₈)alkyl-O—(C₁-C₈)alkyl groups, —O—(C₁-C₈)alkyl-O—(C₁-C₈)alkyl groups, —C(O)OH, —C(O)O(C₁-C₈)alkyl groups, NHOH, NHO(C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₈)alkyl groups (for example but not limited to —SCF₃), —(C₁-C₆) heterocycle (for example but not limited to pyrrolidine, tetrahydrofuran, pyran or morpholine), —(C₁-C₆) heteroaryl (for example but not limited to tetrazole, imidazole, furan, pyrazine or pyrazole), -phenyl, —NHC(O)O—(C₁-C₆)alkyl groups, —N((C₁-C₆)alkyl)C(O)O—(C₁-C₆)alkyl groups, —C(═NH)—(C₁-C₆)alkyl groups, —C(═NOH)—(C₁-C₆)alkyl groups, or —C(═N—O—(C₁-C₆)alkyl)-(C₁-C₆)alkyl groups.

Exemplary carbon atom substituents include, but are not limited to, halogen, —CN, —NO₂, —N₃, hydroxyl, alkoxy, cycloalkoxy, aryloxy, amino, monoalkyl amino, dialkyl amino, amide, sulfonamide, thiol, acyl, carboxylic acid, ester, sulfone, sulfoxide, alkyl, haloalkyl, alkenyl, alkynyl, C₃₋₁₀ carbocyclyl, C₆₋₁₀ aryl, 3-10 membered heterocyclyl, 5-10 membered heteroaryl, etc. For example, exemplary carbon atom substituents can include F, Cl, —CN, —SO₂H, —SO₃H, —OH, —OC₁₋₆ alkyl, —NH₂, N(C₁₋₆ alkyl)₂, —NH(C₁₋₆ alkyl), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal substituents can be joined to form ═O.

Nitrogen atoms can be substituted or unsubstituted as valency permits, and include primary, secondary, tertiary, and quaternary nitrogen atoms. Exemplary nitrogen atom substituents include, but are not limited to, hydrogen, acyl groups, esters, sulfone, sulfoxide, C₁₋₁₀ alkyl, C₁₋₁₀ haloalkyl, C₂₋₁₀ alkenyl, C₂₋₁₀alkynyl, C₃₋₁₀ carbocyclyl, 3-14 membered heterocyclyl, C₆₋₁₄ aryl, and 5-14 membered heteroaryl, or two substituent groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl can be further substituted as defined herein. In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, acyl groups, esters, sulfonates, C₁₋₁₀ alkyl, C₁₋₁₀ haloalkyl, C₂₋₁₀alkenyl, C₂₋₁₀alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl can be further substituted as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trymethylsilyl (TMS), triethylsilyl (TES), triisopropylsilyl (TIPS), t-butyldimethylsilyl (TBDMS), etc., those forming acetals or ketals, such as tetrahydropyranyl (THP), those forming esters such as formate, acetate, chloroacetate, dichloroacetate, trichloroacetate, trifluoroacetate, methoxyacetate, etc., those forming carbonates or sulfonates such as methanesulfonate (mesylate), benzylsulfonate, and tosylate (Ts), etc.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” alkyl, alkenyl, alkynyl, carbocyclic, cycloalkyl, alkoxy, cycloalkoxy, or heterocyclic group herein can be unsubstituted or substituted with 1, 2, 3, or 4 substituents independently selected from F, Cl, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₆ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted Cu alkoxy. In some embodiments, the “optionally substituted” aryl or heteroaryl group herein can be unsubstituted or substituted with 1, 2, 3, or 4 substituents independently selected from F, Cl, —OH, —CN, NH₂, protected amino, NH(C₁₋₄ alkyl) or a protected derivative thereof, N(C₁₋₄ alkyl((C₁₋₄ alkyl), —S(═O)(C₁₋₄ alkyl), —SO₂(C₁₋₄ alkyl), C₁₋₄ alkyl, C₂₋₆ alkenyl, C₂₋₆ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

“Halo” or “halogen” refers to fluorine (fluoro, —F), chlorine (chloro, —Cl), bromine (bromo, —Br), or iodine (iodo, —I).

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, preferably a mammal, most preferably a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein to a subject in need of such treatment.

### EXAMPLES

The various starting materials, intermediates, and compounds of the preferred embodiments can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses. Exemplary embodiments of steps for performing the synthesis of products described herein are described in greater detail infra.

The abbreviations used in the Examples section should be understood as having their ordinary meanings in the art unless specifically indicated otherwise or obviously contrary from context. The following shows a list of some of the abbreviations used in the Examples section:

### AIBN Azobisisobutyronitrile

DCM dichloromethane
DIPEA di-isopropylethylamine
DMF dimethylformamide
DPPP 1,3-bis(diphenylphosphino)propane
EA or EtOAc ethyl acetate
EDCI N-(3-Dimethylaminopropyl)-N′-ethylcarbodiimide
LAH Lithium Aliminium hydride

### NMP N-methylpyrrolidinone

PE petroleum ether
Py. pyridine
THE tetrahydrofuran
TLC thin-layer chromatography

### Example 1. Synthesis of Compound 1

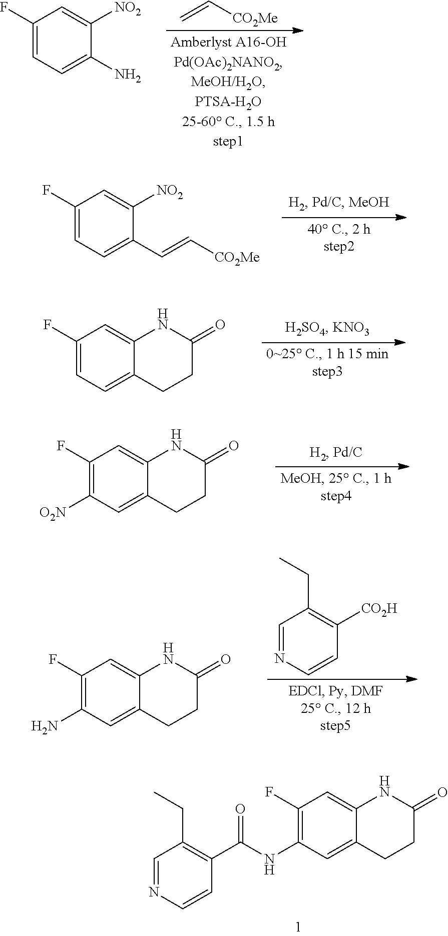

Step 1: To a stirred solution of NaNO₂ (7.41 g, 107.47 mmol, 2 eq) in water H₂O (80 mL) was added Amberlyst A26-OH (28 g). The resulting mixture was stirred at 25° C. for 0.5 h, and then polymer-supported resin was filtered and washed with water until the pH of filtrate became neutral. The polymer-supported nitrite was got. Stage 2: To a solution of 4-fluoro-1,2-dinitro-benzene (10 g, 53.74 mmol, 1 eq) and methyl prop-2-enoate (4.63 g, 53.74 mmol, 4.84 mL, 1 eq) in MeOH (100 mL) was added p-toluenesulfonic acid monohydrate (10.22 g, 53.74 mmol, 1 eq), Pd(OAc)₂ (193.02 mg, 859.77 umol, 0.016 eq) and was slowly added polymer-supported nitrite. The mixture was stirred at 60° C. for 1 hr. The reaction mixture was filtered and filtrate concentrated under reduced pressure to give a residue. The crude product methyl (E)-3-(4-fluoro-2-nitro-phenyl)prop-2-enoate (6 g, crude) was obtained as a yellow solid.

Step 2: To a solution of methyl (E)-3-(4-fluoro-2-nitro-phenyl)prop-2-enoate (6 g, 26.65 mmol, 1 eq) in MeOH (50 mL) was added 10% Pd/C (800 mg, 26.65 mmol, 1.00 eq) under H₂ atmosphere. The suspension was degassed and purged with H₂ for 3 times. The mixture was stirred under H₂ (15 Psi) at 40° C. for 1 hr. The reaction mixture was filtered and filtrate concentrated under reduced pressure to give a residue. The crude product was triturated with petroleum ether/ethyl acetate=50:1 (6 mL) at 25° C. for 10 min. Compound 7-fluoro-3,4-dihydro-1H-quinolin-2-one (3 g, 18.16 mmol, 68.17% yield) was obtained as a white solid.

Step 3: To a solution of 7-fluoro-3,4-dihydro-1H-quinolin-2-one (3 g, 18.16 mmol, 1 eq) in H₂SO₄ (20 mL) was added KNO₃ (1.84 g, 18.16 mmol, 1 eq) at 0° C. The mixture was stirred at 25° C. for 1 hr. The reaction mixture was cooled at 0° C. and the resulting solution was stirred for 15 min at 0° C. The reaction was quenched by adding 100 mL of H₂O/ice. The mixture was filtered and filter cake was concentrated under reduced pressure to give a residue. The crude product 7-fluoro-6-nitro-3,4-dihydro-1H-quinolin-2-one (2.5 g, 11.90 mmol, 65.49% yield) was obtained as a white solid.

Step 4: To a solution of 7-fluoro-6-nitro-3,4-dihydro-1H-quinolin-2-one (1.5 g, 7.14 mmol, 1 eq) in MeOH (10 mL) was added 10% Pd/C (200 mg, 7.14 mmol) under H₂ atmosphere. The suspension was degassed and purged with H₂ for 3 times. The mixture was stirred under H₂ (15 Psi) at 25° C. for 1 hr. The reaction mixture was filtered and filtrate was concentrated under reduced pressure to give a residue. The crude product was triturated with petroleum ether/ethyl acetate=10:1(11 mL) at 25° C. for 10 min. Compound 6-amino-7-fluoro-3,4-dihydro-1H-quinolin-2-one (0.8 g, 4.44 mmol, 62.21% yield) was obtained as a white solid. LCMS: (M+H)⁺: 181.4.

Step 5: To a solution of 6-amino-7-fluoro-3,4-dihydro-1H-quinolin-2-one (100 mg, 555.00 umol, 1 eq) and 3-ethylpyridine-4-carboxylic acid (83.90 mg, 555.00 umol, 1 eq) in N,N-dimethylformamide (“DMF”) (5 mL) was added 1-ethyl-3-(3-dimethylaminopropyl)carbodiimide (“EDCI”) (127.67 mg, 666.01 umol, 1.2 eq) and pyridine (“Py.”) (65.85 mg, 832.51 umol, 67.20 uL, 1.5 eq). The mixture was stirred at 25° C. for 12 hr. The reaction mixture was diluted with H₂O (10 mL) and extracted with EtOAc 15 mL (5 mL*3). The combined organic layers were washed with brine 10 mL, dried over [Na₂SO₄], filtered and concentrated under reduced pressure to give a residue. The residue was purified by prep-TLC (SiO₂, petroleum ether/ethyl acetate=0:1). Compound No. 1, 3-ethyl-N-(7-fluoro-2-oxo-3,4-dihydro-1H-quinolin-6-yl)pyridine-4-carboxamide (65 mg, 203.72 umol, 36.71% yield, 98.2% purity), was obtained as a white solid. The reported purity refers to the area % observed in the LCMS analysis. Unless otherwise specified or contrary from context, other reported purities herein should be understood similarly. LCMS: (M+H)⁺: 314.1. ¹HNMR (400 MHz, MeOD, ppm): δ 8.55 (s, 1H), 8.50 (d, J=5.2 Hz, 1H), 7.60 (d, J=8 Hz, 1H), 7.48 (d, J=4.8 Hz, 1H), 6.75 (d, J=11.211z, 1H), 2.98 (t, J=7.2 Hz, 2H), 2.88 (q, J=7.6 Hz, 2H), 2.60 (t, J=7.2 Hz, 2H), 1.29 (t, J=7.6 Hz, 3H).

### Example 2. Synthesis of Compound 18

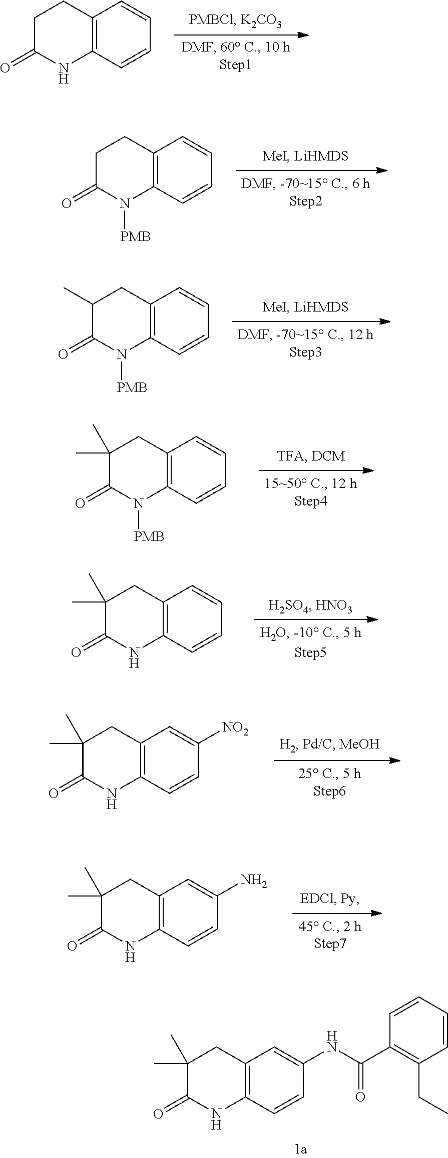

Step 1: To a mixture of 3,4-dihydro-1H-quinolin-2-one (5 g, 33.97 mmol, 1 eq) and 1-(chloromethyl)-4-methoxy-benzene (6.92 g, 44.17 mmol, 6.01 mL, 1.3 eq) in DMF (50 mL) was added K₂CO₃ (7.04 g, 50.96 mmol, 1.5 eq) under N2. The mixture was stirred at 60° C. for 10 hours. The reaction mixture was diluted with H₂O 50 mL and the mixture was cooled to 15° C. The suspension was filtered and the filtrate cake was concentrated under reduced pressure to give a residue. Compound 1-[(4-methoxyphenyl)methyl]-3,4-dihydroquinolin-2-one (6.2 g, 23.19 mmol, 68.27% yield) was obtained as a white solid. LCMS: (M+H)⁺: 268.3.

Step 2: To a mixture of 1-[(4-methoxyphenyl)methyl]-3,4-dihydroquinolin-2-one (2 g, 7.48 mmol, 1 eq) in THE (20 mL) was added LiHMDS (1 M, 8.23 mL, 1.1 eq) in one portion at −70° C. under N2. The mixture was stirred at −70° C. for 30 min. Then MeI (1.17 g, 8.23 mmol, 512.33 uL, 1.1 eq) was added. The mixture was heated to 15° C. and stirred for 5.5 hours. The reaction mixture was quenched by addition H₂O 30 mL and extracted with EtOAc 60 mL (20 mL*3). The combined organic layers were dried over Na₂SO₄, filtered and concentrated under reduced pressure to give a residue. The crude product was used into next step directly. The crude product 1-[(4-methoxyphenyl)methyl]-3-methyl-3,4-dihydroquinolin-2-one (2.1 g, crude) was obtained as yellow oil. LCMS: (M+H)⁺: 282.4.

Step 3: To a mixture of 1-[(4-methoxyphenyl)methyl]-3-methyl-3,4-dihydroquinolin-2-one (2.1 g, 7.46 mmol, 1 eq) in THE (20 mL) was added LiHMDS (1 M, 8.21 mL, 1.1 eq) in one portion at −70° C. under N2. The mixture was stirred at −70° C. for 30 min. Then MeI (1.27 g, 8.96 mmol, 557.60 uL, 1.2 eq) was added. The mixture was heated to 15° C. and stirred for 11.5 hours. The reaction mixture was quenched by addition H₂O 30 mL and extracted with EtOAc 60 mL (20 mL*3). The combined organic layers were dried over Na₂SO₄, filtered and concentrated under reduced pressure to give a residue. The residue was purified by column chromatography (SiO₂, Petroleum ether/Ethyl acetate=100/1 to 0/1). Compound 1-[(4-methoxyphenyl)methyl]-3,3-dimethyl-4H-quinolin-2-one (750 mg, 2.54 mmol, 34.02% yield) was obtained as yellow oil.

Step 4: To a mixture of 1-[(4-methoxyphenyl)methyl]-3,3-dimethyl-4H-quinolin-2-one (750 mg, 2.54 mmol, 1 eq) in dichloromethane (“DCM”) (6 mL) was added trifluoroacetic acid (“TFA”) (2 mL) in one portion at 15° C. under N2. The mixture was stirred at 50° C. for 12 hours. The reaction mixture was diluted with saturated NaHCO₃ aqueous 15 mL and extracted with EtOAc 30 mL (10 mL*3). The combined organic layers were dried over Na₂SO₄, filtered and concentrated under reduced pressure to give a residue. The residue was purified by column chromatography (SiO₂, Petroleum ether/Ethyl acetate=100/1 to 0/1). Compound 3,3-dimethyl-1,4-dihydroquinolin-2-one (300 mg, 1.71 mmol, 67.43% yield) was obtained as white solid. LCMS: (M+H)⁺: 176.5.

Step 5: To a solution of 3,3-dimethyl-1,4-dihydroquinolin-2-one (250 mg, 1.43 mmol, 1 eq) in conc. H₂SO₄ (6.6 mL) and H₂O (2.2 mL) was slowly added at −10° C. The mixture was stirred for 30 min. Then HNO₃ (179.80 mg, 2.85 mmol, 128.43 uL, 2 eq) was added and the reaction stirred at −10° C. for 4.5 h. The reaction mixture was cold at 0° C. and the resulting solution was stirred for 15 min at 0° C. Then the reaction was quenched by adding 100 mL of H₂O/ice. Then the mixture was filtered and filter cake concentrated under reduced pressure to give a residue. The crude product was triturated with petroleum ether/ethyl acetate=10:1 (11 mL). The suspension was filtered and filter cake was concentrated under reduced pressure to give a residue. Compound 3,3-dimethyl-6-nitro-1,4-dihydroquinolin-2-one (260 mg, 1.18 mmol, 82.75% yield) was obtained as a white solid.

Step 6: To a solution of 3,3-dimethyl-6-nitro-1,4-dihydroquinolin-2-one (260 mg, 1.18 mmol, 1 eq) in MeOH (5 mL) was added 10% Pd/C (100 mg). The suspension was degassed and purged with H₂ for 3 times. The mixture was stirred under H₂ (15 Psi) at 25° C. for 5 hr. The reaction mixture was filtered and filter concentrated under reduced pressure to give a residue. The crude product was triturated with petroleum ether/ethyl acetate=10:1 (11 mL). The suspension was filtered and filter cake was concentrated under reduced pressure to give a residue. Compound 6-amino-3,3-dimethyl-1,4-dihydroquinolin-2-one (200 mg, 1.05 mmol, 89.05% yield) was obtained as a white solid.

Step 7: To a solution of 6-amino-3,3-dimethyl-1,4-dihydroquinolin-2-one (83.06 mg, 436.62 umol, 1.2 eq) in pyridine (2 mL) was added EDCI (83.70 mg, 436.62 umol, 1.2 eq) and 3-ethylpyridine-4-carboxylic acid (55 mg, 363.85 umol, 1 eq). The mixture was stirred at 45° C. for 2 hr. The reaction mixture was concentrated under reduced pressure to remove pyridine. The residue was purified by prep-TLC (SiO₂, petroleum ether/ethyl acetate=0:1). Compound No. 18, N-(3,3-dimethyl-2-oxo-1,4-dihydroquinolin-6-yl)-3-ethyl-pyridine-4-carboxamide (106 mg, 319.59 umol, 87.83% yield, 97.5% purity), was obtained as a white solid. LCMS (ES, m/z): [M+H]⁺=324.2; ¹HNMR (400 MHz, MeOD, ppm): δ 8.55 (s, 1H), 8.50 (d, J=4.8 Hz, 1H), 7.54 (s, 1H), 7.46 (m, 2H), 6.87 (d, J=8.4 Hz, 1H), 2.83-2.89 (m, 4H), 1.28 (t, J=7.6 Hz, 3H), 1.17 (s, 6H).

### Example 3. Synthesis of Compound 19

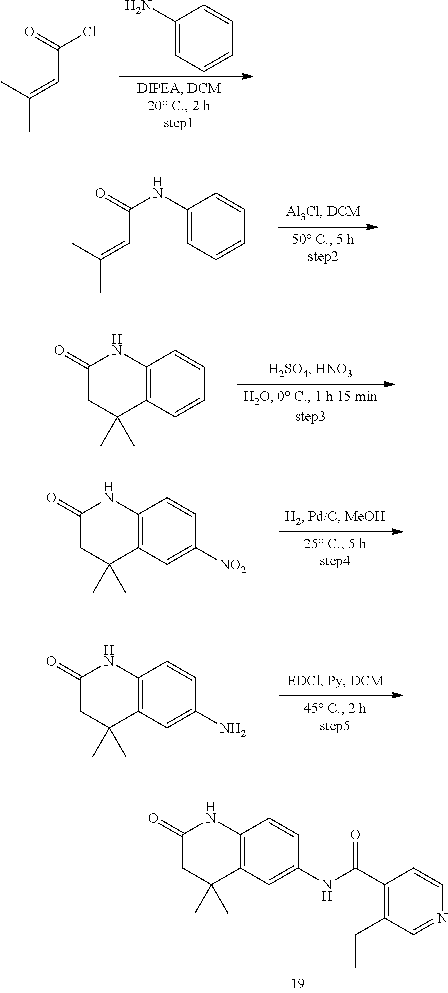

Step 1: To a solution of 3-methylbut-2-enoyl chloride (1.27 g, 10.74 mmol, 1.19 mL, 1 eq) in DCM (200 mL) was added diisopropylethylamine (2.63 g, 20.32 mmol, 3.54 mL, 1.89 eq) and aniline (1 g, 10.74 mmol, 980.39 uL, 1 eq). The mixture was stirred at 20° C. for 2 hr. Saturated sodium bicarbonate was added to quench the reaction. The organic layer was separated and washed with sat. NaHCO₃(50 mL) and water 100 mL (50 mL×2). The resulting solution was dried over Na₂SO₄ and the filtrate was evaporated. The crude product was triturated with petroleum ether/ethyl acetate=20:1 (21 mL) at 20° C. for 20 min. The mixture was filtered to get compound 3-methyl-N-phenyl-but-2-enamide (1.4 g, 7.99 mmol, 74.40% yield) as a brown solid. LCMS: (M+H)⁺: 176.5.

Step 2: To a solution of 3-methyl-N-phenyl-but-2-enamide (1.4 g, 7.99 mmol, 1 eq) in DCM (100 mL) was added AlCl₃ (1.60 g, 12.02 mmol, 656.82 uL, 1.50 eq). The mixture was stirred at 50° C. for 5 hr. The mixture was treated with 1 N HCl (20 mL) and extracted with DCM 60 mL (30 mL×2). This solution was then washed with brine 100 mL (50 mL×2) and dried over Na₂SO₄. The filtrate was evaporated. The crude product was triturated with petroleum ether/ethyl acetate=20:1 (21 mL) at 25° C. for 20 min. The mixture was filtered to get compound 4,4-dimethyl-1,3-dihydroquinolin-2-one (1.2 g, 6.85 mmol, 85.71% yield) as a brown solid.

Step 3: 4,4-dimethyl-1,3-dihydroquinolin-2-one (1.2 g, 6.85 mmol, 1 eq) was dissolved in conc. H₂SO₄ (25 mL) and H₂O (7.5 mL) at 0° C. The mixture was stirred for 10 min. Then HNO₃ (863.06 mg, 13.70 mmol, 616.47 uL, 2 eq) was added and the reaction stirred at 0° C. for 1 h. The reaction mixture was cold at 0° C. and the resulting solution was stirred for 15 min at 0° C. The reaction was quenched by adding 100 mL of H₂O/ice. The mixture was filtered and filter cake was concentrated under reduced pressure to give a residue. The crude product was triturated with petroleum ether/ethyl acetate=20:1 (21 mL) at 25° C. for 20 min. The mixture was filtered to get compound 4,4-dimethyl-6-nitro-1,3-dihydroquinolin-2-one (1 g, 4.54 mmol, 66.31% yield) as a brown solid. LCMS: (M+H)⁺: 221.4.

Step 4: To a solution of 4,4-dimethyl-6-nitro-1,3-dihydroquinolin-2-one (1 g, 4.54 mmol, 1 eq) in MeOH (10 mL) was added 10% Pd/C (200 mg) under H₂ atmosphere. The suspension was degassed and purged with H₂ for 3 times. The mixture was stirred under H₂ (15 Psi) at 25° C. for 2 hr. The reaction mixture was filtered and filter was concentrated under reduced pressure to give a residue. The crude product was triturated with petroleum ether/ethyl acetate=20:1 (42 mL) at 25° C. for 20 min. The mixture was filtered to get compound 6-amino-4,4-dimethyl-1,3-dihydroquinolin-2-one (0.8 g, 4.21 mmol, 92.61% yield) as a white solid.

Step 5: To a solution of 6-amino-4,4-dimethyl-1,3-dihydroquinolin-2-one (200 mg, 1.05 mmol, 1 eq) and 3-ethylpyridine-4-carboxylic acid (158.92 mg, 1.05 mmol, 1 eq) in pyridine (2 mL) was added EDCI (241.84 mg, 1.26 mmol, 1.2 eq). The mixture was stirred at 45° C. for 2 hr. The reaction mixture was diluted with H₂O (10 mL) and extracted with EtOAc 15 mL (5 mL*3). The combined organic layers were washed with brine 10 mL, dried over [Na₂SO₄], filtered and concentrated under reduced pressure to give a residue. The residue was purified by prep-TLC (SiO₂, petroleum ether/ethyl acetate=0:1). Compound No. 19, N-(4,4-dimethyl-2-oxo-1,3-dihydroquinolin-6-yl)-3-ethyl-pyridine-4-carboxamide (217 mg, 664.98 umol, 63.25% yield, 99.1% purity) was obtained as a white solid. LCMS (ES, m/z): [M+H]⁺=324.1; ¹HNMR (400 MHz, MeOD, ppm): δ 8.55 (s, 1H), 8.50 (d, J=5.2 Hz, 1H), 7.68 (d, J=2 Hz, 1H), 7.51 (dd, J=8.8 and 2 Hz, 1H), 7.46 (d, J=4.8 Hz, 1H), 6.90 (d, J=8.8 Hz, 1H), 2.86 (q, J=8 Hz, 2H), 2.46 (s, 2H), 1.32 (s, 6H), 1.28 (t, J=7.6 Hz, 3H).

### Example 4. Synthesis of Compound 20

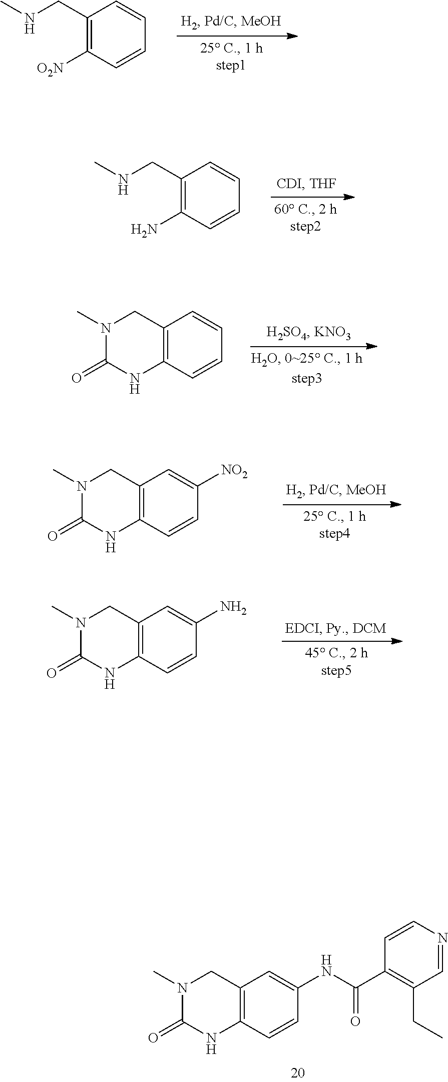

Step 1: To a solution of N-methyl-1-(2-nitrophenyl)methanamine (500 mg, 3.01 mmol, 1 eq) in MeOH (20 mL) was added 10% Pd/C (200 mg) under H₂ atmosphere. The suspension was degassed and purged with H₂ for 3 times. The mixture was stirred under H₂ (15 Psi) at 25° C. for 1 hr. The reaction mixture was filtered and filtrate concentrated under reduced pressure to give a residue. The crude product was triturated with petroleum ether/ethyl acetate=10:1 (11 mL) at 25° C. for 10 min. The mixture was filtered to get the compound 2-(methylaminomethyl)aniline (260 mg, 1.91 mmol, 63.45% yield) as a white solid.

Step 2: To a solution of 2-(methylaminomethyl)aniline (260 mg, 1.91 mmol, 1 eq) in THE (10 mL) was added CDI (174.37 mg, 1.08 mmol, 5.63e-1 eq). The mixture was stirred at 60° C. for 2 hr. The reaction mixture was diluted with H₂O (5 mL) and extracted with EtOAc 6 mL (2 mL*3). The combined organic layers were washed with brine 10 mL, dried over [Na₂SO₄], filtered and concentrated under reduced pressure to give a residue. The residue was purified by prep-TLC (SiO₂, Dichloromethane:Methanol=5/1). Compound 3-methyl-1,4-dihydroquinazolin-2-one (200 mg, 1.23 mmol, 64.59% yield) was obtained as a white solid.

Step 3: To a solution of 3-methyl-1,4-dihydroquinazolin-2-one (200 mg, 1.23 mmol, 1 eq) in conc. H₂SO₄ (8 mL) was added KNO₃ (99.74 mg, 986.51 umol, 0.8 eq) at 0° C. The mixture was stirred at 25° C. for 1 hr. The reaction mixture was cold at 0° C. and quenched by adding 10 mL of H₂O/ice. The mixture was filtered and filter cake was concentrated under reduced pressure to give a residue. The crude product was triturated with petroleum ether/ethyl acetate=10:1 (5 mL) at 25° C. for 10 min. The mixture was filtered to get the compound 3-methyl-6-nitro-1,4-dihydroquinazolin-2-one (195 mg, 941.18 umol, 76.32% yield) as a white solid. LCMS: (M+H)⁺: 208.4.

Step 4: To a solution of 3-methyl-6-nitro-1,4-dihydroquinazolin-2-one (195 mg, 941.18 umol, 1 eq) in MeOH (10 mL) was added 10% Pd/C (50 mg, 941.18 umol, 1 eq) under H₂ atmosphere. The suspension was degassed and purged with H₂ for 3 times. The mixture was stirred under H₂ (15 Psi) at 25° C. for 1 hr. The reaction mixture was filtered and filtrate concentrated under reduced pressure to give a residue. The crude product was triturated with petroleum ether/ethyl acetate=10:1 (11 mL) at 25° C. for 10 min. The mixture was filtered to get the compound 6-amino-3-methyl-1,4-dihydroquinazolin-2-one (150 mg, 846.49 umol, 89.94% yield) as a white solid.

Step 5: To a solution of 6-amino-3-methyl-1,4-dihydroquinazolin-2-one (50 mg, 282.16 umol, 1.07 eq) in pyridine (2 mL) was added EDCI (55.80 mg, 291.08 umol, 1.1 eq) and 3-ethylpyridine-4-carboxylic acid (40 mg, 264.62 umol, 1 eq). The mixture was stirred at 45° C. for 2 hr. The reaction mixture was concentrated under reduced pressure to remove pyridine The residue was purified by prep-TLC (S102, DCM:MeOH=5:1), Then product was triturated with petroleum ether/ethyl acetate=5:1 (12 mL) at 25° C. for 10 min. The mixture was filtered to get the compound No. 20, 3-ethyl-N-(3-methyl-2-oxo-1,4-dihydroquinazolin-6-yl)pyridine-4-carboxamide (43 mg, 0.14 mmol, 49% yield, 98.6% purity), as a white solid. LCMS (ES, m/z): [M+H]⁺=311.1.

### Example 5. Synthesis of Compound 23

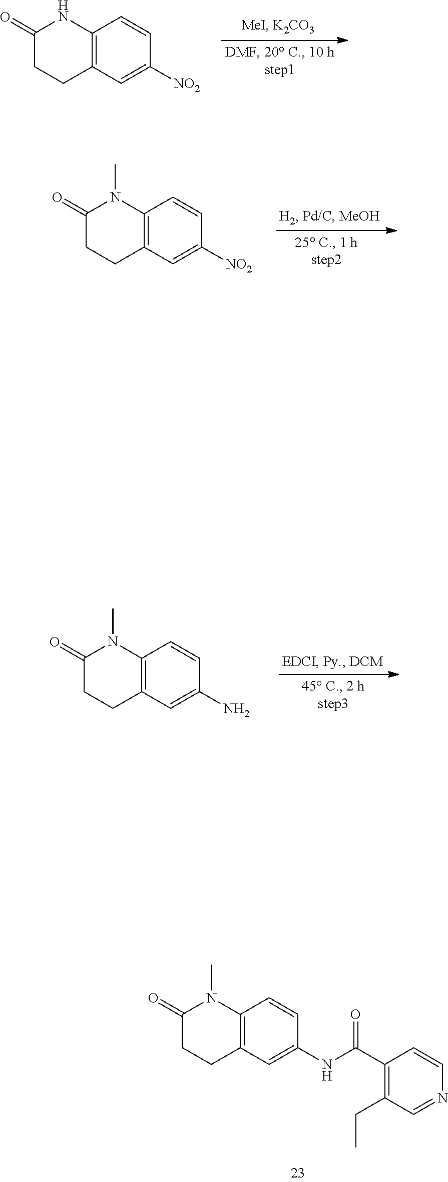 [SMILES: O=C1CCc2cc([N+](=O)[O-])ccc2N1 ; CN1C(=O)CCc2cc([N+](=O)[O-])ccc21 ; CN1C(=O)CCc2cc(N)ccc21 ; CCc1cnccc1C(=O)Nc1ccc2c(c1)CCC(=O)N2C]

Step 1: To a solution of 6-nitro-3,4-dihydro-1H-quinolin-2-one (1 g, 5.20 mmol, 1 eq) in DMF (8 mL) was added MeI (2.95 g, 20.81 mmol, 1.30 mL, 4 eq) and K₂CO₃ (863.02 mg, 6.24 mmol, 1.2 eq). The mixture was stirred at 20° C. for 10 hr. Water (20 mL) was added and the reaction mixture was extracted with EtOAc 40 mL (20 mL*2) and washed with brine (20 mL), dried over [Na₂SO₄], filtered and concentrated under reduced pressure to give a residue. The residue was purified by column chromatography (SiO₂, Petroleum ether/Ethyl acetate=10/1 to 2/1). Compound 1-methyl-6-nitro-3,4-dihydroquinolin-2-one (620 mg, 3.01 mmol, 57.78% yield) was obtained as a white solid. LCMS: (M+H)⁺: 207.4.

Step 2: To a solution of 1-methyl-6-nitro-3,4-dihydroquinolin-2-one (620 mg, 3.01 mmol, 1 eq) in MeOH (10 mL) was added 10% Pd/C (100 mg, 9.70 mmol) under H₂ atmosphere. The suspension was degassed and purged with 112 for 3 times. The mixture was stirred under H₂ (15 Psi) at 25° C. for 1 hr. The reaction mixture was filtered and filtrate was concentrated under reduced pressure to give a residue. The crude product was triturated with petroleum ether/ethyl acetate=10:1 (11 mL) at 25° C. for 10 min. The mixture was filtered to get compound 6-amino-1-methyl-3,4-dihydroquinolin-2-one (450 mg, 2.55 mmol, 84.93% yield) as a white solid. LCMS: (M+H)+: 177.5.

To a solution of 6-amino-1-methyl-3,4-dihydroquinolin-2-one (150 mg, 851.23 umol, 1.12 eq) in pyridine (4 mL) was added EDCI (175.01 mg, 912.93 umol, 1.2 eq) and 3-ethylpyridine-4-carboxylic acid (115 mg, 760.77 umol, 1 eq). The mixture was stirred at 45° C. for 2 hr. The reaction mixture was concentrated under reduced pressure to remove pyridine. The residue was purified by prep-TLC (SiO₂, DCM:MeOH=10:1). Then product was further triturated with petroleum ether/ethyl acetate=10:1 (11 mL) at 25° C. for 10 min. The mixture was filtered to get the compound No. 23, 3-ethyl-N-(1-methyl-2-oxo-3,4-dihydroquinolin-6-yl)pyridine-4-carboxamide (98.4% purity) (235 mg), as a white solid. LCMS: (M+H)⁺: 310.1. ¹HNMR (400 MHz, MeOD, ppm): δ 8.55 (s, 1H), 8.50 (d, J=4.8 Hz, 1H), 7.57-7.59 (m, 2H), 7.46 (d, J=5.2 Hz, 1H), 7.14 (d, J 9.2 Hz, 1H), 3.31 (s, 3H), 2.94 (t, J=7.2 Hz, 2H), 2.86 (q, J=7.6 Hz, 2H), 2.64 (t, J=7.2 Hz, 2H), 1.28 (t, J=7.6 Hz, 3H).

### Example 6. Synthesis of Compound 25

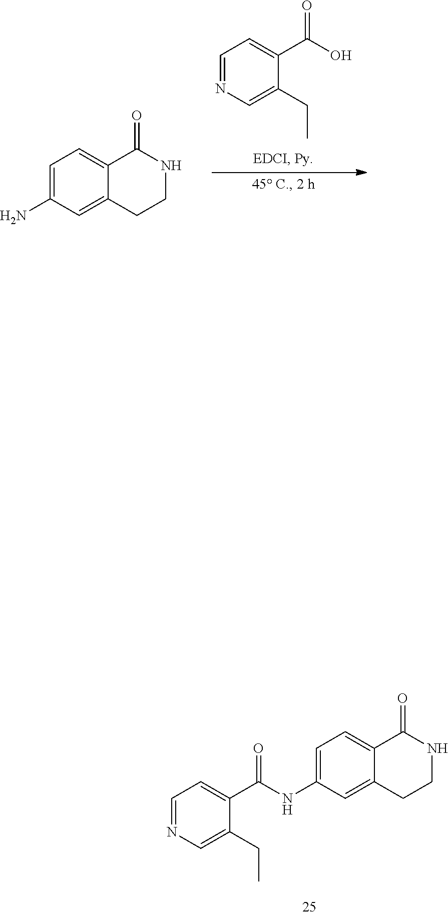 [SMILES: Nc1ccc2c(c1)CCNC2=O ; CCc1cnccc1C(=O)O ; CCc1cnccc1C(=O)Nc1ccc2c(c1)CCNC2=O]

To a solution of 6-amino-3,4-dihydro-2H-isoquinolin-1-one (100 mg, 616.57 umol, 1.17 eq) in pyridine (3 mL) was added EDCI (121.75 mg, 635.08 umol, 1.2 eq) and 3-ethylpyridine-4-carboxylic acid (80 mg, 529.23 umol, 1 eq). The mixture was stirred at 45° C. for 2 hr. The reaction mixture was concentrated under reduced pressure to remove pyridine. The residue was purified by prep-TLC (SiO₂, DCM:MeOH=10:1). Then product was triturated with petroleum ether/ethyl acetate=10:1 (11 mL) at 25° C. for 10 min. The mixture was filtered to get the compound No. 25, 3-ethyl-N-(1-oxo-3,4-dihydro-2H-isoquinolin-6-yl)pyridine-4-carboxamide (155 mg, 0.79 mmol, 85% yield, 99% purity), as a white solid. LCMS: (M+H)⁺: 296.1. ¹HNMR (400 MHz, MeOD, ppm): δ 8.56 (s, 1H), 8.52 (d, J=4.8 Hz, 1H), 7.93 (d, J=7.6 Hz, 1H), 7.92 (s, 1H), 7.59 (d, J=6.8 Hz, 1H), 7.48 (d, J=5.2 Hz, 1H), 3.52 (t, J=6.8 Hz, 2H), 3.00 (t, J=6.4 HzHz, 2H), 2.85 (q, J=7.2 Hz, 2H), 1.28 (t, J=7.6 Hz, 3H).

### Example 7. Synthesis of Compound 26

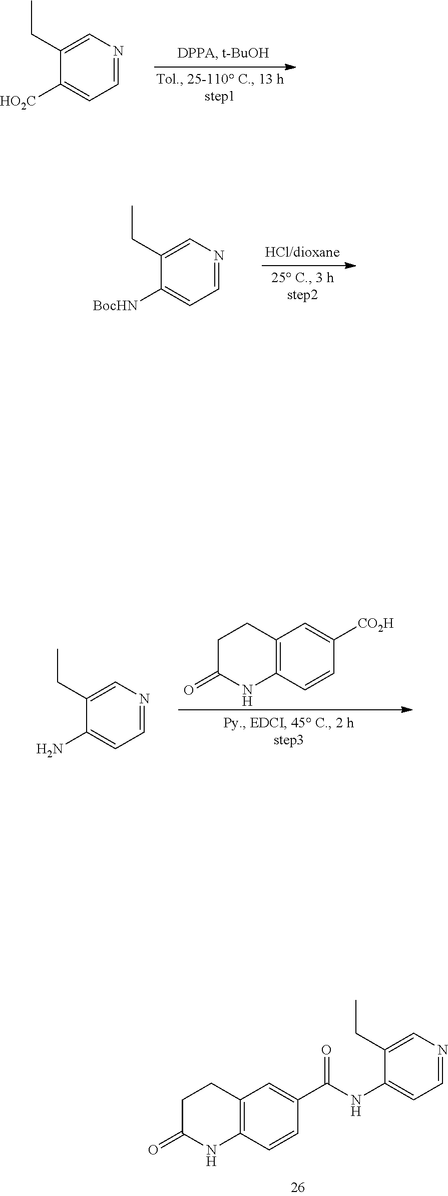

Step 1: To a solution of 3-ethylpyridine-4-carboxylic acid (100 mg, 661.54 umol, 1 eq) in toluene (“Tol.”) (5 mL) was added diphenyl phosphoryl azide (“DPPA”) (218.47 mg, 793.85 umol, 172.02 uL, 1.2 eq) and triethyl amine (“TEA”) (100.41 mg, 992.31 umol, 138.12 uL, 1.5 eq) at 25° C. After addition, the mixture was stirred at this temperature for 1 hr, and then t-BuOH (980.69 mg, 13.23 mmol, 1.27 mL, 20 eq) was added dropwise. The resulting mixture was stirred at 110° C. for 12 hr. The reaction mixture was concentrated under reduced pressure to remove toluene. The residue was purified by prep-TLC (SiO₂, petroleum ether/ethyl acetate=0:1). Compound tert-butyl N-(3-ethyl-4-pyridyl)carbamate (140 mg, 629.83 umol, 95.21% yield) was obtained as a white solid. LCMS: (M+H)⁺: 223.5.

Step 2: To a solution of tert-butyl N-(3-ethyl-4-pyridyl)carbamate (140 mg, 629.83 umol, 1 eq) in HCl/dioxane (4 M, 5 mL, 31.75 eq). The mixture was stirred at 25° C. for 3 hr. The reaction mixture was concentrated under reduced pressure to remove HCl/dioxane (5 mL). The crude product 3-ethylpyridin-4-amine (70 mg, crude) was obtained as a white solid. LCMS: (M+H)⁺: 123.1.

Step 3: To a solution of 3-ethylpyridin-4-amine (50 mg, 409.27 umol, 1 eq) in pyridine (2 mL) was added EDCI (94.15 mg, 491.13 umol, 1.2 eq) and 2-oxo-3,4-dihydro-1H-quinoline-6-carboxylic acid (93.90 mg, 491.13 umol, 1.2 eq). The mixture was stirred at 45° C. for 2 hr. The reaction mixture was concentrated under reduced pressure to remove pyridine (2 mL). The residue was purified by prep-TLC (SiO₂, petroleum ether/ethyl acetate=0:1). Then product was triturated with petroleum ether/ethyl acetate=10:1 (12 mL). The mixture was filtered and filter cake was concentrated under reduced pressure to give a residue. Compound No. 26, N-(3-ethyl-4-pyridyl)-2-oxo-3,4-dihydro-1H-quinoline-6-carboxamide (98.3% purity) (120 mg), was obtained as a white solid. LCMS: (M+H)⁺: 296.1. ¹HNMR (400 MHz, MeOD, ppm): δ 8.43 (s, 1H), 8.36 (d, J=5.6 Hz, 1H), 7.80-7.84 (m, 2H), 7.73 (d, J=5.2 Hz, 1H), 6.99 (d, J=8.4 Hz, 1H), 3.05 (t, J=8 Hz, 2H), 2.80 (q, J=7.6 Hz, 2H), 2.64 (t, J=8 Hz, 2H), 1.24 (t, J=7.6 Hz, 3H).

### Example 8. Synthesis of Compound 22

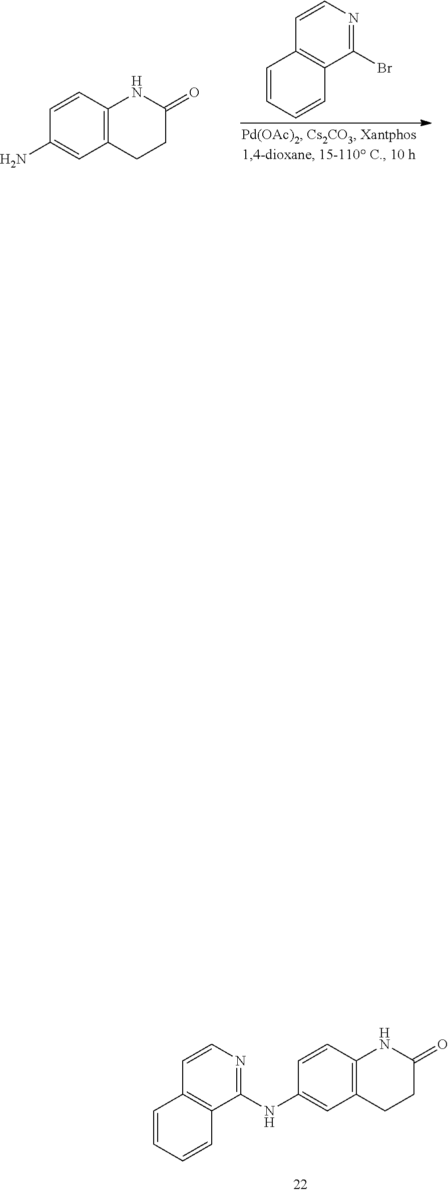 [SMILES: Nc1ccc2c(c1)CCC(=O)N2 ; Brc1nccc2ccccc12 ; O=C1CCc2cc(Nc3nccc4ccccc34)ccc2N1]

To a mixture of 6-amino-3,4-dihydro-1H-quinolin-2-one (100 mg, 616.57 umol, 1 eq) and 1-bromoisoquinoline (153.94 mg, 739.88 umol, 1.2 eq) in 1,4-dioxane (5 mL) was added Pd(OAc)₂ (34.61 mg, 154.14 umol, 0.25 eq), Xantphos (57.08 mg, 98.65 umol, 0.16 eq) and Cs₂CO₃ (401.78 mg, 1.23 mmol, 2 eq) in one portion at 15° C. under N2. The mixture was stirred at 110° C. for 10 hours. The reaction mixture was filtered and the filtrate was diluted with H₂O 6 mL and extracted with EtOAc 15 mL (5 mL*3). The combined organic layers were dried over Na₂SO₄, filtered and concentrated under reduced pressure to give a residue. The residue was purified by prep-TLC (SiO₂, petroleum ether:EtOAc=2:1). Then the crude product was triturated with EtOAc at 15° C. for 2 hours. The mixture was filtered to get the compound No. 22, 6-(1-isoquinolylamino)-3,4-dihydro-1H-quinolin-2-one (32 mg, 108.83 umol, 17.65% yield, 98.4% purity), as yellow solid. LCMS: (M+H)⁺: 290.1. ¹HNMR (400 MHz, DMSO-d6, ppm): δ 9.99 (s, 1H), 9.02 (s, 1H), 8.49 (d, J=4.4 Hz, 1H), 7.93 (d, J=5.6 Hz, 1H), 7.78-7.80 (m, 2H), 7.67-7.69 (m, 1H), 7.57-7.61 (m, 2H), 7.10 (d, J=6 Hz, 1H), 6.81 (d, J=8.4 Hz, 1H), 2.86-2.90 (m, 2H), 2.43-2.47 (m, 2H).

### Example 9. Synthesis of Compound 27

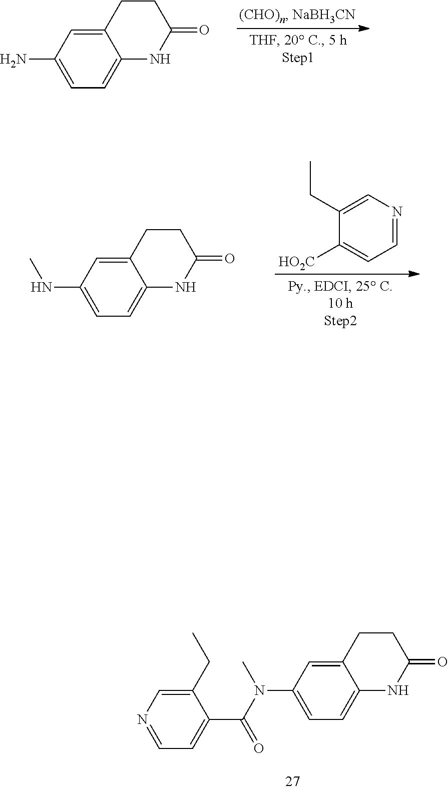 [SMILES: Nc1ccc2c(c1)CCC(=O)N2 ; CNc1ccc2c(c1)CCC(=O)N2 ; CCc1cnccc1C(=O)O ; CCc1cnccc1C(=O)N(C)c1ccc2c(c1)CCC(=O)N2]

Step 1: To a mixture of 6-amino-3,4-dihydro-1H-quinolin-2-one (500 mg, 3.08 mmol, 1 eq) in THE (10 mL) was added paraformaldehyde (194.39 mg, 2.16 mmol, 0.7 eq) in one portion at 20° C. under N2. The mixture was stirred at 20° C. for 3 h. Then NaBH₃CN (135.61 mg, 2.16 mmol, 0.7 eq) was added and the mixture was stirred for another 2 hours. The reaction mixture was filtered and concentrated under reduced pressure to give a residue. The crude product was purified by prep-HPLC [water (10 mM NH₄HCO₃)-acetonitrile (“ACN”)]. Compound 6-(methylamino)-3,4-dihydro-1H-quinolin-2-one (140 mg, 794.49 umol, 25.77% yield) was obtained as yellow solid. LCMS: (M+H)⁺: 177.1.

Step 2: To a mixture of 6-(methylamino)-3,4-dihydro-1H-quinolin-2-one (50 mg, 283.74 umol, 1 eq) and 3-ethylpyridine-4-carboxylic acid (42.89 mg, 283.74 umol, 1 eq) in pyridine (3 mL) was added EDCI (54.39 mg, 283.74 umol, 1 eq) in one portion at 25° C. The mixture was stirred at 25° C. for 10 hours. The reaction mixture was concentrated under reduced pressure to give a residue. The residue was purified by prep-TLC (SiO₂, Ethyl acetate:Methanol=4:1). Then the crude product was triturated with Petroleum ether:Ethyl acetate=8 mL:1 mL at 20° C. for 1 h. Compound No. 27, 3-ethyl-N-methyl-N-(2-oxo-3,4-dihydro-1H-quinolin-6-yl)pyridine-4-carboxamide (17 mg, 100% purity, 19.4% yield), was obtained as a white solid. LCMS: (M+H)⁺: 310.1. ¹HNMR (400 MHz, MeOD, ppm): δ 8.34 (s, 1H), 8.20 (d, J=5.2 Hz, 1H), 7.16 (d, J=5.2 Hz, 1H), 7.08 (s, 1H), 6.96 (dd, J=8.4 and 2 Hz, 1H), 6.68 (d, J=8.4 Hz, 1H), 3.45 (s, 3H), 2.84 (t, J=7.6 Hz, 2H), 2.68 (q, J=7.2 Hz, 2H), 2.48 (t, J=7.6 Hz, 2H), 1.26 (t, J=7.6 Hz, 3H).

### Example 10. Synthesis of Compound 28

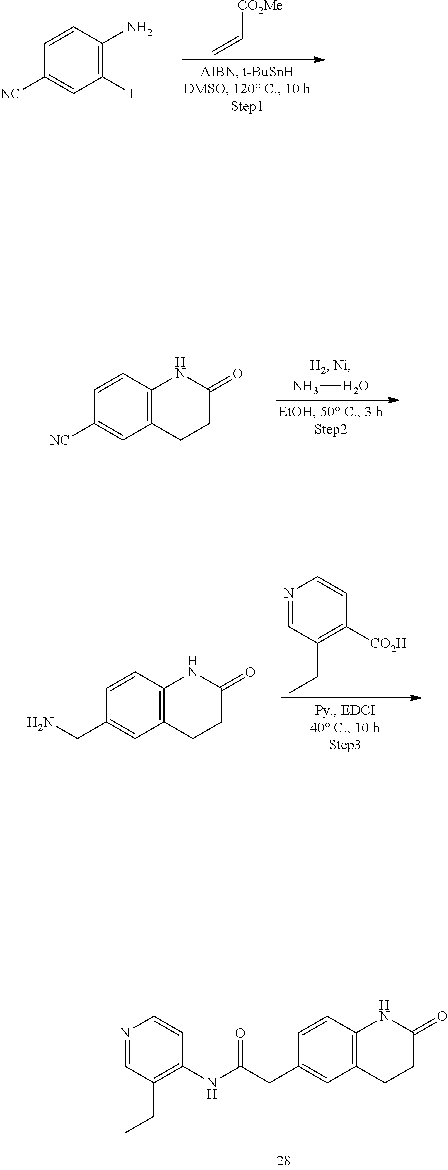

Step 1: To a mixture of 4-amino-3-iodo-benzonitrile (4 g, 16.39 mmol, 1 eq) and methyl prop-2-enoate (5.64 g, 65.57 mmol, 5.90 mL, 4 eq) in DMSO (80 mL) was added AIBN (10.77 g, 65.57 mmol, 4 eq) and Bu₃SnH (7.16 g, 24.59 mmol, 6.51 mL, 1.5 eq) dropwise under N2. The mixture was stirred at 120° C. for 10 hours. The mixture was cooled to 20° C. and poured into ice-water (w/w=1/1) (80 mL) and stirred for 15 min. The aqueous phase was extracted with ethyl acetate (80 mL*3). The combined organic phase was washed with brine (80 mL*2), dried with anhydrous Na₂SO₄, filtered and concentrated in vacuum. The crude product was triturated with Petroleum ether:EtOA=1:1(30 mL) at 20° C. for 60 min. Compound 2-oxo-3,4-dihydro-1H-quinoline-6-carbonitrile (0.42 g, 1.94 mmol, 11.82% yield, 79.4% purity) was obtained as yellow solid. LCMS: (M+H)⁺: 173.4.

Step 2: To a mixture of 2-oxo-3,4-dihydro-1H-quinoline-6-carbonitrile (220 mg, 1.28 mmol, 1 eq) in EtOH (25 mL) and NH₃.H₂O (2 mL) was added Ni (7.50 mg, 127.77 umol) under Are. The mixture was stirred at 50° C. for 3 hours under 50 Psi. The mixture was cooled to 20° C., filtered and concentrated in vacuum to get crude product. The residue was purified by prep-TLC(Ethyl acetate:MeOH 41:1). Compound 6-(aminomethyl)-3,4-dihydro-1H-quinolin-2-one (160 mg, 907.98 umol, 71.06% yield) was obtained as white solid.

Step 3: To a mixture of 6-(aminomethyl)-3,4-dihydro-1H-quinolin-2-one (60 mg, 340.49 umol, 1 eq) and 3-ethylpyridine 4 carboxylic acid (51.47 mg, 340.49 umol, 1 eq) in pyridine (5 mL) was added EDCI (65.27 mg, 340.49 umol, 1 eq) in one portion at 40° C. under N₂. The mixture was stirred at 40° C. for 10 hours. The reaction mixture was filtered and concentrated under reduced pressure to give a residue. The residue was purified by prep-TLC (SiO₂, Ethyl acetate:Methanol=3:1). Compound No. 28, 3-ethyl-N-[(2-oxo-3,4-dihydro-1H-quinolin-6-yl)methyl]pyridine-4-carboxamide (20 mg, 99.7% purity), was obtained as white solid. LCMS: (M+H)⁺: 310.1. ¹HNMR (400 MHz, MeOD, ppm): δ 8.49 (s, 1H), 8.43 (d, J=5.2 Hz, 1H), 7.3 (d, J=5.2 Hz, 1H), 7.17-7.21 (m, 2H), 6.86 (d, J=7.6 Hz, 1H), 4.48 (s, 2H), 2.96 (t, J=7.6 Hz, 2H), 2.77 (q, J=7.2 Hz, 2H), 2.57 (t, J=7.2 Hz, 2H), 1.19 (t, J=7.2 Hz, 3H).

### Example 11. Synthesis of Compound 30

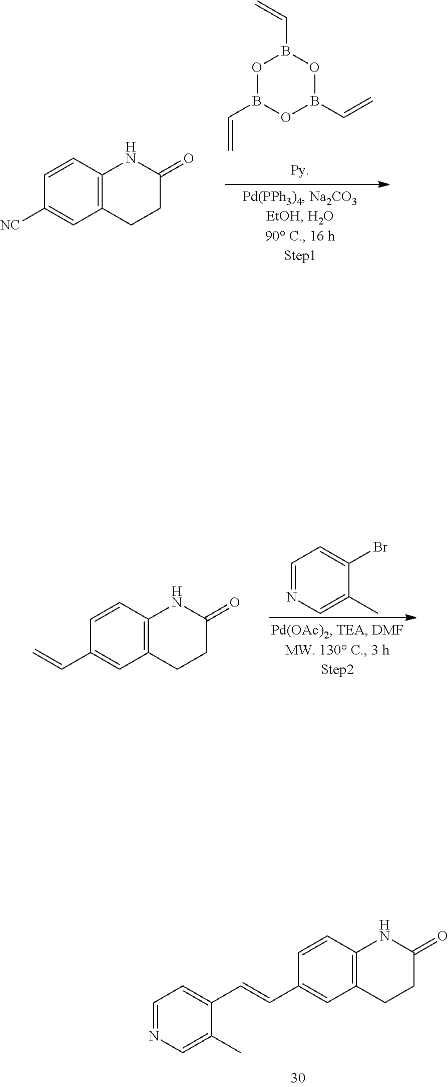

Step 1: To a mixture of 6-bromo-3,4-dihydro-1H-quinolin-2-one (2 g, 8.85 mmol, 1 eq) and pyridine; 2,4,6-trivinyl-1,3,5,2,4,6-trioxatriborinane (2.55 g, 10.62 mmol, 1.2 eq) in toluene (40 mL), EtOH (8 mL) and H₂O (2 mL) was added Pd(PPh₃)₄ (1.02 g, 884.68 umol, 0.1 eq) and Na₂CO₃ (2.81 g, 26.54 mmol, 3 eq) under N₂. The mixture was heated to 90° C. and stirred for 16 hours. The mixture was cooled to 20° C. and poured into ice-water (60 mL) and stirred for 15 min. The aqueous phase was extracted with ethyl acetate (80 mL*3). The combined organic phase was washed with brine (50 mL*2), dried with anhydrous Na₂SO₄, filtered and concentrated in vacuum. The residue was purified by silica gel chromatography (Petroleum ether:Ethyl acetate=50:1-0:1). Compound 6-vinyl-3,4-dihydro-1H-quinolin-2-one (1.05 g, 6.06 mmol, 68.52% yield) was obtained as yellow solid. LCMS: (M+H)⁺: 173.4.

Step 2: 6-vinyl-3,4-dihydro-1H-quinolin-2-one (50 mg, 288.67 umol, 1 eq), 4-bromo-3-methyl-pyridine (30.09 mg, 144.33 umol, 0.5 eq, HCl), Pd(OAc)₂ (5.18 mg, 23.09 umol, 0.08 eq), tris-o-tolylphosphane (17.57 mg, 57.73 umol, 0.2 eq) and TEA (87.63 mg, 866.00 umol, 120.54 uL, 3 eq) were taken up into a microwave tube in DMF (3 mL). The sealed tube was heated at 130° C. for 3 h under microwave. The reaction mixture was concentrated under reduced pressure to give a residue. The residue was purified by prep-HPLC [water (0.05% NH₃H₂O+10 mM NH₄HCO₃)-ACN]. Compound No. 30, 6-[(E)-2-(3-methyl-4-pyridyl)vinyl]-3,4-dihydro-1H-quinolin-2-one (17.6 mg, 100% purity), was obtained as white solid. LCMS: (M+H)⁺: 265.1. ¹HNMR (400 MHz, MeOD, ppm): δ 8.30 8.32 (m, 2H), 7.63 (d, J=5.2 Hz, 1H), 7.51 (s, 1H), 7.46 (dd, J=8.0 and 1.6 Hz, 1H), 7.25-7.34 (2H), 6.90 (d, J=8 Hz, 1H), 3.01 (t, J=8 Hz, 2H), 2.60 (t, J=8 Hz, 2H), 2.44 (s, 3H).

### Example 12. Synthesis of Compound 31

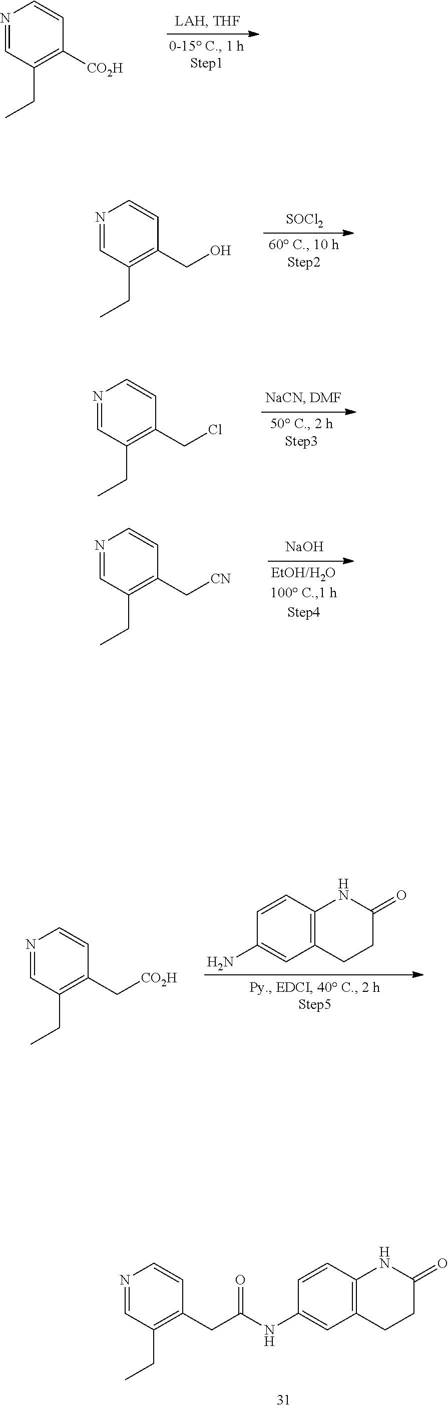

Step 1: A solution of 3-ethylpyridine-4-carboxylic acid (500 mg, 3.31 mmol, 1 eq) in THF (20 mL) was added to the mixture of LAH (125.54 mg, 3.31 mmol, 1 eq) in THF (40 mL) at 0° C. Then the mixture was stirred at 15° C. for 1 h. The reaction mixture was quenched by addition sat.Na₂CO₃ (15 mL) at 0° C., and then diluted with H₂O (15 mL) and extracted with EtOAc (10 mL*5). The combined organic layers were washed with brine (25 mL*2), dried over Na₂SO₄, filtered and concentrated under reduced pressure to give a residue. The residue was purified by column chromatography (SiO₂, Petroleum ether/Ethyl acetate=2/1 to 0/1). Compound (3-ethyl-4-pyridyl) methanol (150 mg, 1.09 mmol, 33.06% yield) was obtained as a white solid. LCMS: (M+H)⁺: 138.1.

Step 2: The solution of (3-ethyl-4-pyridyl) methanol (150 mg, 1.09 mmol, 1 eq) in SOCl₂ (4.92 g, 41.35 mmol, 3 mL, 37.82 eq) was stirred at 60° C. for 10 hr. The reaction mixture was concentrated under reduced pressure to remove SOCl₂. The residue was purified by prep-TLC (SiO₂, DCM:MeOH=10:1). Compound 4-(chloromethyl)-3-ethyl-pyridine (140 mg, 899.60 umol, 82.27% yield) was obtained as a white solid. LCMS: (M+H)⁺: 156.1.

Step 3: To a solution of 4-(chloromethyl)-3-ethyl-pyridine (100 mg, 642.57 umol, 1 eq) in DMF (2 mL) was added NaCN (47.24 mg, 963.86 umol, 1.5 eq). The mixture was stirred at 50° C. for 2 hr. The reaction mixture was cooled to room temperature and extracted with EtOAc (10 mL*3). The combined organic layers were washed with brine (15 mL*2), dried over Na₂SO₄, filtered and concentrated under reduced pressure to give a residue. The crude product 2-(3-ethyl-4-pyridyl) acetonitrile (80 mg, 547.24 umol, 85.16% yield) was obtained as a white solid. LCMS: (M+H)⁺: 147.1.

Step 4: To a solution of 2-(3-ethyl-4-pyridyl) acetonitrile (80 mg, 547.24 umol, 1 eq) in EtOH (2 mL) and H₂O (2 mL) was added NaOH (43.78 mg, 1.09 mmol, 2 eq). The mixture was stirred at 100° C. for 1 hr. The reaction mixture was concentrated under reduced pressure to remove EtOH and H₂O. The residue was purified by prep-HPLC (column: Welch Xtimate C18 150*25 mm*5 um; mobile phase: [water(0.04% HCl)-ACN]; B %: 1%-10%, 8 min). Compound 2-(3-ethyl-4-pyridyl) acetic acid (40 mg, 242.15 umol, 44.25% yield) was obtained as a white solid. LCMS: (M+H)⁺: 166.0.

Step 5: To a mixture of 2-(3-ethyl-4-pyridyl)acetic acid (35 mg, 211.88 umol, 1 eq) and 6-amino-3,4-dihydro-1H-quinolin-2-one (34.36 mg, 211.88 umol, 1 eq) in pyridine (1 mL) was added EDCI (40.62 mg, 211.88 umol, 1 eq) in one portion at 40° C. The mixture was stirred at 40° C. for 2 hrs. The reaction mixture was concentrated under reduced pressure to give a residue. The residue was purified by prep-TLC (SiO₂, Ethyl acetate:Methanol=4:1). Compound 2-(3-ethyl-4-pyridyl)-N-(2-oxo-3, 4-dihydro-1H-quinolin-6-yl) acetamide (14.3 mg, 100% purity) was obtained. LCMS: (M+H)⁺: 310.1.

### Example 13. Synthesis of Compound 38

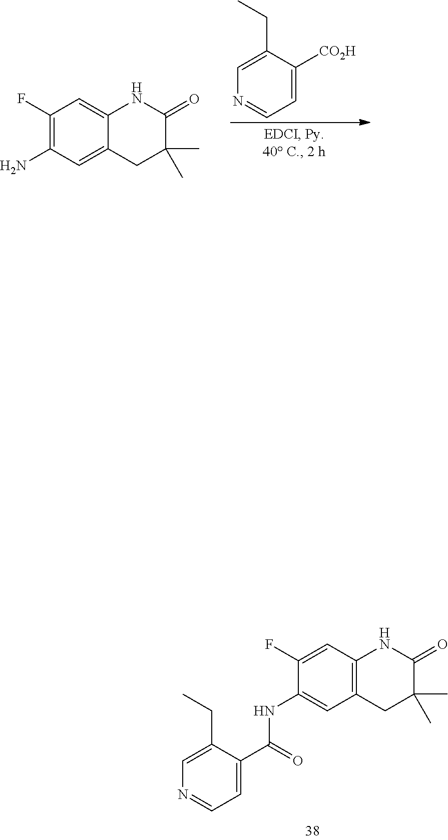

To a solution of 6-amino-7-fluoro-3,3-dimethyl-1,4-dihydroquinolin-2-one (20 mg, 96.05 umol, 1 eq, see Example 21 for the synthesis of this compound) in pyridine (1 mL) was added EDCI (22.09 mg, 115.26 umol, 1.2 eq) and 3-ethylpyridine-4-carboxylic acid (14.52 mg, 96.05 umol, 1 eq). The mixture was stirred at 40° C. for 2 hrs. The reaction mixture was concentrated under reduced pressure to remove pyridine. The residue was purified by prep-TLC (SiO₂, Petroleum ether:EtOAc=0:1). Compound 2-ethyl-N-(7-fluoro-3,3-dimethyl-2-oxo-1,4-dihydroquinolin-6-yl)pyridine-4-carboxamide (27 mg, 100% purity) was obtained. LCMS: (M+H)⁺: 342.1.

### Example 14. Synthesis of Compound 41

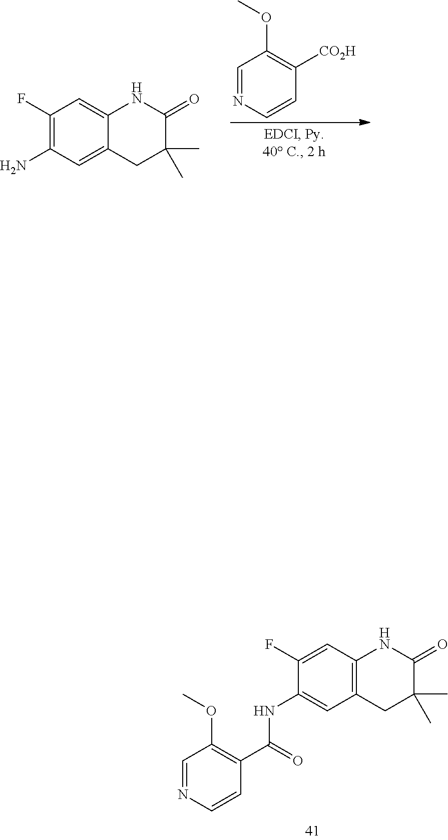

To a mixture of 6-amino-7-fluoro-3,3-dimethyl-1,4-dihydroquinolin-2-one (20 mg, 96.05 umol, 1 eq) and 3-methoxypyridine-4-carboxylic acid (17.65 mg, 115.26 umol, 1.2 eq) in pyridine (2 mL) was added EDCI (22.09 mg, 115.26 umol, 1.2 eq) in one portion at 40° C. The mixture was stirred at 40° C. for 2 hrs. The reaction mixture was concentrated under reduced pressure to give a residue. The residue was purified by prep-TLC (S102, Ethyl acetate:Methanol=4:1). Compound N-(7-fluoro-3,3-dimethyl-2-oxo-1,4-dihydroquinolin-6-yl)-3-methoxy-pyridine-4-carboxamide (20 mg, 100% purity) was obtained. LCMS: (M+H)⁺: 344.1.

### Example 15. Synthesis of Compound 42

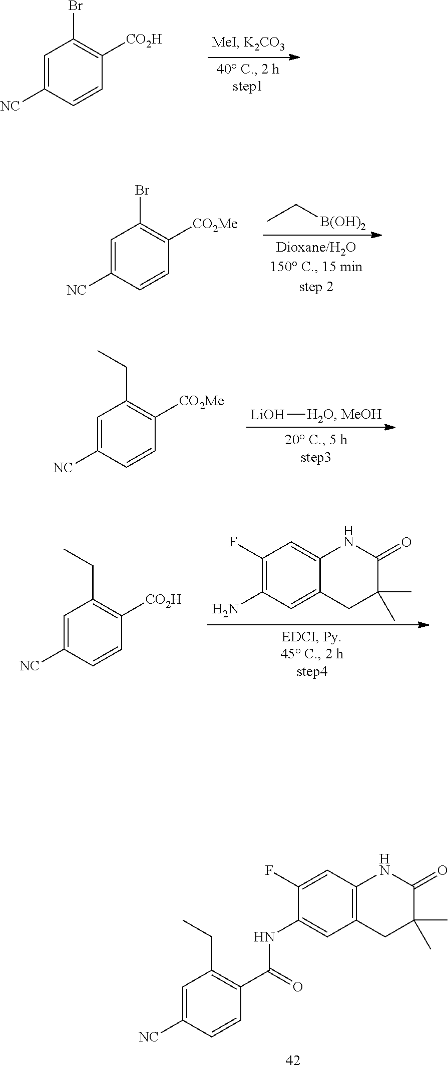

Step 1: To a mixture of 2-bromo-4-cyano-benzoic acid (600 mg, 2.65 mmol, 1 eq), K₂CO₃ (403.56 mg, 2.92 mmol, 1.1 eq) in DMF (10 mL) was added MeI (414.46 mg, 2.92 mmol, 181.78 uL, 1.1 eq) under N₂. The mixture was stirred at 40° C. for 2 hours. The reaction mixture was quenched by addition H₂O (10 mL), and then extracted with EtOAc (10 mL*3). The combined organic layers were washed with brine (15 mL*2), dried over Na₂SO₄, filtered and concentrated under reduced pressure to give a residue. The residue was purified by column chromatography (SiO₂, Petroleum ether/Ethyl acetate=100/1 to 0/1). Compound methyl 2-bromo-4-cyano-benzoate (450 mg, crude) was obtained as white solid.

Step 2: Methyl 2-bromo-4-cyano-benzoate (100 mg, 416.57 umol, 1 eq), ethylboronic acid (61.56 mg, 833.15 umol, 2 eq), Pd(PPh₃)₄ (48.14 mg, 41.66 umol, 0.1 eq) and K₃PO₄ (176.85 mg, 833.15 umol, 2 eq) were taken up into a microwave tube in DME (3 mL). The sealed tube was heated at 150° C. for 15 min under microwave. The reaction mixture was filtered and concentrated under reduced pressure to give a residue. The residue was purified by column chromatography (SiO₂, Petroleum ether/Ethyl acetate=15/1 to 5/1). Compound methyl 4-cyan-2-ethyl-benzoate (53 mg, 280.11 umol, 22.41% yield) was obtained as a white solid.

Step 3: To a mixture of methyl 4-cyano-2-ethyl-benzoate (53 mg, 280.11 umol, 1 eq) in MeOH (2 mL) was added LiOH.H2O (17.63 mg, 420.17 umol, 1.5 eq), H2O (1 mL). The mixture was stirred at 20° C. for 5 hours. The reaction mixture was filtered and concentrated under reduced pressure to remove MeOH. The solution is added with HCl(1N) until the solid is no longer precipitated. The mixture was filtered to get the title compound 4-cyano-2-ethyl-benzoic acid (20 mg, 114.17 umol, 40.76% yield) was obtained as a white solid.

Step 4: To a solution of 6-amino-7-fluoro-3,3-dimethyl-1,4-dihydroquinolin-2-one (15 mg, 72.04 umol, 1 eq) in pyridine (2 mL) was added EDCI (16.57 mg, 86.44 umol, 1.2 eq) and 4-cyano-2-ethyl-benzoic acid (15.00 mg, 85.62 umol, 1.19 eq). The mixture was stirred at 45° C. for 2 hrs. The reaction mixture was concentrated under reduced pressure to remove pyridine. The residue was purified by prep-TLC (SiO₂, Petroleum ether:EtOAc=1:1). Compound 4-cyano-2-ethyl-N-(7-fluoro-3, 3-dimethyl-2-oxo-1,4-dihydroquinolin-6-yl)benzamide (21 mg, 100% purity) was obtained. LCMS: (M+H)⁺: 366.1.

### Example 16. Synthesis of Compound 49

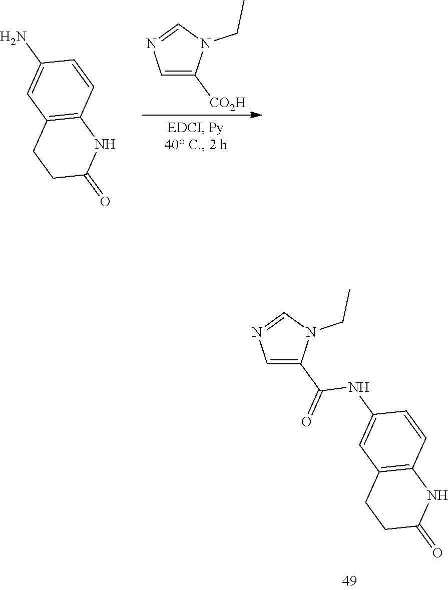

To a mixture of 6-amino-3,4-dihydro-1H-quinolin-2-one (50 mg, 308.28 umol, 1 eq) and 3-ethylimidazole 4 carboxylic acid (43.20 mg, 308.28 umol, 1 eq) in pyridine (3 mL) was added EDCI (70.92 mg, 369.94 umol, 1.2 eq) in one portion. The mixture was stirred at 40° C. for 2 hrs. The reaction mixture was concentrated under reduced pressure to give a residue. The residue was purified by prep-TLC (SiO₂, Ethyl acetate:Methanol=10:1). Compound 3-ethyl-N-(2-oxo-3,4-dihydro-1H-quinolin-6-yl) imidazole-4-carboxamide (75 mg, 100°βo purity) was obtained as white solid. LCMS: (M+H)⁺: 285.1.

### Example 17. Synthesis of Compound 50

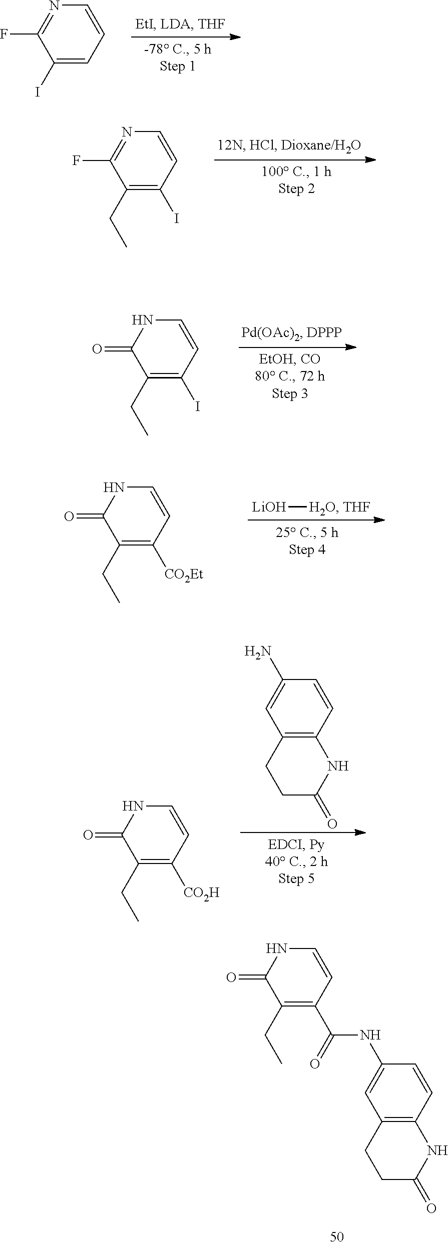

Step 1: To a solution of the 2-fluoro-3-iodo-pyridine (4 g, 17.94 mmol, 1 eq) in THF (20 mL) was added dropwise the solution of LDA (2 M, 8.98 mL, 1 eq) in THE (40 mL) at −78° C. under N₂. The mixture was stirred at the same temperature for 1 h. To the reaction mixture was added dropwise a solution of CH₃CH₂I (2.80 g, 17.94 mmol, 1.43 mL, 1 eq) in THF (20 mL) and the mixture was stirred at −78° C. for 4 hours. After addition of water (5 ml), the reaction mixture was warmed to mom temperature and diluted with brine (10 mL*2). The mixture was extracted with EtOAc (20 mL*3). The organic layers were dried over Na₂SO₄, filtered and then evaporated under reduced pressure to remove the solvent. The residue was purified by column chromatography (SiO₂, Petroleum ether/Ethyl acetate=50/1 to 20/1). Compound 3-ethyl-2-fluoro-4-iodo-pyridine (3.6 g, 14.34 mmol, 79.94% yield) was obtained as colorless oil. LCMS: (M+H)⁺: 252.0.

Step 2: To a solution of 3-ethyl-2-fluoro-4-iodo-pyridine (600 mg, 2.39 mmol, 1 eq) in H₂O (2 mL) and dioxane (2 mL) was added conc. HCl (12 M, 4 mL, 20.08 eq). The mixture was stirred at 100° C. for 1 hr. The reaction mixture was concentrated under reduced pressure. The crude product was triturated with the mixture solution of Petro ether and EtOAc (10:1, 11 mL) at 25° C. for 10 min. The mixture was filtered and filter cake was concentrated under reduced pressure to give a residue. Compound 3-ethyl-4-iodo-1H-pyridin-2-one (570 mg, 2.29 mmol, 95.76% yield) was obtained as a white solid. LCMS: (M+H)⁺: 250.0.

Step 3: The suspension of 3-ethyl-4-iodo-1H-pyridin-2-one (570 mg, 2.29 mmol, 1 eq), DPPP (471.98 mg, 1.14 mmol, 0.5 eq) and Pd(OAc)₂ (256.92 mg, 1.14 mmol, 0.5 eq) in EtOH (15 mL) was degassed and purged with CO for 3 times. The mixture was stirred under CO (50 Psi) at 80° C. for 72 hr. The reaction mixture was diluted with brine (10 mL*2) and then extracted with EtOAc (20 mL*3). The organic layers were dried over Na₂SO₄, filtered and then evaporated under reduced pressure to remove the solvent. The residue was purified by column chromatography (SiO₂, Petroleum ether/Ethyl acetate=10/1 to 2/1). Compound ethyl 3-ethyl-2-oxo-1H-pyridine-4-carboxylate (60 mg, 307.35 umol, 13.43% yield) was obtained as a white solid.

Step 4: To a mixture of ethyl 3-ethyl-2-oxo-1H-pyridine 4 carboxylate (60 mg, 307.35 umol, 1 eq) in THF (5 mL) and H₂O (5 mL) was added LiOH.H₂O (25.80 mg, 614.71 umol, 2 eq) in one portion at 25° C. The mixture was stirred at 25° C. for 5 hrs. The reaction mixture was concentrated under reduced pressure to remove THF. Then the aqueous was adjusted with 3 M aq. HCl until pH=3. The suspension was filtered and filter cake was concentrated under reduced pressure to give a residue. The crude product was triturated with the mixture solution of Petroleum ether and Ethyl acetate (5:1, 6 mL). The suspension was filtered and filter cake was concentrated under reduced pressure to give a residue. The product 3-ethyl-2-oxo-1H-pyridine-4-carboxylic acid (50 mg, 299.11 umol, 97.32% yield) was obtained as a white solid.

Step 5: To a mixture of 3-ethyl-2-oxo-1H-pyridine-4-carboxylic acid (50 mg, 299.11 umol, 1 eq) and 6-amino-3,4-dihydro-1H-quinolin-2-one (53.36 mg, 329.02 umol, 1.1 eq) in pyridine (3 mL) was added EDCI (68.81 mg, 358.93 umol, 1.2 eq) in one portion at 40° C. The mixture was stirred at 40° C. for 2 hrs. The reaction mixture was concentrated under reduced pressure to give a residue. The residue was purified by prep-TLC (SiO₂, Ethyl acetate:Methanol=5:1). Compound 3-ethyl-2-oxo-N-(2-oxo-3,4-dihydro-1H-quinolin-6-yl)-1H-pyridine-4-carboxamide (80 mg, 96.7% purity) was obtained. LCMS: (M+H)⁺: 312.0.

### Example 18. Synthesis of Compound 52

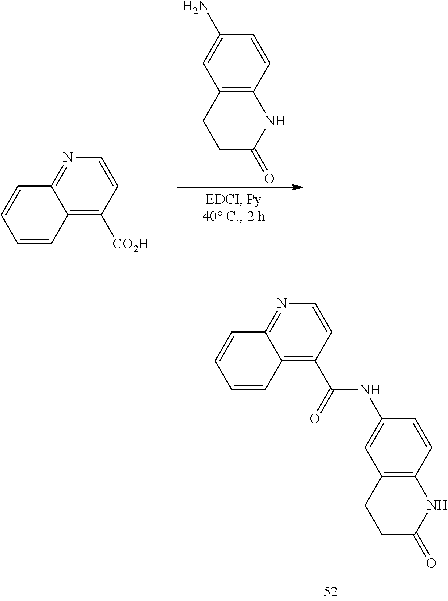 [SMILES: O=C(O)c1ccnc2ccccc12 ; Nc1ccc2c(c1)CCC(=O)N2 ; O=C1CCc2cc(NC(=O)c3ccnc4ccccc34)ccc2N1]

To a mixture of quinoline-4-carboxylic acid (50 mg, 288.74 umol, 1 eq) and 6-amino-3,4-dihydro-1H-quinolin-2-one (46.83 mg, 288.74 umol, 1 eq) in pyridine (1 mL) was added EDCI (66.42 mg, 346.48 umol, 1.2 eq) in one portion at 40° C. The mixture was stirred at 40° C. for 2 hrs. The reaction mixture was concentrated under reduced pressure to give a residue. The residue was purified by prep-HPLC (column: Phenomenex luna C18 80*40 mm*3 um; mobile phase: [water(0.04% HCl)-ACN]; B %: 20%-35%, 7 min). Compound N-(2-oxo-3,4-dihydro-1H-quinolin-6-yl)quinoline-4-carboxamide (21 mg, 95% purity) was obtained as white solid. LCMS: (M+H)⁺: 318.1.

### Example 19. Synthesis of Compound 54

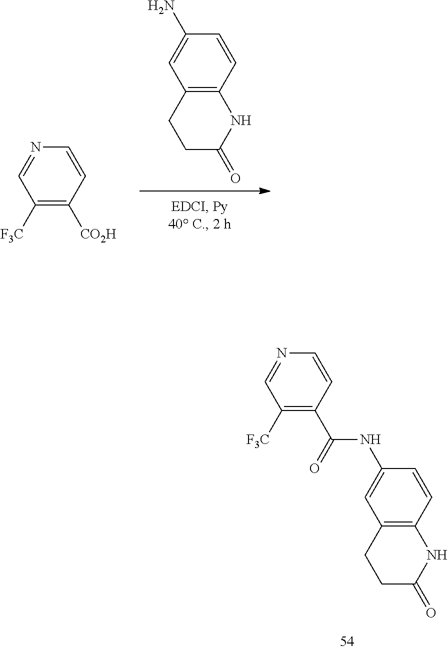 [SMILES: O=C(O)c1ccncc1C(F)(F)F ; Nc1ccc2c(c1)CCC(=O)N2 ; O=C1CCc2cc(NC(=O)c3ccncc3C(F)(F)F)ccc2N1]

To a mixture of 3-(trifluoromethyl)pyridine-4-carboxylic acid (100 mg, 523.27 umol, 1 eq) and 6-amino-3,4-dihydro-1H-quinolin-2-one (84.87 mg, 523.27 umol, 1 eq) in pyridine (2 mL) was added EDCI (120.37 mg, 627.92 umol, 1.2 eq) in one portion at 40° C. The mixture was stirred at 40° C. for 2 hrs. The reaction mixture was concentrated under reduced pressure to give a residue. The residue was purified by prep-HPLC (column: Waters Xbridge Prep OBD C18 150*40 mm*10 um; mobile phase: [water(0.05% NH3H2O+10 mM NH4HCO3)-ACN]; B %: 5%-45%, 8 min). Compound N-(2-oxo-3,4-dihydro-1H-quinolin-6-yl)-3-(trifluoromethyl)pyridine-4-carboxamide (103 mg, 95% purity) was obtained.

### Example 20. Synthesis of Compound 60

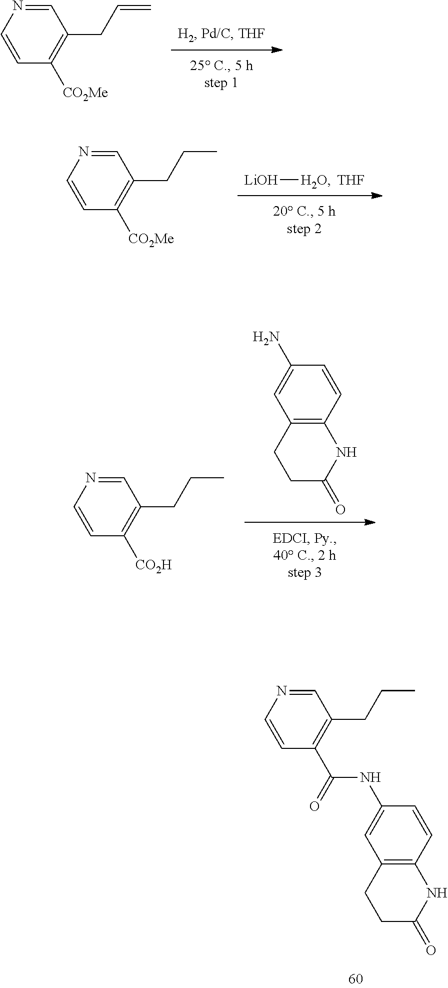

Step 1: The suspension of methyl 3-allylpyridine-4-carboxylate (50 mg, 282.17 umol, 1 eq) and 10% Pd/C (20 mg) in THF (5 mL) was degassed and purged with H₂ for 3 times. The mixture was stirred under H₂ (15 Psi) at 25° C. for 5 hr. The reaction mixture was filtered and filtrate concentrated under reduced pressure to give a residue. The crude product was triturated with the mixture solution of Petroleum ether:EtOAc (10:1.11 mL) at 25° C. for 10 min. The suspension was filtered and filter cake was concentrated under reduced pressure to give a residue. Compound methyl 3-propylpyridine-4-carboxylate (45 mg, 251.09 umol, 88.99% yield) was obtained as a white solid. LCMS: (M+H)⁺: 180.1.

Step 2: To a mixture of methyl 3-propylpyridine-4-carboxylate (45 mg, 251.09 umol, 1 eq) in THF (1 mL) and H₂O (1 mL) was added LiOH.H2O (21.07 mg, 502.19 umol, 2 eq) in one portion at 20° C. under N₂. The mixture was stirred at 20° C. for 5 hrs. The reaction mixture was concentrated under reduced pressure to remove THF. The mixture was adjusted with 3 M (HCl) until pH=3. The suspension was filtered. The crude product was triturated with Petroleum ether. EtOAc (5:1, 6 mL). The suspension was filtered and filter cake was concentrated under reduced pressure to give a residue. The product 3-propylpyridine-4-carboxylic acid (40 mg, 242.15 umol, 96.44% yield) was obtained as a white solid.

Step 3: To a mixture of 3-propylpyridine-4-carboxylic acid (40 mg, 242.15 umol, 1 eq) and 6-amino-3,4-dihydro-1H-quinolin-2-one (43.20 mg, 266.36 umol, 1.1 eq) in pyridine (1 mL) was added EDCI (55.70 mg, 290.58 umol, 1.2 eq) in one portion at 40° C. The mixture was stirred at 40° C. for 2 hours. The reaction mixture was concentrated under reduced pressure to give a residue. The residue was purified by prep-TLC (SiO₂, DCM:MeOH=10:1). Compound N-(2-oxo-3,4-dihydro-1H-quinolin-6-yl)-3-propyl-pyridine-4-carboxamide (26.8 mg, 100% purity) was obtained. LCMS: (M+H)⁺: 310.1.

### Example 21. Synthesis of Compound 37

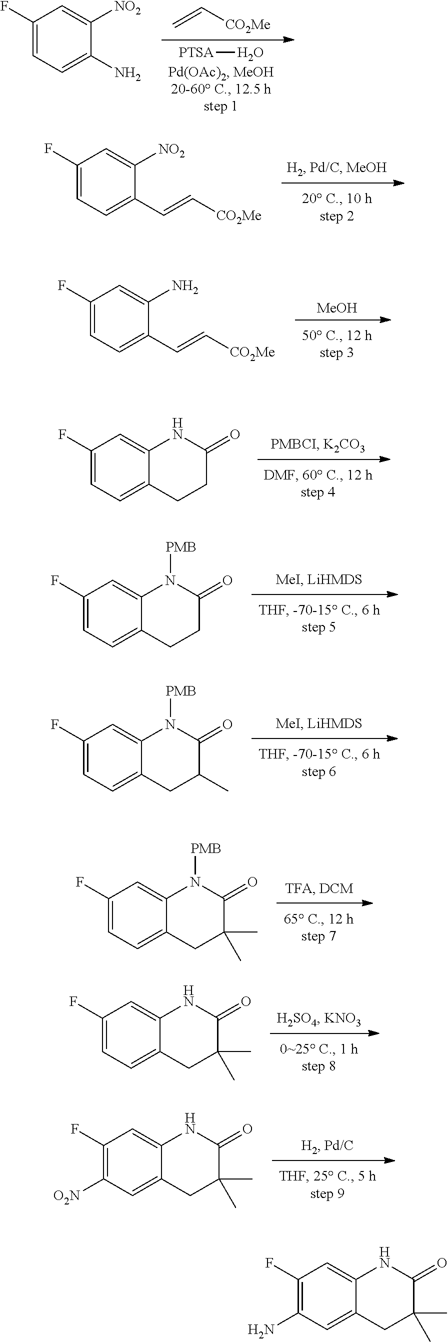

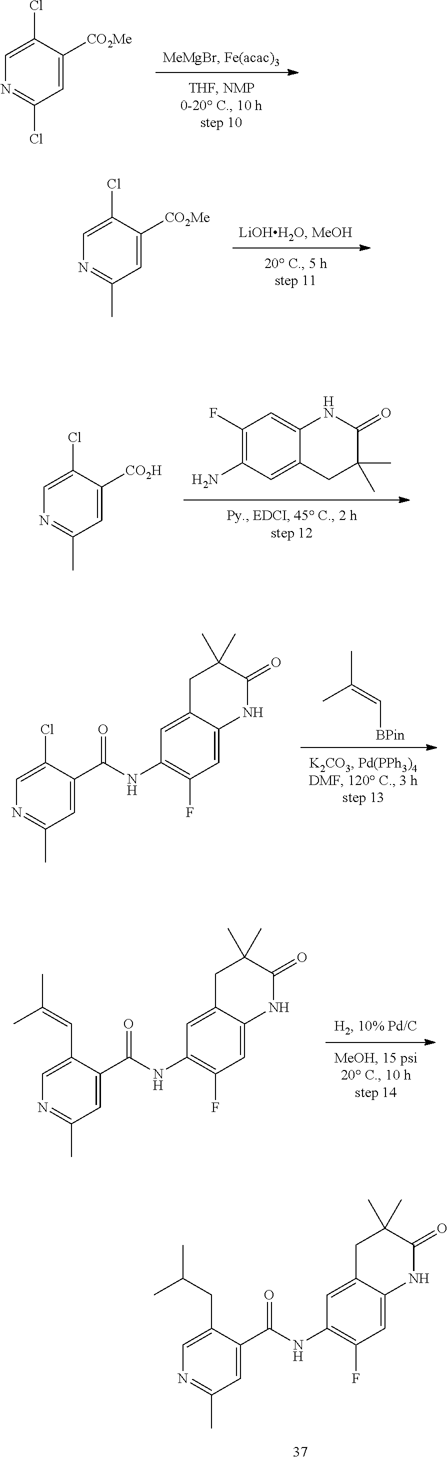

Step 1: Stage 1: To a mixture of NaNO₂ (152 g, 2.20 mol, 8.72 eq) and Amberlyst A26 (286 g) in H₂O (3000 mL) in one portion at 20° C. under N₂. The mixture was stirred at 20° C. for 30 min. Filter the reactants and adjust the pH to 7. Stage 2: The solution of 4-fluoro-1,2-dinitro-benzene (47 g, 252.56 mmol, 1 eq), 4-methylbenzenesulfonic acid (143.52 g, 833.44 mmol, 3.3 eq) and palladium acetate (5.67 g, 25.26 mmol, 0.1 eq) in MeOH (500 mL) was added to the product from stage 1, and then added methyl prop-2-enoate (108.71 g, 1.26 mol, 113.72 mL, 5 eq) in one portion at 60° C. under N₂. The mixture was stirred at 60° C. for 12 hr. The reaction mixture was filtered and concentrated under reduced pressure to give a residue. The residue was purified by column chromatography (SiO2, Petroleum ether/Ethyl acetate=15/1 to 2/1). Compound methyl (E)-3-(4-fluoro-2-nitro-phenyl)prop-2-enoate (9 g, 39.97 mmol, 15.83% yield) was obtained as a white solid.

Step 2: To a mixture of methyl (E)-3-(4-fluoro-2-nitro-phenyl)prop-2-enoate (9 g, 39.97 mmol, 1 eq) in MeOH (100 mL) and THF (20 mL) was added 10% Pd/C (3 g) in one portion at 20° C. under N₂. The mixture was stirred at 20° C. for 10 hr. The reaction mixture was filtered and concentrated under reduced pressure to give a residue. Compound methyl 3-(2-amino-4-fluoro-phenyl)propanoate (9 g, crude) was obtained as a white solid.

Step 3: To a mixture of methyl 3-(2-amino-4-fluoro-phenyl)propanoate (5.3 g, 26.88 mmol, 1 eq) was added MeOH (50 mL) at 60° C. under N₂. The mixture was stirred at 60° C. for 12 hr. The reaction mixture was concentrated under reduced pressure to remove MeOH (50 mL). The residue was purified by column chromatography (SiO2, Petroleum ether/Ethyl acetate=10/1 to 3/1). Compound 7-fluoro-3,4-dihydro-1H-quinolin-2-one (3.4 g, 20.59 mmol, 76.60% yield) was obtained as a white solid.

Step 4: To a solution of 7-fluoro-3,4-dihydro-1H-quinolin-2-one (1 g, 6.05 mmol, 1 eq) in DMF (20 mL) was added PMB-Cl (1.33 g, 8.48 mmol, 1.15 mL, 1.4 eq) and K₂CO₃ (1.67 g, 12.11 mmol, 2 eq). The mixture was stirred at 60° C. for 12 hr. The reaction mixture was cooled to room temperature and extracted with EtOAc 30 mL (15 mL*2). The combined organic layers were washed with brine 30 mL (15 mL*2), dried over Na₂SO₄, filtered and concentrated under reduced pressure to give a residue. The residue was purified by column chromatography (SiO2, Petroleum ether/Ethyl acetate=10/1 to 3/1). Compound 7-fluoro-1-[(4-methoxyphenyl)methyl]-3,4-dihydroquinolin-2-one (1 g, 3.50 mmol, 57.89% yield) was obtained as a yellow oil. LCMS: (M+H)⁺: 286.1.

Step 5: To a mixture of 7-fluoro-1-[(4-methoxyphenyl)methyl]-3,4-dihydroquinolin-2-one (1 g, 3.50 mmol, 1 eq) in THF (5 mL) was added LiHMDS (1 M, 7.71 mL, 2.2 eq) at −70° C. under N₂. The mixture was stirred at −70° C. for 30 min, then MeI (2.98 g, 21.03 mmol, 1.31 mL, 6 eq) was added. The mixture was heated to 15° C. and stirred for 5.5 hours. The reaction mixture was quenched by addition H₂O 10 mL, and then diluted with H₂O 10 mL and extracted with EtOAc 30 mL (10 mL*3). The combined organic layers were dried over Na₂SO₄, filtered and concentrated under reduced pressure to give a residue. The crude product 7-fluoro-1-[(4-methoxyphenyl)methyl]-3-methyl-3,4-dihydroquinolin-2-one (750 mg, 2.51 mmol, 71.49% yield) was obtained as yellow oil. LCMS: (M+H)⁺: 300.2.

Step 6: To a solution of 7-fluoro-1-[(4-methoxyphenyl)methyl]-3-methyl-3,4-dihydroquinolin-2-one (600 mg, 2.00 mmol, 1 eq) in THE (5 mL) was added LiHMDS (1 M, 4.41 mL, 2.2 eq) at −70° C. The mixture was stirred at −70° C. for 30 min. Then MeI (1.71 g, 12.03 mmol, 748.70 uL, 6 eq) was added. The mixture was heated to 15° C. and stirred for 5.5 hours. The reaction mixture was quenched by addition H₂O 5 mL, and then diluted with H₂O 5 mL and extracted with EtOAc 9 mL (3 mL*3). The combined organic layers were dried over Na₂SO₄, filtered and concentrated under reduced pressure to give a residue. The residue was purified by prep-TLC (SiO₂, PE:EA=3:1). Compound 7-fluoro-1-[(4-methoxyphenyl)methyl]-3,3-dimethyl-4H-quinolin-2-one (550 mg, 1.76 mmol, 87.56% yield) was obtained as a yellow oil.

Step 7: To a mixture of 7-fluoro-1-[(4-methoxyphenyl)methyl]-3,3-dimethyl-4H-quinolin-2-one (550 mg, 1.76 mmol, 1 eq) in DCM (2 mL) was added TFA (6.16 g, 54.02 mmol, 4 mL, 30.78 eq). The mixture was stirred at 65° C. for 12 hours. The reaction mixture was concentrated under reduced pressure to give a residue. The residue was purified by prep-TLC (SiO₂, Petroleum ether/Ethyl acetate=2:1). Compound 7-fluoro-3,3-dimethyl-1,4-dihydroquinolin-2-one (310 mg, 1.60 mmol, 91.41% yield) was obtained as white solid. LCMS: (M+H)⁺: 194.1.

Step 8: To a solution of 7-fluoro-3,3-dimethyl-1,4-dihydroquinolin-2-one (200 mg, 1.04 mmol, 1 eq) in conc.H₂SO₄ (5 mL) was added KNO₃ (104.65 mg, 1.04 mmol, 1 eq) at 0° C. The mixture was stirred at 25° C. for 1 h. The reaction mixture was cooled at 0° C. and the resulting solution was stirred for 15 min at 0° C. The reaction was quenched by adding 100 mL of H₂O/ice, filtered and filter cake concentrated under reduced pressure to give a residue. The crude product was triturated with PE:EA=10:1 (11 mL) at 25° C. for 20 min. The mixture was filtered and filter cake was concentrated under reduced pressure to give a residue. Compound 7-fluoro-3,3-dimethyl-6-nitro-1,4-dihydroquinolin-2-one (180 mg, 755.62 umol, 73.00% yield) was obtained as a white solid. LCMS: (M+H)⁺: 239.1.

Step 9: To a solution of 7-fluoro-3,3-dimethyl-6-nitro-1,4-dihydroquinolin-2-one (180 mg, 755.62 umol, 1 eq) in MeOH (10 mL) was added 10% Pd/C (50 mg) under H₂ atmosphere. The suspension was degassed and purged with 112 for 3 times. The mixture was stirred under H₂ (45 Psi) at 25° C. for 1 hr. The reaction was clean according to TLC. The reaction mixture was filtered and filtrate concentrated under reduced pressure to give a residue. The chide product was triturated with PE:EA=10:1(11 mL) at 25° C. for 10 min. The mixture was filtered and filter cake was concentrated under reduced pressure to give a residue. Compound 6-amino-7-fluoro-3,3-dimethyl-1,4-dihydroquinolin-2-one (130 mg, 624.30 umol, 82.62% yield) was obtained as a white solid.

Step 10: To a mixture of methyl 2,5-dichloropyridine-4-carboxylate (2 g, 9.71 mmol, 1 eq), Fe(acac)₃ (171.42 mg, 485.38 umol, 0.05 eq) and NMP (4 mL) in THE (40 mL) was added MeMgBr (3 M, 3.88 mL, 1.2 eq) in one portion at 0° C. under N₂. The mixture was stirred at 20° C. for 10 hours. The reaction mixture was quenched by addition aqueous NaCl 50 mL, and then diluted with aqueous NaCl 30 mL and extracted with EtOAc 300 mL (100 mL*3). The combined organic layers were dried over Na₂SO₄, filtered and concentrated under reduced pressure to give a residue. The residue was purified by column chromatography (SiO₂, Petroleum ether/Ethyl acetate=100/1 to 0/1). Compound methyl 5-chloro-2-methyl-pyridine-4-carboxylate (230 mg, 1.24 mmol, 12.77% yield) was white solid.

Step 11: To a mixture of methyl 5-chloro-2-methyl-pyridine-4-carboxylate (200 mg, 1.08 mmol, 1 eq) in MeOH (2.5 mL) and H₂O (2.5 mL) was added LiOH.H₂O (90.43 mg, 2.16 mmol, 2 eq) in one portion at 20° C. under N₂. The mixture was stirred at 20° C. for 5 hr. The reaction mixture was cooled to room temperature, and concentrated under reduced pressure. 3 M (HCl) was added to adjust the pH=3. The crude product 5-chloro-2-methyl-pyridine-4-carboxylic acid (160 mg, 932.51 umol, 86.54% yield) was obtained as a white solid.

Step 12: To a solution of 5-chloro-2-methyl-pyridine-4-carboxylic acid (128.54 mg, 749.17 umol, 1.2 eq) in pyridine (3 mL) was added EDCI (143.62 mg, 749.17 umol, 1.2 eq) and 6-amino-7-fluoro-3,3-dimethyl-1,4-dihydroquinolin-2-one (130 mg, 624.30 umol, 1 eq). The mixture was stirred at 45° C. for 2 hr. The reaction mixture was concentrated under reduced pressure to remove pyridine(3 mL). The residue was purified by prep-TLC (SiO₂, PE:EA=0:1). Compound 5-chloro-N-(7-fluoro-3,3-dimethyl-2-oxo-1,4-dihydroquinolin-6-yl)-2-methyl-pyridine-4-carboxamide (120 mg, 330.28 umol, 52.90% yield, 99.58% purity) was obtained.

Step 13: S-chloro-N-(7-fluoro-3,3-dimethyl-2-oxo-1,4-dihydroquinolin-6-yl)-2-methyl-pyridine-4-carboxamide (100 mg, 276.40 umol, 1 eq), 4,4,5,5-tetramethyl-2-(2-methylprop-1-enyl)-1,3,2-dioxaborolane (60.39 mg, 331.68 umol, 1.2 eq), K₂CO₃ (76.40 mg, 552.80 umol, 2 eq) and Pd(PPh3)4 (15.97 mg, 13.82 umol, 0.05 eq) were taken up into a microwave tube in dioxane (2 mL) and H₂O (0.4 mL). The sealed tube was heated at 120° C. for 3h under microwave. The reaction mixture was filtered and concentrated under reduced pressure to give a residue. The residue was purified by prep-TLC (SiO₂, Petroleum ether/Ethyl acetate=5:1). Compound N-(7-fluoro-3,3-dimethyl-2-oxo-1,4-dihydroquinolin-6-yl)-2-methyl-5-(2-methylprop-1-enyl)pyridine-4-carboxamide (65 mg, 165.50 umol, 59.88% yield, 97.12% purity) was obtained. LCMS: (M+H)⁺: 382.1.

Step 14: To a solution of N-(7-fluoro-3,3-dimethyl-2-oxo-1,4-dihydroquinolin-6-yl)-2-methyl-5-(2-methylprop-1-enyl)pyridine-4-carboxamide (25 mg, 65.54 umol, 1 eq) in MeOH (2 mL) was added 10% Pd/C (10 mg, 65.54 umol) under H₂ atmosphere. The suspension was degassed and purged with H₂ for 3 times. The mixture was stirred under H₂ (15 Psi) at 25° C. for 1 hr. The reaction mixture was filtered and filtrate concentrated under reduced pressure to give a residue. The residue was purified by prep-TLC (SiO₂, PE:EA=0:1). Then was triturated with PE:EA=10:1(11 mL) at 25° C. for 10 min. And then filtered and filter cake was concentrated under reduced pressure to give a residue. Compound N-(7-fluoro-3,3-dimethyl-2-oxo-1,4-dihydroquinolin-6-yl)-5-isobutyl-2-methyl-pyridine-4-carboxamide (97.2% purity) (31 mg) was obtained. LCMS: (M+H)⁺: 384.1.

### Example 22. Synthesis of Compound 40

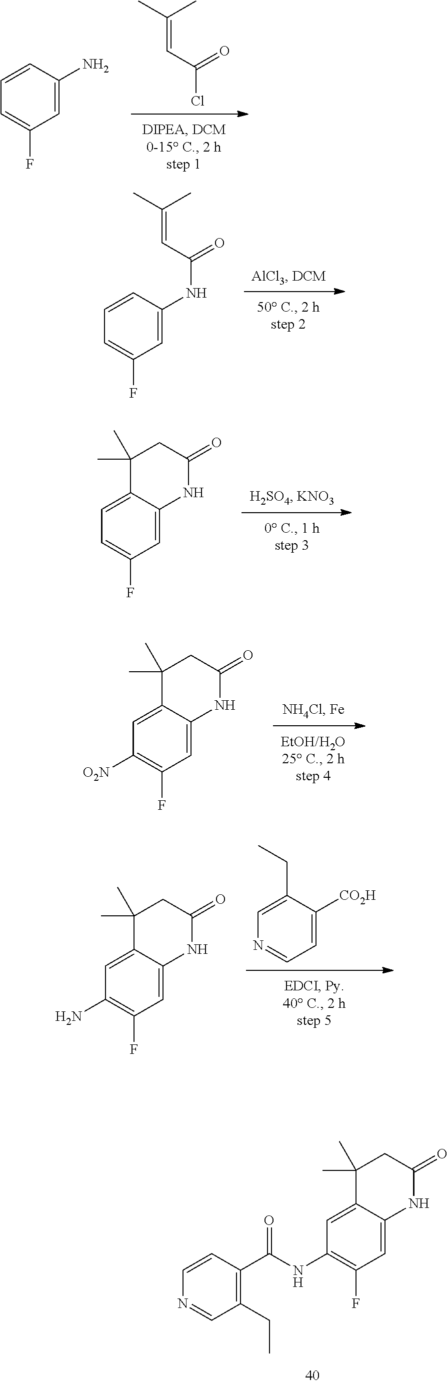

Step 1: To a solution of 3-fluoroaniline (1 g, 9.00 mmol, 862.07 uL, 1 eq) in DCM (10 mL) was added DIPEA (2.33 g, 18.00 mmol, 3.14 mL, 2 eq), 3-methylbut-2-enoyl chloride (1.07 g, 9.00 mmol, 997.19 uL, 1 eq) at 0° C. The resulting mixture was warmed to 15° C. and stirred at 15° C. for 2 h. Then sat NaHCO₃(20 mL) was added slowly to quench the reaction. The organic layer was separated and washed with sat NaHCO₃(50 mL) and water (50 mL*2). The resulting solution was dried over Na₂SO₄ and the filtrate was evaporated. The residue was purified by column chromatography (SiO₂, Petroleum ether/Ethyl acetate=5/1 to 1/1). Compound 3-methyl-N-phenyl-but-2-enamide N-(3-fluorophenyl)-3-methyl-but-2-enamide (800 mg, 4.14 mmol, 46.01% yield) was obtained as a colorless oil.

Step 2: To a solution of N-(3-fluorophenyl)-3-methyl-but-2-enamide (120 mg, 621.06 umol, 1 eq) in DCM (10 mL) was added AlCl₃ (165.63 mg, 1.24 mmol, 67.88 uL, 2 eq). The mixture was stirred at 50° C. for 2 hr. Saturated sodium bicarbonate (50 mL) was added to quench the reaction. The organic layer was separated and washed with sat NaHCO₃ (100 mL) and water (50 mL*3). The resulting solution was dried over Na₂SO₄ and the filtrate evaporated. The residue was purified by prep-TLC (SiO₂, Petroleum ether:EtOAc=0:1). Compound 7-fluor-4,4-dimethyl-1,3-dihydroquinolin-2-one (50 mg, 258.78 umol, 41.67% yield) was obtained as a white solid.

Step 3: To a solution of 7-fluoro-4,4-dimethyl-1,3-dihydroquinolin-2-one (50 mg, 258.78 umol, 1 eq) in conc. H₂SO₄ (2 mL) was added KNO₃ (26.16 mg, 258.78 umol, 1 eq) at 0° C. The mixture was stirred at 0° C. for 1 h. The reaction mixture was cooled at 0° C. and the resulting solution was quenched by adding 10 mL of ice ice/H₂O. The suspension was filtered and filter cake concentrated under reduced pressure to give a residue. The crude product was triturated with the mixture solution of Petroleum ether and EtOAc (10:1, 5.5 mL) at 25° C. for 20 min. The suspension was filtered and filter cake was concentrated under reduced pressure to give a residue. Compound 7-fluoro-4,4-dimethyl-6-nitro-1,3-dihydroquinolin-2-one (44 mg, 184.71 umol, 71.38% yield) was obtained as a white solid.

Step 4: To a solution of 7-fluoro-4,4-dimethyl-6-nitro-1,3-dihydroquinolin-2-one (44 mg, 184.71 umol, 1 eq) in H₂O (1 mL) and EtOH (1 mL) was added NH₄C₁(49.40 mg, 923.54 umol, 5 eq) and Fe (51.57 mg, 923.54 umol, 5 eq). The mixture was stirred at 25° C. for 2 hrs. The reaction mixture was filtered and filtrate was concentrated under reduced pressure to give a residue. The residue was purified by prep-TLC (SiO₂, DCM:MeOH=5:1). Compound 6-amino-7-fluoro-4,4-dimethyl-1,3-dihydroquinolin-2-one (30 mg, 144.07 umol, 78.00% yield) was obtained as a white solid. LCMS: (M+H)⁺: 209.1

Step 5: To a solution of 6-amino-7-fluoro-4,4-dimethyl-1,3-dihydroquinolin-2-one (30 mg, 144.07 umol, 1 eq) in pyridine (2 mL) was added EDCI (33.14 mg, 172.88 umol, 1.2 eq) and 3-ethylpyridine-4-carboxylic acid (21.78 mg, 144.07 umol, 1 eq). The mixture was stirred at 40° C. for 2 hrs. The reaction mixture was concentrated under reduced pressure to remove pyridine. The residue was purified by prep-TLC (SiO₂, Petroleum ether:EtOAc=0:1). Compound 3-ethyl-N-(7-fluoro-4-methyl-2-oxo-3,4-dihydro-1H-quinolin-6-yl)pyridine-4-carboxamide (23.5 mg, 100% purity) was obtained. LCMS: (M+H)⁺: 342.1.

### Example 23. Synthesis of Compound 62

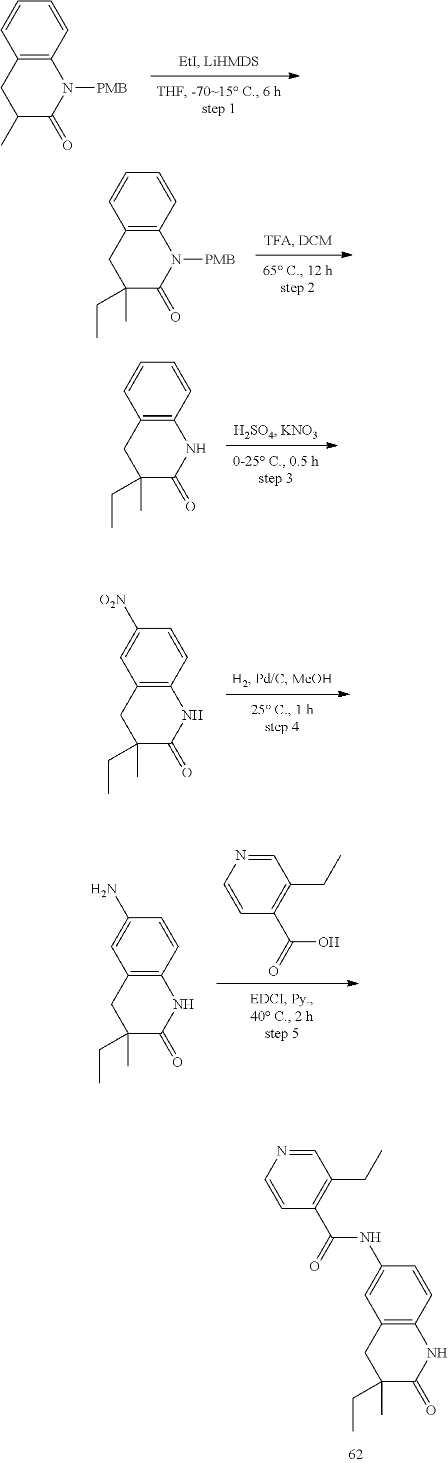

Step 1: To a solution of 1-[(4-methoxyphenyl)methyl]-3-methyl-3,4-dihydroquinolin-2-one (400 mg, 1.42 mmol, 1 eq) in THE (2 mL) was added LiHMDS (2 M, 1.56 mL, 2.2 eq) at −70° C. The mixture was stirred for 30 min at −70° C. Then EtI (1.33 g, 8.53 mmol, 682.28 uL, 6 eq) was added at −70° C. The mixture was allowed to warm to 15° C. and stirred for 5.5 hours. The reaction mixture was quenched by addition H₂O (15 mL), and then diluted with H₂O (15 mL) and extracted with EtOAc (5 mL*3). The combined organic layers were dried over Na₂SO₄, filtered and concentrated under reduced pressure to give a residue. The residue was purified by column chromatography (SiO₂, Petroleum ether/Ethyl acetate=10/1 to 2/1). Compound 3-ethyl-1-[(4-methoxyphenyl)methyl]-3-methyl-4H-quinolin-2-one (300 mg, 969.61 umol, 68.20% yield) was obtained as a yellow oil.

Step 2: The mixture of 3-ethyl-1-[(4-methoxyphenyl)methyl]-3-methyl-4H-quinolin-2-one (300 mg, 969.61 umol, 1 eq) in DCM (1 mL) and TFA (15.40 g, 135.06 mmol, 10.00 mL, 139.29 eq) was stirred at 65° C. for 12 hours. The reaction mixture was concentrated under reduced pressure to give a residue. The residue was purified by prep-TLC (SiO₂, Petroleum ether/Ethyl acetate=2:1). Compound 3-ethyl-3-methyl-1,4-dihydroquinolin-2-one (152 mg, 803.16 umol, 82.83% yield) was obtained as white solid.

Step 3: To a solution of 3-ethyl-3-methyl-1,4-dihydroquinolin-2-one (152 mg, 803.16 umol, 1 eq) in conc.H₂SO₄ (2 mL) was added KNO₃ (81.20 mg, 803.16 umol, 1 eq) at 0° C. The mixture was stirred at 25° C. for 30 min. The reaction mixture was cooled at 0° C. and the resulting solution was stirred for 15 min at 0° C. Then the mixture was quenched by adding 50 mL of H₂O/ice. The suspension was filtered and filter cake concentrated under reduced pressure to give a residue. The crude product was triturated with the mixture solution of Petroleum ether and EtOAc(10:1, 11 mL) at 25° C. for 20 min. The suspension was filtered and filter cake was concentrated under reduced pressure to give a residue. Compound 3-ethyl-3-methyl-6-nitro-1,4-dihydroquinolin-2-one (156 mg, 665.95 umol, 82.92% yield) was obtained as a white solid. LCMS: (M+H)⁺: 235.1.

Step 4: The suspension of 3-ethyl-3-methyl-6-nitro-1,4-dihydroquinolin-2-one (156 mg, 665.95 umol, 1 eq) and 10% Pd/C (50 mg) in THE (2 mL) was degassed and purged with H₂ for 3 times. The mixture was stirred under H₂ (45 Psi) at 25° C. for 1 hr. The reaction mixture was filtered and filtrate concentrated under reduced pressure to give a residue. The crude product was triturated with the mixture solution of Petroleum ether and EtOAc(10:1, 11 mL) at 25° C. for 10 min. The suspension was filtered and filter cake was concentrated under reduced pressure to give a residue. Compound 6-amino-3-ethyl-3-methyl-1,4-dihydroquinolin-2-one (100 mg, 489.55 umol, 73.51% yield) was obtained as a white solid.

Step 5: To a mixture of 6-amino-3-ethyl-3-methyl-1,4-dihydroquinolin-2-one (100 mg, 489.55 umol, 1 eq) and 3-ethylpyridine-4-carboxylic acid (81.40 mg, 538.51 umol, 1.1 eq) in Pyridine (3 mL) was added EDCI (112.62 mg, 587.46 umol, 1.2 eq) in one portion at 40° C. The mixture was stirred at 40° C. for 2 hours. The reaction mixture was concentrated under reduced pressure to give a residue. The residue was purified by prep-TLC (SiO₂, DCM:MeOH=10:1). Compound 3-ethyl-N-(3-ethyl-3-methyl-2-oxo-1,4-dihydroquinolin-6-yl)pyridine-4-carboxamide (7 mg, 95.4% purity) was obtained. LCMS: (M+H)⁺: 338.1

### Example 24. Synthesis of Compound 63

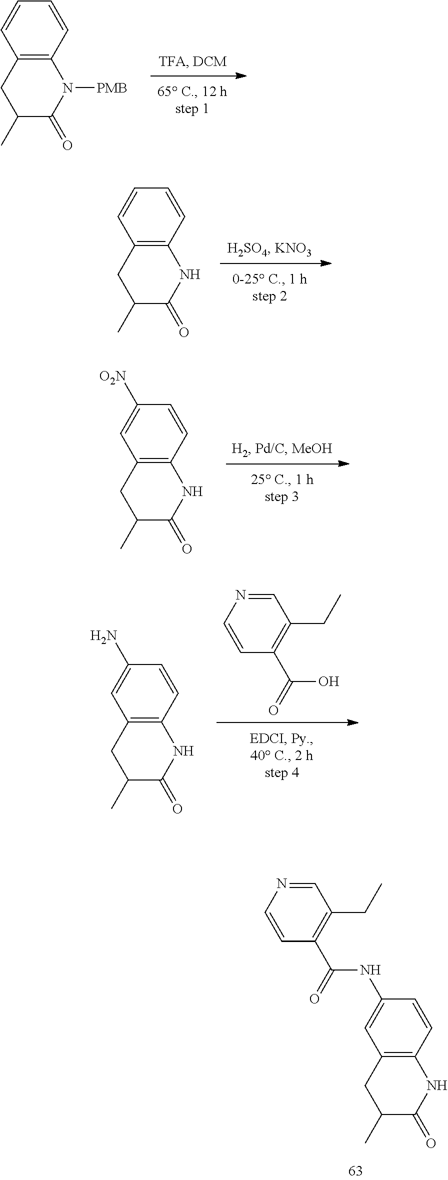

Step 1: To a mixture of 1-[(4-methoxyphenyl)methyl]-3-methyl-3,4-dihydroquinolin-2-one (500 mg, 1.78 mmol, 1 eq) in DCM (5 mL) and TFA (7.70 g, 67.53 mmol, 5 mL, 38.00 eq). The mixture was stirred at 65° C. for 12 hrs. The reaction mixture was concentrated under reduced pressure to give a residue. The residue was purified by column chromatography (SiO₂, Petroleum ether/Ethyl acetate=10/1 to 2/1). Compound 3-methyl-3,4-dihydro-1H-quinolin-2-one (250 mg, 1.55 mmol, 87.27% yield) was obtained as white solid.

Step 2: To a solution of 3-methyl-3,4-dihydro-1H-quinolin-2-one (250 mg, 1.55 mmol, 1 eq) in conc. H₂SO₄ (2 mL) was added KNO₃ (156.80 mg, 1.55 mmol, 1 eq) at 0° C. The mixture was stirred at 25° C. for 1 hr. The reaction mixture was cooled at 0° C. and the resulting solution was stirred for 15 min at 0° C. Then the mixture was quenched by adding 50 mL of H₂O/ice. The suspension was filtered and filter cake concentrated under reduced pressure to give a residue. The crude product was triturated with Petroleum ether: EtOAc (10:1, 11 mL) at 25° C. for 20 min. The suspension was filtered and filter cake was concentrated under reduced pressure to give a residue. Compound 3-methyl-6-nitro-3,4-dihydro-1H-quinolin-2-one (260 mg, 1.26 mmol, 81.30% yield) was obtained as a white solid. LCMS: (M+H)⁺: 207.1.

Step 3: The suspension of 3-methyl-6-nitro-3,4-dihydro-1H-quinolin-2-one (260 mg, 1.26 mmol, 1 eq) and 10% Pd/C (100 mg) in THE (5 mL) was degassed and purged with H₂ for 3 times. The mixture was stirred under H₂ (15 Psi) at 25° C. for 1 hr. The reaction mixture was filtered and filtrate concentrated under reduced pressure to give a residue. The crude product was triturated with Petroleum ether:EtOAc (10:1.11 mL) at 25° C. for 10 min. The suspension was filtered and filter cake was concentrated under reduced pressure to give a residue. Compound 6-amino-3-methyl-3,4-dihydro-1H-quinolin-2-one (150 mg, 851.23 umol, 67.51% yield) was obtained as a white solid.

Step 4: To a mixture of 6-amino-3-methyl-3,4-dihydro-1H-quinolin-2-one (150 mg, 851.23 umol, 1 eq) and 3-ethylpyridine-4-carboxylic acid (141.54 mg, 936.36 umol, 1.1 eq) in pyridine (2 mL) was added EDCI (195.82 mg, 1.02 mmol, 1.2 eq) in one portion at 40° C. The mixture was stirred at 40° C. for 2 hours. The reaction mixture was concentrated under reduced pressure to give a residue. The residue was purified by prep-TLC (SiO₂, DCM:MeOH=10:1). Compound 3-ethyl-N-(3-methyl-2-oxo-3,4-dihydro-1H-quinolin-6-yl)pyridine-4-carboxamide (35.8 mg, 96.7% purity) was obtained. LCMS: (M+H)⁺: 310.1.

### Example 25. Synthesis of Compound 45

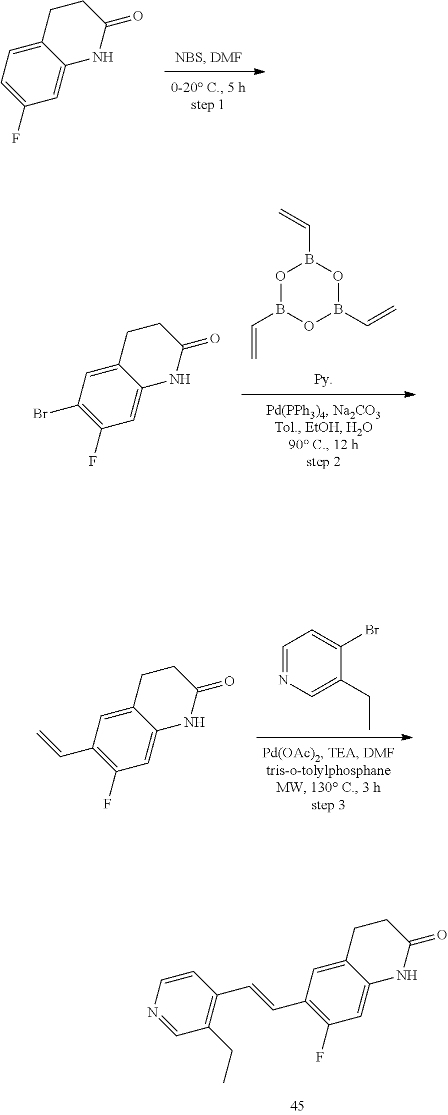

Step 1: To a solution of 7-fluoro-3,4-dihydro-1H-quinolin-2-one (150 mg, 908.19 umol, 1 eq) in DMF (5 mL) was added NBS (177.81 mg, 999.01 umol, 1.1 eq) in portions at 0° C. The mixture was stirred at 20° C. for 5 hrs. The reaction mixture was poured into water (15 mL) to give a suspension. The white solid was filtered, washed with H₂O (5 mL). The filter cake was diluted with EtOAc (10 mL), and extracted with EtOAc (5 mL*2). The combined organic layers were washed with brine (5 mL*2), dried over Na₂SO₄, filtered and concentrated under reduced pressure to give a residue. The residue was purified by prep-TLC (SiO₂, Petroleum ether. EtOAc=2:1). Compound 6-bromo-7-fluoro-3,4-dihydro-1H-quinolin-2-one (120 mg, 491.68 umol, 54.14% yield) was obtained as a white solid. LCMS: (M+H)⁺: 244.0.

Step 2: To a mixture of 6-bromo-7-fluoro-3,4-dihydro-1H-quinolin-2-one (120 mg, 491.68 umol, 1 eq) and 2,4,6-trivinyl-1,3,5,2,4,6-trioxatriborinane (95.33 mg, 590.02 umol, 1.2 eq), Na₂CO₃ (156.34 mg, 1.48 mmol, 3 eq) in toluene (20 mL), EtOH (4 mL), and H₂O (1 mL) was added Pd(PPh₃)₄ (56.82 mg, 49.17 umol, 0.1 eq) in one portion under N₂. The mixture was heated to 90° C. and stirred for 12 hours. The mixture was cooled to 20° C. and poured into ice-water (15 mL). The aqueous phase was extracted with ethyl acetate (20 mL*3). The combined organic phase was washed with brine (30 mL*2), dried with anhydrous Na₂SO₄, filtered and concentrated in vacuum. The residue was purified by prep-TLC (Petroleum ether: EtOAc=0.1). 7-fluoro-6-vinyl-3,4-dihydro-1H-quinolin-2-one (81 mg, 423.64 umol, 86.16% yield) was obtained as yellow solid. LCMS: (M+H)⁺: 192.1.

Step 3: 4-bromo-3-ethyl-pyridine (78.82 mg, 423.64 umol, 1 eq), 7-fluoro-6-vinyl-3,4-dihydro-1H-quinolin-2-one (81 mg, 423.64 umol, 1 eq), tris-o-tolylphosphane (64.47 mg, 211.82 umol, 0.5 eq), TEA (128.60 mg, 1.27 mmol, 176.89 uL, 3 eq) and Pd(OAc)₂ (7.61 mg, 33.89 umol, 0.08 eq) were taken up into a microwave tube in DMF (5 mL). The sealed tube was heated at 130° C. for 3 hours under microwave. The reaction mixture was filtered and concentrated under reduced pressure to give a residue. The residue was purified by prep-TLC (SiO₂, Petroleum ether. EtOAc=1:1) to provide Compound 45, 6-[(E)-2-(3-ethyl-4-pyridyl)vinyl]-7-fluoro-3,4-dihydro-1H-quinolin-2-one (36 mg, 97.5% purity). LCMS: (M+H)⁺: 297.1. ¹HNMR (400 MHz, MeOD, ppm): δ 8.32-8.34 (m, 2H), 7.58-7.64 (m, 2H), 7.39 (s, 2H), 6.68 (d, J=12.0 Hz, 1H), 3.0 (t, J=7.2 Hz, 2H), 2.85 (q, J=7.6 Hz, 2H), 2.61 (t, J=7.2 Hz, 2H), 1.26 (t, J=7.6 Hz, 3H).

### Example 26. Synthesis of Compound 83

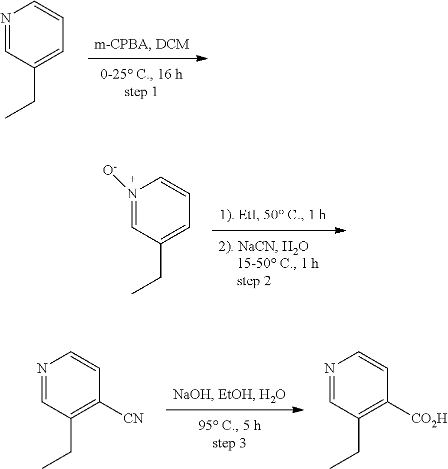

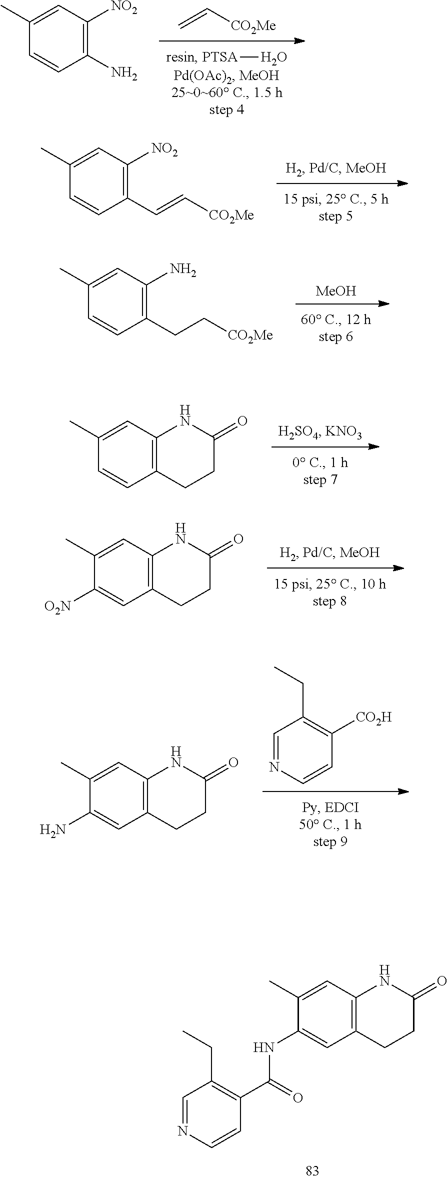

Step 1: To the mixture of 3-ethylpyridine (2 g, 18.66 mmol, 2.10 mL, 1 eq) in DCM (40 mL) was added m-CPBA (3.79 g, 18.66 mmol, 85% purity, 1 eq) in portions at 0° C. Then the mixture was stirred at 25° C. for 16 hr. To the mixture was added sat. Na₂SO₃ (50 mL). Then the mixture was stirred at 25° C. for 1 hr. The mixture was extracted with DCM (25 mL*3). The combined organic phase was washed with brine (30 mL*2), dried with anhydrous Na₂SO₄, filtered and the filtrate was concentrated in vacuum to give compound 3-ethyl-1-oxido-pyridin-1-ium (1.2 g, crude) as white solid.

Step 2: The mixture of 3-ethyl-1-oxido-pyridin-1-ium (1.2 g, 9.74 mmol, 1 eq) and CH₃CH₂I (4.56 g, 29.23 mmol, 2.34 mL, 3 eq) was stirred at 50° C. for 1 hr. Then the mixture was cooled to 15° C. To the mixture was added Petroleum ether (50 mL) and filtered. The filter cake was added to H₂O (30 mL). Then to the mixture was added NaCN (955.06 mg, 19.49 mmol, 2 eq) in H₂O (10 mL) drop-wise at 15° C. The mixture was stirred at 50° C. for 1 h. The mixture was adjusted to pH=12 with 1 M NaOH. The aqueous phase was extracted with ethyl acetate (15 mL*3). The combined organic phase was washed with brine (10 mL*2), dried with anhydrous Na₂SO₄, filtered and concentrated in vacuum. The residue was purified by prep-TLC (SiO₂, Petroleum ether: Ethyl acetate=0:1) to give compound 3-ethylpyridine-4-carbonitrile (0.6 g, 4.54 mmol, 46.59% yield) as white solid.

Step 3: To the mixture of 3-ethylpyridine-4-carbonitrile (0.6 g, 4.54 mmol, 1 eq) in EtOH (6 mL) and H₂O (3 mL) was added NaOH (363.16 mg, 9.08 mmol, 2 eq). The mixture was stirred at 95° C. for 5 hr. The mixture was adjusted pH=5-6 with 1 N HCl then concentrated in vacuum. The residue was purified by prep-TLC (SiO₂, Petroleum ether. Ethyl acetate=0:1) to give 3-ethylpyridine-4-carboxylic acid (0.45 g, 2.98 mmol, 65.57% yield) as off-white solid.

Step 4: Section A: Amberyst A-26(OH) (60 g) and NaNO₂ (35 g, 507.28 mmol, 7.72 eq) in H₂O (1300 mL) was stirred at 25° C. for 0.5 h. The mixture was filtered, then the filter cake was washed with H₂O (500 mL). Section B: To the mixture of 4-methyl-2-nitro-aniline (10 g, 65.72 mmol, 1 eq) and TsOH.H₂O (37.51 g, 197.17 mmol, 3 eq), Pd(OAc)₂ (1.48 g, 6.57 mmol, 0.1 eq) in MeOH (150 mL) was added the product from Section A. Then to the mixture was added methyl prop-2-enoate (28.29 g, 328.62 mmol, 29.59 mL, 5 eq) drop-wise at 0° C., then the mixture was warmed to 60° C. and stirred at 60° C. for 1 h. The mixture was filtered. The filter cake was washed with EtOAc (100 mL). The combined organic phase was concentrated in reduced pressure. The residue was diluted with H₂O (200 mL). The aqueous phase was extracted with ethyl acetate (50 mL*3). The combined organic phase was washed with brine (30 mL), dried with anhydrous Na₂SO₄, filtered and concentrated in vacuum. The residue was purified by column chromatography (SiO₂, Petroleum ether/Ethyl acetate=100/1 to 1/1) to give compound methyl (E)-3-(4-methyl-2-nitro-phenyl)prop-2-enoate (11 g, 49.03 mmol, 74.60% yield, 98.6% purity) as light yellow solid.

Step 5: To a solution of methyl (E)-3-(4-methyl-2-nitro-phenyl)prop-2-enoate (11 g, 49.73 mmol, 1 eq) in MeOH (110 mL) was added 10% Pd/C (1 g) under N₂. The suspension was degassed under vacuum and purged with H₂ several times. The mixture was stirred under H₂ (15 Psi) at 25° C. for 5 hours. The reaction mixture was filtered and the filtrate was concentrated to give compound methyl 3-(2-amino-4-methyl-phenyl)propanoate (7.5 g, 38.81 mmol, 78.05% yield) as off-white solid without further purification. LCMS: (M+H)⁺: 194.1 @ 0.270 min.

Step 6: The mixture of methyl 3-(2-amino-4-methyl-phenyl)propanoate (7.5 g, 38.81 mmol, 1 eq) in MeOH (200 mL) was stirred at 60° C. for 12 hr. The mixture was concentrated in reduced pressure. The crude product was triturated with the solution (Petroleum ether:Ethyl acetate=10:1, 50 mL) at 25° C. for 30 min. The mixture was filtered. The filter cake was concentrated in reduced pressure. 7-methyl-3,4-dihydro-1H-quinolin-2-one (4.8 g, 29.78 mmol, 76.72% yield) was obtained as off-white solid.

Step 7: To the mixture of 7-methyl-3,4-dihydro-1H-quinolin-2-one (2 g, 12.41 mmol, 1 eq) in H₂SO₄ (20 mL) was added KNO₃ (1.51 g, 14.89 mmol, 1.2 eq) drop-wise at 0° C. Then the mixture was stirred at 0° C. for 1 hr. The mixture was poured into ice-water (100 mL). Then the mixture was filtered. The filter cake was triturated with the solution (30 mL, Petroleum ether:Ethyl acetate=2:1) at 25° C. for 30 min, then filtered, the filtrate was concentrated in reduced pressure to give compound 7-methyl-6-nitro-3,4-dihydro-1H-quinolin-2-one (1.1 g, 5.33 mmol, 43.00% yield) as off-white solid. LCMS: (M+H)⁺: 207.1.

Step 8: To a solution of 7-methyl-6-nitro-3,4-dihydro-1H-quinolin-2-one (1.1 g, 5.33 mmol, 1 eq) in MeOH (15 mL) was added 10% Pd/C (0.1 g) under N₂. The suspension was degassed under vacuum and purged with H₂ several times. The mixture was stirred under H₂ (15 psi) at 25° C. for 10 hours. The mixture was filtered and concentrated in vacuum. 6-amino-7-methyl-3,4-dihydro-1H-quinolin-2-one (800 mg, 4.54 mmol, 85.10% yield) was obtained as purple solid without further purification. LCMS: (M+H)⁺: 177.1.

Step 9: The mixture of 6-amino-7-methyl-3,4-dihydro-1H-quinolin-2-one (60 mg, 340.49 umol, 1 eq) and 3-ethylpyridine-4-carboxylic acid (51.47 mg, 340.49 umol, 1.0 eq), EDCI (84.86 mg, 442.64 umol, 1.3 eq) in pyridine (2 mL) was stirred at 50° C. for 1 h. The mixture was concentrated in vacuum. The residue was purified by prep-TLC (SiO₂, Petroleum ether: Ethyl acetate=0:1). 3-ethyl-N-(7-methyl-2-oxo-3,4-dihydro-1H-quinolin-6-yl)pyridine-4-carboxamide (36 mg, 100% purity) was obtained. LCMS: (M+H)⁺: 310.1.

### Example 27. Synthesis of Compound 84

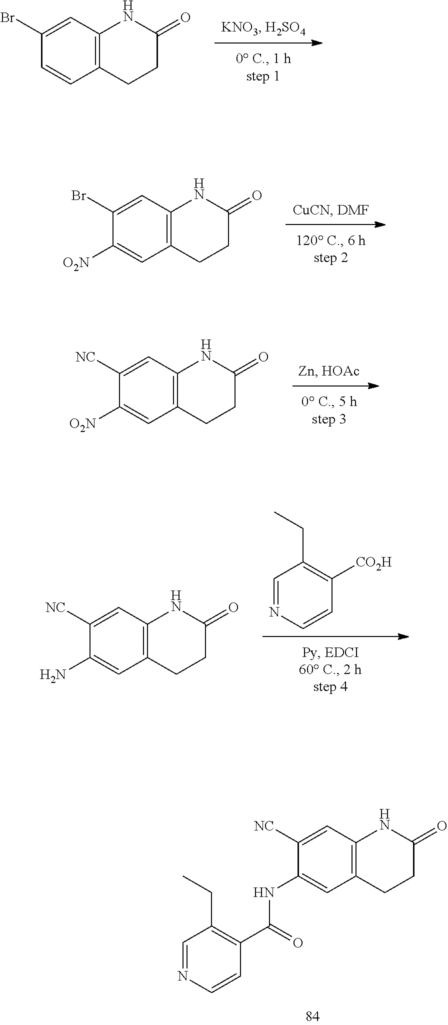

Step 1: To the mixture of 7-bromo-3,4-dihydro-1H-quinolin-2-one (1 g, 4.42 mmol, 1 eq) in H₂SO₄ (10 mL) was added KNO₃ (536.65 mg, 5.31 mmol, 1.2 eq) in portions at 0° C. Then the mixture was stirred at 0° C. for 1 hr. The reaction mixture was poured to ice (100 mL). The mixture was filtered and filter cake was washed with ice-water (20 mL). The filter cake was concentrated in vacuum to give 7-bromo-6-nitro-3,4-dihydro-1H-quinolin-2-one (1 g, 3.69 mmol, 83.40% yield) as off-white solid. LCMS: (M+H)⁺: 272.9.

Step 2: The mixture of 7-bromo-6-nitro-3,4-dihydro-1H-quinolin-2-one (1 g, 3.69 mmol, 1 eq) and CuCN (660.82 mg, 7.38 mmol, 1.61 mL, 2 eq) in DMF (10 mL) was stirred at 120° C. for 6 hr. The mixture was cooled to 25° C. To the mixture was added H₂O (50 mL) and filtered. The filter cake was washed with H₂O (10 mL*2). The filter cake was added to the solution (100 mL, THF:DCM=3:1). The mixture was stirred at 25° C. for 1 hr and filtered. The filtrate was concentrated in vacuum. 6-nitro-2-oxo-3,4-dihydro-1H-quinoline-7-carbonitrile (550 mg, 2.53 mmol, 68.65% yield) was obtained as yellow solid. LCMS: (M+H)⁺: 218.0.

Step 3: The mixture of 6-nitro-2-oxo-3,4-dihydro-1H-quinoline-7-carbonitrile (50 mg, 230.22 umol, 1 eq) in HOAc (2 mL) was added Zn (75.27 mg, 1.15 mmol, 5 eq) at 0° C. The mixture was stirred at 0° C. for 5 hr. The reaction mixture was adjusted to pH=7-8 with sat.NaHCO₃ aq., then extracted with EtOAc 30 mL (10 mL*3). The combined organic layers were washed with brine 10 mL, dried over Na₂SO₄, filtered and concentrated under reduced pressure to give compound 6-amino-2-oxo-3,4-dihydro-1H-quinoline-7-carbonitrile (40 mg, crude) as yellow solid. LCMS: (M+H)⁺: 188.0.

Step 4: To a solution of 6-amino-2-oxo-3,4-dihydro-1H-quinoline-7-carbonitrile (40 mg, 213.68 umol, 1 eq) and 3-ethylpyridine-4-carboxylic acid (32.30 mg, 213.68 umol, 1 eq) in Pyridine (5 mL) was added EDCI (45.06 mg, 235.05 umol, 1.1 eq). The mixture was stirred at 60° C. for 2 hr. The reaction mixture was filtered. The filtrate was concentrated under reduced pressure to give a residue. The residue was purified by prep-TLC (SiO₂, DCM:MeOH=10:1) to give compound N-(7-cyano-2-oxo-3,4-dihydro-1H-quinolin-6-yl)-3-ethyl-pyridine-4-carboxamide (23 mg, 98% purity). LCMS: (M+H)⁺: 321.3.

### Example 28. Synthesis of Compound 34

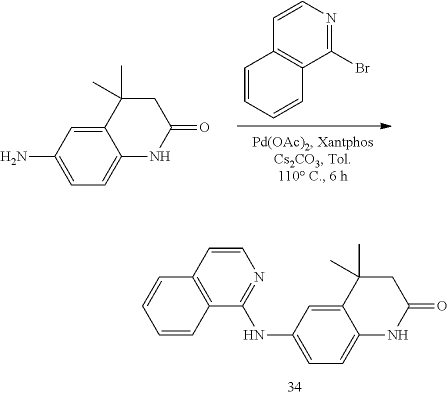 [SMILES: CC1(C)CC(=O)Nc2ccc(N)cc21 ; Brc1nccc2ccccc12 ; CC1(C)CC(=O)Nc2ccc(Nc3nccc4ccccc34)cc21]

To a solution of 6-amino-4,4-dimethyl-1,3-dihydroquinolin-2-one (100 mg, 525.65 umol, 1 eq) and 1-bromoisoquinoline (131.24 mg, 630.78 umol, 1.2 eq) in toluene (3 mL) was added Pd(OAc)₂ (29.50 mg, 131.41 umol, 0.25 eq), Cs₂CO₃ (342.53 mg, 1.05 mmol, 2 eq) and Xantphos (48.66 mg, 84.10 umol, 0.16 eq). The mixture was stirred at 110° C. for 6 hr. The reaction mixture was filtered, and the filtrate was diluted with H₂O 6 mL and extracted with EtOAc 15 mL (5 mL*3). The combined organic layers were dried over Na₂SO₄, filtered and concentrated under reduced pressure to give a residue. The residue was purified by prep-HPLC (column: Waters Xbridge Prep OBD C18 150*40 mm*10 um; mobile phase: [water(0.05% NH3H2O+10 mM NH4HCO3)-ACN]; B %: 30%-60%, 8 min). Compound 6-(1-isoquinolylamino)-4,4-dimethyl-1,3-dihydroquinolin-2-one (41.3 mg, 100% purity) was obtained. LCMS: (M+H)⁺: 318.1.

### Example 29. Synthesis of Compound 35

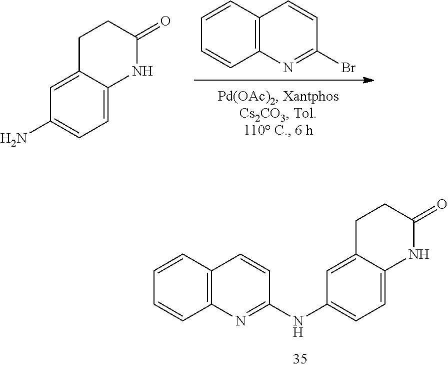

To a solution of 6-amino-3,4-dihydro-1H-quinolin-2-one (100 mg, 616.57 umol, 1 eq) and 2-bromoquinoline (153.94 mg, 739.88 umol, 1.2 eq) in toluene (5 mL) was added Pd(OAc)₂ (34.61 mg, 154.14 umol, 0.25 eq), Xantphos (57.08 mg, 98.65 umol, 0.16 eq) and Cs₂CO₃ (401.78 mg, 1.23 mmol, 2 eq). The mixture was stirred at 110° C. for 6 hr. The reaction mixture was filtered, and the filtrate was diluted with H₂O 6 mL and extracted with EtOAc 15 mL (5 mL*3). The combined organic layers were dried over Na₂SO₄, filtered and concentrated under reduced pressure to give a residue. The residue was purified by prep-HPLC (column: Waters Xbridge Prep OBD C18 150*40 mm*10 um; mobile phase: [water(0.05% NH₃H₂O+10 mM NH₄HCO₃)-ACN]; B %: 25%-55%, 8 min). Compound 6-(2-quinolylamino)-3,4-dihydro-1H-quinolin-2-one (38.6 mg, 100% purity) was obtained. LCMS: (M+H)⁺: 290.1

### Example 30. Synthesis of Compound 36

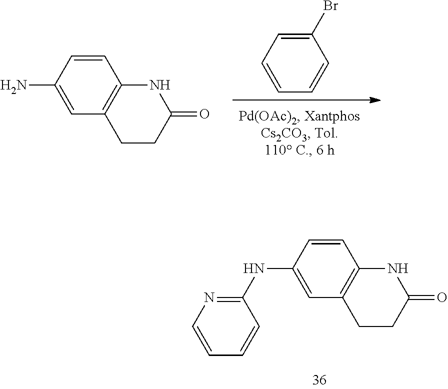 [SMILES: Nc1ccc2c(c1)CCC(=O)N2 ; Brc1ccccc1 ; O=C1CCc2cc(Nc3ccccn3)ccc2N1]

To a solution of 6-amino-3,4-dihydro-1H-quinolin-2-one (100 mg, 616.57 umol, 1 eq) and 2-bromopyridine (116.90 mg, 739.88 umol, 70.42 uL, 1.2 eq) in toluene (5 mL) was added Pd(OAc)₂ (34.61 mg, 154.14 umol, 0.25 eq), Xantphos (57.08 mg, 98.65 umol, 0.16 eq) and Cs₂CO₃ (401.78 mg, 1.23 mmol, 2 eq). The mixture was stirred at 110° C. for 6 hr. The reaction mixture was filtered and filtrate concentrated under reduced pressure to give a residue. The residue was purified by prep-TLC (SiO₂, PE:EA=1:1). Compound 6-(2-pyridylamino)-3,4-dihydro-1H-quinolin-2-one (97.5 mg, 100% purity) was obtained. LCMS: (M+H)⁺: 240.1.

### Example 31. Synthesis of Compound 46

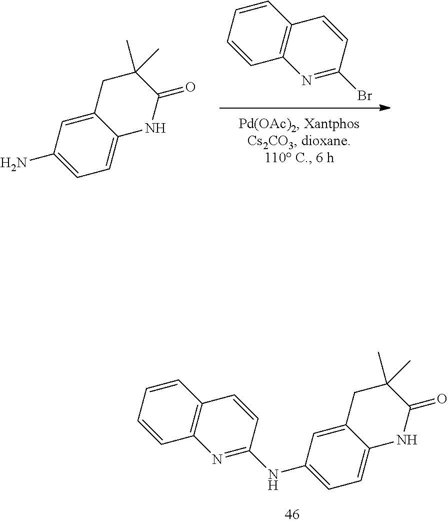 [SMILES: CC1(C)Cc2cc(N)ccc2NC1=O ; Brc1ccc2ccccc2n1 ; CC1(C)Cc2cc(Nc3ccc4ccccc4n3)ccc2NC1=O]

To a solution of 6-amino-3,3-dimethyl-1,4-dihydroquinolin-2-one (50 mg, 262.82 umol, 1 eq) and 2-bromoquinoline (65.62 mg, 315.39 umol, 1.2 eq), Cs₂CO₃ (171.27 mg, 525.65 umol, 2 eq) and Xantphos (24.33 mg, 42.05 umol, 0.16 eq) in dioxane (5 mL) was added Pd(OAc)₂ (14.75 mg, 65.71 umol, 0.25 eq) under N₂ atmosphere. The mixture was stirred at 110° C. for 6 hrs under N₂. The reaction mixture was concentrated under reduced pressure to give a residue. The residue was purified by prep-TLC (SiO2, Petroleum ether. EtOAc=0:1). Then the product was triturated with the mixture solution of Petroleum ether and EtOAc (10:1, 11 mL). The suspension was filtered and filter cake was concentrated under reduced pressure to give a residue. Compound 3, 3-dimethyl-6-(2-quinolylamino)-1,4-dihydroquinolin-2-one (25 mg, 100% purity) was obtained. LCMS: (M+H)⁺: 318.1.

### Example 32. Synthesis of Compound 47

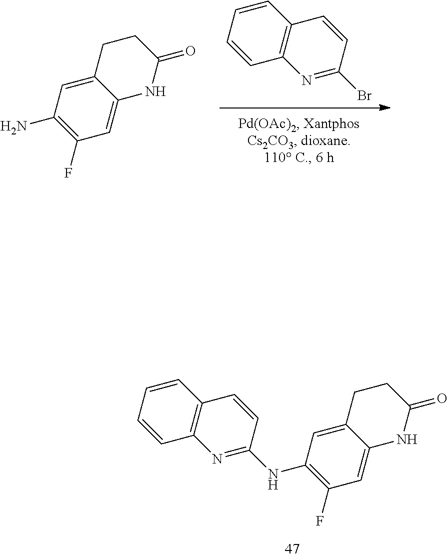

To a solution of 6-amino-7-fluoro-3,4-dihydro-1H-quinolin-2-one (100 mg, 555.00 umol, 1 eq) and 2-bromoquinoline (138.57 mg, 666.01 umol, 1.2 eq), Cs₂CO₃ (361.66 mg, 1.11 mmol, 2 eq) and Xantphos (51.38 mg, 88.80 umol, 0.16 eq) in dioxane (5 mL) was added Pd(OAc)₂ (31.15 mg, 138.75 umol, 0.25 eq) under N₂. The mixture was stirred at 110° C. for 6 hrs. The reaction mixture was filtered, and the filtrate was diluted with H₂O (6 mL) and extracted with EtOAc (5 mL*3). The combined organic layers were dried over Na₂SO₄, filtered and concentrated under reduced pressure to give a residue. The residue was purified by prep-TLC (SiO2, DCM:MeOH=10:1). Compound 7-fluoro-6-(2-quinolylamino)-3,4-dihydro-1H-quinolin-2-one (38.3 mg, 100% purity) was obtained. LCMS: (M+H)⁺: 308.1.

### Example 33. Synthesis of Compound 48

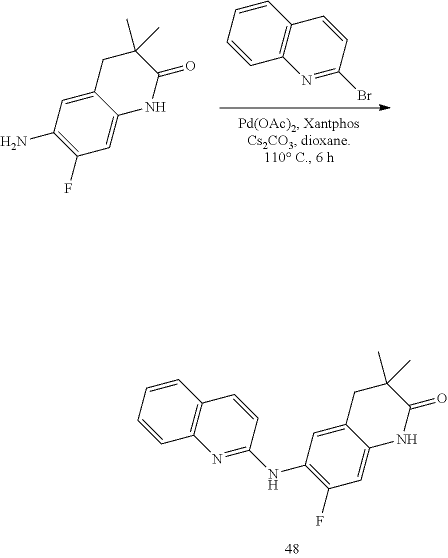

To a solution of 6-amino-7-fluoro-3,3-dimethyl-1,4-dihydroquinolin-2-one (40 mg, 192.09 umol, 1 eq) and 2-bromoquinoline (47.96 mg, 230.51 umol, 1.2 eq), Cs₂CO₃ (125.18 mg, 384.19 umol, 2 eq) and Xantphos (17.78 mg, 30.74 umol, 0.16 eq) in dioxane (5 mL) was added Pd(OAc)₂ (10.78 mg, 48.02 umol, 0.25 eq) in one portion under N₂. The mixture was stirred at 110° C. for 6 hrs. The reaction mixture was filtered, and the filtrate was diluted with H₂O (6 mL) and extracted with EtOAc (5 mL*3). The combined organic layers were dried over Na₂SO₄, filtered and concentrated under reduced pressure to give a residue. The residue was purified by prep-TLC (SiO2, DCM:MeOH=10:1). Compound 7-fluoro-3,3-dimethyl-6-(2-quinolylamino)-1,4-dihydroquinolin-2-one (25 mg, 95.9% purity) was obtained. LCMS: (M+H)⁺: 336.1.

### Example 34. Synthesis of Compounds 86 and 100

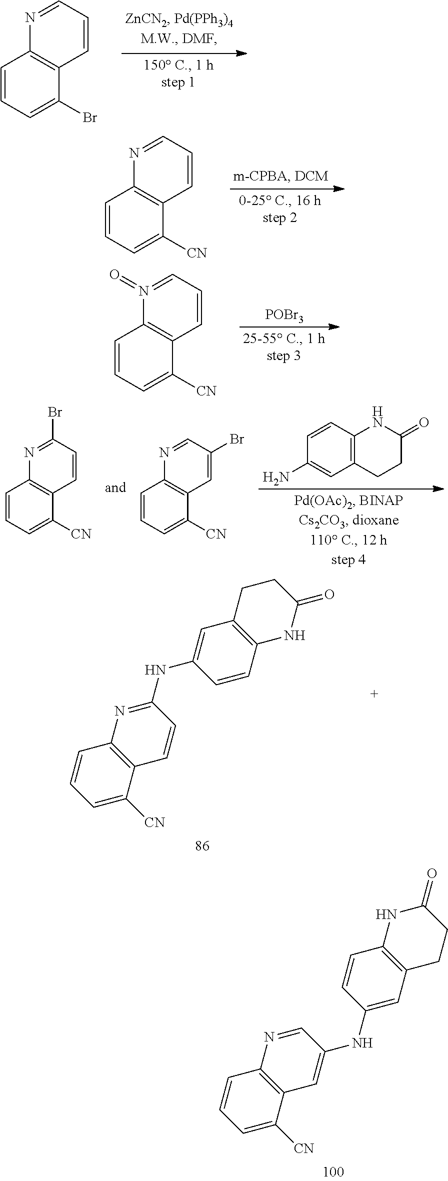

Step 1: 5-bromoquinoline (1 g, 4.81 mmol, 1 eq) and Pd(PPh₃)₄ (1.11 g, 961.29 umol, 0.2 eq), Zn(CN)₂ (846.59 mg, 7.21 mmol, 457.62 uL, 1.5 eq) were taken up into a microwave tube in DMF (10 mL). The sealed tube was heated at 150° C. for 60 min under microwave. To the mixture was added H₂O (250 mL). The mixture was adjusted to pH=12 by 1 N NaOH aq. The aqueous phase was extracted with ethyl acetate (100 mL*3). The combined organic phase was washed with brine (100 mL*4), dried with anhydrous Na₂SO₄, filtered and concentrated in vacuum. The residue was purified by column chromatography (SiO₂, Petroleum ether/Ethyl acetate=25/1 to 3/1). Quinoline-5-carbonitrile (2 g, 12.97 mmol, 53.98%) was obtained as yellow solid.

Step 2: To the mixture of quinoline-5-carbonitrile (2 g, 12.97 mmol, 1 eq) in DCM (60 mL) was added m-CPBA (3.08 g, 14.27 mmol, 80% purity, 1.1 eq) in portions at 0° C. Then the mixture was stirred at 25° C. for 16 hr. To the mixture was added sat. Na₂SO₃ (100 mL). The mixture was stirred at 25° C. for 1 h. Then the mixture was extracted with DCM (30 mL*3). The combined organic phase was washed with brine (30 mL), dried with anhydrous Na₂SO₄, filtered and concentrated in vacuum. The crude product was triturated with the solution (30 mL, Petroleum ether:Ethyl acetate=3:1) at 25° C. for 30 min. 1-oxidoquinolin-1-ium-5-carbonitrile (0.7 g, 4.11 mmol, 31.71% yield) was obtained as white solid. LCMS: (M+H)⁺: 171.0.

Step 3: To the solution of POBr₃ (3.03 g, 10.58 mmol, 1.08 mL, 3 eq) was added 1-oxidoquinolin-1-ium-5-carbonitrile (0.6 g, 3.53 mmol, 1 eq) at 25° C. Then the mixture was stirred at 55° C. for 1 hr. The mixture was cooled to 25° C. and poured into ice-water (100 mL). The mixture was stirred at 25° C. for 1 hr. The mixture was extracted with ethyl acetate (50 mL*3). The combined organic phase was washed with brine (30 mL*2), dried with anhydrous Na₂SO₄, filtered and concentrated in vacuum. The residue was purified by column chromatography (SiO₂, Petroleum ether/Ethyl acetate=10/1 to 0/1). A mixture of 3-bromoquinoline-5-carbonitrile and 2-bromoquinoline-5-carbonitrile (0.2 g, crude) was obtained as yellow solid. LCMS: (M+H)⁺: 234.2

Step 4: To the mixture of 3-bromoquinoline-5-carbonitrile (643.60 umol, 1 eq) and 2-bromoquinoline-5-carbonitrile (150 mg, 643.60 umol, 1 eq), 6-amino-3,4-dihydro-1H-quinolin-2-one (93.95 mg, 579.24 umol, 0.9 eq) in 1,4-dioxane (10 mL) was added (5-diphenylphosphanyl-9,9-dimethyl-xanthen-4-yl)-diphenyl-phosphane (74.48 mg, 128.72 umol, 0.2 eq), Cs₂CO₃ (419.39 mg, 1.29 mmol, 2 eq) and Pd(OAc)₂ (28.90 mg, 128.72 umol, 0.2 eq). Then the mixture was stirred at 110° C. for 12 hr. The mixture was filtered. The filter cake was washed with EtOAc (20 mL). The combined organic phase was concentrated in vacuum. The residue was purified by prep-HPLC (column: Waters Xbridge Prep OBD Cis 150*40 mm*10 um; mobile phase: [water(0.05% NH₃H₂O+10 mM NH₄HCO₃)-ACN]; B %: 20%-50%, 8 min). 2-[(2-oxo-3,4-dihydro-1H-quinolin-6-yl)amino]quinoline-5-carbonitrile (86, 12 mg, 98% purity) and 3-[(2-oxo-3,4-dihydro-1H-quinolin-6-yl)amino]quinoline-5-carbonitrile (100, 13 mg, 94% purity) were obtained.

### Example 35. Synthesis of Compounds 87 and 101

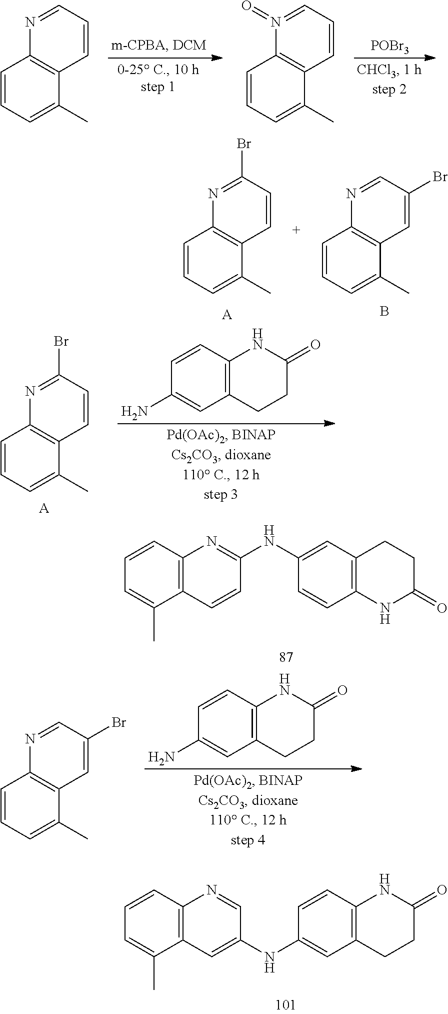

Step 1: To a solution of 5-methylquinoline (1 g, 6.98 mmol, 1 eq) in DCM (20 mL) was added m-CPBA (1.66 g, 7.68 mmol, 80% purity, 1.1 eq) in portions at 0° C. The mixture was stirred at 25° C. for 10 hr. To the mixture was added sat. Na₂SO₃(50 mL). Then the mixture was stirred at 25° C. for 1 hr. The mixture was extracted with DCM (10 mL*3).The combined organic phase was washed with brine (10 mL), dried with anhydrous Na₂SO₄, filtered and concentrated in vacuum. Compound 5-methyl-1-oxido-quinolin-1-ium (1.1 g, crude) was obtained as a white solid. LCMS: (M+H)⁺: 160.1.

Step 2: To the mixture of 5-methyl-1-oxido-quinolin-1-ium (0.9 g, 5.65 mmol, 1 eq) in CHCl₃ (15 mL) was added POBr₃ (2.43 g, 8.48 mmol, 862.16 uL, 1.5 eq) at 0° C. Then the mixture was stirred at 0° C. for 1 hr. The reaction mixture was poured into sat. Na₂CO₃ (50 mL) slowly. Then the mixture was extracted with CH₂Cl₂ 60 mL (20 mL*3). The combined organic layers were washed with brine 40 mL (20 mL*2), dried over Na₂SO₄, filtered and concentrated under reduced pressure to give a residue. The residue was purified by prep-TLC (SiO₂, PE:EA=10:1). Compound 2-bromo-5-methyl-quinoline (A: 170 mg) and 3-bromo-5-methyl-quinoline (B: 190 mg) were obtained.

Step 3: To a solution of 2-bromo-5-methyl-quinoline (200 mg, 900.57 umol, 1 eq), 6-amino-3,4-dihydro-1H-quinolin-2-one (146.06 mg, 900.57 umol, 1 eq) and Cs₂CO₃ (586.85 mg, 1.80 mmol, 2 eq) in 1,4-dioxane (20 mL) was added Pd(OAc)₂ (40.44 mg, 180.11 umol, 0.2 eq) and (5-diphenylphosphanyl-9,9-dimethyl-xanthen-4-yl)-diphenyl-phosphane (104.22 mg, 180.11 umol, 0.2 eq). The mixture was stirred at 110° C. for 12h. The reaction mixture was filtered. The filtrate was concentrated under reduced pressure to give a residue. The residue was purified by prep-TLC (SiO₂, PE:EA=0:1). Compound 6-[(5-methyl-2-quinolyl)amino]-3,4-dihydro-1H-quinolin-2-one (87: 60 mg, 95% purity) was obtained. LCMS: (M+H)⁺: 304.1.

Step 4: To a solution of 3-bromo-5-methyl-quinoline (210 mg, 945.60 umol, 1 eq), 6-amino-3,4-dihydro-1H-quinolin-2-one (153.37 mg, 945.60 umol, 1 eq) and Cs₂CO₃ (616.19 mg, 1.89 mmol, 2 eq) in 1,4-dioxane (5 mL) was added Pd(OAc)₂ (42.46 mg, 189.12 umol, 0.2 eq) and (5-diphenylphosphanyl-9,9-dimethyl-xanthen-4-yl)-diphenyl-phosphane (109.43 mg, 189.12 umol, 0.2 eq). The mixture was stirred at 110° C. for 12h. The reaction mixture was filtered. The filtrate was concentrated under reduced pressure to give a residue. The residue was purified by prep-TLC (SiO₂, PE:EA=0:1). Compound 6-[(5-methyl-3-quinolyl)amino]-3,4-dihydro-1H-quinolin-2-one (101, 70 mg, crude) was obtained. LCMS: (M+H)⁺: 304.1.

### Example 36. Synthesis of Compound 88

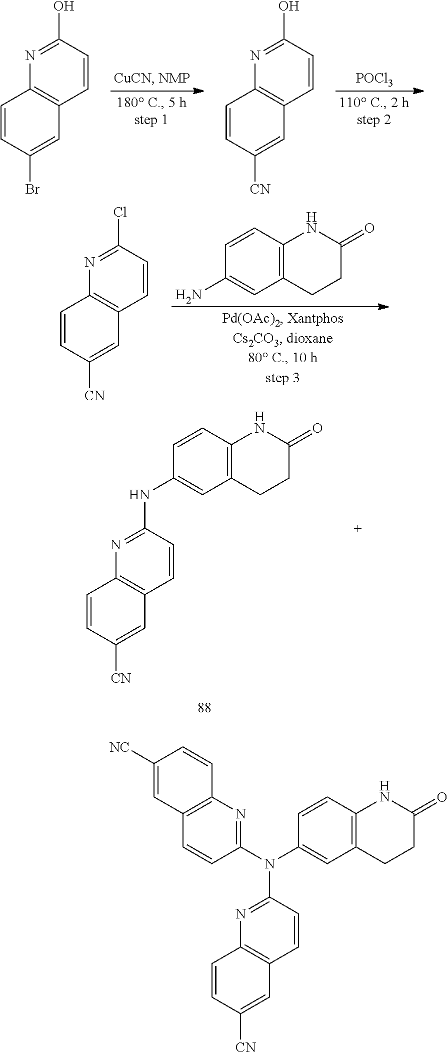

Step 1: To a solution of 6-bromoquinolin-2-ol (2 g, 8.93 mmol, 1 eq) in NMP (30 mL) was added CuCN (1.60 g, 17.85 mmol, 3.90 mL, 2 eq). The mixture was stirred at 180° C. for 5 hr. The reaction mixture was cooled to 25° C. Then the mixture was added H₂O 100 mL. The mixture was filtered and the filter cake was washed with H₂O to give a residue. Compound 2-hydroxyquinoline-6-carbonitrile (2.5 g, crude) was obtained as black solid.

Step 2: 2-hydroxyquinoline-6-carbonitrile (1.3 g, 7.64 mmol, 1 eq) in POCl₃ (13.20 g, 86.09 mmol, 8 mL, 11.27 eq) was stirred at 110° C. for 2 h. The reaction mixture was concentrated under reduced pressure. The residue was added to H₂O 100 mL at 25° C. slowly. The mixture was extracted with EtOAc 150 mL (50 mL*3). The combined organic layers were washed with brine 30 mL, dried over Na₂SO₄, filtered and concentrated under reduced pressure to give a residue. Compound 2-chloroquinoline-6-carbonitrile (400 mg, crude) was obtained as black solid without further purification. LCMS: (M+H)⁺: 189.0.

Step 3: To a solution of 2-chloroquinoline-6-carbonitrile (200 mg, 1.06 mmol, 1 eq) and 6-amino-3,4-dihydro-1H-quinolin-2-one (171.98 mg, 1.06 mmol, 1 eq) in dioxane (5 mL) was added Pd(OAc)₂ (47.61 mg, 212.07 umol, 0.2 eq), Cs₂CO₃ (690.98 mg, 2.12 mmol, 2 eq) and Xantphos (122.71 mg, 212.07 umol, 0.2 eq). The mixture was stirred at 80° C. for 10 hr under N₂. The reaction mixture was filtered and the filtrate was concentrated under reduced pressure to give a residue. The residue was purified by prep-HPLC (column: Waters Xbridge Prep OBD C18 150*40 mm*10 um; mobile phase: [water(0.05% NH₃H₂O+10 mM NH₄HCO₃)-ACN]; B %: 35%-65%, 8 min) to give 2-[(2-oxo-3,4-dihydro-1H-quinolin-6-yl)amino]quinoline-6-carbonitrile (88.19 mg, 99% purity), LCMS: (M+H)⁺: 315.1, and 2-[[1-(6-cyano-2-quinolyl)-2-oxo-3,4-dihydroquinolin-6-yl]amino]quinoline-6-carbonitrile (20 mg, 98% purity), LCMS: (M+H)⁺: 467.2.

### Example 37. Synthesis of Compound 89

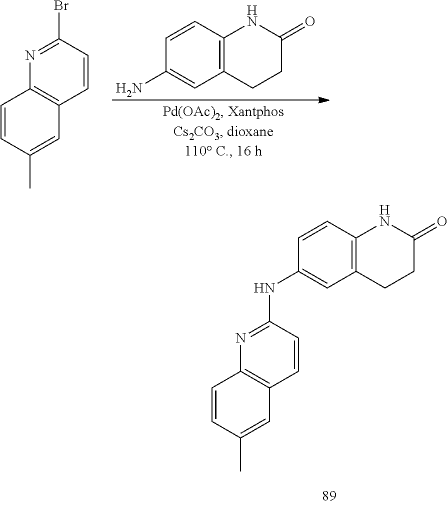 [SMILES: Cc1ccc2nc(Br)ccc2c1 ; Nc1ccc2c(c1)CCC(=O)N2 ; Cc1ccc2nc(Nc3ccc4c(c3)CCC(=O)N4)ccc2c1]

The mixture of 2-bromo-6-methyl-quinoline (100 mg, 450.29 umol, 1 eq), Cs₂CO₃ (293.42 mg, 900.57 umol, 2 eq), 6-amino-3,4-dihydro-1H-quinolin-2-one (73.03 mg, 450.29 umol, 1 eq) Pd(OAc)₂ (20.22 mg, 90.06 umol, 0.2 eq) and (5-diphenylphosphanyl-9,9-dimethyl-xanthen-4-yl)-diphenyl-phosphane (52.11 mg, 90.06 umol, 0.2 eq) in 1,4-dioxane (5 mL) was stirred at 110° C. for 16 hr. The reaction mixture was filtered. The filtrate was concentrated under reduced pressure. The residue was purified by prep-TLC (SiO₂, PE:EA=0:1). Compound 6-[(6-methyl-2-quinolyl)amino]-3,4-dihydro-1H-quinolin-2-one (30 mg, 100% purity) was obtained. LCMS: (M+H)⁺: 304.1.

### Example 38. Synthesis of Compound 90

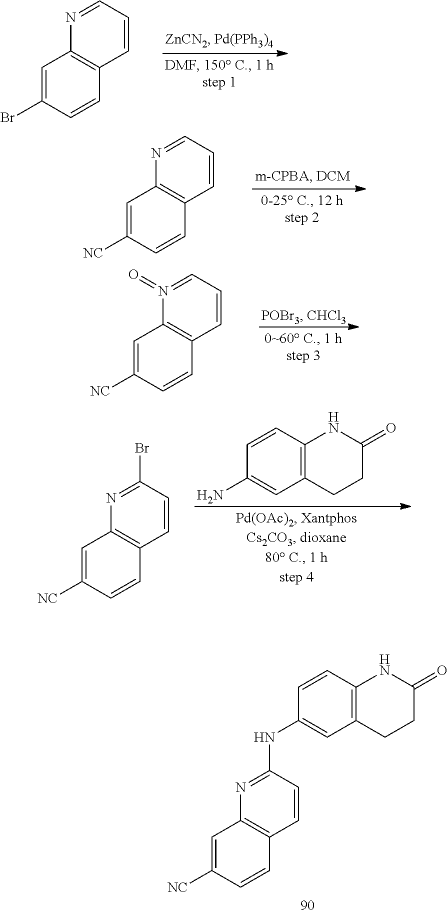

Step 1: 7-bromoquinoline (1 g, 4.81 mmol, 1 eq) Pd(PPh₃)₄ (555.41 mg, 480.64 umol, 0.1 eq) and Zn(CN)₂ (846.59 mg, 7.21 mmol, 457.62 uL, 1.5 eq) were taken up into a microwave tube in DMF (10 mL). The sealed tube was heated at 150° C. for 60 min under microwave. The mixture was filtered. The filter cake was washed with EtOAc (50 mL). To the filtrate was added H₂O (100 mL). Then the mixture was extracted with ethyl acetate (50 mL*2). The combined organic phase was washed with brine (30 mL*4), dried with anhydrous Na₂SO₄, filtered and concentrated in vacuum. The residue was purified by column chromatography (SiO₂, Petroleum ether/Ethyl acetate=100/1 to 5/1). Quinoline-7-carbonitrile (1.35 g) was obtained as off-white solid. LCMS: (M+H)⁺: 155.0.

Step 2: To a solution of quinoline-7-carbonitrile (1.35 g, 8.76 mmol, 1 eq) in DCM (20 mL) was added m-CPBA (2.08 g, 9.63 mmol, 80% purity, 1.1 eq) in portion at 0° C. The mixture was stirred at 25° C. for 12 h. The reaction mixture was added to sat.Na₂SO₃ (50 mL). The mixture was stirred for 30 min. The residue was extracted with CH₂Cl₂ 50 mL (25 mL*2). The combined organic layers were washed with brine 15 mL, dried over Na₂SO₄, filtered and concentrated under reduced pressure to give a residue. Compound 1-oxidoquinolin-1-ium-7-carbonitrile (1.3 g, crude) was obtained as yellow solid. LCMS: (M+H)⁺: 171.0.

Step 3: To a solution of 1-oxidoquinolin-1-ium-7-carbonitrile (0.7 g, 4.11 mmol, 1 eq) in CHCl₃ (8 mL) was added POBr₃ (1.77 g, 6.17 mmol, 627.29 uL, 1.5 eq) at 0° C. The mixture was stirred at 60° C. for 1 hr. The reaction mixture was added sat. Na₂CO₃ (30 mL) slowly. The mixture was extracted with EtOAc 20 mL (10 mL*2). The combined organic layers were washed with brine 20 mL (10 mL*2), dried over Na₂SO₄, filtered and concentrated under reduced pressure to give a residue. The residue was purified by column chromatography (SiO₂, Petroleum ether/Ethyl acetate=100/1 to 30/1) to give 2-bromoquinoline-7-carbonitrile (200 mg) as a white solid.

Step 4: To a solution of 2-bromoquinoline-7-carbonitrile (100 mg, 429.07 umol, 1 eq) and 6-amino-3,4-dihydro-1H-quinolin-2-one (69.59 mg, 429.07 umol, 1 eq) in dioxane (5 mL) was added Pd(OAc)₂ (19.27 mg, 85.81 umol, 0.2 eq), Xantphos (49.65 mg, 85.81 umol, 0.2 eq) and Cs₂CO₃ (279.60 mg, 858.13 umol, 2 eq). The mixture was stirred at 80° C. for 1 hr. The reaction mixture was filtered and the filtrate was concentrated under reduced pressure to give a residue. The residue was purified by prep-HPLC (column: Waters Xbridge Prep OBD C18 150*40 mm*10 um; mobile phase: [water(0.05% NH₃H₂O+10 mM NH₄HCO₃)-ACN]; B %: 30%-60%, 8 min) to give 2-[(2-oxo-3,4-dihydro-1H-quinolin-6-yl)amino]quinoline-7-carbonitrile (35 mg, 100% purity). LCMS: (M+H)⁺: 315.1.

### Example 39. Synthesis of Compound 91

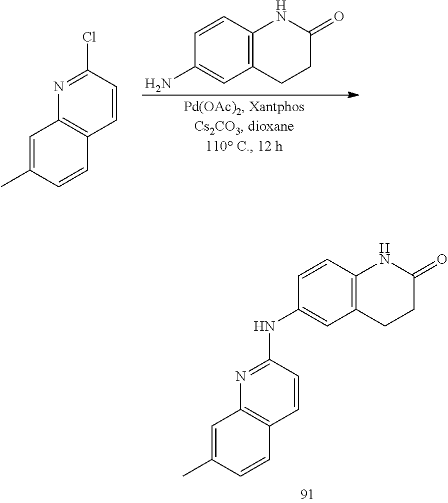 [SMILES: Cc1ccc2ccc(Cl)nc2c1 ; Nc1ccc2c(c1)CCC(=O)N2 ; Cc1ccc2ccc(Nc3ccc4c(c3)CCC(=O)N4)nc2c1]

To the mixture of 2-chloro-7-methyl-quinoline (90 mg, 506.67 umol, 1 eq) and 6-amino-3,4-dihydro-1H-quinolin-2-one (82.18 mg, 506.67 umol, 1 eq) in 1,4-dioxane (10 mL) was added Pd(OAc)₂ (22.75 mg, 101.33 umol, 0.2 eq), (5-diphenylphosphanyl-9,9-dimethyl-xanthen-4-yl)-diphenyl-phosphane (58.63 mg, 101.33 umol, 0.2 eq) and Cs₂CO₃ (330.17 mg, 1.01 mmol, 2 eq). The mixture was stirred at 110° C. for 12 hr. The mixture was cooled to 25° C. The mixture was filtered. The filtrate was concentrated in vacuum to get the residue. The residue was purified by prep-TLC (SiO₂, Petroleum ether. Ethyl acetate=0:1). 6-[(7-methyl-2-quinolyl)amino]-3,4-dihydro-1H-quinolin-2-one (33 mg, 98% purity) was obtained. LCMS: (M+H)⁺: 304.1.

### Example 40. Synthesis of Compound 92

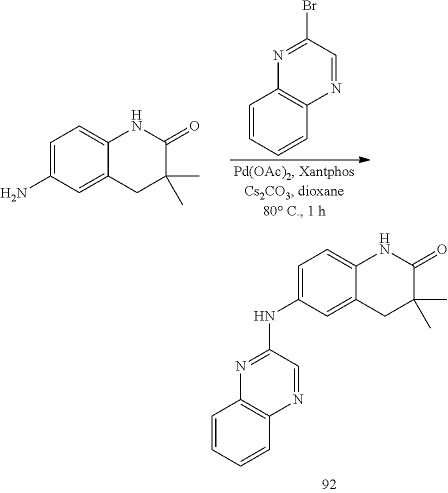 [SMILES: CC1(C)Cc2cc(N)ccc2NC1=O ; Brc1cnc2ccccc2n1 ; CC1(C)Cc2cc(Nc3cnc4ccccc4n3)ccc2NC1=O]

To a solution of 2-bromoquinoxaline (54.94 mg, 262.82 umol, 1 eq) and 6-amino-3,3-dimethyl-1,4-dihydroquinolin-2-one (100 mg, 525.65 umol, 2 eq) in dioxane (2 mL) was added Pd(OAc)₂ (11.80 mg, 52.56 umol, 0.2 eq), Cs₂CO₃ (256.90 mg, 788.47 umol, 3 eq) and Xantphos (30.41 mg, 52.56 umol, 0.2 eq). The mixture was stirred at 80° C. for 1 hr. The reaction mixture was filtered and the filtrate was concentrated under reduced pressure to give a residue. The residue was purified by prep-HPLC (column: Waters Xbridge Prep OBD C18 150*40 mm*10 um; mobile phase: [water(0.05% NH₃H₂O+10 mM NH₄HCO₃)-ACN]; B %: 25%-55%, 8 min) to give 3,3-dimethyl-6-(quinoxalin-2-ylamino)-1,4-dihydroquinolin-2-one (46 mg, 100% purity). LCMS: (M+H)⁺: 319.2

### Example 41. Synthesis of Compound 93

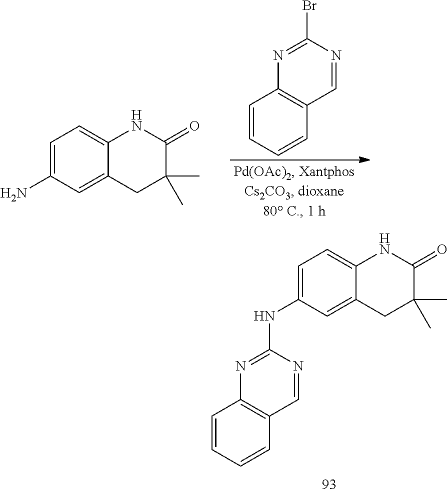 [SMILES: CC1(C)Cc2cc(N)ccc2NC1=O ; Brc1ncc2ccccc2n1 ; CC1(C)Cc2cc(Nc3ncc4ccccc4n3)ccc2NC1=O]

To a solution of 2-bromoquinazoline (54.94 mg, 262.83 umol, 1 eq) and 6-amino-3,3-dimethyl-1,4-dihydroquinolin-2-one (100 mg, 525.65 umol, 2 eq) in dioxane (2 mL) was added Pd(OAc)₂ (11.80 mg, 52.57 umol, 0.2 eq), Cs₂CO₃ (256.90 mg, 788.48 umol, 3 eq) and Xantphos (30.41 mg, 52.57 umol, 0.2 eq). The mixture was stirred at 80° C. for 1 hr. The reaction mixture was filtered and the filtrate was concentrated under reduced pressure to give a residue. The residue was purified by prep-HPLC (column: Waters Xbridge Prep OBD C18 150*40 mm*10 um; mobile phase: [water(0.05% NH₃H₂O+10 mM NH₄HCO₃)-ACN]; B %: 25%-55%, 8 min) to give 3,3-dimethyl-6-(quinazolin-2-ylamino)-1,4-dihydroquinolin-2-one (49 mg, 100% purity). LCMS: (M+H)⁺: 319.1

### Example 42. Synthesis of Compound 94

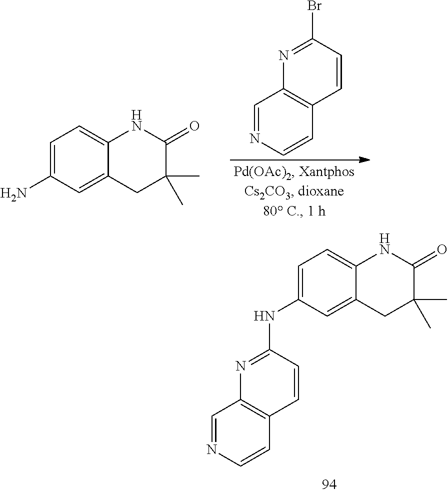 [SMILES: CC1(C)Cc2cc(N)ccc2NC1=O ; Brc1ccc2ccncc2n1 ; CC1(C)Cc2cc(Nc3ccc4ccncc4n3)ccc2NC1=O]

To a solution of 2-bromo-1,7-naphthyridine (54.94 mg, 262.82 umol, 1 eq) and 6-amino-3,3-dimethyl-1,4-dihydroquinolin-2-one (100 mg, 525.65 umol, 2 eq) in dioxane (2 mL) was added Pd(OAc)₂ (11.80 mg, 52.56 umol, 0.2 eq), Cs₂CO₃ (256.90 mg, 788.47 umol, 3 eq) and Xantphos (30.42 mg, 52.56 umol, 0.2 eq). The mixture was stirred at 80° C. for 1 hr. The reaction mixture was filtered and the filtrate was concentrated under reduced pressure to give a residue. The residue was purified by prep-HPLC (column: Waters Xbridge Prep OBD C18 150*40 mm*10 um; mobile phase: [water(0.05% NH₃H₂O+10 mM NH₄HCO₃)-ACN]; B %: 25%-55%, 8 min) to give 3,3-dimethyl-6-(1,7-naphthyridin-2-ylamino)-1,4-dihydroquinolin-2-one (29 mg, 100% purity). LCMS: (M+H)⁺: 319.2

### Example 43. Synthesis of Compound 95

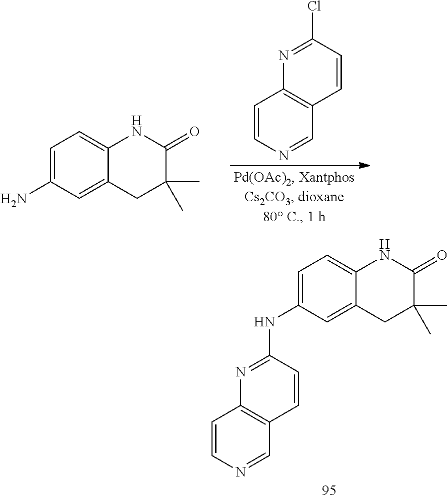 [SMILES: CC1(C)Cc2cc(N)ccc2NC1=O ; Clc1ccc2cnccc2n1 ; CC1(C)Cc2cc(Nc3ccc4cnccc4n3)ccc2NC1=O]

To a solution of 2-chloro-1,6-naphthyridine (43.26 mg, 262.82 umol, 1 eq) and 6-amino-3,3-dimethyl-1,4-dihydroquinolin-2-one (100 mg, 525.65 umol, 2 eq) in dioxane (2 mL) was added Pd(OAc)₂ (11.80 mg, 52.56 umol, 0.2 eq), Cs₂CO₃ (256.90 mg, 788.47 umol, 3 eq) and Xantphos (30.42 mg, 52.56 umol, 0.2 eq). The mixture was stirred at 80° C. for 1 hr. The reaction mixture was filtered and the filtrate was concentrated under reduced pressure to give a residue. The residue was purified by prep-HPLC (column: Welch Xtimate C18 150*25 mm*5 um; mobile phase: [water(0.04% HCl)-ACN]; B %: 5%-15%, 8 min) to give 3,3-dimethyl-6-(1,6-naphthyridin-2-ylamino)-1,4-dihydroquinolin-2-one (9 mg, 100% purity, HCl). LCMS: (M+H)⁺: 319.1.

### Example 44. Synthesis of Compound 96

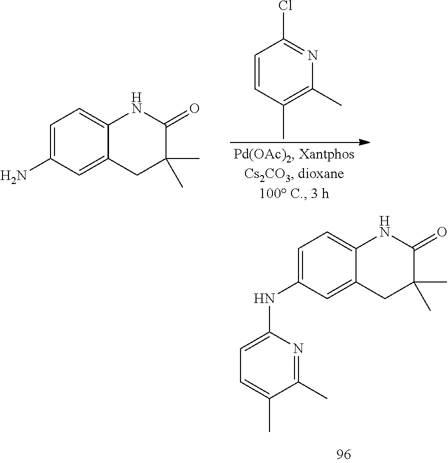

To a solution of 6-chloro-2,3-dimethyl-pyridine (74.43 mg, 525.65 umol, 1 eq) and 6-amino-3,3-dimethyl-1,4-dihydroquinolin-2-one (100 mg, 525.65 umol, 1 eq) in dioxane (2 mL) was added Pd(OAc)₂ (23.60 mg, 105.13 umol, 0.2 eq), Cs₂CO₃ (513.80 mg, 1.58 mmol, 3 eq) and Xantphos (60.83 mg, 105.13 umol, 0.2 eq). The mixture was stirred at 100° C. for 3 hr. The reaction mixture was filtered and the filtrate was concentrated under reduced pressure to give a residue. The residue was purified by prep-HPLC (column: Waters Xbridge Prep OBD C18 150*40 mm*10 um; mobile phase: [water(0.05% NH₃H₂O+10 mM NH₄HCO₃)-ACN]; B %: 25%-55%, 8 min) to give 6-[(5,6-dimethyl-2-pyridyl)amino]-3,3-dimethyl-1,4-dihydroquinolin-2-one (59 mg, 99% purity). LCMS: (M+H)⁺: 296.1.

### Example 45. Synthesis of Compound 97

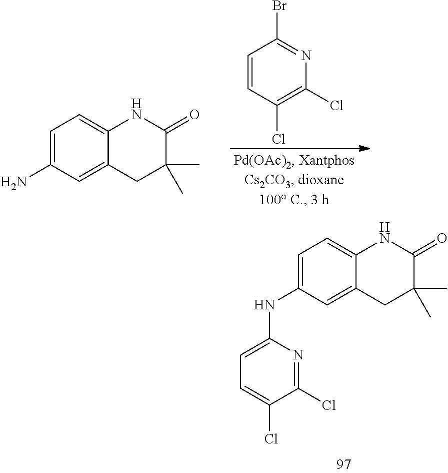 [SMILES: CC1(C)Cc2cc(N)ccc2NC1=O ; Clc1ccc(Br)nc1Cl ; CC1(C)Cc2cc(Nc3ccc(Cl)c(Cl)n3)ccc2NC1=O]

To a solution of 6-bromo-2,3-dichloro-pyridine (59.63 mg, 262.82 umol, 1 eq) and 6-amino-3,3-dimethyl-1,4-dihydroquinolin-2-one (50 mg, 262.82 umol, 1 eq) in dioxane (2 mL) was added Pd(OAc)₂ (11.80 mg, 52.56 umol, 0.2 eq), Cs₂CO₃ (256.90 mg, 788.47 umol, 3 eq) and Xantphos (30.42 mg, 52.56 umol, 0.2 eq). The mixture was stirred at 100° C. for 3 hr. The reaction mixture was filtered and the filtrate was concentrated under reduced pressure to give a residue. The residue was purified by prep-HPLC (column: Waters Xbridge Prep OBD C18 150*40 mm*10 um; mobile phase: [water(0.05% NH₃H₂O+10 mM NH₄HCO₃)-ACN]; B %: 30%-60%, 8 min) to give 6-[(5,6-dichloro-2-pyridyl)amino]-3,3-dimethyl-1,4-dihydroquinolin-2-one (31 mg, 97% purity). LCMS: (M+H)⁺: 336.1.

### Example 46. Synthesis of Compound 98

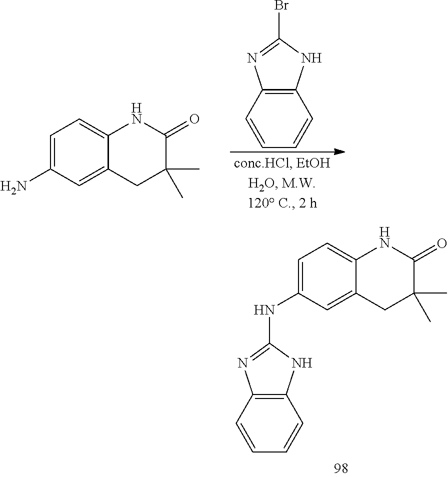 [SMILES: CC1(C)Cc2cc(N)ccc2NC1=O ; Brc1nc2ccccc2[nH]1 ; CC1(C)Cc2cc(Nc3nc4ccccc4[nH]3)ccc2NC1=O]

To a solution of 2-bromo-1H-benzimidazole (103.57 mg, 525.65 umol, 1 eq) and 6-amino-3,3-dimethyl-1,4-dihydroquinolin-2-one (100 mg, 525.65 umol, 1 eq) in H₂O (0.4 mL) and EtOH (2 mL) was added conc. HCl (0.1 mL). The mixture was taken up into a microwave tube. The sealed tube was heated at 120° C. for 2 hr under microwave. The reaction mixture was concentrated under reduced pressure to give a residue. The residue was purified by prep-HPLC (column: Waters Xbridge Prep OBD C18 150*40 mm*10 um; mobile phase: [water(0.04% NH₃H₂O+10 mM NH₄HCO₃)-ACN]; B %: 20%-50%, 8 min) to give 6-(1H-benzimidazol-2-ylamino)-3,3-dimethyl-1,4-dihydroquinolin-2-one (56 mg, 93% purity). LCMS: (M+H)⁺: 307.1.

### Example 47. Synthesis of Compound 99

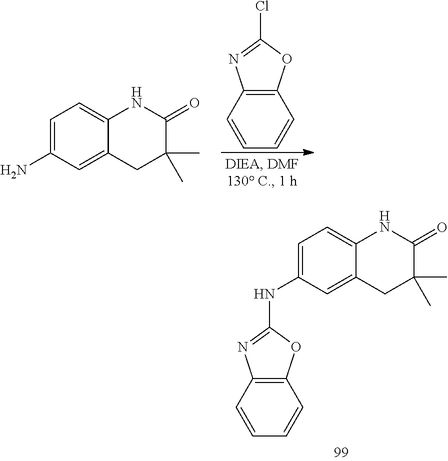

To a solution of 2-chloro-1,3-benzoxazole (40.36 mg, 262.82 umol, 29.90 uL, 1 eq) and 6-amino-3,3-dimethyl-1,4-dihydroquinolin-2-one (50 mg, 262.82 umol, 1 eq) in DMF (2 mL) was added DIPEA (67.94 mg, 525.65 umol, 91.56 uL, 2 eq). The mixture was stirred at 130° C. for 1 hr. The reaction mixture was quenched by addition H₂O mL at 20° C., and then extracted with EtOAc 15 mL (5 mL*3). The combined organic layers were washed with brine 10 mL (5 mL*2), dried over Na₂SO₄, filtered and the filtrate was concentrated under reduced pressure to give a residue. The residue was purified by prep-TLC (SiO₂, PE:EA=1:1) to give 6-(1,3-benzoxazol-2-ylamino)-3,3-dimethyl-1,4-dihydroquinolin-2-one (19 mg, 97% purity). LCMS: (M+H)⁺: 308.1.

### Example 48. Synthesis of Compound 103

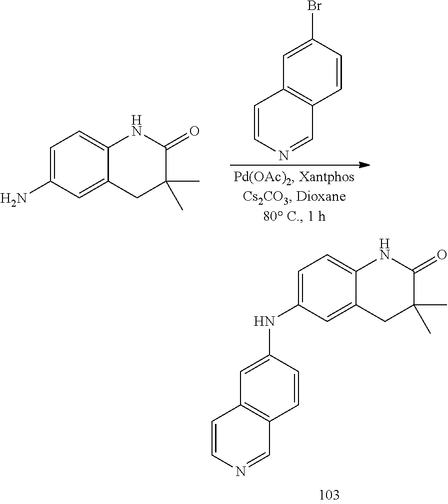 [SMILES: CC1(C)Cc2cc(N)ccc2NC1=O ; Brc1ccc2cnccc2c1 ; CC1(C)Cc2cc(Nc3ccc4cnccc4c3)ccc2NC1=O]

To a solution of 6-bromoisoquinoline (54.68 mg, 262.82 umol, 1 eq) and 6-amino-3,3-dimethyl-1,4-dihydroquinolin-2-one (100 mg, 525.65 umol, 2 eq) in dioxane (3 n1L) was added Pd(OAc)₂ (11.80 mg, 52.56 umol, 0.2 eq) and Xantphos (30.41 mg, 52.56 umol, 0.2 eq) and Cs₂CO₃ (256.90 mg, 788.47 umol, 3 eq). The mixture was stirred at 80° C. for 1 hr. The reaction mixture was filtered and the filtrate was concentrated under reduced pressure to give a residue. The residue was purified by prep-HPLC (column: Waters Xbridge Prep OBD C18 150*40 mm*10 um; mobile phase: [water(0.04% NH₃H₂O+10 mM NH₄HCO₃)-ACN]; B %: 25%-55%, 8 min) to give 6-(6-isoquinolylamino)-3,3-dimethyl-1,4-dihydroquinolin-2-one (29 mg, 93% purity). LCMS: (M+H)⁺: 318.2.

### Example 49. Synthesis of Compound 104

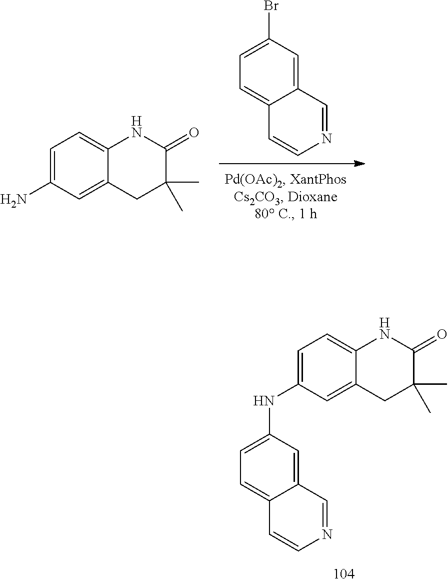 [SMILES: CC1(C)Cc2cc(N)ccc2NC1=O ; Brc1ccc2ccncc2c1 ; CC1(C)Cc2cc(Nc3ccc4ccncc4c3)ccc2NC1=O]

To a solution of 7-bromoisoquinoline (54.68 mg, 262.83 umol, 1 eq) and 6-amino-3,3-dimethyl-1,4-dihydroquinolin-2-one (100 mg, 525.65 umol, 2 eq) in Dioxane (3 mL) was added Pd(OAc)₂ (11.80 mg, 52.57 umol, 0.2 eq) and XantPhos (30.42 mg, 52.57 umol, 0.2 eq) and Cs₂CO₃ (256.90 mg, 788.48 umol, 3 eq). The mixture was stirred at 80° C. for 1 hr. The reaction mixture was filtered and the filtrate was concentrated under reduced pressure to give a residue. The residue was purified by prep-TLC (SiO₂, PE:EA=0:1) to give 6-(7-isoquinolylamino)-3,3-dimethyl-1,4-dihydroquinolin-2-one (13 mg, 98% purity). LCMS: (M+H)⁺: 318.1.

### Example 50. Synthesis of Compound 53

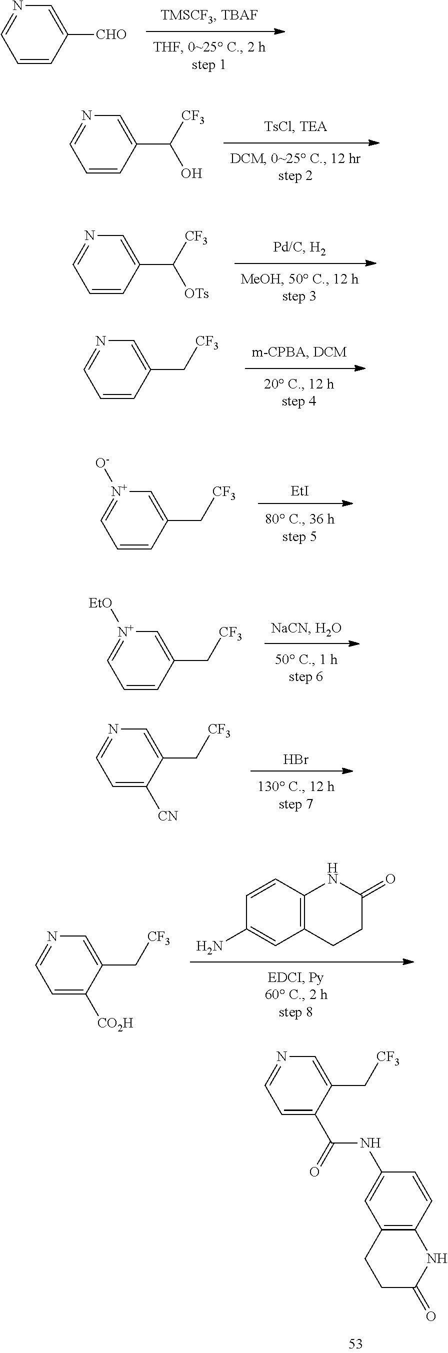

Step 1: To a solution of pyridine-3-carbaldehyde (20 g, 186.72 mmol, 17.54 mL, 1 eq) in THE (200 mL) was added trimethyl(trifluoromethyl)silane (29.21 g, 205.40 mmol, 1.1 eq). Then the mixture was added tetrabutylammonium fluoride trihydrate (1 M, 18.67 mL, 0.1 eq) in THE (50 mL) at 0° C. The mixture was stirred at 25° C. for 2 hrs. The reaction was quenched by 1N HCl(10 ml) slowly and then the mixture was neutralized by sodium bicarbonate (50 mL). The mixture was extracted with Ethyl acetate (150 mL*3). The combined organic phase was washed with brine (50 mL*3), dried over anhydrous Na₂SO₄, filtered and concentrated in vacuo. Compound 2,2,2-trifluoro-1-(3-pyridyl)ethanol (35 g, crude) was obtained as a brown oil.

Step 2: To a solution of 2,2,2-trifluoro-1-(3-pyridyl)ethanol (25 g, 141.14 mmol, 1 eq) and TEA (21.42 g, 211.72 mmol, 29.47 mL, 1.5 eq) in DCM (250 mL) was added TsCl (40.36 g, 211.72 mmol, 1.5 eq) drop-wise at 0° C. The mixture was stirred at 25° C. for 12 hrs. The reaction mixture was poured into ice water (150 mL). The aqueous phase was extracted with DCM (100 mL*3). The combined organic phase was washed with brine (30 mL*3), dried with anhydrous Na₂SO₄, filtered and concentrated in vacuum. The residue was purified by column chromatography (SiO₂, Petroleum ether/Ethyl acetate=15/1 to 0/1). Compound [2,2,2-trifluoro-1-(3-pyridyl)ethyl] 4-methylbenzenesulfonate (26 g, 78.48 mmol, 55.60% yield) was obtained as a yellow solid.

Step 3: To a solution of [2,2,2-trifluoro-1-(3-pyridyl)ethyl] 4-methylbenzenesulfonate (26 g, 78.48 mmol, 1 eq) in MeOH (300 mL) was added 10% Pd/C (0.2 g) under N₂. The suspension was degassed under vacuum and purged with H₂ several times. The mixture was stirred under H₂ (50 Psi) at 50° C. for 12 hours. The reaction mixture was filtered and concentrated under reduced pressure to get a residue. The residue was dissolved with H₂O (80 mL) and adjusted to pH=8 with NaOH, Then the mixture was extracted with DCM 150 mL (50 mL*3). The combined organic layers were washed with brine 60 mL (20 mL*3), dried over with Na₂SO₄, filtered and concentrated under reduced pressure to give a residue. Compound 3-(2,2,2-trifluoroethyl)pyridine (7.5 g, crude) was obtained as a brown oil.

Step 4: To a solution of 3-(2,2,2-trifluoroethyl)pyridine (8 g, 49.65 mmol, 1 eq) in DCM (150 mL) was added m-CPBA (9.07 g, 44.69 mmol, 85% purity, 0.9 eq). The mixture was stirred at 20° C. for 12 hrs. The reaction mixture was quenched by addition saturated Na₂SO₃ 150 mL at 20° C. and stirred at 20° C. for 0.5 hr. Then the mixture was extracted with DCM (100 mL*2). The combined organic phase was washed with brine (50 mL), dried with anhydrous Na₂SO₄, filtered and concentrated in vacuum. Compound 1-oxido-3-(2,2,2-trifluoroethyl)pyridin-1-ium (9 g, crude) was obtained as a yellow solid.

Step 5: A mixture of 1-oxido-3-(2,2,2-trifluoroethyl)pyridin-1-ium (7 g, 39.52 mmol, 1 eq), Ell (18.49 g, 118.56 mmol, 9.48 mL, 3 eq) was degassed and purged with N₂ for 3 times, and then the mixture was stirred at 60° C. for 36 hrs under N₂ atmosphere. The reaction mixture was washed with Petroleum ether (150 ml), and then the mixture was concentrated under reduced pressure. Compound 1-ethoxy-3-(2,2,2-trifluoroethyl)pyridin-1-ium iodide (10 g, crude) was obtained as a brown oil.

Step 6: To a solution of 1-ethoxy-3-(2,2,2-trifluoroethyl)pyridin-1-ium iodide (6 g, 29.10 mmol, 1 eq) in H₂O (60 mL) was added NaCN (2.41 g, 49.17 mmol, 1.69 eq) in H₂O (20 mL) drop-wise at 50° C. The mixture was stirred at 50° C. for 1 hr. The mixture was cooled to 25° C. The mixture was extracted with ethyl acetate (60 mL*3). The combined organic phase was washed with brine (40 mL*2), dried with anhydrous Na₂SO₄, filtered and concentrated in vacuum. The residue was purified by flash silica gel chromatography (ISCO®; 12 g SepaFlash® Silica Flash Column, Eluent of 0-9% Ethyl acetate/Petroleum ethergradient @ 40 mL/min). Compound 3-(2,2,2-trifluoroethyl)pyridine-4-carbonitrile (0.35 g, 1.88 mmol, 6.46% yield) was obtained as a yellow oil.

Step 7: A mixture of 3-(2,2,2-trifluoroethyl)pyridine-4-carbonitrile (50 mg, 268.62 umol, 1 eq) in HBr (3 mL) was degassed and purged with N₂ for 3 times, and then the mixture was stirred at 130° C. for 12 hrs under N₂ atmosphere. The reaction mixture was concentrated under reduced pressure to give a residue. Compound 3-(2,2,2-trifluoroethyl)pyridine-4-carboxylic acid (0.1 g, crude, HBr) was obtained as a brown solid.

Step 8: To a mixture of 3-(2,2,2-trifluoroethyl)pyridine-4-carboxylic acid (80 mg, 389.99 umol, 2.00 eq) and 6-amino-3,4-dihydro-1H-quinolin-2-one (31.63 mg, 194.99 umol, 1 eq) in Pyridine (1 mL) was added EDCI (44.86 mg, 233.99 umol, 1.2 eq). The mixture was stirred at 60° C. for 2 hrs. The reaction mixture was concentrated under reduced pressure to give a residue. The residue was purified by prep-TLC (SiO₂, Petroleum ether. Ethyl acetate=0:1). Compound N-(2-oxo-3,4-dihydro-1H-quinolin-6-yl)-3-(2,2,2-trifluoroethyl)pyridine-4-carboxamide (0.04 g, 96.6% purity) was obtained. LCMS: (M+H)⁺: 350.0.

### Example 51. Synthesis of Compound 82

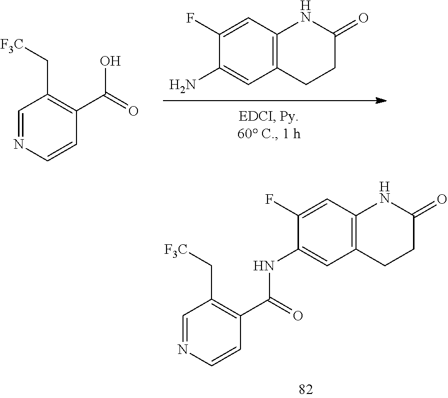 [SMILES: O=C(O)c1ccncc1CC(F)(F)F ; Nc1cc2c(cc1F)NC(=O)CC2 ; O=C1CCc2cc(NC(=O)c3ccncc3CC(F)(F)F)c(F)cc2N1]

To a mixture of 3-(2,2,2-trifluoroethyl)pyridine-4-carboxylic acid (50 mg, 243.74 umol, 1.00 eq) and 6-amino-7-fluoro-3,4-dihydro-1H-quinolin-2-one (43.92 mg, 243.74 umol, 1 eq) in Pyridine (1 mL) was added EDCI (56.07 mg, 292.49 umol, 1.2 eq). The mixture was stirred at 60° C. for 1 hr. The reaction mixture was concentrated under reduced pressure to give a residue. The residue was purified by prep-TLC (SiO₂, Ethyl acetate:MeOH=10:1). Compound N-(7-fluoro-2-oxo-3,4-dihydro-1H-quinolin-6-yl)-3-(2,2,2-trifluoroethyl)pyridine-4-carboxamide (25 mg, 100% purity) was obtained. LCMS: (M+H)⁺: 318.1.

### Example 52. Synthesis of Compounds 59 and 71

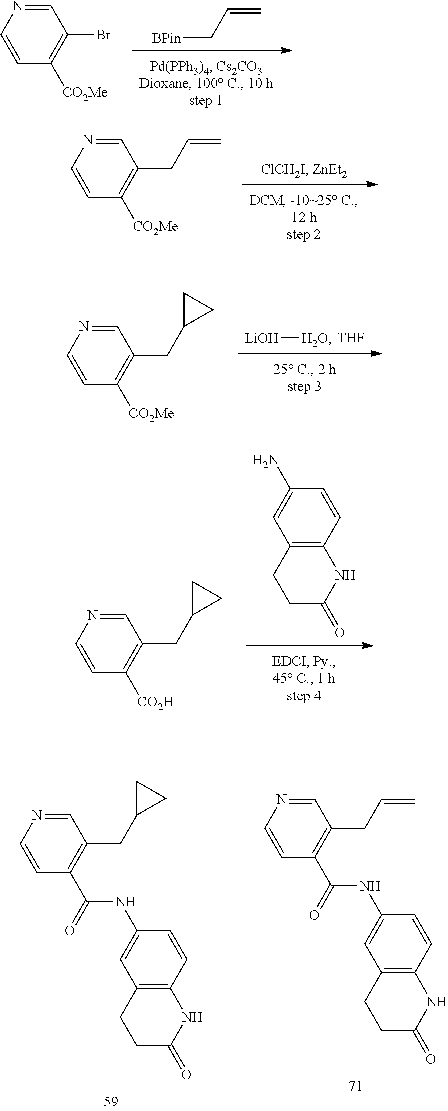

Step 1: To a solution of methyl 3-bromopyridine-4-carboxylate (2 g, 9.26 mmol, 1 eq) in dioxane (35 mL) was added 2-allyl-4,4,5,5-tetramethyl-1,3,2-dioxaborolane (3.11 g, 18.52 mmol, 2 eq), Cs₂CO₃ (6.03 g, 18.52 mmol, 2 eq) and Pd(PPh₃)₄ (1.07 g, 925.79 umol, 0.1 eq). The mixture was stirred at 100° C. for 10 hr under N₂. The reaction mixture filtered and reduced pressure to give residue. The crude product methyl 3-allylpyridine-4-carboxylate (2 g, crude) was obtained as yellow oil.

Step 2: To the mixture of methyl 3-allylpyridine-4-carboxylate (0.2 g, 1.13 mmol, 1 eq) in DCM (10 mL) was added ZnEt2 (1 M, 11.29 mL, 10 eq) drop-wise, stirred at −10° C. Then to the mixture was added chloro(iodo)methane (4.18 g, 23.70 mmol, 1.72 mL, 21 eq) in DCM (10 mL) at −10° C., then stirred at −10° C. for 0.5 hr. Then the mixture was stirred at 25° C. for 11.5 hr. The reaction mixture was quenched by sat.NH₄Cl (5 mL) at 0° C. The mixture was extracted with ethyl acetate (10 mL*3). The combined organic phase was washed with 2N NaOH (20 mL), brine (5 mL*2), dried with anhydrous Na₂SO₄, filtered and concentrated in vacuum. The residue was purified by prep-TLC (SiO₂, Petroleum ether: Ethyl acetate=3:1). Compound methyl 3-allylpyridine-4-carboxylate and methyl 3-(cyclopropylmethyl)pyridine-4-carboxylate (45 mg) was obtained as a white solid. LCMS (M+H⁺): 346.1 @2.212 min

Step 3: To a solution of methyl 3-allylpyridine-4-carboxylate and methyl 3-(cyclopropylmethyl)pyridine-4-carboxylate (45 mg, 1 eq) in THE (2 mL) and H₂O (2 mL) was added LiOH.H₂O (19.75 mg, 470.64 umol, 2 eq). The mixture was stirred at 25° C. for 2 hrs. The reaction mixture was adjusted to pH=4 by 2N HCl, Then the mixture was concentrated under reduced pressure to give a residue. The crude product 3-allylpyridine-4-carboxylic acid and 3-(cyclopropylmethyl)pyridine-4-carboxylic acid (40 mg, 225.73 umol, 95.92% yield) was used in the next step without further purification. LCMS (M+H⁺): 178.1

Step 4: To the mixture of 40 mg of 3-(cyclopropylmethyl)pyridine-4-carboxylic acid and 3-allylpyridine-4-carboxylic acid and 6-amino-3,4-dihydro-1H-quinolin-2-one (36.61 mg, 225.73 umol, 1 eq) in pyridine (1 mL) was added EDCI (51.93 mg, 270.88 umol, 1.2 eq). The mixture was stirred at 45° C. for 1 hr. The reaction mixture was filtered and concentrated under reduced pressure to give a residue. The residue was purified by prep-HPLC (column: Phenomenex luna C18 80*40 mm*3 um; mobile phase: [water(0.04% HCl)-ACN]; B %: 14%-25%, 5.5 min). Compound 59, 3-(cyclopropylmethyl)-N-(2-oxo-3,4-dihydro-1H-quinolin-6-yl)pyridine-4-carboxamide (3.9 mg, HCl) was obtained, LCMS (M+H⁺):322.1. Compound 71, 3-allyl-N-(2-oxo-3,4-dihydro-1H-quinolin-6-yl)pyridine-4-carboxamide (3.3 mg, HCl) was obtained, LCMS (M+H⁴):308.1.

### Example 53. Synthesis of Compound 81

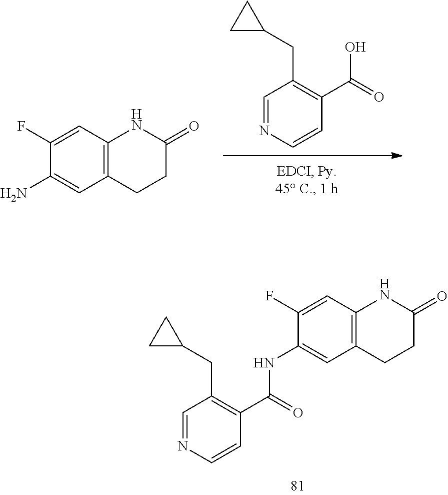 [SMILES: Nc1cc2c(cc1F)NC(=O)CC2 ; O=C(O)c1ccncc1CC1CC1 ; O=C1CCc2cc(NC(=O)c3ccncc3CC3CC3)c(F)cc2N1]

To a solution of 6-amino-7-fluoro-3,4-dihydro-1H-quinolin-2-one (100 mg, 555.00 umol, 1 eq) and 3-(cyclopropylmethyl)pyridine-4-carboxylic acid (177.87 mg, 832.51 umol, 1.5 eq, HCl) in Pyridine (1 mL) was added EDCI (127.67 mg, 666.01 umol, 1.2 eq). The mixture was stirred at 45° C. for 1 hr. The reaction mixture was concentrated under reduced pressure to give a residue. The residue was purified by prep-TLC (SiO₂, Ethyl acetate:MeOH=10:1). Compound 3-(cyclopropylmethyl)-N-(7-fluoro-2-oxo-3,4-dihydro-1H-quinolin-6-yl)pyridine-4-carboxamide (0.065 g, 98% purity) was obtained. LCMS: (M+H)⁺: 340.2.

### Example 54. Synthesis of Compound 55

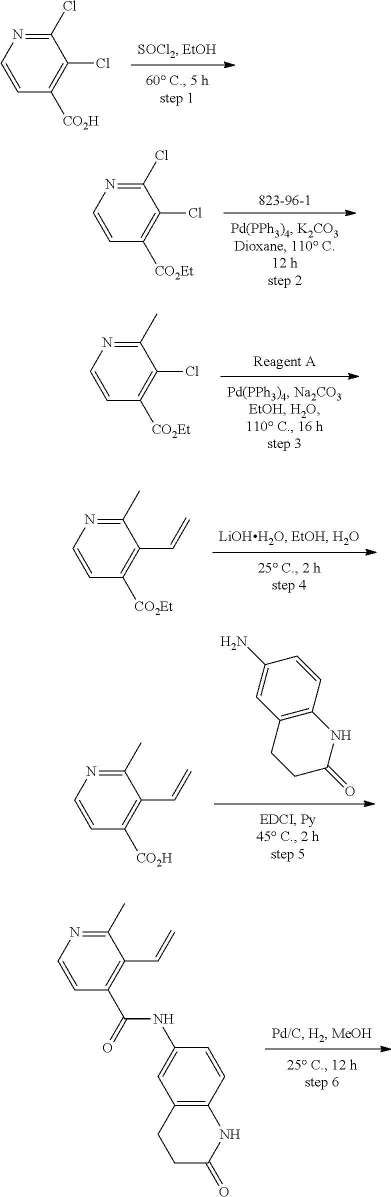

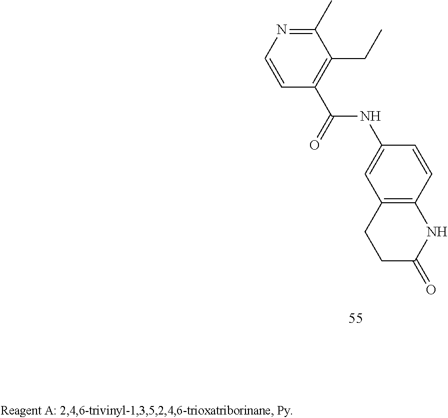 [SMILES: CCc1c(C(=O)Nc2ccc3c(c2)CCC(=O)N3)ccnc1C]

Step 1: To the mixture of 2,3-dichloropyridine-4-carboxylic acid (5 g, 26.04 mmol, 1 eq) in EtOH (50 mL) was added SOCl₂ (6.20 g, 52.08 mmol, 3.78 mL, 2 eq) drop-wise at 0° C. The mixture was stirred at 60° C. for 5 hr. The mixture was poured into sat.NaHCO₃(150 mL). The aqueous phase was extracted with ethyl acetate (30 mL*3). The combined organic phase was washed with brine (20 mL*4), dried with anhydrous Na₂SO₄, filtered and concentrated in vacuum. Compound ethyl 2,3-dichloropyridine-4-carboxylate (4.6 g, 20.90 mmol, 80.27% yield) was obtained as a yellow liquid. LCMS: (M+H)⁺: 219.9

Step 2: To the mixture of ethyl 2,3-dichloropyridine-4-carboxylate (2 g, 9.09 mmol, 1 eq), 2,4,6-trimethyl-1,3,5,2,4,6-trioxatriborinane (2.74 g, 10.91 mmol, 3.05 mL, 50% purity, 1.2 eq), K₂CO₃ (1.88 g, 13.63 mmol, 1.5 eq) in dioxane (40 mL) was added Pd(PPh₃)₄ (1.05 g, 908.87 umol, 0.1 eq) in one portion at 25° C. under N₂. The mixture was stirred at 110° C. for 12 hours. Then the mixture was filtered. The filter cake was washed with EtOAc (50 mL). The combined organic phase was concentrated in vacuum. The residue was purified by column chromatography (SiO₂, Petroleum ether/Ethyl acetate=100/1 to 15/1). Ethyl 3-chloro-2-methyl-pyridine-4-carboxylate (360 mg, crude) was obtained as colorless oil. LCMS: (M+H)⁺: 200.1.

Step 3: To a mixture of ethyl 3-chloro-2-methyl-pyridine-4-carboxylate (0.36 g, 1.80 mmol, 1 eq) in H₂O (1 mL) and dioxane (10 mL) was added K₂CO₃ (747.68 mg, 5.41 mmol, 3 eq) and pyridine; 2,4,6-trivinyl-1,3,5,2,4,6-trioxatriborinane (2.17 g, 9.02 mmol, 5 eq) in one portion at 25° C. under N₂. Then Pd(PPh₃)₄ (208.38 mg, 180.33 umol, 0.1 eq) was added, the mixture was stirred at 110° C. for 16 hours. The reaction mixture was poured in brine (30 mL). The aqueous phase was extracted by EtOAc 150 mL (50 mL*3), the organic phase was dried with anhydrous Na₂SO₄, filtered and concentrated under reduced pressure to give a residue. The residue was purified by column chromatography (SiO₂, Petroleum ether/Ethyl acetate=100/1 to 10/1) and by prep-TLC (SiO₂, Petroleum ether/Ethyl acetate=3:1). Ethyl 2-methyl-3-vinyl-pyridine-4-carboxylate (20 mg, 104.59 umol, 5.80% yield).

Step 4: To the mixture of ethyl 2-methyl-3-vinyl-pyridine-4-carboxylate (20 mg, 104.59 umol, 1 eq) in H₂O (2 mL) and EtOH (4 mL) was added LiOH.H₂O (8.78 mg, 209.18 umol, 2 eq) at 25° C. The mixture was stirred at 25° C. for 2 hr. The reaction mixture was concentrated under reduced pressure to give a residue. The crude product 2-methyl-3-vinyl-pyridine-4-carboxylic acid (46 mg, crude) was obtained as a white solid and used into the next step without further purification. LCMS: (M+H)⁺: 164.1.

Step 5: To a mixture of 2-methyl-3-vinyl-pyridine-4-carboxylic acid (20 mg, 122.57 umol, 1 eq) and 6-amino-3,4-dihydro-1H-quinolin-2-one (19.88 mg, 122.57 umol, 1 eq) in Pyridine (1 mL) was added EDCI (28.20 mg, 147.08 umol, 1.2 eq) in one portion at 25° C. The mixture was stirred at 45° C. for 2 hours. The reaction mixture was concentrated under reduced pressure to give a residue. The crude product 2-methyl-N-(2-oxo-3,4-dihydro-1H-quinolin-6-yl)-3-vinyl-pyridine-4-carboxamide (70 mg, crude) was obtained as a yellow solid. LCMS: (M+H)⁺: 308.2

Step 6: To a solution of 2-methyl-N-(2-oxo-3,4-dihydro-1H-quinolin-6-yl)-3-vinyl-pyridine-4-carboxamide (69.55 mg, 226.30 umol, 1 eq) in MeOH (3 mL) was added 10% Pd/C (10 mg) under N₂. The suspension was degassed under vacuum and purged with H₂ several times. The mixture was stirred under H₂ (15 Psi) at 25° C. for 12 hours. The reaction mixture was filtered and the filter was concentrated. The residue was purified by prep-HPLC (column: Welch Xtimate C18 100*25 mm*3 um; mobile phase: [water (0.05% HCl)-ACN]; B %: 15%-35%, 8 min). Compound 3-ethyl-2-methyl-N-(2-oxo-3,4-dihydro-1H-quinolin-6-yl)pyridine-4-carboxamide (11 mg, HCl) was obtained. LCMS (M+H⁺): 310.1.

### Example 55. Synthesis of Compound 58

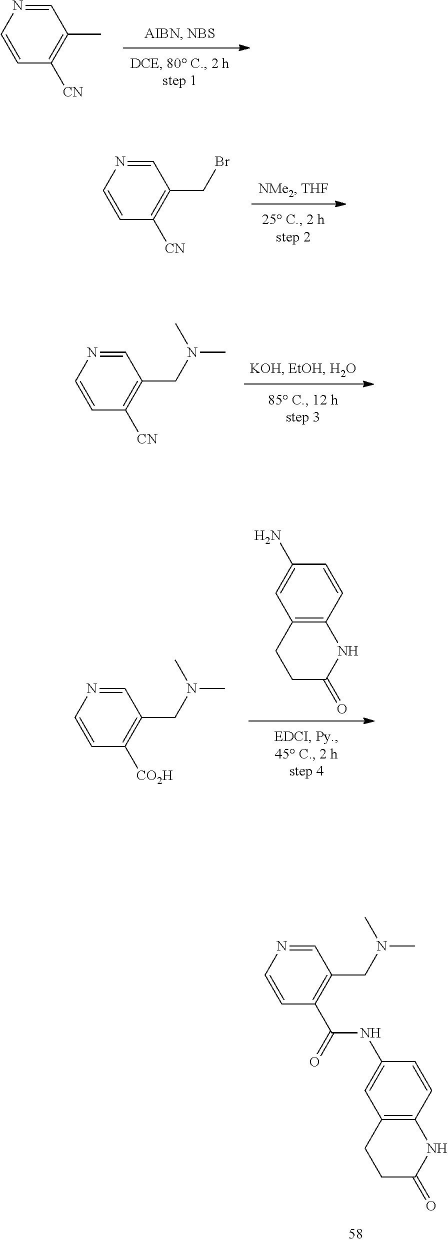

Step 1: To a mixture of 3-methylpyridine-4-carbonitrile (2 g, 16.93 mmol, 1 eq) and NBS (3.01 g, 16.93 mmol, 1 eq), AIBN (347.50 mg, 2.12 mmol, 0.125 eq) was added 1,2-dichloroethane (10 mL). Then the mixture was stirred at 80° C. for 2 hrs. The reaction mixture was filtered and concentrated under reduced pressure to give a residue. The crude product 3-(bromomethyl)pyridine-4-carbonitrile (2 g, crude) as yellow oil was used into the next step without further purification. LCMS: (M+H)⁺: 197.0, 199.0.

Step 2: 3-(bromomethyl)pyridine-4-carbonitrile (2 g, 10.15 mmol, 1 eq) in Me₂NH (2 M in THF, 25.38 mL, 5 eq) was stirred for 2 h at 25° C. The reaction mixture was concentrated under reduced pressure to give a residue. The residue was purified by column chromatography (SiO₂, Petroleum ether/Ethyl acetate=100/1 to 30/1). The desired 3-[(dimethylamino)methyl]pyridine-4-carbonitrile (0.98 g, 6.08 mmol, 59.89% yield) was obtained as yellow oil.

Step 3: To a mixture of 3-[(dimethylamino)methyl]pyridine-4-carbonitrile (920 mg, 5.71 mmol, 1 eq) in EtOH (10 mL) and H₂O (1 mL) was added KOH (3.20 g, 57.07 mmol, 10 eq) in one portion at 25° C. under N₂. The mixture was stirred at 85° C. for 12 hours. The reaction mixture was concentrated under reduced pressure to give a residue. 3-[(dimethylamino)methyl]pyridine-4-carboxylic acid (4 g, crude) was obtained as yellow solid. LCMS: (M+H)⁺: 181.0.

Step 4: To a mixture of 6-amino-3,4-dihydro-1H-quinolin-2-one (163.64 mg, 1.01 mmol, 1 eq) and 3-[(dimethylamino)methyl]pyridine-4-carboxylic acid (200 mg, 1.11 mmol, 1.1 eq) in Pyridine (3 mL) was added EDCI (232.10 mg, 1.21 mmol, 1.2 eq) in one portion at 25° C. under N₂. The mixture was stirred at 45° C. for 2 hours. The reaction mixture was filtered and concentrated under reduced pressure to give a residue. The residue was purified by prep-TLC (SiO₂, DCM:MeOH=10:1). Compound 3-[(dimethylamino)methyl]-N-(2-oxo-3,4-dihydro-1H-quinolin-6-yl)pyridine-4-carboxamide (37 mg, 93% purity) was obtained. LCMS (M+H⁺):325.1.

### Example 56. Synthesis of Compound 78

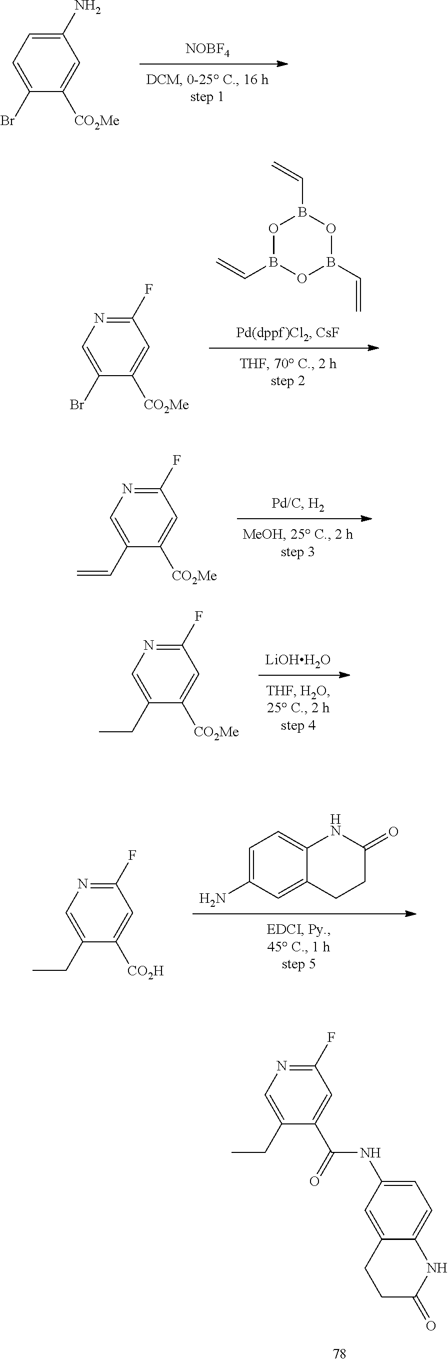

Step 1: To a solution of nitridooxonium tetrafluoroborate (3.25 g, 27.82 mmol, 1.29 eq) in DCM (50 mL) was added methyl 2-amino-5-bromo-pyridine-4-carboxylate (5 g, 21.64 mmol, 1 eq) in DCM (15 mL) drop-wise at 0° C. The mixture was stirred at 25° C. for 16 hrs. The reaction mixture was then quenched by the addition of water (60 mL) slowly at 0° C. The reaction mixture was extracted with DCM 100 mL (50 mL*2). The combined organic layers were washed with brine 30 mL (10 mL*3), dried over with Na₂SO₄, filtered and concentrated under reduced pressure to give a residue. The residue was purified by flash silica gel chromatography (ISCO®; 12 g SepaFlash® Silica Flash Column, Eluent of 0-10% Ethyl acetate/Petroleum ethergradient @40 mL/min). Compound methyl 5-bromo-2-fluoro-pyridine-4-carboxylate (3.6 g, 15.38 mmol, 71.08% yield) was obtained as colorless oil. LCMS: (M+H)⁺: 235.9.

Step 2: To a solution of methyl 5-bromo-2-fluoro-pyridine-4-carboxylate (0.5 g, 2.14 mmol, 1 eq) in THE (5 mL) was added pyridine; 2,4,6-trivinyl-1,3,5,2,4,6-trioxatriborinane (1.29 g, 5.34 mmol, 2.5 eq) and CsF (973.64 mg, 6.41 mmol, 236.32 uL, 3 eq). And then the mixture was added Pd(dppf)Cl₂ (156.33 mg, 213.65 umol, 0.1 eq). The mixture was stirred at 70° C. for 2 hrs under N₂. The reaction mixture was filtered. The filter cake was washed with EtOAc (15 mL). The combined organic layers were washed with brine 10 mL, dried over with Na₂SO₄, filtered and concentrated under reduced pressure to give a residue. The residue was purified by flash silica gel chromatography (ISCO®; 12 g SepaFlash® Silica Flash Column, Eluent of 0-10% Ethyl acetate/Petroleum ethergradient @ 40 mL/min). Compound methyl 2-fluoro-5-vinyl-pyridine-4-carboxylate (0.24 g, 1.32 mmol, 62.01% yield) was obtained as a white solid. LCMS: (M+H)⁺: 182.1.

Step 3: To a solution of methyl 2-fluoro-5-vinyl-pyridine-4-carboxylate (150 mg, 827.98 umol, 1 eq) in MeOH (10 mL) was added 10% Pd/C (0.02 g). The mixture was stirred at 25° C. for 2 hrs under H₂(15 Psi). The reaction mixture was filtered and concentrated under reduced pressure to give a residue. Compound methyl 5-ethyl-2-fluoro-pyridine-4-carboxylate (100 mg, crude) was obtained as a white solid. LCMS: (M+H)⁺: 184.0.

Step 4: To a solution of methyl 5-ethyl-2-fluoro-pyridine-4-carboxylate (100 mg, 545.91 umol, 1 eq) in H₂O (1 mL) and THE (1 mL) was added LiOH.H₂O (34.36 mg, 818.87 umol, 1.5 eq). The mixture was stirred at 25° C. for 2 hr. The mixture was adjusted to pH=3 with HCl(1N). Then the mixture was concentrated under reduced pressure to give a residue. Compound 5-ethyl-2-fluoro-pyridine-4-carboxylic acid (50 mg, crude) was obtained as a white solid.

Step 5: To a solution of 5-ethyl-2-fluoro-pyridine-4-carboxylic acid (40 mg, 194.54 umol, 1 eq) in Pyridine (1 mL) was added EDCI (44.75 mg, 233.45 umol, 1.2 eq). The mixture was stirred at 45° C. for 1 hr. The reaction mixture was concentrated under reduced pressure to give a residue. The residue was purified by prep-TLC (SiO₂, ethyl acetate:MeOH=10:1). Compound 5-ethyl-2-fluoro-N-(2-oxo-3,4-dihydro-1H-quinolin-6-yl)pyridine-4-carboxamide (18 mg, 97.4% purity) was obtained. LCMS: (M+H)⁺: 314.1.

### Example 57. Synthesis of Compound 79

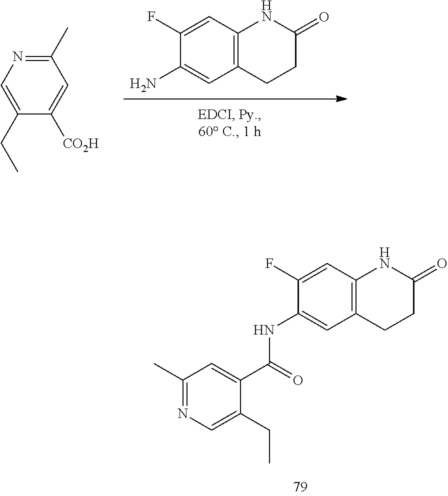 [SMILES: CCc1cnc(C)cc1C(=O)O ; Nc1cc2c(cc1F)NC(=O)CC2 ; CCc1cnc(C)cc1C(=O)Nc1cc2c(cc1F)NC(=O)CC2]

To a solution of 5-ethyl-2-methyl-pyridine-4-carboxylic acid (0.035 g, 173.57 umol, 1 eq, HCl) and 6-amino-7-fluoro-3,4-dihydro-1H-quinolin-2-one (31.27 mg, 173.57 umol, 1 eq) in Pyridine (1 mL) was added EDCI (39.93 mg, 208.28 umol, 1.2 eq). The mixture was stirred at 60° C. for 1 hr. The reaction mixture was concentrated under reduced pressure to give a residue. The residue was purified by prep-TLC (SiO₂, Petroleum ether: Ethyl acetate=1:0). Compound 5-ethyl-N-(7-fluoro-2-oxo-3,4-dihydro-1H-quinolin-6-yl)-2-methyl-pyridine-4 carboxamide (3 mg, 98.7% purity) was obtained. LCMS: (M+H)⁺: 328.2.

### Example 58. Synthesis of Compound 80

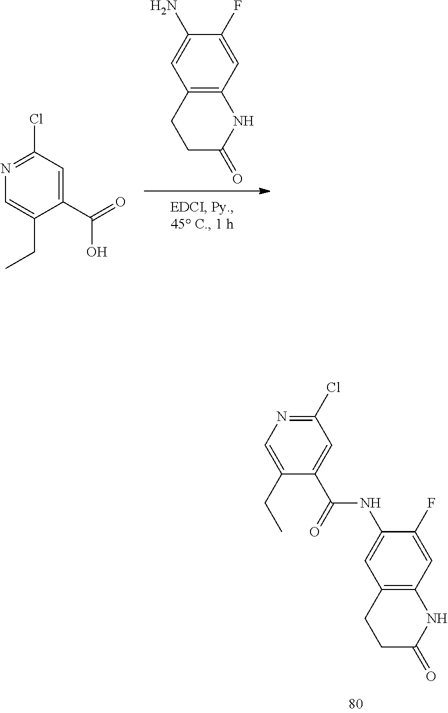 [SMILES: CCc1cnc(Cl)cc1C(=O)O ; Nc1cc2c(cc1F)NC(=O)CC2 ; CCc1cnc(Cl)cc1C(=O)Nc1cc2c(cc1F)NC(=O)CC2]

To a solution of 6-amino-7-fluoro-3,4-dihydro-1H-quinolin-2-one (100 mg, 555.00 umol, 1 eq) and 2-chloro-5-ethyl-pyridine-4-carboxylic acid (147.90 mg, 666.01 umol, 1.2 eq, HCl) in pyridine (1 mL) was added EDCI (127.67 mg, 666.01 umol, 1.2 eq). The mixture was stirred at 45° C. for 1 hr. The reaction mixture was concentrated under reduced pressure to give a residue. The residue was purified by prep-TLC (SiO₂, ethyl acetate:MeOH=10:1). Compound 2-chloro-5-ethyl-N-(7-fluoro-2-oxo-3,4-dihydro-1H-quinolin-6-yl)pyridine-4-carboxamide (22 mg, 100% purity) was obtained. LCMS: (M+H)⁺: 348.1.

### Example 59. Synthesis of Compound 126

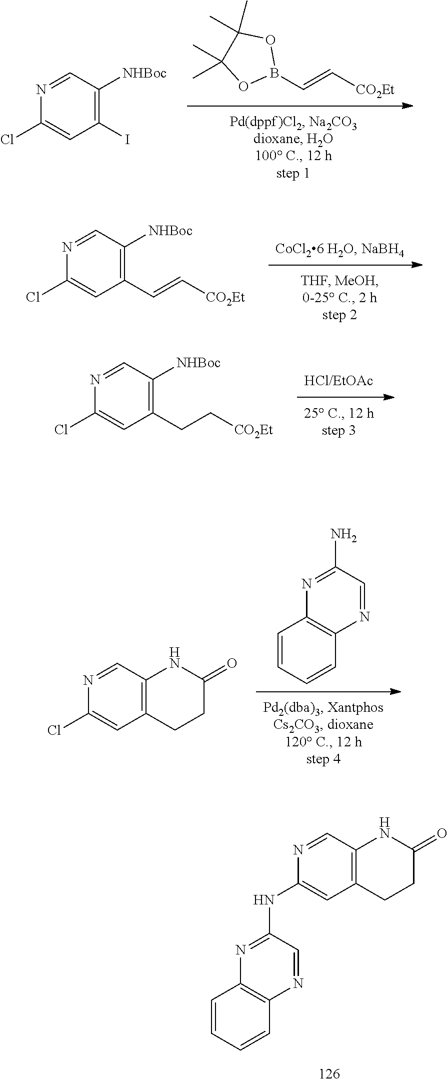

Step 1: To the mixture of tert-butyl N-(6-chloro-4-iodo-3-pyridyl)carbamate (0.5 g, 1.41 mmol, 1 eq), ethyl (E)-3-(4,4,5,5-tetramethyl-1,3,2-dioxaborolan-2-yl)prop-2-enoate (478.21 mg, 2.12 mmol, 1.5 eq) and Na₂CO₃ (298.92 mg, 2.82 mmol, 2 eq) in dioxane (5 mL) and H₂O (1 mL) was added Pd(dppf)Cl₂ (103.18 mg, 141.02 umol, 0.1 eq). The mixture was stirred at 100° C. for 12 h under N₂. The mixture was filtered. The filter cake was washed with EtOAc (50 mL). The combined organic phase was washed with brine (10 mL*2), dried with anhydrous Na₂SO₄, filtered and concentrated in vacuum. The residue was purified by flash silica gel chromatography (ISCO®; 12 g SepaFlash® Silica Flash Column, Eluent of 0-12% Ethyl acetate/Petroleum ether gradient @ 40 mL/min). Ethyl (E)-3-[5-(tert-butoxycarbonylamino)-2-chloro-4-pyridyl]prop-2-enoate (0.35 g, 1.07 mmol, 75.95% yield) was obtained as yellow solid.

Step 2: To a solution of ethyl (E)-3-[5-(tert-butoxycarbonylamino)-2-chloro-4-pyridyl]prop-2-enoate (0.2 g, 612.04 umol, 1 eq) and CoCl₂.6H₂O (14.56 mg, 61.20 umol, 0.1 eq) in MeOH (10 mL) and THE (5 mL) was added NaBH₄ (140 mg, 3.70 mmol, 6.05 eq) in portions under N₂ at 0° C. The mixture was stirred at 25° C. for 2 hr. To the mixture was added water (10 ml) drop-wise at 0° C. The mixture was stirred at 25° C. for 0.5 h and concentrated in vacuum to remove THE and MeOH. The aqueous phase was extracted with ethyl acetate (10 mL*2). The combined organic phase was washed with brine (5 mL), dried with anhydrous Na₂SO₄, filtered and concentrated in vacuum. Ethyl 3-[5-(tert-butoxycarbonylamino)-2-chloro-4-pyridyl]propanoate (100 mg, crude) was obtained as yellow solid. LCMS: (M+H)⁺: 329.2.

Step 3: The mixture of ethyl 3-[5-(tert-butoxycarbonylamino)-2-chloro-4-pyridyl]propanoate (100 mg, 304.15 umol, 1 eq) in HCl/EtOAc (4 M, 2 mL) was stirred at 25° C. for 12 hr. The mixture was filtered. The filter cake was concentrated in vacuum. 6-chloro-3,4-dihydro-1H-1,7-naphthyridin-2-one (35 mg, 191.67 umol, 63.02% yield) was obtained as white solid. LCMS: (M+H)⁺: 183.1.

Step 4: To the mixture of quinoxalin-2-amine (27.82 mg, 191.67 umol, 1 eq) and 6-chloro-3,4-dihydro-1H-1,7-naphthyridin-2-one (35 mg, 191.67 umol, 1 eq) in dioxane (5 mL) was added Pd₂(dba)₃ (17.55 mg, 19.17 umol, 0.1 eq), Cs₂CO₃ (187.35 mg, 575.01 umol, 3 eq) and Xantphos (11.09 mg, 19.17 umol, 0.1 eq). The mixture was stirred at 120° C. for 12 hr. The mixture was filtered. The filter cake was washed with EtOAc (50 mL). The filtrate was concentrated in vacuum. The residue was purified by prep-HPLC (column: Phenomenex Luna C18 100*30 mm*5 um; mobile phase: [water (0.04% HCl)-ACN]; B %: 10%-40%, 10 min). 6-(quinoxalin-2-ylamino)-3,4-dihydro-1H-1,7-naphthyridin-2-one (5 mg, HCl salt, 100% purity) was obtained. LCMS: (M+H)⁺: 292.1.

### Example 60. Synthesis of Compound 51

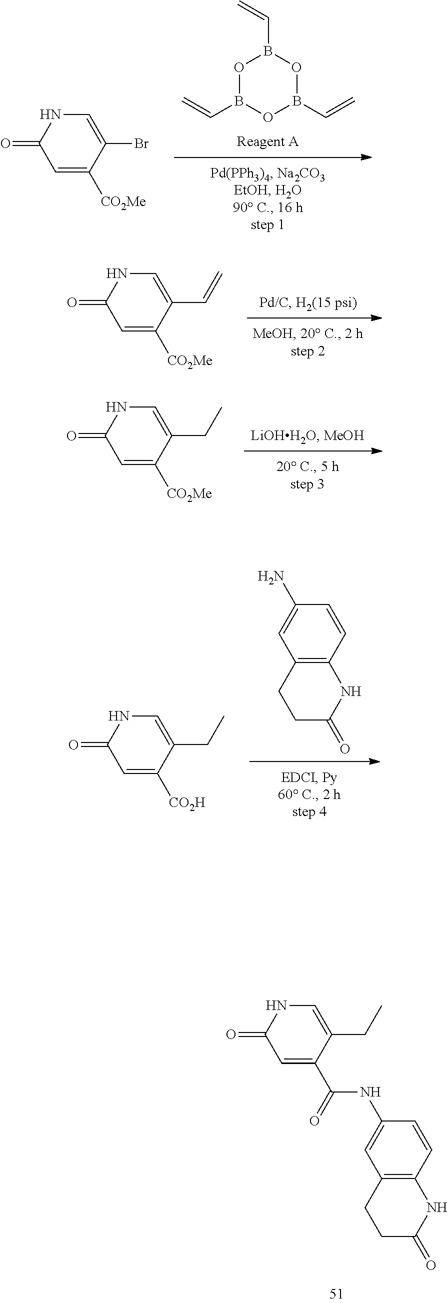

Step 1: To the mixture of methyl 5-bromo-2-oxo-1H-pyridine-4-carboxylate (1 g, 4.31 mmol, 1 eq) and pyridine; 2,4,6-trivinyl-1,3,5,2,4,6-trioxatriborinane (2.07 g, 8.62 mmol, 2 eq) in the solution of DMF (10 mL) and H₂O (0.5 mL) was added Pd(PPh₃)₄ (498.02 mg, 430.98 umol, 0.1 eq), Na₂CO₃ (1.37 g, 12.93 mmol, 3 eq). The mixture was stirred at 90° C. for 5 hr. The mixture was filtered. The filter cake was washed with EtOAc (20 mL). To the solution was added water (40 mL). The aqueous phase was extracted with ethyl acetate (20 mL*3). The combined organic phase was washed with brine (10 mL*3), dried with anhydrous Na₂SO₄, filtered and concentrated in vacuum. The residue was purified by column chromatography (SiO₂, Petroleum ether/Ethyl acetate=25/1 to 10/1), methyl 2-oxo-5-vinyl-1H-pyridine-4-carboxylate (150 mg) was obtained as yellow oil.

Step 2: To a solution of methyl 2-oxo-5-vinyl-1H-pyridine-4-carboxylate (110 mg, 613.93 umol, 1 eq) in MeOH (10 mL) was added 10% Pd/C (0.05 g) under N₂. The suspension was degassed under vacuum and purged with H₂ several times. The mixture was stirred under H₂ (15 psi) at 25° C. for 1 hours. The mixture was filtered and concentrated in vacuum. Methyl 5-ethyl-2-oxo-1H-pyridine-4-carboxylate (90 mg, 496.72 umol, 80.91% yield) was obtained as yellow solid.

Step 3: To the mixture of methyl 5-ethyl-2-oxo-1H-pyridine-4-carboxylate (90 mg, 496.72 umol, 1 eq) in H₂O (3 mL) and THF (3 mL) was added LiOH.H₂O (41.69 mg, 993.44 umol, 2 eq). The mixture was stirred at 25° C. for 1 hr. The mixture was concentrated in vacuum to remove THF. Then the mixture was adjusted to pH=6 by 3N HCl. The mixture was filtered. The filter cake was washed with EtOAc (5 mL) and concentrated in vacuum. [5-ethyl-2-oxo-1H-pyridine-4-carboxylic acid (47 mg, crude) was obtained as yellow solid.

Step 4: The mixture of 5-ethyl-2-oxo-1H-pyridine-4-carboxylic acid (37 mg, 221.34 umol, 1 eq), 6-amino-3,4-dihydro-1H-quinolin-2-one (35.90 mg, 221.34 umol, 1 eq) and EDCI (50.92 mg, 265.61 umol, 1.2 eq) in pyridine (1 mL) was stirred at 45° C. for 1 hr. The mixture was concentrated in vacuum. The residue was purified by prep-TLC (SiO₂, Petroleum ether: Ethyl acetate=0:1). Then the crude product was purified by prep-HPLC (column: Phenomenex Gemini-NX C18 75*30 mm*3 um; mobile phase: [water (10 mM NH4HCO₃)-ACN]; B %: 10%-30%, 8 min). 5-ethyl-2-oxo-N-(2-oxo-3,4-dihydro-1H-quinolin-6-yl)-1H-pyridine-4-carboxamide (3.4 mg, 100% purity) was obtained, LCMS: (M+H⁺) 312.0.

### Example 61. Synthesis of Compound 56

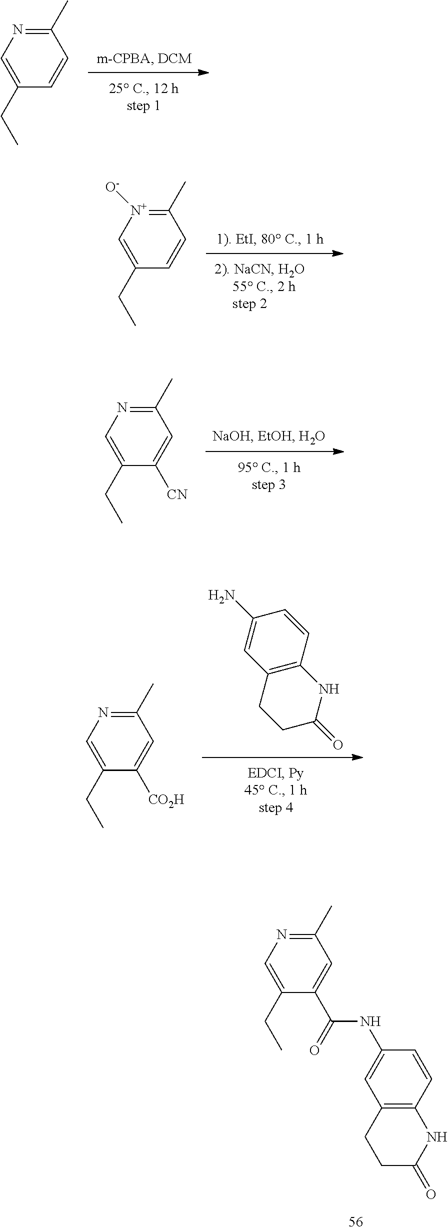

Step 1: To the mixture of 5-ethyl-2-methyl-pyridine (10 g, 82.52 mmol, 10.88 mL, 1 eq) in DCM (100 mL) was added MCPBA (19.58 g, 90.77 mmol, 80% purity, 1.1 eq) in portions at 25° C. Then the mixture was stirred at 25° C. for 12 hr. To the mixture was added sat. Na₂SO₃ (200 mL). The mixture was stirred at 25° C. for 1 hr. Then the mixture was extracted with DCM (50 mL*3) The combined organic phase was washed with brine (100 mL*2), dried with anhydrous Na₂SO₄, filtered and concentrated in vacuum. 5-ethyl-2-methyl-1-oxido-pyridin-1-ium (15 g, crude) was obtained as yellow oil.

Step 2: The mixture of 5-ethyl-2-methyl-1-oxido-pyridin-1-ium (10 g, 72.90 mmol, 1 eq) in EtI (34.11 g, 218.69 mmol, 17.49 mL, 3 eq) was stirred at 60° C. for 1 hr. The solution was cooled to 25° C. The mixture was added Petroleum ether (100 mL). The mixture was filtered. The filter cake was added to H₂O (100 mL) and then added NaCN (5.95 g, 121.41 mmol, 1.67 eq) in H₂O (30 mL) was added at 55° C. in portions. Then the mixture was stirred at 55° C. for 2 hr. The reaction mixture was cooled to room temperature and extracted with EtOAc 150 mL (50 mL*3). The combined organic layers were washed with brine 60 mL (30 mL*2), dried over Na₂SO₄, filtered and concentrated under reduced pressure to give a residue. The residue was purified by column chromatography (SiO₂, Petroleum ether/Ethyl acetate=100/1 to 10/1). 5-ethyl-2-methyl-pyridine-4-carbonitrile (1.2 g, crude) was obtained as colorless oil.

Step 3: To the mixture of 5-ethyl-2-methyl-pyridine-4-carbonitrile (0.3 g, 2.05 mmol, 1 eq) in EtOH (3 mL) and H₂O (3 mL) was added NaOH (164.16 mg, 4.10 mmol, 2 eq). The mixture was stirred at 95° C. for 1 hr. The mixture was concentrated in vacuum to remove EtOH. Then the mixture was extracted with ethyl acetate (2 mL*3). The aqueous phase was adjusted to pH=3 and concentrated in vacuum. The crude product was added to the mixture of THE (5 mL) and EtOH (3 ml). The mixture was filtered. 5-ethyl-2-methyl-pyridine-4-carboxylic acid (37 mg, 223.99 umol, 10.91% yield) was obtained as white solid.

Step 4: The mixture of 6-amino-3,4-dihydro-1H-quinolin-2-one (24.55 mg, 151.34 umol, 1 eq), 5-ethyl-2-methyl-pyridine-4-carboxylic acid (25 mg, 151.34 umol, 1 eq) and EDCI (34.82 mg, 181.61 umol, 1.2 eq) in pyridine (0.5 mL) was stirred at 45° C. for 1 hr. The mixture was concentrated in vacuum. The residue was purified by prep-TLC (SiO₂, Petroleum ether: Ethyl acetate=0:1). 5-ethyl-2-methyl-N-(2-oxo-3,4-dihydro-1H-quinolin-6-yl)pyridine-4-carboxamide (10.5 mg, 97.1% purity) was obtained, LCMS: (M+H⁺) 310.1.

### Example 62. Synthesis of Compound 57

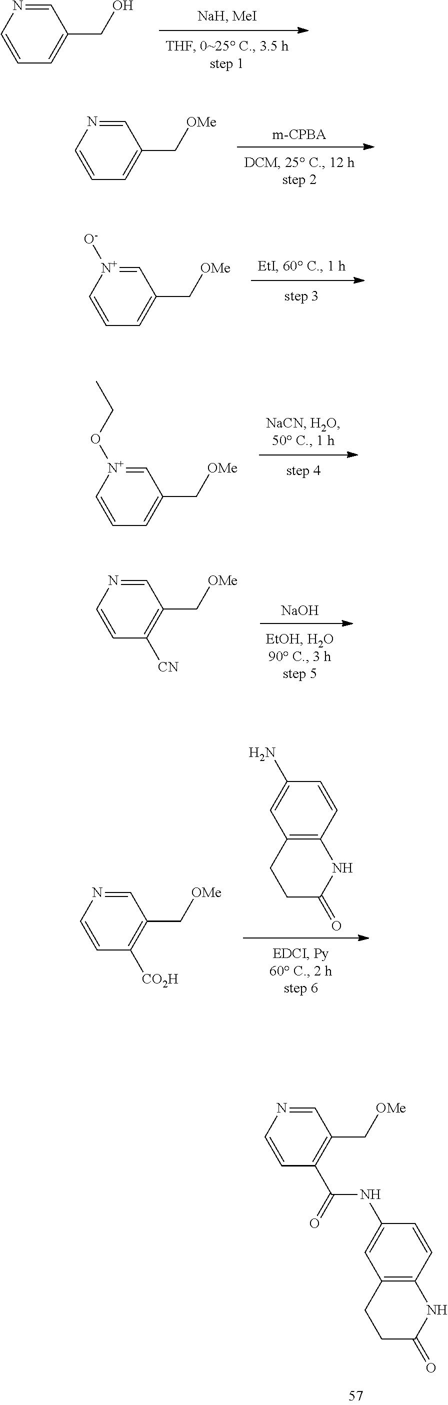

Step 1: To the mixture of 3-pyridylmethanol (5 g, 45.82 mmol, 4.39 mL, 1 eq) in THE (100 mL) was added NaH (2.02 g, 50.40 mmol, 60% purity, 1.1 eq) in portions at 0° C. Then the mixture was stirred at 0° C. for 0.5 hr. To the mixture was added MeI (7.15 g, 50.40 mmol, 3.14 mL, 1.1 eq) drop-wise at 0° C. Then the mixture was stirred at 25° C. for 3 hr. To the mixture was added H₂O (20 mL) drop-wise at 0° C. Then the mixture was extracted with ethyl acetate (50 mL*3). The combined organic phase was washed with brine (50 mL), dried with anhydrous Na₂SO₄, filtered and concentrated in vacuum. 3-(methoxymethyl)pyridine (5 g, 40.60 mmol, 88.61% yield) was obtained as yellow oil.

Step 2: To the mixture of 3-(methoxymethyl)pyridine (2 g, 16.24 mmol, 1 eq) in DCM (30 mL) was added m-CPBA (5.25 g, 24.36 mmol, 80% purity, 1.5 eq) at 25° C. Then the mixture was stirred at 25° C. for 12 h. To the mixture was added sat. Na₂SO₃ (50 mL). The mixture was stirred at 25° C. for 0.5 hr. Then the aqueous phase was extracted with ethyl acetate (30 mL*4). The combined organic phase was washed with brine (25 mL), dried with anhydrous Na₂SO₄, filtered and concentrated in vacuum. The residue was purified by column chromatography (SiO₂, Petroleum ether/Ethyl acetate=20/1 to 0/1). 3-(methoxymethyl)-1-oxido-pyridin-1-ium (2 g, 14.37 mmol, 88.50% yield) was obtained as yellow oil.

Step 3: The mixture of 3-(methoxymethyl)-1-oxido-pyridin-1-ium (2.25 g, 16.17 mmol, 1 eq) in EtI (7.57 g, 48.51 mmol, 3.88 mL, 3 eq) was stirred at 60° C. for 1 h. The mixture was cooled to 25° C. To the mixture was added Petroleum ether (30 mL). The mixture was filtered. 1-ethoxy-3-(methoxymethyl)pyridin-1-ium (3.0 g, crude) was obtained as yellow solid.

Step 4: 1-ethoxy-3-(methoxymethyl)pyridin-1-ium (3.0 g, 1 eq) was added to H₂O (20 mL). To the mixture was added the solution of NaCN (1.16 g, 23.67 mmol, 1.46 eq) in H₂O (5 mL) at 50° C. The mixture was stirred at 50° C. for 1 hr. The mixture was cooled to 25° C. and extracted with ethyl acetate (50 mL*4). The combined organic phase was washed with brine (20 mL*2), dried with anhydrous Na₂SO₄, filtered and concentrated in vacuum. The residue was purified by flash silica gel chromatography (ISCO®; 12 g SepaFlash® Silica Flash Column, Eluent of 0-13% Ethyl acetate/Petroleum ether gradient @ 40 mL/min). 3-(methoxymethyl)pyridine-4-carbonitrile (0.36 g, 2.43 mmol, 15.03% yield) was obtained as yellow oil.

Step 5: The mixture of 3-(methoxymethyl)pyridine-4-carbonitrile (0.36 g, 2.43 mmol, 1 eq) and NaOH (291.55 mg, 7.29 mmol, 3 eq) in EtOH (3 mL) and H₂O (3 mL) was stirred at 90° C. for 3 hr. The mixture was concentrated in vacuum to remove EtOH. The aqueous phase was adjusted to pH=5-6 by 6 N HCl. Then the mixture was filtered. The filter cake was concentrated in vacuum. 3-(methoxymethyl)pyridine-4-carboxylic acid (0.24 g, 1.18 mmol, 48.51% yield, HCl) was obtained as yellow solid.

Step 6: To the mixture of 6-amino-3,4-dihydro-1H-quinolin-2-one (39.83 mg, 245.55 umol, 1 eq) and 3-(methoxymethyl)pyridine-4-carboxylic acid (50 mg, 245.55 umol, 1 eq, HCl) in pyridine (1 mL) was added EDCI (56.49 mg, 294.66 umol, 1.2 eq). The mixture was stirred at 45° C. for 1 hr. The mixture was concentrated in vacuum. The residue was purified by prep-TLC (SiO₂, Petroleum ether:Ethyl acetate=0:1). 3-(methoxymethyl)-N-(2-oxo-3,4-dihydro-1H-quinolin-6-yl)pyridine 4 carboxamide (30 mg, 100% purity) was obtained. LCMS: (M+H⁺) 312.3.

### Example 63. Synthesis of Compound 73

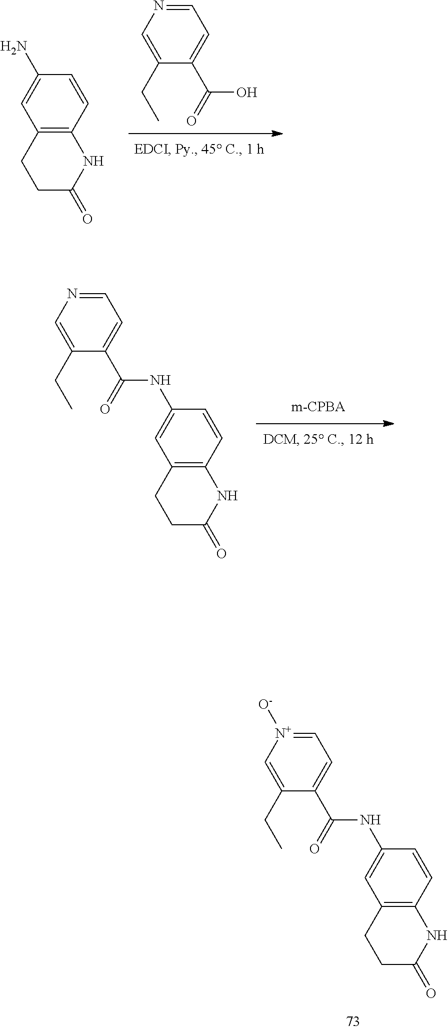 [SMILES: Nc1ccc2c(c1)CCC(=O)N2 ; CCc1cnccc1C(=O)O ; CCc1cnccc1C(=O)Nc1ccc2c(c1)CCC(=O)N2 ; CCc1c[n+]([O-])ccc1C(=O)Nc1ccc2c(c1)CCC(=O)N2]

Step 1: To the solution of 6-amino-3,4-dihydro-1H-quinolin-2-one (150 mg, 924.85 umol, 1 eq) in pyridine (5 mL) was added EDCI (212.75 mg, 1.11 mmol, 1.2 eq) and 3-ethylpyridine-4-carboxylic acid (208.23 mg, 1.11 mmol, 1.2 eq, HCl). The mixture was stirred at 45° C. for 1 hr. The reaction mixture was concentrated under reduced pressure to give a residue. The residue was purified by flash silica gel chromatography (ISCO®; 200 mg SepaFlash® Silica Flash Column, Eluent of 0-100% (Ethyl acetate:MeOH=10:1)/Petroleum ethergradient @ 40 mL/min). Compound 3-ethyl-N-(2-oxo-3,4-dihydro-1H-quinolin-6-yl)pyridine-4-carboxamide (90 mg, 304.74 umol, 32.95% yield) was obtained.

Step 2: To the solution of 3-ethyl-N-(2-oxo-3,4-dihydro-1H-quinolin-6-yl)pyridine-4-carboxamide (90 mg, 304.74 umol, 1 eq) in DCM (10 mL) was added MCPBA (72.31 mg, 335.21 umol, 80% purity, 1.1 eq). The mixture was stirred at 25° C. for 12 hr. The reaction mixture was quenched by addition sat.Na₂SO₃ 5 mL at 25° C. and stirred at 25° C. for 0.5 h. Then the mixture was extracted with DCM (10 mL*2). The combined organic phase was washed with brine (2 mL), dried with anhydrous Na₂SO₄, filtered and concentrated in vacuum. The residue was purified by prep-HPLC (column: Phenomenex Gemini-NX C18 75*30 mm*3 um; mobile phase: [water(10 mM NH4HCO3)-ACN]; B %:10%-45%, 8 min). Compound 3-ethyl-1-oxido-N-(2-oxo-3,4-dihydro-1H-quinolin-6-yl)pyridin-1-ium-4-carboxamide (3 mg, 100% purity) was obtained. LCMS, (M+H⁺) 312.2.

### Example 64. Synthesis of Compound 76

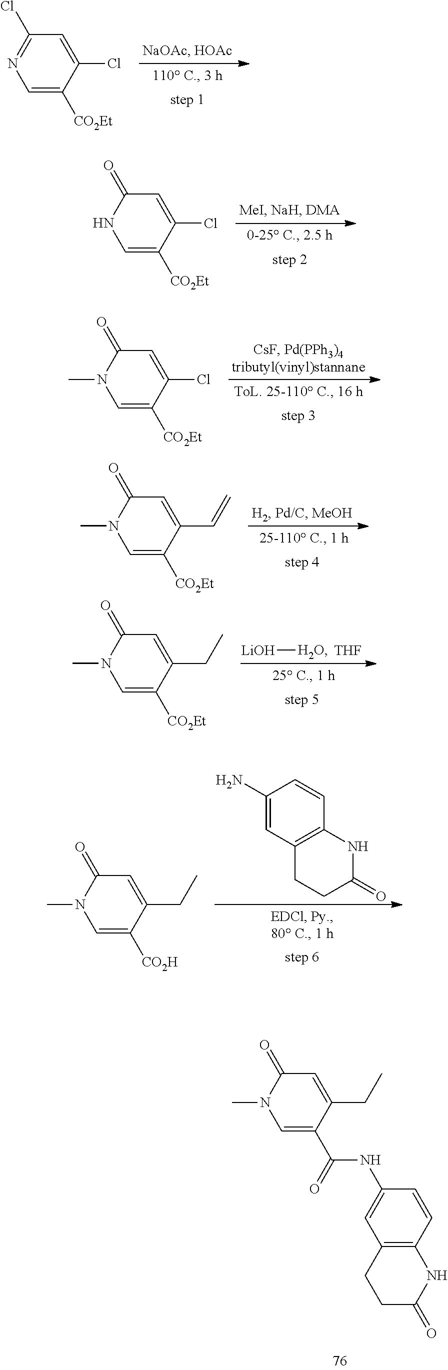

Step 1: To a solution of ethyl 4,6-dichloropyridine-3-carboxylate (5 g, 22.72 mmol, 1 eq) in HOAc (50 mL) was added NaOAc (9.32 g, 113.61 mmol, 5 eq). The mixture was stirred at 110° C. for 3 hr. To the reaction mixture was added water 30 mL (10 ml*3), and then filtered to give a residue. The residue was purified by flash silica gel chromatography (ISCO®; 12 g SepaFlash® Silica Flash Column, Eluent of 0-10% Ethyl acetate/Petroleum ether gradient @ 40 mL/min). Compound ethyl 4-chloro-6-oxo-1H-pyridine-3-carboxylate (1.5 g, 7.44 mmol, 32.74% yield) was obtained as a white solid.

Step 2: To a solution of ethyl 4-chloro-6-oxo-1H-pyridine-3-carboxylate (1 g, 4.96 mmol, 1 eq) in DMA (15 mL) was added NaH (257.90 mg, 6.45 mmol, 60% purity, 1.3 eq) at 0° C. The mixture was stirred at 25° C. for 0.5 h. To the mixture was added MeI (915.24 mg, 6.45 mmol, 401.42 uL, 1.3 eq) at 0° C. The mixture was stirred at 25° C. for 2 hr. The mixture was poured into sat.NH₄Cl (15 mL) at 0° C. The aqueous phase was extracted with ethyl acetate (5 mL*3). The combined organic phase was washed with brine (5 mL*4), dried with anhydrous Na₂SO₄, filtered and concentrated in vacuum. Compound ethyl 4-chloro-1-methyl-6-oxo-pyridine-3-carboxylate (0.8 g, crude) was obtained as a white solid.

Step 3: To a mixture of ethyl 4-chloro-1-methyl-6-oxo-pyridine-3-carboxylate (0.8 g, 3.71 mmol, 1 eq) in Toluene (10 mL) was added tributyl(vinyl)stannane (3.53 g, 11.13 mmol, 3.24 mL, 3 eq), CsF (1.13 g, 7.42 mmol, 273.57 uL, 2 eq) and Pd(PPh3)4 (428.71 mg, 371.00 umol, 0.1 eq) in one portion at 25° C. under N₂,then the mixture was stirred at 110° C. for 16 hours. The mixture was cooled to 25° C. To the mixture was added sat. KF (30 mL in H2O). Then the mixture was extracted with ethyl acetate (10 mL*3).The combined organic phase was washed with brine (20 mL), dried with anhydrous Na₂SO₄, filtered and concentrated in vacuum. The residue was purified by flash silica gel chromatography (ISCO®; 12 g SepaFlash® Silica Flash Column, Eluent of 0-100% Ethyl acetate/Petroleum ethergradient @ 40 mL/min). Compound ethyl 1-methyl-6-oxo-4-vinyl-pyridine-3-carboxylate (140 mg, 675.59 umol, 18.21% yield) was obtained as a yellow solid.

Step 4: To a solution of ethyl 1-methyl-6-oxo-4-vinyl-pyridine-3-carboxylate (156 mg, 752.80 umol, 1 eq) in MeOH (10 mL) was added 10% Pd/C (0.05 g) under N₂. The suspension was degassed under vacuum and purged with H₂ several times. The mixture was stirred at 25° C. for 1 h under H₂. The reaction mixture was filtered and concentrated under reduced pressure to give a residue. Compound ethyl 4-ethyl-1-methyl-6-oxo-pyridine-3-carboxylate (133 mg, crude) was obtained as a white solid.

Step 5: To a solution of ethyl 4-ethyl-1-methyl-6-oxo-pyridine-3-carboxylate (133 mg, 635.63 umol, 1 eq) in THE (3 mL) and H₂O (3 mL) was added LiOH.H₂O (53.35 mg, 1.27 mmol, 2 eq). The mixture was stirred at 25° C. for 1 hr. The reaction mixture was adjusted to pH=4 with HCl (6N). Then the crude product was extracted with acetate ethyl 15 mL (5 mL*3). Then the water layer was concentrated under reduced pressure to give a residue. Compound 4-ethyl-1-methyl-6-oxo-pyridine-3-carboxylic acid (78.5 mg, crude) was obtained as a white solid.

Step 6: To a solution of 4-ethyl-1-methyl-6-oxo-pyridine-3-carboxylic acid (48.00 mg, 220.56 umol, 1.1 eq, HCl) in Pyridine (5 mL) was added EDCI (46.12 mg, 240.61 umol, 1.2 eq) and 6-amino-3,4-dihydro-1H-quinolin-2-one (32.52 mg, 200.51 umol, 1 eq). The mixture was stirred at 60° C. for 1 hr. The reaction mixture was concentrated under reduced pressure to give a residue. The residue was purified by prep-TLC (SiO₂, Petroleum ether. Ethyl acetate=0:1). Compound 4-ethyl-1-methyl-6-oxo-N-(2-oxo-3,4-dihydro-1H-quinolin-6-yl)pyridine-3-carboxamide (17 mg, 97.419% purity) was obtained. LCMS (M+H⁺): 326.1.

### Example 65. Synthesis of Compound 77

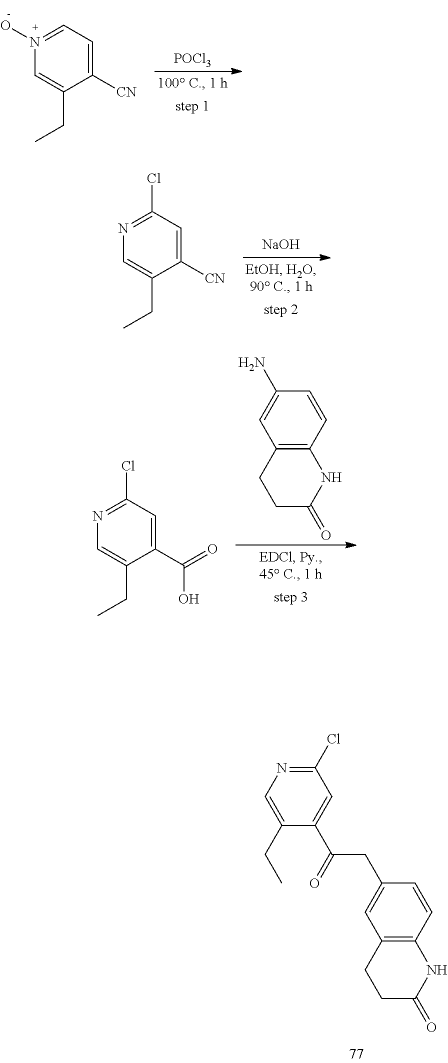

Step 1: The mixture of 3-ethyl-1-oxido-pyridin-1-ium-4-carbonitrile (0.1 g, 674.94 umol, 1 eq) in POCl₃ (1.65 g, 10.76 mmol, 1 mL, 15.94 eq) was stirred at 100° C. for 1 hr. The mixture was concentrated in vacuum. Then to the residue was added EtOAc (5 mL) and water (5 mL). The mixture was adjusted to pH=7-8 by Na₂CO₃. The mixture was extracted with ethyl acetate (5 mL*4). The combined organic phase was washed with brine (10 mL), dried with anhydrous Na₂SO₄, filtered and concentrated in vacuum. The residue was purified by prep-TLC (SiO₂, Petroleum ether: Ethyl acetate=3:1). 2-chloro-5-ethyl-pyridine-4-carbonitrile (30 mg, crude) was obtained as yellow oil.

Step 2: To a solution of 2-chloro-5-ethyl-pyridine-4-carbonitrile (0.03 g, 180.06 umol, 1 eq) in EtOH (2 mL) and H2O (2 mL) was added NaOH (21.61 mg, 540.19 umol, 3 eq). The mixture was stirred at 90° C. for 1 hr. The reaction mixture was concentrated under reduced pressure to remove EtOH. And then the reaction mixture was adjusted pH=3 with HCl (6N) aqueous. The reaction mixture was filtered to give a residue. Compound 2-chloro-5-ethyl-pyridine-4-carboxylic acid (15 mg, crude) was obtained as a white solid.

Step 3: To a solution of 6-amino-3,4-dihydro-1H-quinolin-2-one (4.87 mg, 30.02 umol, 1 eq) and 2-chloro-5-ethyl-pyridine-4-carboxylic acid (10 mg, 45.03 umol, 1.5 eq, HCl) in pyridine (1 mL) was added EDCI (6.91 mg, 36.02 umol, 1.2 eq). The mixture was stirred at 45° C. for 1 hr. The reaction mixture was concentrated under reduced pressure to give a residue. The residue was purified by prep-TLC (SiO₂, Petroleum ether: Ethyl acetate=0:1). Compound 2-chloro-5-ethyl-N-(2-oxo-3,4-dihydro-1H-quinolin-6-yl)pyridine-4 carboxamide (3.2 mg, 96.758% purity) was obtained. LCMS (M+H⁺): 330.1.

### Example 66. Synthesis of Compounds 70 and 72

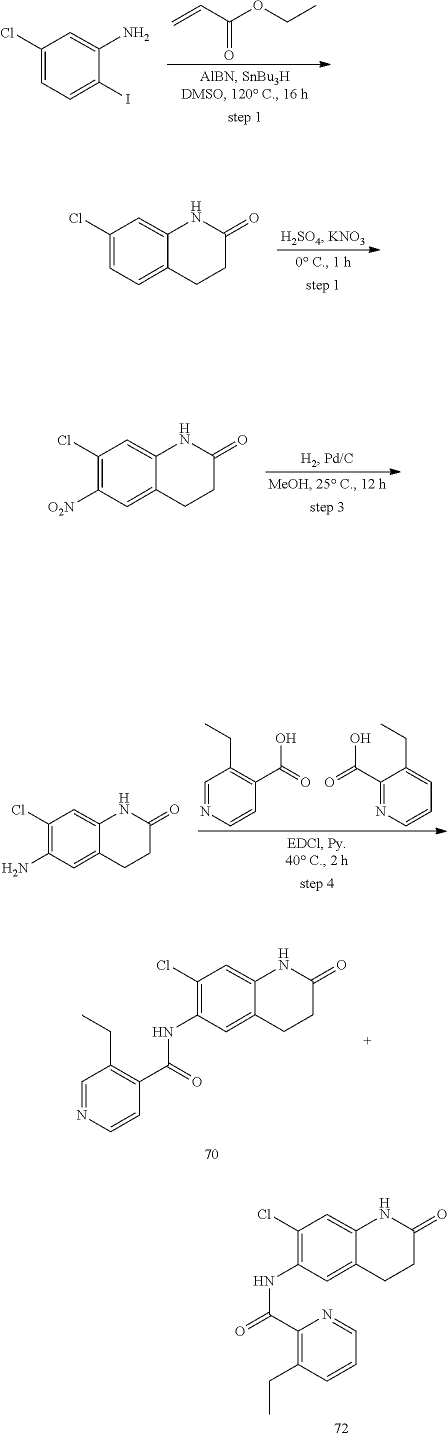

Step 1: To the mixture of 5-chloro-2-iodo-aniline (2 g, 7.89 mmol, 1 eq), ethyl prop-2-enoate (5 g, 49.94 mmol, 5.43 mL, 6.33 eq), Bu₃SnH (3.53 g, 12.13 mmol, 3.21 mL, 1.54 eq) in DMSO (30 mL) was added AIBN (518.28 mg, 3.16 mmol, 0.4 eq). The mixture was stirred at 120° C. for 16 hr. The mixture was cooled to 25° C. To the mixture was added water (120 mL). The aqueous phase was extracted with ethyl acetate (30 mL*4).The combined organic phase was washed with brine (20 mL*3), dried with anhydrous Na₂SO₄, filtered and concentrated in vacuum. The residue was purified by flash silica gel chromatography (ISCO®; 20 g SepaFlash® Silica Flash Column, Eluent of 0-20% Ethyl acetate/Petroleum ethergradient @ 80 mL/min). 7-chloro-3,4-dihydro-1H-quinolin-2-one (0.7 g, 3.85 mmol, 48.85% yield) was obtained as yellow solid.

Step 2: To the mixture of 7-chloro-3,4-dihydro-1H-quinolin-2-one (0.5 g, 2.75 mmol, 1 eq) in H₂SO₄ (4 mL) was added KNO₃ (330 mg, 3.26 mmol, 1.19 eq) in portions at 0° C. Then the mixture was stirred at 0° C. for 1 hr. The mixture was added to ice (20 mL) slowly. The mixture was filtered. 7-chloro-6-nitro-3,4-dihydro-1H-quinolin-2-one (0.5 g, 2.21 mmol, 80.14% yield) was obtained as yellow solid.

Step 3: To a solution of 7-chloro-6-nitro-3,4-dihydro-1H-quinolin-2-one (0.5 g, 2.21 mmol, 1 eq) in MeOH (30 mL) was added 10% Pd/C (0.1 g) under N₂. The suspension was degassed under vacuum and purged with H₂ several times. The mixture was stirred under H₂ (15 psi) at 25° C. for 12 hours. The reaction mixture was filtered and the filter was concentrated. The crude product was triturated with the solution of (5 mL, Petroleum ether:Ethyl acetate=3:1) at 25° C. for 15 min. 6-amino-7-chloro-3,4-dihydro-1H-quinolin-2-one (0.26 g, 1.32 mmol, 59.93% yield) was obtained as purple solid.

Step 4: To the mixture of 6-amino-7-chloro-3,4-dihydro-1H-quinolin-2-one (60 mg, 305.14 umol, 1 eq) and 3-ethylpyridine-4-carboxylic acid (92.25 mg, 610.27 umol, 2 eq, contains some isomer) in Pyridine (1 mL) was added EDCI (70.19 mg, 366.16 umol, 1.2 eq). The mixture was stirred at 45° C. for 1 hr. The mixture was concentrated in vacuum. The residue was purified by prep-TLC (SiO2, Petroleum ether: Ethyl acetate=0:1). Compound 70, N-(7-chloro-2-oxo-3,4-dihydro-1H-quinolin-6-yl)-3-ethyl-pyridine-4-carboxamide (13 mg, 97.1% purity) was obtained LCMS (M+H): 330.0. Compound 72, N-(7-chloro-2-oxo-3,4-dihydro-1H-quinolin-6-yl)-3-ethyl-pyridine-2-carboxamide (3 mg, 100% purity) was obtained, LCMS (M+H⁺): 330.0.

### Example 67. Synthesis of Compound 61

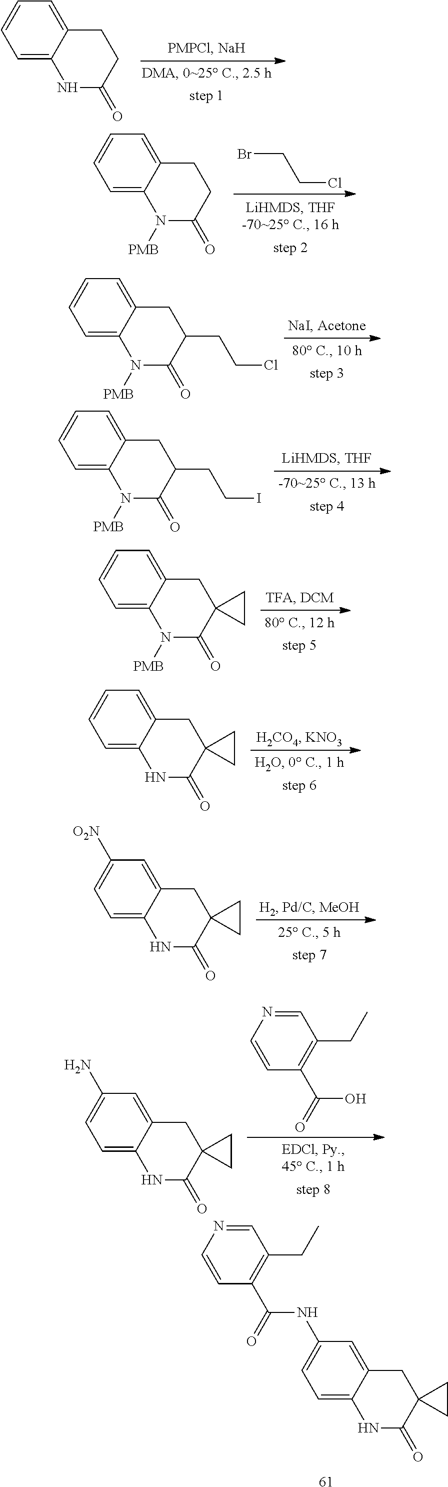

Step 1: To the mixture of NaH (4.42 g, 110.41 mmol, 60% purity, 1.3 eq) in DMA (70 mL) was added 3,4-dihydro-1H-quinolin-2-one (12.5 g, 84.93 mmol, 1 eq) in DMA (50 mL) drop-wise at 0° C. The mixture was stirred at 0° C. for 0.5 h. Then the mixture was added PMB-Cl (14.63 g, 93.43 mmol, 12.72 mL, 1.1 eq) drop-wise at 0° C. The mixture was stirred at 25° C. for 2 hr. The mixture was poured into H₂O (500 mL). The mixture was filtered. The filter cake was washed with H₂O (50 mL*2) and concentrated in vacuum. 1-[(4-methoxyphenyl)methyl]-3,4-dihydroquinolin-2-one (20.5 g, 76.7 mmol, 90.29% yield) was obtained as off-white solid.

Step 2: To the mixture of 1-[(4-methoxyphenyl)methyl]-3,4-dihydroquinolin-2-one (10 g, 37.41 mmol, 1 eq) in THF (150 mL) was added LiHMDS (1 M, 56.11 mL, 1.5 eq) drop-wise at −70° C. Then the mixture was stirred at −70° C. for 0.5 h. Then the mixture was added 1-bromo-2-chloro-ethane (16.09 g, 112.22 mmol, 9.30 mL, 3 eq) drop-wise at 10° C. The mixture was stirred at 25° C. for 15.5 hr under N₂. To the mixture was added H₂O (50 mL) drop-wise at 0° C. The mixture was extracted with ethyl acetate (50 mL*3). The combined organic phase was washed with brine (50 mL*2), dried with anhydrous Na₂SO₄, filtered and concentrated in vacuum. The residue was purified by flash silica gel chromatography (ISCO®; 40 g SepaFlash® Silica Flash Column, Eluent of 0-25% Ethyl acetate/Petroleum ether gradient @ 100 mi./min). 1-[(4-methoxyphenyl)methyl]spiro[4H-quinoline-3,1′-cyclopropane]-2-one and 3-(2-chloroethyl)-1-[(4-methoxyphenyl)methyl]-3,4-dihydroquinolin-2-one (3 g, 9.10 mmol, 24.33% yield) was obtained as yellow solid.

Step 3: To a solution of 1-[(4-methoxyphenyl)methyl]spiro[4H-quinoline-3,1′-cyclopropane]-2-one and 3-(2-chloroethyl)-1-[(4-methoxyphenyl)methyl]-3,4-dihydroquinolin-2-one (2.5 g, 7.58 mmol, 1 eq) in acetone (30 mL) was added NaI (2.27 g, 15.16 mmol, 2 eq). And then the mixture was stirred at 80° C. for 10 hr. The reaction mixture was extracted with ethyl acetate 30 mL (10 mL*3). The combined organic layers were washed with brine 15 mL (5 mL*3), dried over with Na₂SO₄, filtered and concentrated under reduced pressure to give a residue. The mixture 3-(2-iodoethyl)-1-[(4-methoxyphenyl)methyl]-3,4-dihydroquinolin-2-one and 1-[(4-methoxyphenyl)methyl]spiro[4H-quinoline-3,1′-cyclopropane]-2-one (2.8 g, crude) was obtained as a yellow solid.

Step 4: To the solution of 3-(2-iodoethyl)-1-[(4-methoxyphenyl)methyl]-3,4-dihydroquinolin-2-one and 1-[(4-methoxyphenyl)methyl]spiro[4H-quinoline-3,1′-cyclopropane]-2-one (2.8 g, 6.65 mmol, 1 eq) in THF (30 mL) was added drop-wise LiHMDS (1 M, 6.65 mL, 1 eq) at −70° C. and stirred at 1 hr at −70° C. Then the mixture was stirred at 25° C. for 12 hr. To the mixture was added water (10 mL) at 0° C. drop-wise. Then the mixture was extracted with ethyl acetate (30 mL*3). The combined organic phase was washed with brine (20 mL), dried with anhydrous Na₂SO₄, filtered and concentrated in vacuum. The residue was purified by flash silica gel chromatography (ISCO®; 3 g SepaFlash® Silica Flash Column, Eluent of 0-10% Ethyl acetate/Petroleum ethergradient @ 40 mL/min). Compound 1-[(4-methoxyphenyl)methyl]spiro[4H-quinoline-3,1′-cyclopropane]-2-one (1.35 g, 4.60 mmol, 69.24% yield) was obtained as a white solid.

Step 5: To a solution of 1-[(4-methoxyphenyl)methyl]spiro[4H-quinoline-3,1′-cyclopropane]-2-one (0.5 g, 1.70 mmol, 1 eq) in DCM (5 mL) was added TFA (7.70 g, 67.53 mmol, 5 mL, 39.62 eq). The mixture was stirred at 60° C. for 12 hr. The reaction mixture was concentrated under reduced pressure to give a residue. The residue was purified by column chromatography (SiO₂, Petroleum ether/Ethyl acetate=30/1 to 10/1). Compound spiro[1,4-dihydroquinoline-3,1′-cyclopropane]-2-one (0.185 g, 1.07 mmol, 62.67% yield) was obtained as a white solid.

Step 6: To the mixture of spiro[1,4-dihydroquinoline-3,1′-cyclopropane]-2-one (185 mg, 1.07 mmol, 1 eq) in H₂SO₄ (2 mL) was added KNO₃ (160 mg, 1.58 mmol, 1.48 eq) in portions at 0° C. Then the mixture was stirred at 0° C. for 1 hr. The mixture was added to ice (20 mL). Then the mixture was filtered. The filter cake was washed with water (5 mL) and concentrated in vacuum. 6-nitrospiro[1,4-dihydroquinoline-3,1′-cyclopropane]-2-one (0.2 g, 916.56 umol, 85.81% yield) was obtained as yellow solid.

Step 7: To a solution of 6-nitrospiro[1,4-dihydroquinoline-3,1′-cyclopropane]-2-one (60 mg, 274.97 umol, 1 eq) in MeOH (5 mL) was added 10% Pd/C (0.05 g) under N₂. The suspension was degassed under vacuum and purged with H₂ several times. The mixture was stirred under H₂ (15 psi) at 25° C. for 3 hours. The mixture was filtered and concentrated in vacuum. 6-aminospiro[1,4-dihydroquinoline-3,1′-cyclopropane]-2-one (50 mg, 265.64 umol, 96.61% yield) was obtained as yellow solid.

Step 8: To the mixture of 6-aminospiro[1,4-dihydroquinoline-3,1′-cyclopropane]-2-one (40 mg, 212.51 umol, 1 eq) and 3-ethylpyridine-4-carboxylic acid (47.85 mg, 255.01 umol, 1.2 eq, HCl) in Pyridine (1 mL) was added EDCI (48.89 mg, 255.01 umol, 1.2 eq). The mixture was stirred at 45° C. for 1 h. The mixture was concentrated in vacuum. The residue was purified by prep-TLC (SiO₂, Petroleum ether:Ethyl acetate=0:1). 3-ethyl-N-(2-oxospiro[1,4-dihydroquinoline-3,1′-cyclopropane]-6-yl)pyridine-4-carboxamide (8 mg, 94.7% purity) was obtained. LCMS: (M+H⁺) 322.1.

### Example 68. Synthesis of Compound 64

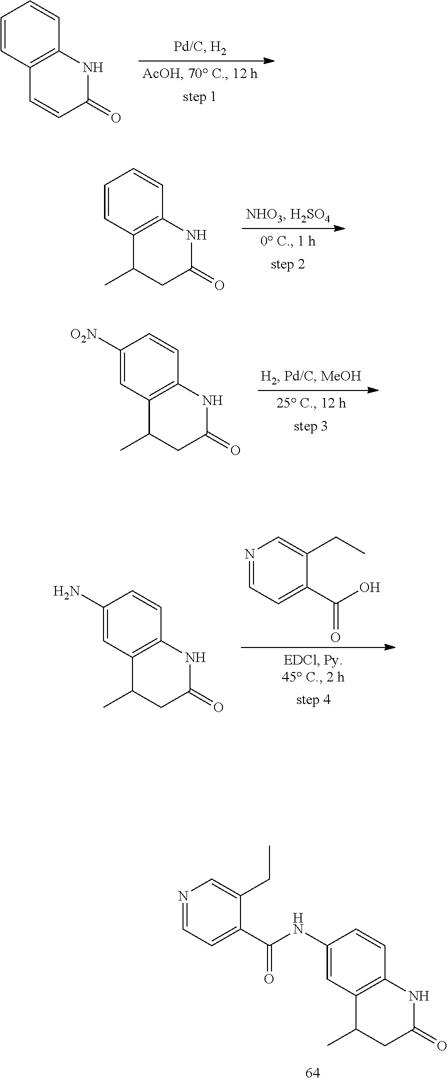 [SMILES: O=c1ccc2ccccc2[nH]1 ; CC1CC(=O)Nc2ccccc21 ; CC1CC(=O)Nc2ccc([N+](=O)[O-])cc21 ; CC1CC(=O)Nc2ccc(N)cc21 ; CCc1cnccc1C(=O)O ; CCc1cnccc1C(=O)Nc1ccc2c(c1)C(C)CC(=O)N2]

Step 1: To a solution of 4-methyl-1H-quinolin-2-one (5 g, 31.41 mmol, 1 eq) in AcOH (50 mL was added 10% Pd/C (0.5 g) under Ar. The suspension was degassed under vacuum and purged with H₂ several times. The mixture was stirred under H₂ (50 Psi) at 70° C. for 12 hours. The reaction mixture was filtered and the filter was concentrated. To the mixture was added sat. Na₂CO₃ (50 mL). The aqueous phase was extracted with ethyl acetate (20 mL*4). The combined organic phase was washed with brine (30 mL), dried with anhydrous Na₂SO₄, filtered and concentrated in vacuum. 4-methyl-3,4-dihydro-1H-quinolin-2-one (3 g, 18.61 mmol, 59.25% yield) was obtained as light yellow solid.

Step 2: To the mixture of 4-methyl-3,4-dihydro-1H-quinolin-2-one (0.5 g, 3.10 mmol, 1 eq) in H₂SO₄ (5 mL) was added HNO₃ (320.89 mg, 3.41 mmol, 229.20 uL, 67% purity, 1.1 eq) drop-wise at 0° C. The mixture was stirred at 0° C. for 1 hr. The mixture was added to ice (50 mL) slowly. The mixture was filtered. The filter cake was washed with H₂O (5 mL). The crude product was triturated with Petroleum ether (10 mL) at 25° C. for 30 min. 4-methyl-6-nitro-3,4-dihydro-1H-quinolin-2-one (0.7 g, crude) was obtained as yellow solid.

Step 3: To a solution of 4-methyl-6-nitro-3,4-dihydro-1H-quinolin-2-one (0.3 g, 1.45 mmol, 1 eq) in MeOH (10 mL) was added 10% Pd/C (0.1 g) under N₂. The suspension was degassed under vacuum and purged with H₂ several times. The mixture was stirred under H₂ (15 Psi) at 25° C. for 12 hours. The reaction mixture was filtered and the filter was concentrated. The crude product was triturated with EtOAc (5 mL) at 25° C. for 30 min. 6-amino-4-methyl-3,4-dihydro-1H-quinolin-2-one (70 mg, 397.24 umol, 27.30% yield) was obtained as light yellow solid.

Step 4: To the mixture of 6-amino-4-methyl-3,4-dihydro-1H-quinolin-2-one (55 mg, 312.12 umol, 1 eq) and 3-ethylpyridine-4-carboxylic acid (56.62 mg, 374.54 umol, 1.2 eq) in Py (1 mL) was added EDCI (71.80 mg, 374.54 umol, 1.2 eq). The mixture was stirred at 45° C. for 2 hr. The reaction was concentrated in vacuum. The residue was purified by prep-TLC (SiO₂, Petroleum ether: Ethyl acetate=0:1). 3-ethyl-N-(4-methyl-2-oxo-3,4-dihydro-1H-quinolin-6-yl)pyridine-4-carboxamide (9 mg, 100% purity) was obtained. LCMS: (M+H⁺) 310.1.

### Example 69. Synthesis of Compound 105

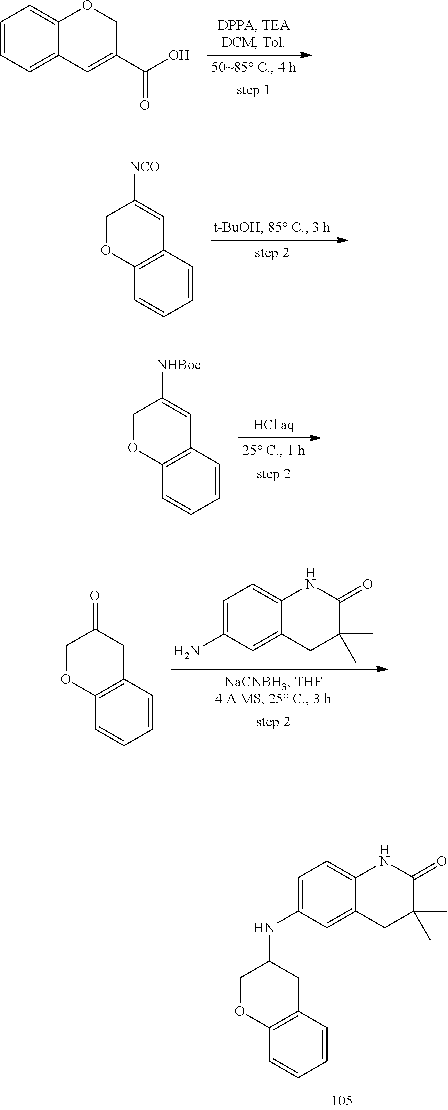

Step 1: To a solution of 2H-chromene-3-carboxylic acid (700 mg, 3.97 mmol, 1 eq) in DCM (14 mL) and TEA (0.7 mL) was added DPPA (1.20 g, 4.37 mmol, 947.12 uL, 1.1 eq) in Toluene (7 mL). The mixture was stirred at 50° C. for 1 hr. Toluene (35 mL) was added to the mixture. The mixture was stirred at 85° C. for 3 hr. The reaction mixture was concentrated under reduced pressure to give a residue 3-isocyanato-2H-chromene (340 mg, 1.96 mmol, 49.41% yield) as a yellow oil.

Step 2: A mixture of 3-isocyanato-2H-chromene (340 mg, 1.96 mmol, 1 eq) in t-BuOH (20 mL) was degassed and purged with N₂ for 3 times, and then the mixture was stirred at 85° C. for 3 hr under N₂ atmosphere. The reaction mixture was concentrated under reduced pressure to give tert-butyl N-(2H-chromen-3-yl)carbamate (0.5 g, crude) as a yellow oil.

Step 3: A mixture of tert-butyl N-(2H-chromen-3-yl)carbamate (0.5 g, 2.02 mmol, 1 eq) in 1M HCl (10 mL) was degassed and purged with N₂ for 3 times, and then the mixture was stirred at 25° C. for 1 hr under N₂ atmosphere. The reaction mixture was adjusted pH to 7-8 with sat.NaHCO₃, then extracted with EtOAc 30 mL (10 mL*3). The combined organic layers were dried over Na₂SO₄, filtered. The filtrate was concentrated under reduced pressure to give chroman-3-one (0.2 g, 1.35 mmol, 66.76% yield) as yellow oil.

Step 4: To a solution of chroman-3-one (90 mg, 607.46 umol, 2 eq), 6-amino-3,3-dimethyl-1,4-dihydroquinolin-2-one (57.78 mg, 303.73 umol, 1 eq) and 4A MS (50 mg, 67.50 umol) in THE (3 mL) was added sodium; cyanoboramide (28.63 mg, 455.59 umol, 1.5 eq). The mixture was stirred at 25° C. for 3 hr. The reaction mixture was filtered and the filtrate was concentrated under reduced pressure to give a residue. The residue was purified by prep-HPLC (column: Phenomenex luna C18 100*40 mm*5 um; mobile phase: [water (0.1% TFA)-ACN]; B %: 20%-53%, 8 min) and triturated with PE:EA=100:1 (2 mL) at 25° C. for 30 min to give 6-(chroman-3-ylamino)-3,3-dimethyl-1,4-dihydroquinolin-2-one (22 mg, 96% purity, TFA). LCMS: (M+H)⁺: 323.1.

### Example 70. Synthesis of Compound 108

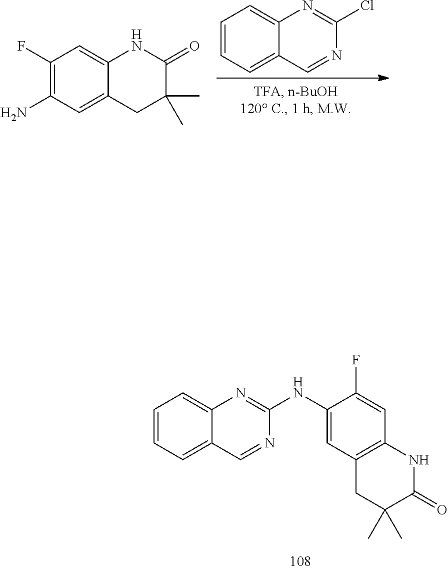

A mixture of 6-amino-7-fluoro-3,3-dimethyl-1,4-dihydroquinolin-2-one (40 mg, 192.09 umol, 2 eq), 2-chloroquinazoline (15.81 mg, 96.05 umol, 1 eq) in TFA (0.01 mL) and n-BuOH (2 mL) was degassed and purged with N₂ for 3 times, and then the mixture was stirred at 120° C. for 1 hr under N₂ atmosphere under microwave condition. The reaction mixture was concentrated under reduced pressure to give a residue. The residue was purified by prep-HPLC (column: Phenomenex Gemini-NX C18 75*30 mm*3 um; mobile phase: [water(0.05% NH₃H₂O+10 mM NH₄HCO₃)-ACN]; B %: 35%-60%, 8 min) to give 7-fluoro-3,3-dimethyl-6-(quinazolin-2-ylamino)-1,4-dihydroquinolin-2-one (11 mg, 96% purity). LCMS: (M+H)⁺: 337.0.

### Example 71. Synthesis of Compound 109

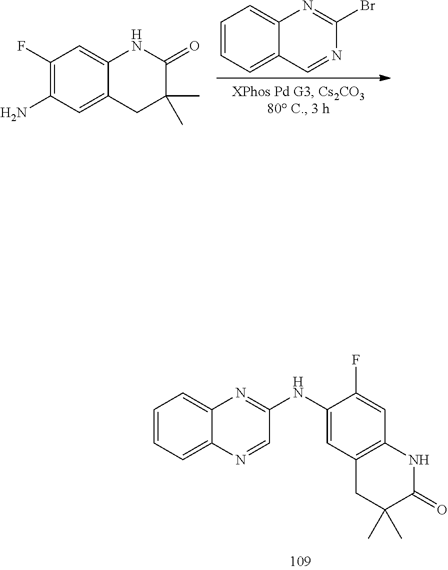

A mixture of 6-amino-7-fluoro-3,3-dimethyl-1,4-dihydroquinolin-2-one (60 mg, 288.14 umol, 1.7 eq), 2-bromoquinoxaline (35.43 mg, 169.49 umol, 1 eq), [2-(2-aminophenyl)phenyl]palladium(2+)-dicyclohexyl-[2-(2,4,6-triisopropylphenyl)phenyl]phosphane methanesulfonate (14.35 mg, 16.95 umol, 0.1 eq), Cs₂CO₃ (110.45 mg, 338.99 umol, 2 eq) in 2-methylbutan-2-ol (4 mL) was degassed and purged with N₂ for 3 times, and then the mixture was stirred at 80° C. for 3 hr under N₂ atmosphere. The reaction mixture was filtered and the filtrate was concentrated under reduced pressure to give a residue. The residue was purified by prep-HPLC (column: Phenomenex Gemini-NX C18 75*30 mm*3 um; mobile phase: [water (0.05% NH₃H₂O+10 mM NH₄HCO₃)-ACN]; B %: 20%-65%, 8 min) to give 7-fluoro-3,3-dimethyl-6-(quinoxalin-2-ylamino)-1,4-dihydroquinolin-2-one (22 mg, 95% purity). LCMS: (M+H)⁺: 337.0.

### Example 72. Synthesis of Compound 112

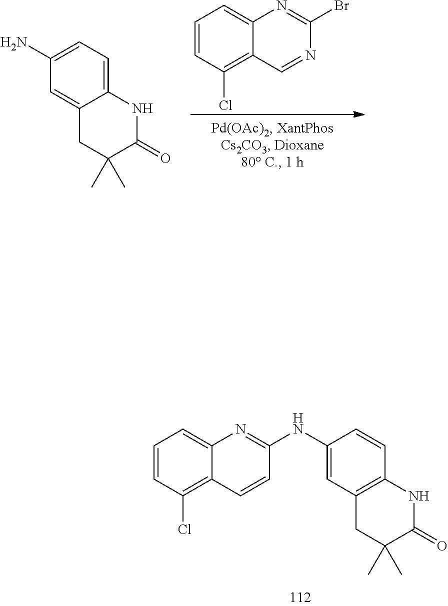 [SMILES: CC1(C)Cc2cc(N)ccc2NC1=O ; Clc1cccc2nc(Br)ncc12 ; CC1(C)Cc2cc(Nc3ccc4c(Cl)cccc4n3)ccc2NC1=O]

A mixture of 6-amino-3,3-dimethyl-1,4-dihydroquinolin-2-one (100 mg, 525.65 umol, 2 eq), 2-bromo-5-chloro-quinoline (63.73 mg, 262.82 umol, 1 eq), Pd(OAc)₂ (11.80 mg, 52.56 umol, 0.2 eq), Cs₂CO₃ (256.90 mg, 788.47 umol, 3 eq) and (5-diphenylphosphanyl-9,9-dimethyl-xanthen-4-yl)-diphenyl-phosphane (30.41 mg, 52.56 umol, 0.2 eq) in dioxane (3 mL) was degassed and purged with N₂ for 3 times, and then the mixture was stirred at 80° C. for 1 hr under N₂ atmosphere. The reaction mixture was filtered and the filtrate was concentrated under reduced pressure to give a residue. The residue was purified by prep-HPLC (column: Phenomenex Gemini-NX C18 75*30 mm*3 um; mobile phase: [water(0.05% NH₃H₂O+10 mM NH₄HCO₃)-ACN]; B %: 45%-75%, 8 min) to give 6-[(5-chloro-2-quinolyl)amino]-3,3-dimethyl-1,4-dihydroquinolin-2-one (43 mg, 98% purity). LCMS: (M+H)⁺: 352.1.

### Example 73. Synthesis of Compound 113

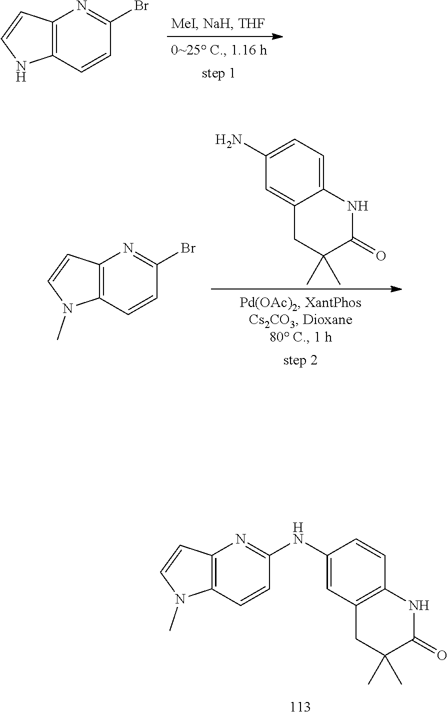 [SMILES: Brc1ccc2[nH]ccc2n1 ; Cn1ccc2nc(Br)ccc21 ; CC1(C)Cc2cc(N)ccc2NC1=O ; Cn1ccc2nc(Nc3ccc4c(c3)CC(C)(C)C(=O)N4)ccc21]

Step 1: To a mixture of 5-bromo-1H-pyrrolo[3,2-b]pyridine (500 mg, 2.54 mmol, 1 eq) in DMF (10 mL) was added NaH (101.50 mg, 2.54 mmol, 60% purity, 1 eq) and stirred for 10 min, then added MeI (360.19 mg, 2.54 mmol, 157.98 uL, 1 eq) in one portion at 0° C. under N₂. The mixture was stirred at 25° C. for 1 hr. The reaction mixture was quenched by addition sat.sodium bicarbonate solution 20 mL at 0° C., and then extracted with EtOAc 60 mL (20 mL*3). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 0/1) to give 5-bromo-1-methyl-pyrrolo[3,2-b]pyridine (250 mg, 1.18 mmol, 46.68% yield) as a yellow solid.

Step 2: A mixture of 6-amino-3,3-dimethyl-1,4-dihydroquinolin-2-one (0.1 g, 525.65 umol, 2 eq), 5-bromo-1-methyl-pyrrolo[3,2-b]pyridine (55.47 mg, 262.82 umol, 1 eq), (5-diphenylphosphanyl-9,9-dimethyl-xanthen-4-yl)-diphenyl-phosphane (30.41 mg, 52.56 umol, 0.2 eq), Pd(OAc)₂ (11.80 mg, 52.56 umol, 0.2 eq) and Cs₂CO₃ (256.90 mg, 788.47 umol, 3 eq) in dioxane (5 mL) was degassed and purged with N₂ for 3 times, and then the mixture was stirred at 80° C. for 1 hr under N₂ atmosphere. The reaction mixture was filtered and the filtrate was concentrated under reduced pressure to give a residue. The residue was purified by prep-HPLC (column: Phenomenex Gemini-NX C18 75*30 mm*3 um; mobile phase: [water(0.05% NH₃H₂O+10 mM NH₄HCO₃)-ACN]; B %: 20%-45%, 8 min) to give 3,3-dimethyl-6-[(1-methylpyrrolo[3,2-b]pyridin-5-yl)amino]-1,4-dihydroquinolin-2-one (20 mg, 96% purity). LCMS: (M+H)⁺: 321.2.

### Example 74. Synthesis of Compound 114

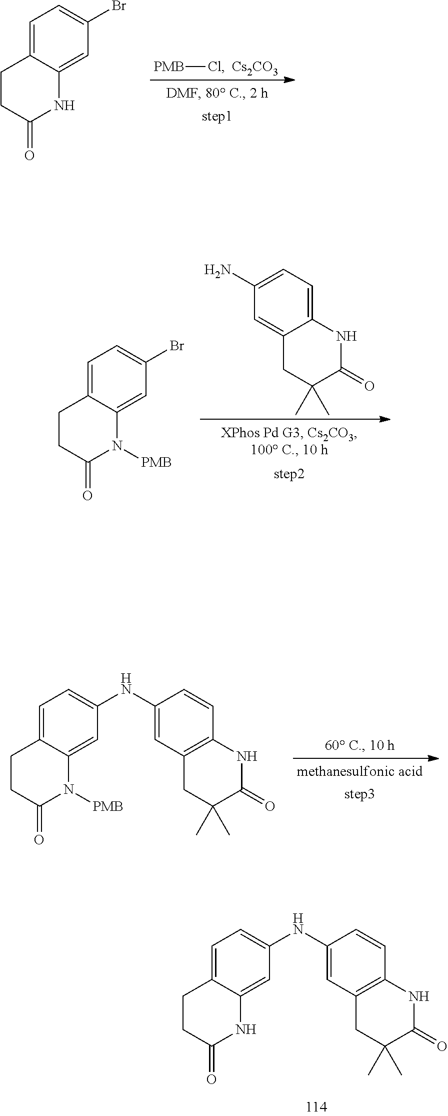

Step 1: To a solution of 7-bromo-3,4-dihydro-1H-quinolin-2-one (100 mg, 442.34 umol, 1 eq) in DMF (2 mL) was added PMB-Cl (76.20 mg, 486.57 umol, 66.26 uL, 1.1 eq) and Cs₂CO₃ (216.18 mg, 663.51 umol, 1.5 eq). The mixture was stirred at 80° C. for 2 hr. The reaction mixture was quenched by addition water 5 mL at 25° C., and filtered and the filter cake was concentrated under reduced pressure to give a residue. The crude product was triturated with the solution (PE:EA=10:1, 2 mL) at 25° C. for 30 min to give 7-bromo-1-[(4-methoxyphenyl)methyl]-3,4-dihydroquinolin-2-one (110 mg, 317.72 umol, 71.83% yield) as a white solid.

Step 2: A mixture of 7-bromo-1-[(4-methoxyphenyl)methyl]-3,4-dihydroquinolin-2-one (110 mg, 317.72 umol, 1 eq), 6-amino-3,3-dimethyl-1,4-dihydroquinolin-2-one (72.53 mg, 381.26 umol, 1.2 eq), [2-(2-aminophenyl)phenyl]palladium(2+)-dicyclohexyl-[2-(2,4,6-triisopropylphenyl)phenyl]phosphane methanesulfonate (26.89 mg, 31.77 umol, 0.1 eq), Cs₂CO₃ (207.04 mg, 635.44 umol, 2 eq) in 2-methylbutan-2-ol (2 mL) was degassed and purged with N₂ for 3 times, and then the mixture was stirred at 100° C. for 10 hr under N₂ atmosphere. The reaction mixture was filtered and the filtrate was concentrated under reduced pressure. The residue was purified by prep-HPLC (column: Phenomenex Gemini-NX C18 75*30 mm*3 um; mobile phase: [water(0.05% NH₃H₂O+10 mM NH₄HCO₃)-ACN]; B %: 35%-55%, 8 min) to give 6-[[1-[(4-methoxyphenyl)methyl]-2-oxo-3,4-dihydroquinolin-7-yl]amino]-3,3-dimethyl-1,4-dihydroquinolin-2-one (40 mg, 87.81 umol, 27.64% yield) as a yellow solid.

Step 3: A mixture of 6-[[1-[(4-methoxyphenyl)methyl]-2-oxo-3,4-dihydroquinolin-7-yl]amino]-3,3-dimethyl-1,4-dihydroquinolin-2-one (140 mg, 307.32 umol, 1 eq), in methanesulfonic acid (0.5 mL) was degassed and purged with N₂ for 3 times, and then the mixture was stirred at 60° C. for 10 hr under N₂ atmosphere. The reaction mixture was adjusted pH to 7-8 with sat.NaHCO₃, and extracted with EA 15 mL (5 mL*3). The organic layers was dried over Na₂SO₄, concentrated under reduced pressure to give a residue. The residue was purified by prep-HPLC (column: Phenomenex Gemini-NX C18 75*30 mm*3 um; mobile phase: [water(0.05% NH₃H₂O+10 mM NH₄HCO₃)-ACN]; B %: 20%-50%, 8 min) to give 3,3-dimethyl-6-[(2-oxo-3,4-dihydro-1H-quinolin-7-yl)amino]-1,4-dihydroquinolin-2-one (51 mg, 95% purity). LCMS: (M+H)⁺: 336.1.

### Example 75. Synthesis of Compounds 115 and 135

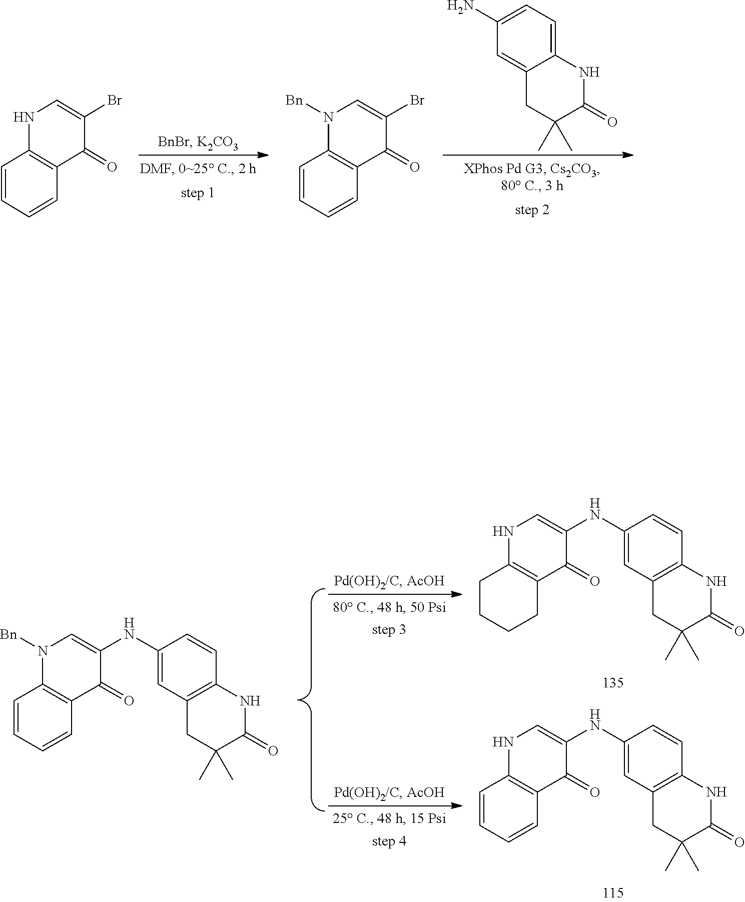 [SMILES: O=c1c(Br)c[nH]c2ccccc12 ; O=c1c(Br)cn(Cc2ccccc2)c2ccccc12 ; CC1(C)Cc2cc(N)ccc2NC1=O ; CC1(C)Cc2cc(Nc3cn(Cc4ccccc4)c4ccccc4c3=O)ccc2NC1=O ; CC1(C)Cc2cc(Nc3c[nH]c4c(c3=O)CCCC4)ccc2NC1=O ; CC1(C)Cc2cc(Nc3c[nH]c4ccccc4c3=O)ccc2NC1=O]

Step 1: To a solution of 3-bromo-1H-quinolin-4-one (0.3 g, 1.34 mmol, 1 eq) in DMF (10 mL) was added K₂CO₃ (277.58 mg, 2.01 mmol, 1.5 eq) at 0° C. and stirred at 0° C. for 0.5 hr. Then benzyl bromide (BnBr) (240.46 mg, 1.41 mmol, 166.99 uL, 1.05 eq) was added to the mixture at 0° C. and stirred at 25° C. for 1.5 hr. The reaction mixture was added H₂O 20 mL and filtered. The filter cake was concentrated under reduced pressure to give 1-benzyl-3-bromo-quinolin-4-one (0.4 g, 1.27 mmol, 95.09% yield) as a yellow solid.

Step 2: A mixture of 1-benzyl-3-bromo-quinolin-4-one (183.50 mg, 584.05 umol, 1 eq), 6-amino-3,3-dimethyl-1,4-dihydroquinolin-2-one (0.2 g, 1.05 mmol, 1.8 eq), [2-(2-aminophenyl)phenyl]palladium(1+)-dicyclohexyl-[2-(2,4,6-triisopropylphenyl)phenyl]-phosphane methanesulfonate (49.44 mg, 58.41 umol, 0.1 eq), Cs₂CO₃ (380.59 mg, 1.17 mmol, 2 eq) in tert-Amyl alcohol 4 mL) was degassed and purged with N₂ for 3 times, and then the mixture was stirred at 80° C. for 3 hr under N₂ atmosphere. The reaction mixture was filtered and the filtrate was concentrated under reduced pressure to give a residue. The residue was purified by column chromatography (SiO₂, Petroleum ether/Ethyl acetate=l/0 to 0/1) to give 1-benzyl-3-[(3,3-dimethyl-2-oxo-1,4-dihydroquinolin-6-yl)amino]quinolin-4 one (0.13 g, 306.96 umol, 52.56% yield) as a yellow solid.

Step 3: A mixture of 1-benzyl-3-[(3,3-dimethyl-2-oxo-1,4-dihydroquinolin-6-yl)amino]quinolin-4-one (50 mg, 118.06 umol, 1 eq), 15% Pd(OH)₂/C (110.54 mg, 1.00 eq) in AcOH (10 mL) was degassed and purged with H₂ for 3 times, and then the mixture was stirred at 80° C. for 48 hr under H₂ (50 Psi) atmosphere. The reaction mixture was filtered and the filtrate was concentrated under reduced pressure to give a residue. The residue was purified by prep-HPLC (column: Phenomenex Gemini-NX C18 75*30 mm*3 um; mobile phase: [water(10 mM NH₄HCO₃)-ACN]; B %: 10%-40%, 12 min) to give Compound 135, 3,3-dimethyl-6-[(4-oxo-5,6,7,8-tetrahydro-1H-quinolin-3-yl)amino]-1,4-dihydroquinolin-2-one (4 mg, 11.50 umol, 97% purity). LCMS: (M+H)⁺: 338.1.

Step 4: A mixture of 1-benzyl-3-[(3,3-dimethyl-2-oxo-1,4-dihydroquinolin-6-yl)amino]quinolin-4-one (60 mg, 141.67 umol, 1 eq), 15% Pd(OH)₂/C (20 mg) in THE (10 mL) was degassed and purged with H₂ for 3 times, and then the mixture was stirred at 25° C. for 48 hr under H₂ (15 Psi) atmosphere. The reaction mixture was filtered and the filtrate was concentrated under reduced pressure to give a residue. The residue was purified by prep-HPLC (column: Phenomenex Gemini-NX C18 75*30 mm*3 um; mobile phase: [water(0.05% NH₃H₂O+10 mM NH₄HCO₃)-ACN]; B %: 10%-40%, 8 min) to give 3,3-dimethyl-6-[(4-oxo-1H-quinolin-3-yl)amino]-1,4-dihydroquinolin-2-one (3 mg, 100% purity). LCMS: (M+H)⁺: 334.1.

### Example 76. Synthesis of Compound 116

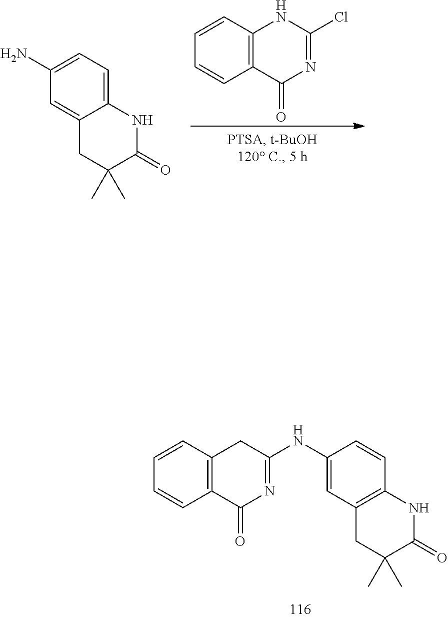 [SMILES: CC1(C)Cc2cc(N)ccc2NC1=O ; O=c1nc(Cl)[nH]c2ccccc12 ; CC1(C)Cc2cc(NC3=NC(=O)c4ccccc4C3)ccc2NC1=O]

A mixture of 6-amino-3,3-dimethyl-1,4-dihydroquinolin-2-one (80 mg, 420.52 umol, 2 eq), 2-chloro-1H-quinazolin-4-one (37.97 mg, 210.26 umol, 1 eq), 4-methylbenzenesulfonic acid (36.21 mg, 210.26 umol, 1 eq), in t-BuOH (2 mL) was taken up into a microwave tube. The sealed tube was heated at 120° C. for 30 min under microwave. The reaction mixture was filtered and the filter cake was concentrated under reduced pressure to give a residue. The residue was purified by prep-HPLC (column: Phenomenex Gemini-NX C18 75*30 mm*3 um; mobile phase: [water(0.05% NH₃H₂O+10 mM NH₄HCO₃)-ACN]; B %: 10%-50%, 8 min) to give 2-[(3,3-dimethyl-2-oxo-1,4-dihydroquinolin-6-yl)amino]-1H-quinazolin-4-one (30 mg, 100% purity), LCMS: (M+H)⁺: 335.2.

### Example 77. Synthesis of Compounds 118 and 120

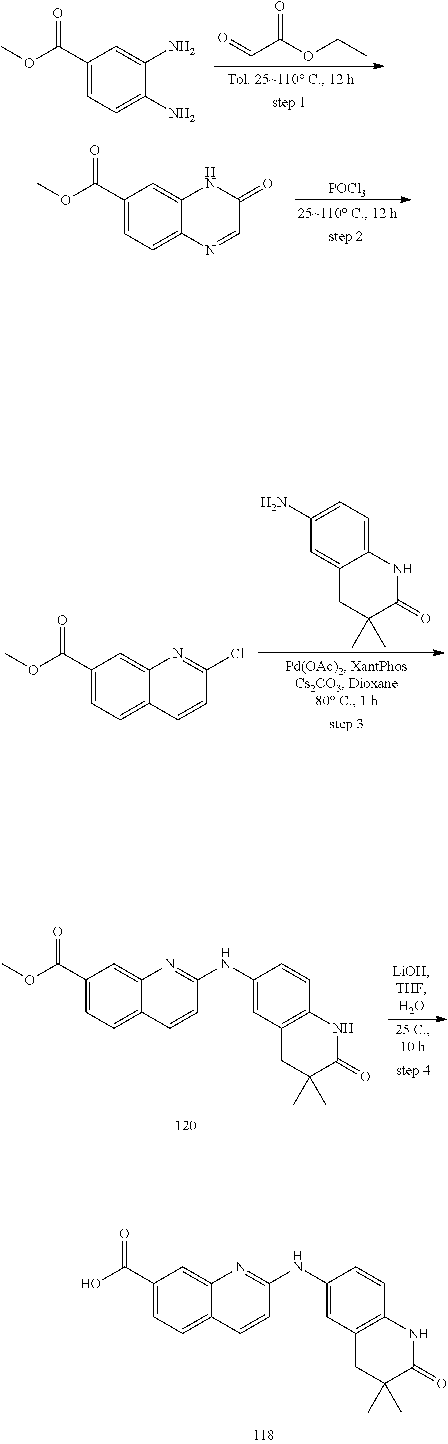

Step 1: To a solution of methyl 3,4-diaminobenzoate (2 g, 12.04 mmol, 1 eq) and ethyl 2-oxoacetate (2.46 g, 12.04 mmol, 50% purity, 1 eq) in Toluene (30 mL) was stirred at 25° C. for 1 hr and stirred at 110° C. for 11 hr. The reaction mixture was quenched by addition H₂O 30 mL at 25° C., and then diluted with EtOAc 30 mL and extracted with EtOAc 60 mL (30 mL*2). The combined organic layers were washed with dried over Na₂SO₄, filtered and the filtrate was concentrated under reduced pressure to give a residue. The residue was purified by column chromatography (SiO2, Petroleum ether/Ethyl acetate=1/0 to 0/1) to give methyl 3-oxo-4H-quinoxaline-6-carboxylate (0.5 g, 2.45 mmol, 20.35% yield) as a yellow solid.

Step 2: A mixture of methyl 3-oxo-4H-quinoxaline-6-carboxylate (0.5 g, 2.45 mmol, 1 eq) in POCl₃ (5 mL) was degassed and purged with N₂ for 3 times, and then the mixture was stirred at 25° C. for 11 hr and stirred at 110° C. for 1 hr. The reaction mixture was concentrated under reduced pressure to give methyl 3-chloroquinoxaline-6-carboxylate (0.5 g, 2.25 mmol, 91.71% yield) as a yellow solid.

Step 3: A mixture of methyl 3-chloroquinoxaline-6-carboxylate (58.51 mg, 262.82 umol, 1 eq), 6-amino-3,3-dimethyl-1,4-dihydroquinolin-2-one (100 mg, 525.65 umol, 2 eq), Pd(OAc)₂ (11.80 mg, 52.56 umol, 0.2 eq), Xantphos (30.41 mg, 52.56 umol, 0.2 eq) and Cs₂CO₃ (256.90 mg, 788.47 umol, 3 eq) in dioxane (3 mL) was degassed and purged with N₂ for 3 times, and then the mixture was stirred at 80° C. for 1 hr under N₂ atmosphere. The reaction mixture was filtered and the filtrate was concentrated under reduced pressure to give a residue. The residue was purified by prep-HPLC (column: Waters Xbridge BEH C18 100*30 mm*10 um; mobile phase: [water(10 mM NH₄HCO₃)-ACN]; B %: 25%-60%, 8 min) to give Compound 120, methyl 3-[(3,3-dimethyl-2-oxo-1,4-dihydroquinolin-6-yl)amino]quinoxaline-6-carboxylate (20 mg, 98% purity). LCMS: (M+H)⁺: 377.0.

Step 4: A mixture of methyl 3-[(3,3-dimethyl-2-oxo-1,4-dihydroquinolin-6-yl)amino]quinoxaline-6-carboxylate (15 mg, 39.85 umol, 1 eq), LiOH.H₂O (3.34 mg, 79.70 umol, 2 eq) in THF (0.5 mL) and H₂O (0.25 mL) was degassed and purged with N₂ for 3 times, and then the mixture was stirred at 25° C. for 10 hr under N₂ atmosphere. The reaction mixture was concentrated under reduced pressure to remove THF. The mixture was adjusted pH to 7-8 with 1M HCl, then filtered and the filter cake was concentrated under reduced pressure to give Compound 118, 3-[(3,3-dimethyl-2-oxo-1,4-dihydroquinolin-6-yl)amino]quinoxaline-6-carboxylic acid (12 mg, 96% purity). LCMS: (M−H)⁻: 361.1.

### Example 78. Synthesis of Compound 119

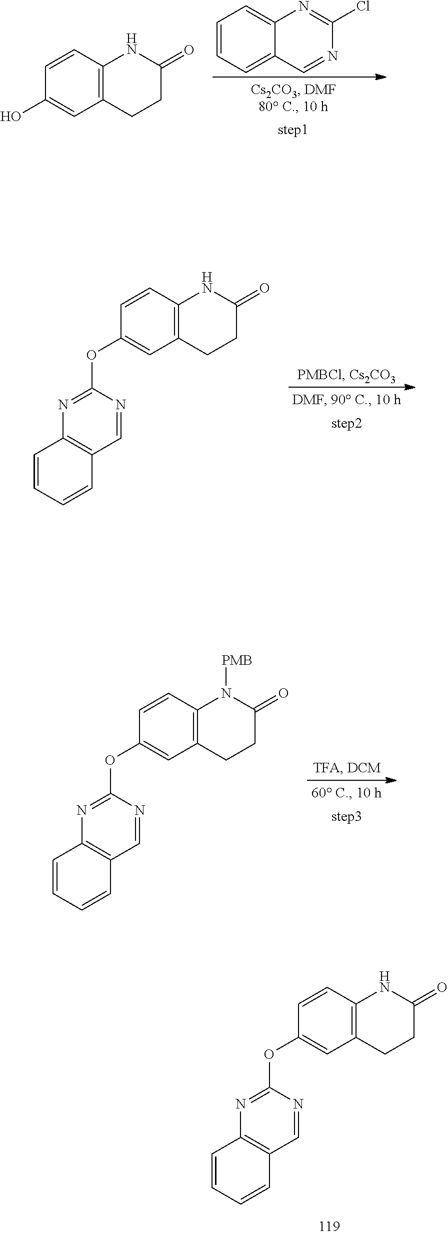

Step 1: To a solution of 6-hydroxy-3,4-dihydro-1H-quinolin-2-one (0.5 g, 3.06 mmol, 1 eq) in DMF (5 mL) was added Cs₂CO₃ (998.39 mg, 3.06 mmol, 1 eq) and 2-chloroquinazoline (504.35 mg, 3.06 mmol, 1 eq). The mixture was stirred at 80° C. for 10 hr. The reaction mixture was quenched by addition H₂O 10 mL, and then diluted with EtOAc 20 mL and extracted with EtOAc (10 mL*2). The combined organic layers were washed with brine (15 mL), dried over Na₂SO₄, filtered and concentrated under reduced pressure to give a residue. The residue was diluted with Petro ether (3 mL) and EtOAc(1 mL). The mixture was stirred at 20° C. for 0.5 h. Then the reaction mixture was filtered and the filter cake was washed with petro ether (4 mL), dried in vacuum to give 6-quinazolin-2-yloxy-3,4-dihydro-1H-quinolin-2-one (300 mg, 1.03 mmol, 33.61% yield) as a white solid.

Step 2: To the mixture of 6-quinazolin-2-yloxy-3,4-dihydro-1H-quinolin-2-one (0.3 g, 1.03 mmol, 1 eq) and Cs₂CO₃ (671.09 mg, 2.06 mmol, 2 eq) in DMF (5 mL) was added 1-(chloromethyl)-4-methoxy-benzene (322.61 mg, 2.06 mmol, 280.53 uL, 2 eq). Then the mixture was stirred at 90° C. for 10 h. The reaction mixture was diluted with H₂O 5 mL and extracted with EtOAc (20 mL*2). The combined organic layers were washed with brine 20 mL, dried over Na₂SO₄, filtered and concentrated under reduced pressure to give a residue. The residue was purified by column chromatography (SiO₂, Petroleum ether/Ethyl acetate=15/1 to 3/1) to give 1-[(4-methoxyphenyl)methyl]-6-quinazolin-2-yloxy-3,4-dihydroquinolin-2-one (0.3 g, 729.13 umol, 70.79% yield) as a yellow oil.

Step 3: A mixture of 1-[(4-methoxyphenyl)methyl]-6-quinazolin-2-yloxy-3,4-dihydroquinolin-2-one (140 mg, 340.26 umol, 1 eq), in TFA (2 mL) and DCM (1 mL) was degassed and purged with N₂ for 3 times, and then the mixture was stirred at 60° C. for 10 hr under N₂ atmosphere. The reaction mixture was concentrated under reduced pressure to give a residue. The residue was adjusted pH to ˜7-8 with sat.NaHCO₃, and extracted with EtOAc 30 mL (10 mL*3). The combined organic layers were dried over Na₂SO₄, filtered and the filtrate was concentrated under reduced pressure. The residue was purified by prep-TLC (SiO₂, PE:EtOAc=3:1) to give 6-quinazolin-2-yloxy-3,4-dihydro-1H-quinolin-2-one (52 mg, 97% purity). LCMS: (M+H)⁺: 292.1.

### Example 79. Synthesis of Compound 122

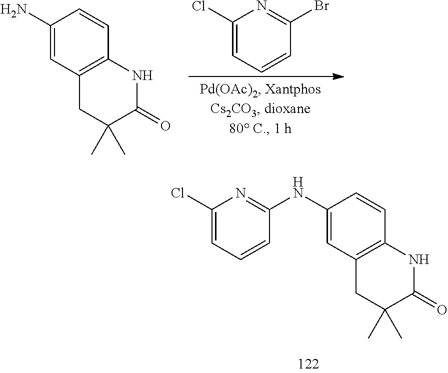 [SMILES: CC1(C)Cc2cc(N)ccc2NC1=O ; Clc1cccc(Br)n1 ; CC1(C)Cc2cc(Nc3cccc(Cl)n3)ccc2NC1=O]

A mixture of 2-bromo-6-chloro-pyridine (44.96 mg, 233.62 umol, 1 eq), 6-amino-3,3-dimethyl-1,4-dihydroquinolin-2-one (80 mg, 420.52 umol, 1.8 eq), Pd(OAc)₂ (10.49 mg, 46.72 umol, 0.2 eq), Xantphos (27.04 mg, 46.72 umol, 0.2 eq) and Cs₂CO₃ (228.36 mg, 700.87 umol, 3 eq) in dioxane (4 mL) was degassed and purged with N₂ for 3 times, and then the mixture was stirred at 80° C. for 1 hr under N₂ atmosphere. The reaction mixture was filtered and the filtrate was concentrated under reduced pressure to give a residue. The residue was purified by prep-HPLC (column: Phenomena(Gemini-NX C18 75*30 mm*3 um; mobile phase: [water(0.05% NH₃H₂O+10 mM NH₄HCO₃)-ACN]; B %: 30%-60%, 8 min) to give 6-[(6-chloro-2-pyridyl)amino]-3,3-dimethyl-1,4-dihydroquinolin-2-one (39 mg, 96% purity). LCMS: (M+H)⁺: 302.0.

### Example 80. Synthesis of Compound 123

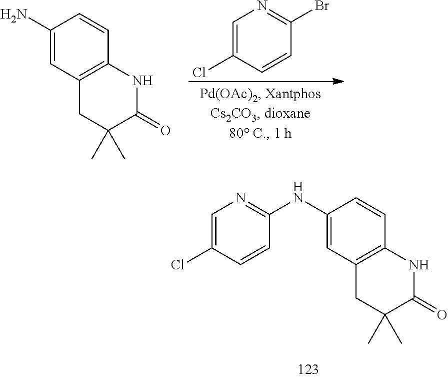 [SMILES: CC1(C)Cc2cc(N)ccc2NC1=O ; Clc1ccc(Br)nc1 ; CC1(C)Cc2cc(Nc3ccc(Cl)cn3)ccc2NC1=O]

A mixture of 6-amino-3,3-dimethyl-1,4-dihydroquinolin-2-one (80 mg, 420.52 umol, 1.8 eq), 2-bromo-5-chloro-pyridine (44.96 mg, 233.62 umol, 1 eq), Pd(OAc)₂ (10.49 mg, 46.72 umol, 0.2 eq), Xantphos (27.04 mg, 46.72 umol, 0.2 eq) and Cs₂CO₃ (228.36 mg, 700.87 umol, 3 eq) in dioxane (4 mL) was degassed and purged with N₂ for 3 times, and then the mixture was stirred at 80° C. for 1 hr under N₂ atmosphere. The reaction mixture was filtered and the filtrate was concentrated under reduced pressure to give a residue. The residue was purified by prep-HPLC (column: Waters Xbridge Prep OBD C18 150*40 mm*10 um; mobile phase: [water(0.05% NH₃H₂O+10 mM NH₄HCO₃)-ACN]; B %: 20%-50%, 8 min) to give 6-[(5-chloro-2-pyridyl)amino]-3,3-dimethyl-1,4-dihydroquinolin-2-one (10 mg, 100% purity). LCMS: (M+H)⁺: 302.0.

### Example 81. Synthesis of Compound 125

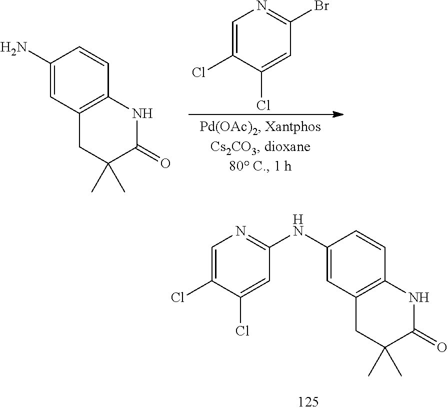 [SMILES: CC1(C)Cc2cc(N)ccc2NC1=O ; Clc1cnc(Br)cc1Cl ; CC1(C)Cc2cc(Nc3cc(Cl)c(Cl)cn3)ccc2NC1=O]

A mixture of 2-bromo-4,5-dichloro-pyridine (53.01 mg, 233.62 umol, 1 eq), 6-amino-3,3-dimethyl-1,4-dihydroquinolin-2-one (80 mg, 420.52 umol, 1.8 eq), Pd(OAc)₂ (10.49 mg, 46.72 umol, 0.2 eq), Cs₂CO₃ (228.36 mg, 700.87 umol, 3 eq) and Xantphos (27.04 mg, 46.72 umol, 0.2 eq) in dioxane (4 mL) was degassed and purged with N₂ for 3 times, and then the mixture was stirred at 80° C. for 1 hr under N₂ atmosphere. The reaction mixture was filtered and the filtrate was concentrated under reduced pressure to give a residue. The residue was purified by prep-HPLC (column: Phenomenex Gemini-NX C18 75*30 mm*3 um; mobile phase: [water(0.05% NH₃H₂O+10 mM NH₄HCO₃)-ACN]; B %: 45%-70%, 8 min) to give 6-[(4,5-dichloro-2-pyridyl)amino]-3,3-dimethyl-1,4-dihydroquinolin-2-one (49 mg, 95% purity). LCMS: (M+H)⁺: 336.0.

### Example 82. Synthesis of Compound 127

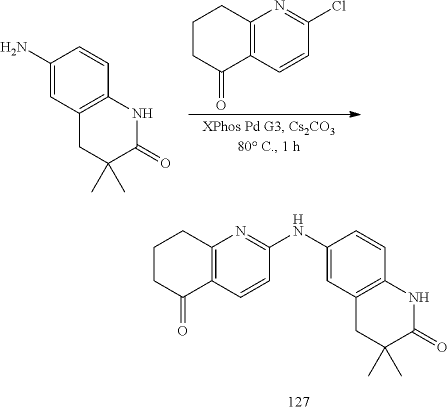 [SMILES: CC1(C)Cc2cc(N)ccc2NC1=O ; O=C1CCCc2nc(Cl)ccc21 ; CC1(C)Cc2cc(Nc3ccc4c(n3)CCCC4=O)ccc2NC1=O]

A mixture of 2-chloro-7,8-dihydro-6H-quinolin-5-one (42.43 mg, 233.62 umol, 1 eq), 6-amino-3,3-dimethyl-1,4-dihydroquinolin-2-one (80 mg, 420.52 umol, 1.8 eq), [2-(2-aminophenyl)phenyl]palladium(2+)-dicyclohexyl-[2-(2,4,6-triisopropylphenyl)phenyl]-phosphane methanesulfonate (19.77 mg, 23.36 umol, 0.1 eq), Cs₂CO₃ (152.24 mg, 467.24 umol, 2 eq) in 2-methylbutan-2-ol (4 mL) was degassed and purged with N₂ for 3 times, and then the mixture was stirred at 80° C. for 1 hr under N₂ atmosphere. The reaction mixture was filtered and the filtrate was concentrated under reduced pressure to give a residue. The residue was purified by prep-HPLC (column: Waters Xbridge Prep OBD C18 150*40 mm*10 um; mobile phase: [water(0.05% NH₃H₂O+10 mM NH₄HCO₃)-ACN]; B %: 15%-45%, 8 min) to give 3,3-dimethyl-6-[(5-oxo-7,8-dihydro-6H-quinolin-2-yl)amino]-1,4-dihydroquinolin-2-one (35 mg, 100% purity). LCMS: (M+H)⁺: 336.1.

### Example 83. Synthesis of Compound 132

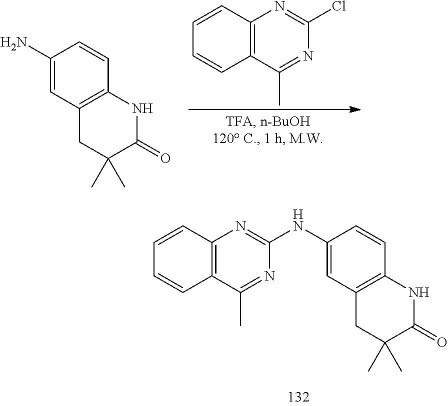 [SMILES: CC1(C)Cc2cc(N)ccc2NC1=O ; Cc1nc(Cl)nc2ccccc12 ; Cc1nc(Nc2ccc3c(c2)CC(C)(C)C(=O)N3)nc2ccccc12]

A mixture of 2-chloro-4-methyl-quinazoline (41.73 mg, 233.62 umol, 1 eq), 6-amino-3,3-dimethyl-1,4-dihydroquinolin-2-one (80 mg, 420.52 umol, 1.8 eq) in TFA (0.03 mL) and n-BuOH (6 mL) was degassed and purged with N₂ for 3 times, and then the mixture was stirred at 120° C. for 1 hr under N₂ atmosphere under microwave condition. The reaction mixture was filtered and the filtrate was concentrated under reduced pressure to give a residue. The residue was purified by prep-HPLC (column: Phenomenex Gemini-NX C18 75*30 mm*3 um; mobile phase: [water(0.05% NH₃H₂O+10 mM NH₄HCO₃)-ACN]; B %: 30%-60%, 8 min) to give 3,3-dimethyl-6-[(4-methylquinazolin-2-yl)amino]-1,4-dihydroquinolin-2-one (38 mg, 100% purity). LCMS: (M+H)⁺: 333.1.

### Example 84. Synthesis of Compound 43

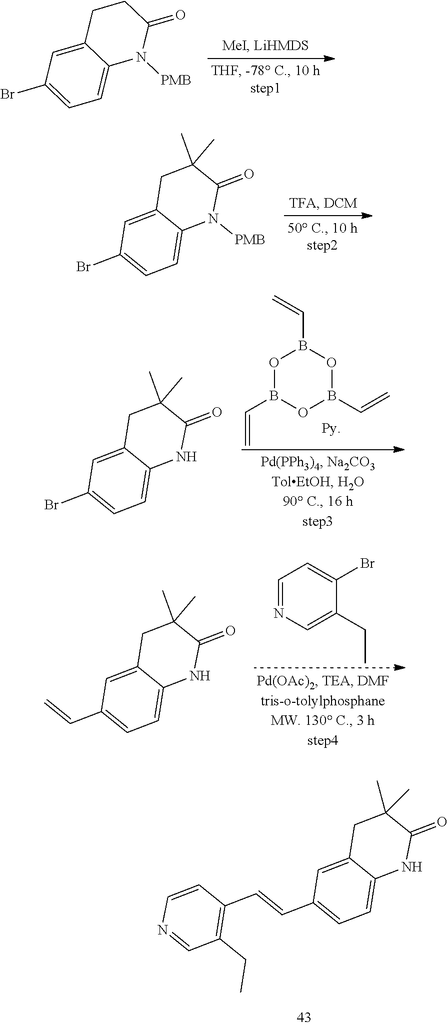

Step 1: To a mixture of 6-bromo-1-[(4-methoxyphenyl)methyl]-3,4-dihydroquinolin-2-one (1 g, 2.89 mmol, 1 eq) in THE (15 mL) was added LiHMDS (1 M, 6.64 mL, 2.3 eq) in one portion at −78° C. under N₂. The mixture was stirred at −78° C. for 30 min. Then MeI (2.46 g, 17.33 mmol, 1.08 mL, 6 eq) was added to the mixture at −78° C. and stirred at −78° C. for 9.5 hours. The reaction mixture was quenched by H₂O (30 mL) and extracted with EtOAc (50 mL*3). The organic layers were dried over Na₂SO₄ and filtered and concentrated under reduced pressure to give a residue. The residue was purified by column chromatography (SiO₂, Petroleum ether/Ethyl acetate=15/1 to 1/1) Compound 6-bromo-1-[(4-methoxyphenyl)methyl]-3,3-dimethyl-4H-quinolin-2-one (300 mg, 801.56 umol, 27.75% yield) was obtained as a white solid.

Step 2: The mixture of 6-bromo-1-[(4-methoxyphenyl)methyl]-3,3-dimethyl-4H-quinolin-2-one (300 mg, 801.56 umol, 1 eq) in TFA (4 mL) and DCM (4 mL) was stirred at 50° C. for 10 hrs under N₂. The reaction mixture was concentrated under reduced pressure to give a residue. The residue was purified by column chromatography (SiO₂, Petroleum ether/Ethyl acetate=10/1 to 2/1). Compound 6-bromo-3,3-dimethyl-1,4-dihydroquinolin-2-one (180 mg, 708.32 umol, 88.37% yield) was obtained as a white solid. LCMS: (M+H)⁺: 254.0, 256.0

Step 3: To a mixture of 6-bromo-3,3-dimethyl-1,4-dihydroquinolin-2-one (150 mg, 590.27 umol, 1 eq) and 2,4,6-trivinyl-1,3,5,2,4,6-trioxatriborinane (114.44 mg, 708.32 umol, 1.2 eq), Na₂CO₃ (187.69 mg, 1.77 mmol, 3 eq) in toluene (5 mL), EtOH (1 mL) and H₂O (0.5 mL) was added Pd(PPh₃)₄ (68.21 mg, 59.03 umol, 0.1 eq) in one portion at 90° C. under N₂. The mixture was stirred at 90° C. for 16 hrs. The reaction mixture was concentrated under reduced pressure to give a residue. The residue was purified by column chromatography (SiO₂, Petroleum ether/Ethyl acetate=15/1 to 10/1). Crude compound 3,3-dimethyl-6-vinyl-1,4-dihydroquinolin-2-one (100 mg, 496.86 umol, 84.18% yield) was obtained as a white solid. LCMS: (M+H)⁺: 202.2

Step 4: 3,3-dimethyl-6-vinyl-1,4-dihydroquinolin-2-one (100 mg, 496.86 umol, 1 eq), 4-bromo-3-ethyl-pyridine (138.66 mg, 745.29 umol, 1.5 eq), tris-o-tolylphosphane (75.61 mg, 248.43 umol, 0.5 eq), TEA (150.83 mg, 1.49 mmol, 207.47 uL, 3 eq) and Pd(OAc)₂ (8.92 mg, 39.75 umol, 0.08 eq) were taken up into a microwave tube in DMF (5 mL). The sealed tube was heated at 130° C. for 3 hours under microwave. The reaction mixture was concentrated under reduced pressure to give a residue. The residue was purified by prep-TLC (SiO₂, Petroleum ether. EtOAc=1:1). Compound 6-[(E)-2-(3-ethyl-4-pyridyl)vinyl]-3,3-dimethyl-1,4-dihydroquinolin-2-one (59 mg, 100% purity) was obtained. LCMS: (M+H)⁺: 307.2. ¹HNMR (400 MHz, MeOD, ppm): δ 8.31-8.33 (m, 2H), 7.65 (d, J=7.2 Hz, 1H), 7.32-7.49 (m, 2H), 7.32 on, 2H), 6.69 (d, J=8.8 Hz, 1H), 2.84-2.89 (m, 4H), 1.24-1.28 (m, 3H), 1.18 (s, 6H).

### Example 85. Synthesis of Compound 33

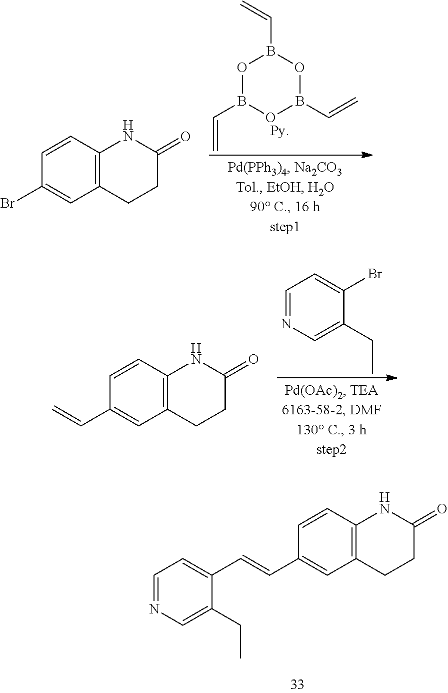

Step 1: To a mixture of 6-bromo-3,4-dihydro-1H-quinolin-2-one (2 g, $0.85 mmol, 1 eq) and pyridine, 2,4,6-trivinyl-1,3,5,2,4,6-trioxatriborinane (2.55 g, 10.62 mmol, 1.2 eq), Na₂CO₃ (2.81 g, 26.54 mmol, 3 eq) in Toluene (40 mL), EtOH (8 mL), and H₂O (2 mL) was added Pd(PPh₃)₄ (1.02 g, 884.68 umol, 0.1 eq) in one portion under N₂. The mixture was heated to 90° C. and stirred for 16 hours. The mixture was cooled to 20° C. and poured into ice-water (˜60 mL). The mixture was extracted with ethyl acetate (80 mL*3). The combined organic phase was washed with brine (50 mL*2), dried with anhydrous Na₂SO₄, filtered and concentrated in vacuum. The residue was purified by silica gel chromatography(Petroleum ether/Ethyl acetate=5/1 to 1/1) 6-vinyl-3,4-dihydro-1H-quinolin-2-one (1.05 g, 6.06 mmol, 68.52% yield) was obtained as yellow solid.

Step 2: 4-bromo-3-ethyl-pyridine (71.61 mg, 384.89 umol, 1 eq), 6-vinyl-3,4-dihydro-1H-quinolin-2-one (100 mg, 577.33 umol, 1.5 eq), tris-o-tolylphosphane (23.43 mg, 76.98 umol, 0.2 eq), TEA (116.84 mg, 1.15 mmol, 160.71 uL, 3 eq) and Pd(OAc)₂ (6.91 mg, 30.79 umol, 0.08 eq) were taken up into a microwave tube in DMF (5 mL). The sealed tube was heated at 130° C. for 3 hours under microwave. The reaction mixture was filtered and washed with EtOAc (3 mL) and filtrate was concentrated under reduced pressure to give a residue. The residue was purified by prep-HPLC (column: Waters Xbridge Prep OBD C18 150*40 mm*10 um; mobile phase: [water(0.05% NH₃.H₂O+10 mM NH₄HCO₃)-ACN]; B %: 20%-35%, 8 min). Compound 6-[(E)-2-(3-ethyl-4-pyridyl)vinyl]-3,4-dihydro-1H-quinolin-2-one (50.6 mg) was obtained. LCMS: (M+H)⁺: 279.1.

### Example 86. Synthesis of Compound 39

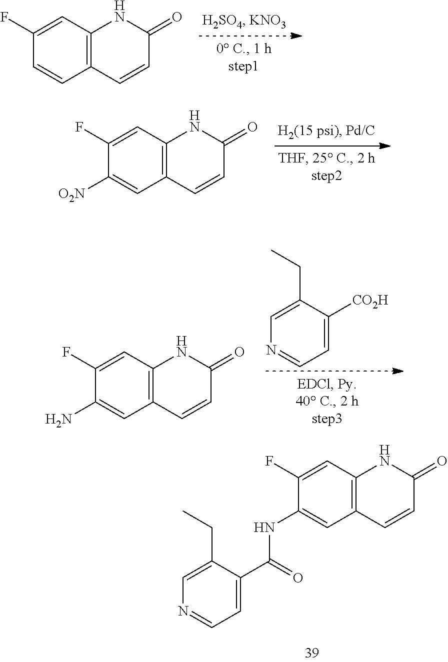

Step 1: To the solution of 7-fluoro-1H-quinolin-2-one (100 mg, 612.94 umol, 1 eq) in conc. H₂SO₄ (8 mL) was added KNO₃ (92.96 mg, 919.41 umol, 1.5 eq) at 0° C. The mixture was stirred at 0° C. for 1 hr. The reaction mixture was cooled at 0° C. and the resulting solution was quenched by adding 10 mL of H₂O/ice. The suspension was filtered and filter cake was concentrated under reduced pressure to give a residue. The crude product 7-fluoro-6-nitro-1H-quinolin-2-one (80 mg, 384.35 umol, 62.71% yield) was obtained as a yellow solid. LCMS: (M+H)⁺: 209.0.

Step 2: The suspension of 7-fluoro-6-nitro-1H-quinolin-2-one (80 mg, 384.35 umol, 1 eq) and 10% Pd/C (20 mg) in THE (5 mL) was degassed and purged with H₂ for 3 times. The mixture was stirred under H₂ (15 Psi) at 25° C. for 2 hrs. The reaction mixture was filtered and filtrates were concentrated under reduced pressure to give a residue. The residue was purified by prep-HPLC (column: Phenomenex luna C18 80*40 mm*3 um; mobile phase: [water(0.04% HCl)-ACN]; B %: 1%-15%, 7 min). Compound 6-amino-7-fluoro-1H-quinolin-2-one (25 mg) was obtained as a white solid.

Step 3: To a mixture of 6-amino-7-fluoro-1H-quinolin-2-one (20 mg, 112.26 umol, 1 eq) and 3-ethylpyridine-4-carboxylic acid (16.97 mg, 112.26 umol, 1 eq) in Pyridine (2 mL) was added EDCI (25.82 mg, 134.71 umol, 1.2 eq) in one portion at 40° C. The mixture was stirred at 40° C. for 2 hours. The reaction mixture was concentrated under reduced pressure to give a residue. The residue was purified by prep-TLC (SiO₂, Ethyl acetate:Methanol=5:1). Compound 3-ethyl-N-(7-fluoro-2-oxo-1H-quinolin-6-yl) pyridine-4-carboxamide (19.6 mg, 100% purity) was obtained. LCMS: (M+H)⁺: 312.1. ¹H NMR (400 MHz, MeOD, ppm): δ 8.58 (s, 1H), 8.53 (d, J=5.2 Hz, 1H), 8.21 (d, J=7.6 Hz, 1H), 7.98 (d, J=9.2 Hz, 1H), 7.52 (d, J=5.2 Hz, 1H), 7.22 (d, J=11.2 Hz, 1H), 6.62 (d, J=9.6 Hz, 1H), 2.90 (q, J=7.6 Hz, 2H), 1.31 (t, J=7.6 Hz, 3H).

Compounds of the present disclosure can be generally prepared by those skilled in the art in view of the present disclosure. See also methods described in PCT/US2019/044278, which has an international filing date of Jul. 31, 2019, the content of which is incorporated by reference in its entirety. By following similar procedures in Examples 1-86, other disclosed compounds herein were or can be prepared. For example, Compound No. 9, 3-ethyl-N-(2-oxo-3,4-dihydro-1H-quinolin-6-yl)pyridine-4-carboxamide, was prepared similarly by using 6-amino-3,4-dihydro-1H-quinolin-2-one in step 5 of Example 1 for amide formation. Compound No. 4, 2-ethyl-N-(7-fluoro-2-oxo-3,4-dihydro-1H-quinolin-6-yl)benzamide, was prepared similarly by using 2-ethylbenzoic acid in step 5 of Example 1 for amide formation. Compound No. 21 was prepared similarly by coupling 3-methylpyridine-4-carboxylic acid with 6-amino-3,4-dihydro-1H-quinolin-2-one similarly as step 5 of Example 1. Compound No. 24 was prepared similarly by coupling 2-methylpyridine-4-carboxylic acid with 6-(methylamino)-3,4-dihydro-1H-quinolin-2-one similarly as step 5 of Example 1. Compound No. 29 was prepared similarly by coupling 2-ethylpyridine-4-carboxylic acid with 6-amino-3,4-dihydro-1H-quinolin-2-one similarly as step 5 of Example 1.

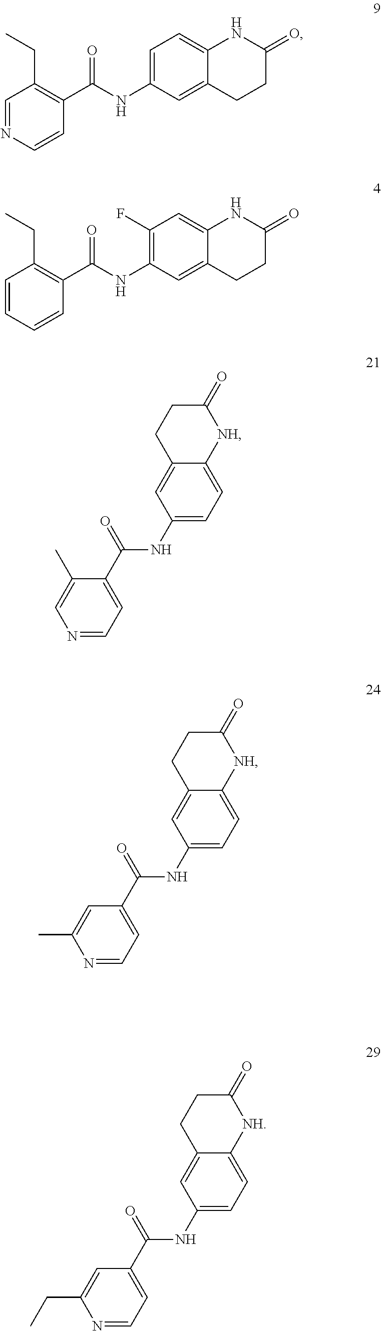 [SMILES: CCc1cnccc1C(=O)Nc1ccc2c(c1)CCC(=O)N2 ; CCc1ccccc1C(=O)Nc1cc2c(cc1F)NC(=O)CC2 ; Cc1cnccc1C(=O)Nc1ccc2c(c1)CCC(=O)N2 ; Cc1cc(C(=O)Nc2ccc3c(c2)CCC(=O)N3)ccn1 ; CCc1cc(C(=O)Nc2ccc3c(c2)CCC(=O)N3)ccn1]

TABLE A
Characterization of Selected Compounds of the Present Disclosure

| Compound | | |
|---|---|---|
| No. | [M + H]⁺ | ¹HNMR |
| 33 | 279.1 | HNMR (400 MHz, MeOD, ppm): δ 8.33 (s, 1H), 8.31 (d, J = 5.2 Hz, 1H), 7.65 |
| | | (d, J = 5.2 Hz, 1H), 7.5 (s, 1H), 7.46 (d, J = 6.8 Hz, 1H), 7.27-7.36 (m, 2H), |
| | | 6.90 (d, J = 8.4 Hz, 1H), 6.90 (d, J = 8.4 Hz, 1H), 3.02 (t, J = 8.0 Hz, 2H), 2.86 |
| | | (q, J = 7.6 Hz, 2H), 2.61 (t, J = 7.6 Hz, 2H), 1.26 (t, J = 7.6 Hz, 2H) |
| 35 | 290.1 | HNMR (400 MHz, MeOD, ppm): 7.95 (d, J = 8.8 Hz, 1H), 7.64-7.68 (m, 3H), |
| | | 7.60 (dd, J = 8.4, 2.4 Hz, 1H), 7.54 (t, J = 8.4 Hz, 1H), 7.26 (t, J = 8.0 Hz, 1H), |
| | | 6.94 (d, J = 8.8 Hz, 1H), 6.86 (d, J = 8.8 Hz, 1H), 2.99 (t, J = 7.2 Hz, 2H), 2.59 |
| | | (t, J = 8.0 Hz, 2H), |
| 38 | 342.1 | HNMR (400 MHz, MeOD, ppm): δ 8.56 (s, 1H), 8.51 (d, J = 5.2 Hz, 1H), 7.60 |
| | | (d, J = 8.0 Hz, 1H), 7.49 (d, J = 4.8 Hz, 1H), 6.75 (d, J = 11.2 Hz, 1H), 2.88 (q, |
| | | J = 15.6 Hz, 2H), 2.83 (s, 2H), 1.26-1.35 (m, 3H), 1.18 (s, 6H) |
| 47 | 308.1 | HNMR (400 MHz, MeOD, ppm): δ8.09 (d, J = 8.4 Hz, 1H), 7.99 (d, J = 8.8 Hz, |
| | | 1H), 7.67 (t, J = 6.4 Hz, 2H), 7.55 (t, J = 6.8 Hz, 1H), 7.28 (t, J = 8.0 Hz, 1H), |
| | | 7.01 (d, J = 9.2 Hz, 1H), 6.74 (d, J = 11.6 Hz, 1H), 2.99 (t, J = 7.6 Hz, 2H), 2.61 |
| | | (t, J = 8.0 Hz, 2H) |
| 48 | 336.1 | HNMR (400 MHz, MeOD, ppm): δ8.07 (d, J = 8.0 Hz, 1H), 7.99 (d, J = 8.8 Hz, |
| | | 1H), 7.67 (t, J = 6.8 Hz, 2H), 7.55 (t, J = 7.6 Hz, 1H), 7.28 (t, J = 7.6 Hz, |
| | | 1H), 7.02 (d, J = 8.8 Hz, 1H), 6.73 (d, J = 11.2 Hz, 1H), 2.84 (s, 2H), 1.20 (s, |
| | | 6H) |
| 50 | 312.0 | HNMR (400 MHz, MeOD, ppm): δ 7.51 (s, 1H), 7.43 (d, J = 8.8 Hz, 1H), 7.37 |
| | | (d, J = 6.8 Hz, 1H), 6.87 (d, J = 8.8 Hz, 1H), 6.36 (d, J = 6.8 Hz, 1H), 2.97 |
| | | (t, J = 7.2 Hz, 2H), 2.56-2.66 (m, 4H), 1.17 (t, J = 7.2 Hz, 3H) |
| 52 | 318.1 | HNMR (400 MHz, MeOD, ppm): δ9.33 (d, J = 5.6 Hz, 1H), 8.48 (d, J = 8.4 Hz, |
| | | 1H), 8.31 (d, J = 8.8 Hz, 1H), 8.19-8.24 (m, 2H), 8.03 (t, J = 8.4 Hz, 1H), 7.66 |
| | | (s, 1H), 7.57 (dd, J = 8.4, 2.0 Hz, 1H), 6.94 (d, J = 8.4 Hz, 1H), 3.02 (t, J = 7.6 |
| | | Hz, 2H), 2.61 (t, J = 6.4 Hz, 2H) |
| 53 | 350.0 | HNMR (400 MHz, MeOD, ppm): δ8.67-8.70 (m, 2H), 7.63 (d, J = 4.8 Hz, |
| | | 1H), 7.54 (s, 1H), 7.45 (d, J = 7.6 Hz, 1H), 6.88 (d, J = 8.4 Hz, 1H), 3.94 (q, J = |
| | | 21.6 Hz, 2H), 2.98 (t, J = 7.2 Hz, 2H), 2.59 (t, J = 8.0 Hz, 2H), |
| 54 | Not | HNMR (400 MHz, MeOD, ppm): δ 9.01 (s, 1H), 8.94 (d, J = 4.4 Hz, 1H), 7.68 |
| | observed | (d, J = 4.8 Hz, 1H), 7.51 (s, 1H), 7.42 (dd, J = 8.4, 2.4 Hz, 1H), 6.88 (d, J = 8.4 |
| | | Hz, 1H), 2.98 (t, J = 7.2 Hz, 2H), 2.59 (t, J = 6.0 Hz, 2H) |
| 55 | 310.1 | HNMR (400 MHz, MeOD, ppm): δ8.55 (d, J = 5.6 Hz, 1H), 7.67 (d, J = 6.0 Hz, |
| | | 1H), 7.55 (s, 1H), 7.46 (dd, J = 8.0, 2.0 Hz, 1H), 6.89 (d, J = 8.4 Hz, 1H), 2.98 |
| | | (t, J = 7.2 Hz, 2H), 2.91 (q, J = 15.2 Hz, 2H), 2.76 (s, 3H), 2.59 (t, J = 7.6 Hz, |
| | | 2H), 1.27 (t, J = 7.6 Hz, 3H) |
| 56 | 310.1 | HNMR (400 MHz, MeOD, ppm): δ 8.39 (s, 1H), 7.53 (s, 1H), 7.46 (dd, J = 8.8, |
| | | 2.4 Hz, 1H), 7.33 (s, 1H), 6.87 (d, J = 8.4 Hz, 1H), 2.98 (t, J = 7.2 Hz, 2H), 2.80 |
| | | (q, J = 14.8 Hz, 2H), 2.56-2.61(m, 5H), 1.25 (t, J = 7.6 Hz, 3H) |
| 59 | 322.1 | HNMR (400 MHz, MeOD, ppm): δ 8.98 (s, 1H), 8.85 (d, J = 6.0 Hz, 1H), 8.10 |
| | | (d, J = 5.6 Hz, 1H), 7.57 (s, 1H), 7.48 (d, J = 8.4 Hz, 1H), 6.90 (d, J = 8.4 Hz, |
| | | 1H), 3.99 (t, J = 7.6 Hz, 2H), 2.89 (d, J = 7.2 Hz, 2H), 2.59 (t, J = 7.6 Hz, 2H), |
| | | 1.08-1.14 (m, 1H), 0.60-0.64 (m, 2H), 0.30-0.35 (m, 2H) |
| 60 | 310.1 | HNMR (400 MHz, MeOD, ppm): δ 8.52 (s, 1H), 8.50 (d, J = 5.2 Hz, 1H), 7.53 |
| | | (s, 1H), 7.43-7.47 (m, 2H), 6.88 (d, J = 8.4 Hz, 1H), 2.98 (t, J = 7.2 Hz, 2H), |
| | | 2.81 (t, J = 7.6 Hz, 2H), 2.59 (t, J = 8.0 Hz, 2H), 1.63-1.73 (m, 2H), 0.95 (t, J = |
| | | 7.2 Hz, 3H) |
| 61 | 322.1 | HNMR (400 MHz, MeOD, ppm): δ 8.55 (s, 1H), 8.50 (d, J = 5.2 Hz, 1H), 7.49 |
| | | (s, 1H), 7.43-7.47 (m, 2H), 6.89 (d, J = 8.4 Hz, 1H), 2.90 (s, 2H), 2.85 (q, J = |
| | | 15.2 Hz, 2H), 1.22-1.30 (m, 5H), 0.79-0.83 (m, 2H) |
| 63 | 310.1 | HNMR (400 MHz, MeOD, ppm): δ 8.55 (s, 1H), 8.50 (d, J = 4.8 Hz, 1H), 7.54 |
| | | (s, 1H), 7.45 (d, J = 5.2 Hz, 2H), 6.87 (d, J = 8.8 Hz, 1H), 3.01-3.07 (m, 1H), |
| | | 2.85 (q, J = 15.2 Hz, 2H), 2.84-2.77 (m, 2H), 1.28 (t, J = 7.6 Hz, 3H), 1.23 (t, J = |
| | | 6.8 Hz, 3H) |
| 64 | 310.1 | HNMR (400 MHz, CDCl3, ppm): δ 8.55-8.63 (m, 2H), 7.57 (s, 1H), 7.54 (s, |
| | | 1H), 7.44 (s, 1H), 7.33-7.39 (m, 2H), 6.76 (d, J = 8.4 Hz, 1H), 3.18 (q, J = |
| | | 14.0 Hz, 1H), 2.88 (q, J = 15.2 Hz, 2H), 2.71-2.78 (m, 1H), 2.41-2.48 (m, |
| | | 1H), 1.25-1.36 (m, 6H) |
| 70 | 330.0 | HNMR (400 MHz, MeOD, ppm): δ 8.56 (s, 1H), 8.52 (d, J = 5.2 Hz, 1H), 7.54 |
| | | (d, J = 4.4 Hz, 1H), 7.51 (s, 1H), 7.04 (s, 1H), 3.00 (t, J = 7.2 Hz, 2H), 2.92 (q, |
| | | J = 15.6 Hz, 2H), 2.61(t, J = 8.4 Hz, 2H), 1.31(t, J = 7.6 Hz, 3H) |
| 72 | 330.0 | HNMR (400 MHz, DMSO, ppm): δ 10.54 (s, 1H), 10.17 (s, 1H), 8.56 (d, J = |
| | | 3.6 Hz, 1H), 8.03 (s, 1H), 7.88 (d, J = 7.2 Hz, 1H), 7.57-7.62 (m, 1H), 7.00 (s, |
| | | 1H), 3.11 (q, J = 14.4 Hz, 2H), 2.92 (t, J = 7.6 Hz, 2H), 2.37-2.56 (m, 2H), |
| | | 1.22 (t, J = 7.2 Hz, 3H) |
| 77 | 330.0 | HNMR (400 MHz, MeOD, ppm): δ 8.36 (s, 1H), 7.54 (s, 1H), 7.52 (s, 1H), 7.46 |
| | | (dd, J = 8.8, 2.4 Hz, 1H), 6.88 (d, J = 8.8 Hz, 1H), 2.98 (t, J = 7.2 Hz, 2H), 2.82 |
| | | (q, J = 14.8 Hz, 2H), 2.58 (t, J = 8.0 Hz, 2H), 1.26 (t, J = 7.6 Hz, 3H) |
| 78 | 314.1 | HNMR (400 MHz, MeOD, ppm): δ 8.18 (s, 1H), 7.54 (s, 1H), 7.46 (dd, J = 8.8, |
| | | 2.0 Hz, 1H), 7.15 (d, J = 2.4 Hz, 1H), 6.88 (d, J = 8.4 Hz, 1H), 2.98 (t, J = 7.2 |
| | | Hz, 2H), 2.82 (q, J = 15.2 Hz, 2H), 2.59 (t, J = 8.0 Hz, 2H), 1.25 (t, J = 7.6 Hz, |
| | | 3H) |
| 79 | 328.2 | HNMR (400 MHz, MeOD, ppm): δ 8.39 (s, 1H), 7.59(d, J = 8.4 Hz, 1H), 7.36 |
| | | (s, 1H), 6.76(d, J = 10.4 Hz, 1H), 2.97(t, J = 7.6 Hz, 2H), 2.82(q, J = 15.6 Hz, |
| | | 2H), 2.60(t, J = 8.0 Hz, 2H), 2.57 (s, 3H), 1.27(t, J = 7.6 Hz, 3H) |
| 80 | 348.1 | HNMR (400 MHz, MeOD, ppm): δ 8.37 (s, 1H), 7.62(d, J = 7.6 Hz, 1H), 7.53 |
| | | (s, 1H), 6.76 (d, J = 11.2 Hz, 1H), 2.97 (t, J = 7.2 Hz, 2H), 2.84 (q, J = 15.2 Hz, |
| | | 2H), 2.60 (t, J = 8.0 Hz, 2H), 1.28 (t, J = 7.2 Hz, 3H) |
| 81 | 340.1 | HNMR (400 MHz, MeOD, ppm): δ 8.64 (s, 1H), 8.53 (d, J = 4.4 Hz, 1H), 7.61 |
| | | (d, J = 8.0 Hz, 1H), 7.50 (d, J = 5.2 Hz, 1H), 6.76 (d, J = 11.2 Hz, 1H), 2.98 (t, J = |
| | | 7.6 Hz, 2H), 2.77 (d, J = 7.2 Hz, 2H), 2.60 (t, J = 7.6 Hz, 2H), 1.03-1.13 (m, |
| | | 1H), 0.52-0.57 (m, 2H), 0.24-0.29 (m, 2H) |
| 82 | 368.2 | HNMR (400 MHz, MeOD, ppm): δ 8.72 (d, J = 5.2 Hz, 1H), 8.71 (s, 1H), 7.68 |
| | | (d, J = 5.2 Hz, 1H), 7.58 (d, J = 7.6 Hz, 1H), 6.78 (d, J = 10.8 Hz, 1H), 3.97 (q, |
| | | J = 21.6 Hz, 2H), 3.00 (t, J = 8.0 Hz, 2H), 2.62 (t, J = 8.0 Hz, 2H) |
| 83 | 310.1 | HNMR (400 MHz, DMSO, ppm): δ 10.07 (s, 1H), 9.90 (s, 1H), 8.58 (s, 1H), |
| | | 8.54 (d, J = 4.8 Hz, 1H), 7.43 (d, J = 5.2 Hz, 1H), 7.16 (s, 1H), 6.72 (s, 1H), |
| | | 2.85 (t, J = 7.2 Hz, 2H), 2.78 (q, J = 7.6 Hz, 2H), 2.45 (t, J = 7.2 Hz, 2H), 2.18 |
| | | (s, 3H), 1.22 (t, J = 7.6 Hz, 3H) |
| 84 | 321.3 | HNMR (400 MHz, DMSO, ppm): δ 10.68 (s, 1H), 10.35 (s, 1H), 8.61 (s, 1H), |
| | | 8.58 (d, J = 4.8 Hz, 1H), 7.42-7.45 (m, 2H), 7.19 (s, 1H), 3.00 (t, J = 6.8 Hz, |
| | | 2H), 2.81 (q, J = 7.2 Hz, 2H), 2.45-2.78 (m, 2H), 1.22 (t, J = 7.6 Hz, 3H) |
| 87 | 304.1 | HNMR (400 MHz, DMSO, ppm): δ 9.96 (s, 1H), 9.24 (s, 1H), 8.14 (d, J = 9.2 |
| | | Hz, 1H), 7.81 (s, 1H), 7.70 (dd, J = 8.8, 2.4 Hz, 1H), 7.50 (d, J = 8.0 Hz, 1H), |
| | | 7.43 (t, J = 7.6 Hz, 1H), 7.09 (d, J = 6.8 Hz, 1H), 7.02 (d, J = 8.8 Hz, 1H), 6.82 |
| | | (d, J = 8.8 Hz, 1H), 2.90 (t, J = 7.2 Hz, 2H), 2.56 (s, 3H), 2.45 (t, J = 8.0 Hz, |
| | | 2H) |
| 92 | 319.2 | HNMR (400 MHz, DMSO, ppm): δ 9.97 (s, 1H), 9.81 (s, 1H), 8.50 (s, 1H), |
| | | 7.81-7.83 (m, 2H), 7.71-7.81 (m, 2H), 7.69 (t, J = 7.2 Hz, 1H), 7.45 (t, J = 8.4 |
| | | Hz, 1H), 6.87 (d, J = 8.4 Hz, 1H), 2.80 (s, 2H), 1.08 (s, 6H) |
| 93 | 319.1 | HNMR (400 MHz, DMSO, ppm): δ 9.90 (s, 1H), 9.69 (s, 1H), 9.26 (s, 1H), |
| | | 7.88 (d, J = 7.6 Hz, 1H), 7.74-7.79 (m, 3H), 7.63 (d, J = 8.4 Hz, 1H), 7.35 (t, J = |
| | | 7.2 Hz, 1H), 6.82 (d, J = 4.0 Hz, 1H), 2.76 (s, 2H), 1.08 (s, 6H) |
| 99 | 308.1 | HNMR (400 MHz, DMSO, ppm): δ 10.42 (s, 1H), 9.94 (s, 1H), 7.58 (d, J = 2.0 |
| | | Hz, 1H), 7.40-7.50 (m, 3H), 7.20 (t, J = 7.6 Hz, 1H), 7.12 (t, J = 7.6 Hz, 1H), |
| | | 6.85 (d, J = 8.4 Hz, 1H), 2.77 (s, 2H), 1.07 (s, 6H) |
| 109 | 337.0 | HNMR (400 MHz, DMSO, ppm): δ 10.08 (s, 1H), 9.44 (s, 1H), 8.68 (s, 1H), |
| | | 8.21 (d, J = 8.4 Hz, 1H), 7.85 (d, J = 8.0 Hz, 1H), 7.61-7.68 (m, 2H), 7.45 (t, J = |
| | | 6.8 Hz, 1H), 6.78 (d, J = 12.0 Hz, 1H), 2.80 (s, 2H), 1.09 (s, 6H) |

### Biological Example 1. Material and General Methods

### Cell Lines and Cell Culture

MDA-MB-468, MCF7, 293T, B16F10, HCT116, HepG2, LN229, HTB 140 and SW480 cells were cultured in DMEM+10% FBS; DU 145 cells and its derivatives, PC3, AsPC-1, NCI-H358 were cultured in RPMI-1640+10% FBS; and SUM159 cells and its derivatives were cultured in F12 media supplemented with 10% FBS, 10 μg/mL Insulin and 20 ng/mL EGF. Cell lines were labeled with retroviral vectors with bi-cistronic expression of GFP/firefly luciferase to facilitate imaging and flow cytometry experiments. All cell lines were verified negative for mycoplasma contamination by monthly PCR analysis. No cells lines used here appear in the database of commonly misidentified cell lines (ICLAC). All cell lines were validated with STR analysis and compared to NCBI repository data.

### Cloning, Viral Production, and Transduction

The coding sequences of Aldh1a1, Aldh1a2, Aldh1a3 and Aldh3a1 were cloned from cDNA made from pooled human reference RNA samples. Cloned sequences flanked by Age1 and Xho1 restriction sites were inserted into the pLex lentiviral plasmid. Clones were sequenced and compared against NCBI expressed sequence tags (SSTs) for accuracy. Viral production of each enzyme was performed by transfection into the 293T packaging cell line using PEI along with PsPax2 and VSVG packaging vectors. Viruses were collected and filtered at 0.45 um, then cells were transduced using polybrene (8 μg/mL) for 12 hours, followed by culture with 1 μg/mL puromycin for the duration of experiments. All viral transduction and selection was performed on a cell population-wide basis.

### Biological Example 2. Genetic Knockout Studies

CRISPR-Cas9 vectors containing both the Cas9 gene and gRNA sequences containing homologous sequences to genomic Aldh1a3 were transduced into MDA-MB-468 cells by lentiviral infection followed by puromycin selection for viral integration, and the resulting cells were analyzed by ALDEFLUOR™ assay. Two ALDH1a3-targeting gRNA vectors were used to create two derivative cell lines, and one gRNA with a scrambled sequence was used to generate a control derivative cell line. ALDH1a3 targeting gRNA vectors were compared to a non-target gRNA.

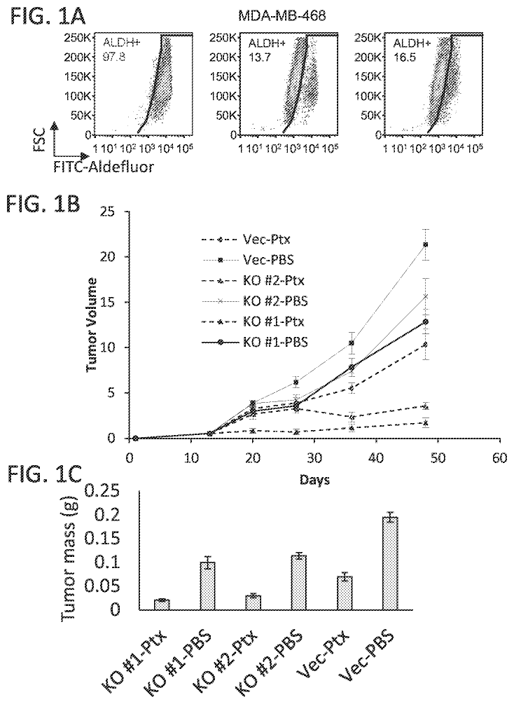

Aldh1a3 knockout or control cells were implanted into the mammary fat pad of female NSG mice and were treated with either PBS or Paclitaxel at 25 mg/kg for 6 doses. Tumor mass from each group was measured at the experimental endpoint. The results were shown in FIGS. 1A-1C.

FIG. 1A is flow cytometry spectra, and shows that genetic knockout of ALDH1a3 (middle and rightmost spectra) in MDA-MB-468 breast cancer cells substantially decreases ALDEFLUOR™ activity compared to control MDA-MB-468 cells (leftmost spectra). FIG. 1B is a line graph of tumor volume (mm³) versus time (days), and shows that genetic knockout of ALDH1a3 in MDA-MB-468 breast cancer cells slows primary tumor growth and sensitizes tumors to paclitaxel. FIG. 1C is a bar graph of tumor mass (g) versus genetic knockout, and shows that genetic knockout of ALDH1a3 in MDA-MB-468 breast cancer cells slows primary tumor growth and sensitizes tumors to paclitaxel. Thus, these results show that genetic knockout of Aldh1a3 in MDA-MB-468 breast cancer cells slows primary tumor growth and sensitizes tumors to paclitaxel.

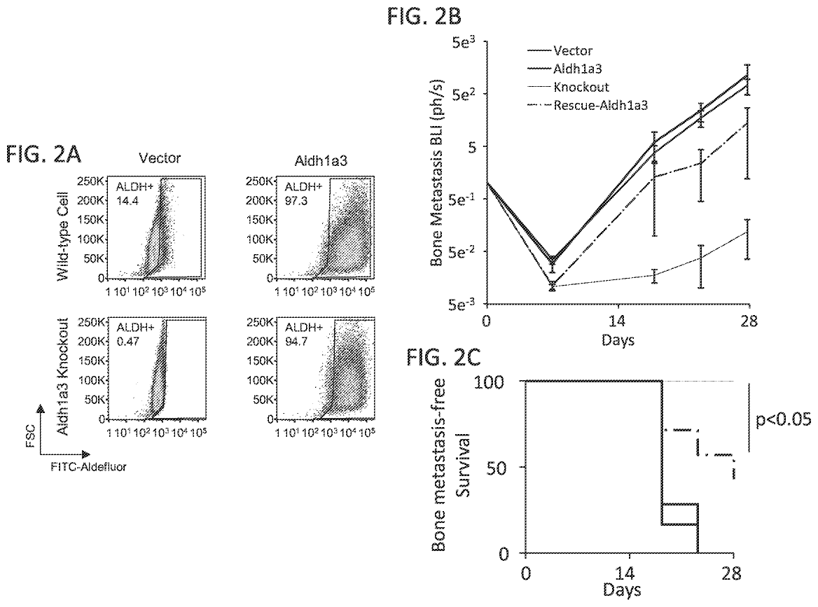

In another experiment, CRISPR-Cas9 vectors targeting the ALDH1a3 gene were transduced into the Sum159-M1a cell line followed by a rescue or control vector and analyzed by the Aldefluor assay. CRISPR-Cas9 vectors containing both the Cas9 gene and gRNA sequences containing homologous sequences to genomic Aldh1a3 were transduced into Sum159-M1a cells followed by selection for positively transfected cells by flow cytometry. Positively transfected cells were then virally transduced with lentiviral vectors containing either the vector backbone or full-length human Aldh1a3. Positive transductants were selected by puromycin, and the resulting cells were analyzed by ALDEFLUOR™ assay. In a xenograft experiment, the knockout-vector or knockout-Aldh 1a3 or wild-type vector or wild-type-Aldh1a3 cells were injected by intracardiac injection into mice, and bone metastasis growth was tracked by intravital bioluminescent imaging. Bone metastasis-free survival was tracked by bioluminescence, and plotted using the Kaplan-Meier model. The results were shown in FIGS. 2A-2C.

FIG. 2A is flow cytometry spectra, and shows that genetic knockout of ALDH1a3 in Sum159-M1a breast cancer cells nearly abolishes ALDEFLUOR™ activity in the cells, and that ALDEFLUOR™ activity can be rescued by transducing the cells with a rescue vector. FIG. 2B is a line graph of bone metastasis, as measured by bioluminescence (ph/s), versus time (days), and shows that knockout of ALDH1a3 in Sum159-M1a breast cancer cells slows bone metastasis growth. FIG. 2C is a Kaplan-Meier plot of bone metastasis-free survival over time, and shows that knockout of ALDH1a3 in Sum159-M1a breast cancer cells significantly increases survival time. Thus, genetic knockout of Aldh1a3 in Sum159-M1a breast cancer cells slows bone metastasis growth.

In sum, the above results show that genetic knockout of Aldh1a3 in cancer cells can slow primary tumor growth, sensitize tumors to chemotherapy, slow metastasis, and enhance survival time.

### Biological Example 3. Genetic Expression Studies

### Genetic Expression of ALDH1a3

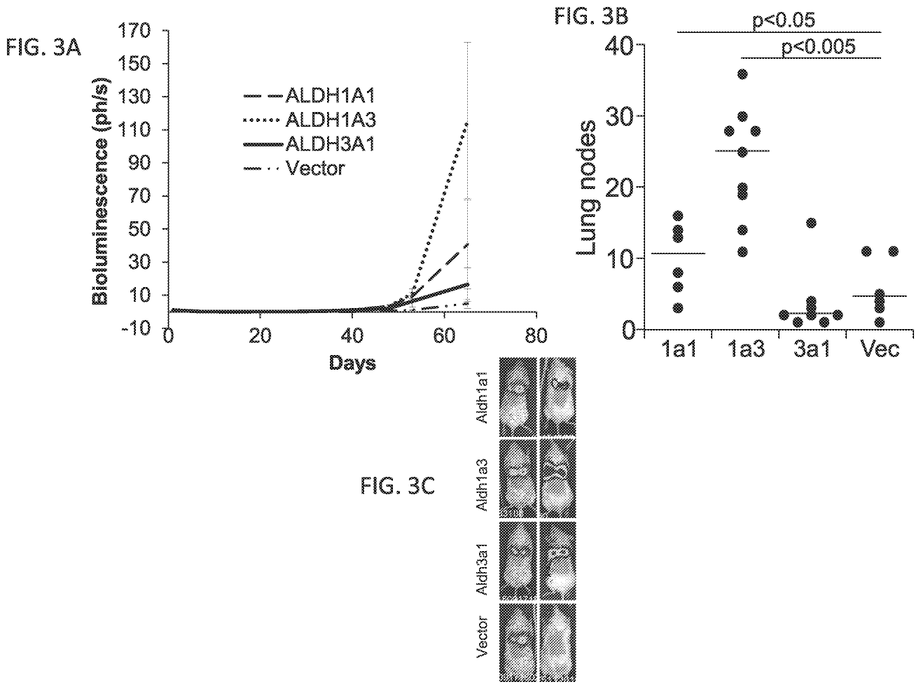

Lentiviral vectors encoding one of three human ALDH genes, ALDH1a1, ALDH1a3 or ALDH3a1, were introduced by viral transduction into luciferase-labeled Sum159-M1b cells followed by positive selection with puromycin, and the transduced cells were injected by tail-vein injection into mice. Growth of lung metastasis was tracked by intravital bioluminescence imaging once weekly. Lung nodes were counted ex vivo. The results were shown in FIGS. 3A-3C.

FIG. 3A is a line graph of bioluminescence (phis) versus time (days), and shows the development of lung metastasis in mice injected with SUM 159-M1b cells transfected with vectors encoding three ALDH enzymes, ALDH1a1, ALDH1a3 and ALDH3a1. FIG. 3B is a plot of lung nodes counted ex vivo at the endpoint of the experiment described in FIG. 3A. FIG. 3C shows sample images of bioluminescence at Day 1 (left) and endpoint (right). As can be seen from the figures, genetic expression of Aldh1a3 in Sum159-M1b breast cancer cells enhances lung metastasis growth.

### Biological Example 4. Survival Predictions

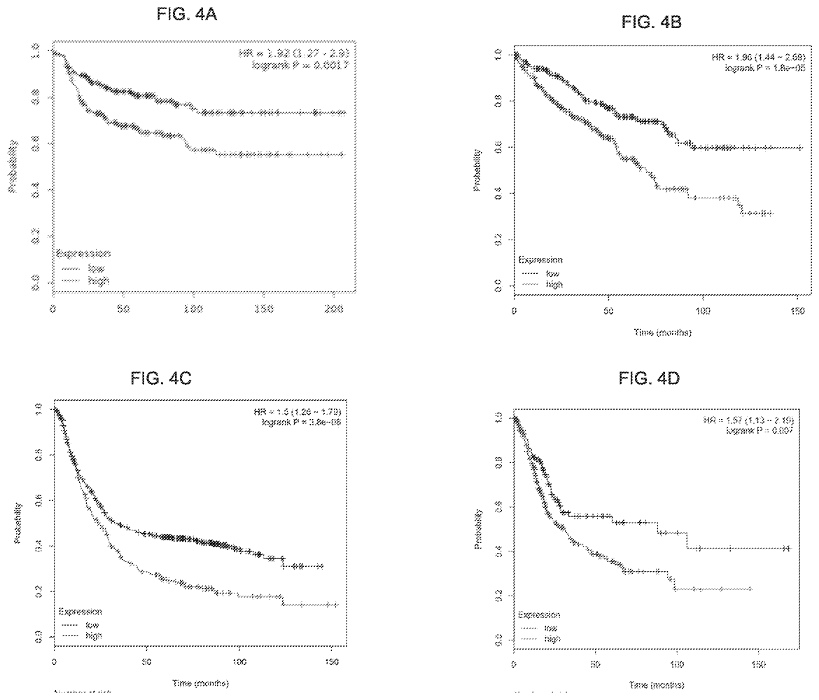

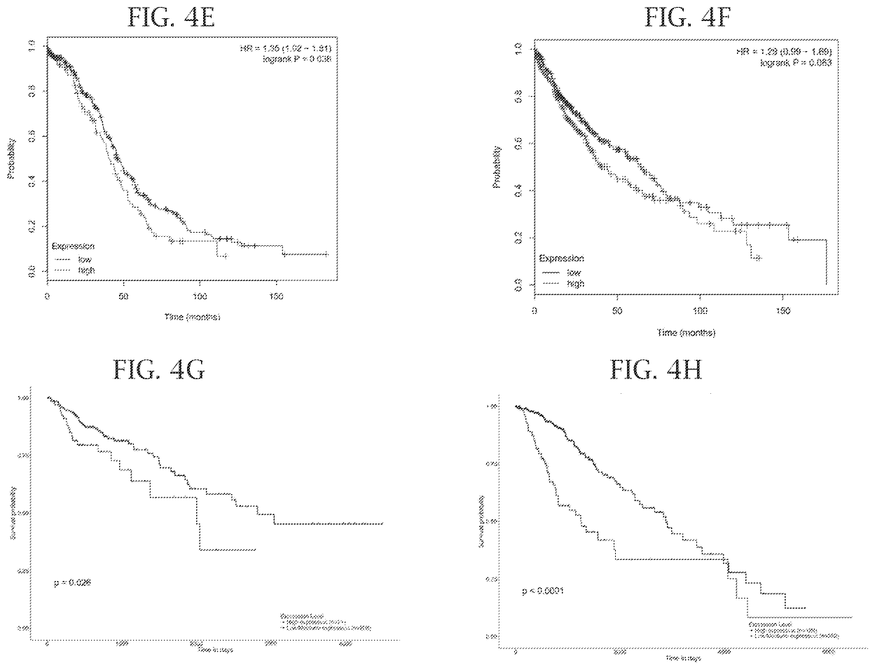

TCGA and Kaplan-Meier plotter (kmplot.com) data was used to generate expression data and survival curves for various cancers as a function of ALDH1a3 expression level. Cancer patients from each dataset were stratified by high or low Aldh1a3 expression according to either median expression value or the optimal stratification value. Kaplan-Meier analysis was then used to plot patient survival, whether measuring distant metastasis-free or overall survival, to assess the relationship between relative levels of Aldh1a3 between patients and corresponding survival metrics. The results were shown in FIGS. 4A-4H.

FIGS. 4A-4H are prognostic patient survival curves stratified by high (red) and low (black) Aldh1a3 expression based on the data analysis tool hosted at kmplot.com, and show the distant metastasis free survival for triple negative breast cancer patients (FIG. 4A) and overall survival for renal clear cell, gastric, bladder cancer, ovarian cancer, lung squamous cancer, colorectal cancer and low-grade glioma cancer patients (FIGS. 4B-4H, respectively) as a function of ALDH1a3 expression level. The data shows that high Aldh1a3 expression predicts worse overall survival in cancer patients.

In another set of predictions, mRNA expression of Aldh1a3 from the METABRIC clinical breast cancer dataset was segregated by tumor type and prior treatment with chemotherapy. Survival curves in the EMC-MSK dataset were generated by splitting patients according to subtype, and stratifying by median Aldh1a3 expression.

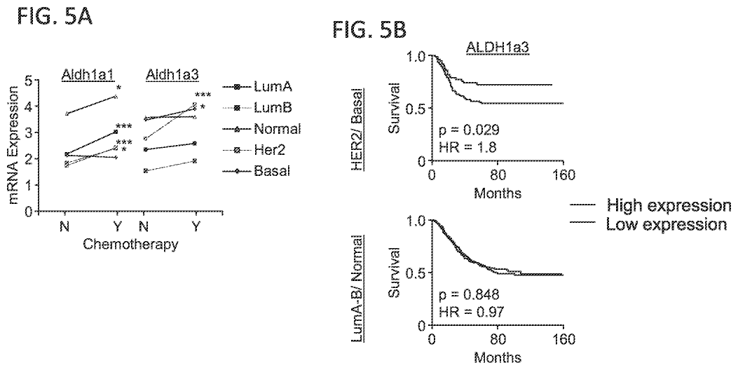

FIG. 5A is graph of mRNA expression of ALDH1a3 from the METABRIC clinical breast cancer dataset, and shows expression of ALDH1a3 by breast cancer subtype and history of chemotherapy. FIG. 5B is predicted survival curves based on the EMC-MSK dataset, and shows the predicted survival time of breast cancer patients by subtype and median ALDH1a3 expression level. The data shows that high Aldh1a3 expression predicts worse overall survival in breast cancer patients.

Table 1 reports the hazard ratio (p-value) of patients expressing high ALDH1a1 or ALDH1a3 in estrogen receptor-negative (ER) breast cancer derived from the Kaplan-Meier plotter database, and includes the Her2 and triple negative breast cancer (TNBC) populations that are at high risk for developing metastasis. ALDH1a3 is a poor prognosis predictor in ER-negative breast cancer patients, the population most likely to develop metastasis.

TABLE 1
Hazard ratio (p-value) of patients expressing high ALDH1a1
or ALDH1a3 in ER-negative breast cancer patient populations
derived from the Kaplan-Meier plotter database

| Gene | All | Chemotherapy | No Chemotherapy |
|---|---|---|---|
| ALDH1a1 | 0.48 | 2.34 | 0.48 |
| | (0.00039) | (0.065) | (0.0071) |
| ALDH1a3 | 1.85 | 3.3 | 1.81 |
| | (0.004) | (0.026) | (0.032) |

### Biological Example 5A. ALDEFLUOR™ Assay

The ALDEFLUOR™ assay assesses the ability of cells to oxidize bodipy-aminoacetaldehyde (BAAA) to bodipy-aminoacetate (BAA). This activity can be used to sort live cells and thereby discriminate between ALDH activity levels within heterogenous populations. When it was first discovered in 2007 that ALDEFLUOR™-positive cancer cells were more tumorigenic and predicted worse clinical outcome, it was assumed that ALDEFLUOR™ activity was a marker of a broader transcriptional program that promoted tumor aggressiveness. Since these early studies, ALDEFLUOR™ activity has become the most cited method for assessing the “sternness” or aggressiveness of tumor cell populations.

Following this seminal discovery, ALDEFLUOR™ activity was often assessed with little consideration for the function of ALDH1 enzymes. Rather, publications showed that the ALDEFLUOR™ assay isolated aggressive or metastatic cancer cells, regardless of the site of the primary tumor. Since, hundreds of papers have used the ALDEFLUOR™ assay in assessing cancer cell traits across almost all cancer types. Only beginning with Marcato and colleagues in 2011 was it shown that ALDH1a3 is responsible for ALDEFLUOR™ activity in most breast cancer cell lines.

Since Marcato's and colleagues' publication, an accelerating rate of emerging studies has established that ALDH1a3 is not only responsible for ALDEFLUOR™ activity across most cancer types, but that it also functionally promotes cancer growth, therapeutic resistance, and metastasis. Research of varying quality has established that ALDH1a3 is expressed and important for growth in melanoma patient-derived xenografts or cell lines, metabolism, chemoresistance and radioresistance in mesenchymal-like glioma or glioblastoma, tumorigenicity and cisplatin resistance in lung cancer, growth and radio-resistance in pancreatic cancer, FAK inhibitor resistance in colon and thyroid cells, cisplatin resistance in mesothelioma, Gleason score and growth in prostate cancer, apoptosis-resistance and metastasis in breast cancer and prognosis in cholangiocarcinoma. It is shown herein that ALDH1a3 is the dominant ALDEFLUOR™-inducing enzyme across most solid tumor types.

Expression and prognosis studies have further shown that ALDH1a3 is strongly predictive of poor outcomes across cancer types. Hypermethylation of the ALDH1a3 promoter leading to lower ALDH1a3 expression was the strongest predictor of favorable outcome in a set of primary glioblastoma patients. High ALDH1a3 predicted lymph node metastasis in cholangiocarcinoma patients. ALDH1a3 expression is driven by androgen in prostate cancer, where androgen is the major mitogen for prostate cancer cells, while mir187 targets ALDH1a3 in prostate cancer and high mir187 was correlated to favorable prognosis.

In the ALDEFLUOR™ assay used herein, cells were grown until they reached 50-80% confluence, harvested with 0.25% Trypsin/EDTA (Sigma), and washed once with PBS by centrifugation/resuspension (190 g for 5 min at 4° C.). Cells were counted, centrifuged and resuspended at 1,000,000 cells/mL in ALDEFLUOR™ buffer (Stemcell Technologies). ALDEFLUOR™ substrate (Stemcell Technologies, 1:200) and test compound or 1 mM DEAB were added to cell suspension and incubated at 37° C. for 45 minutes with vortexing every 15 minutes. Cells were centrifuged and resuspended in ALDEFLUOR™ buffer with DAPI at 5 μg/mL. Samples were analyzed with the BD LSR2 flow cytometry platform. Gating was performed using DEAB as a negative control.

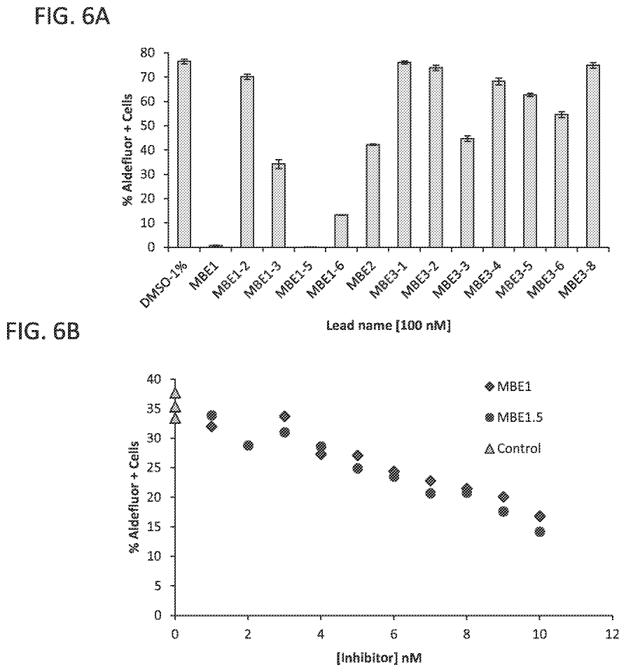

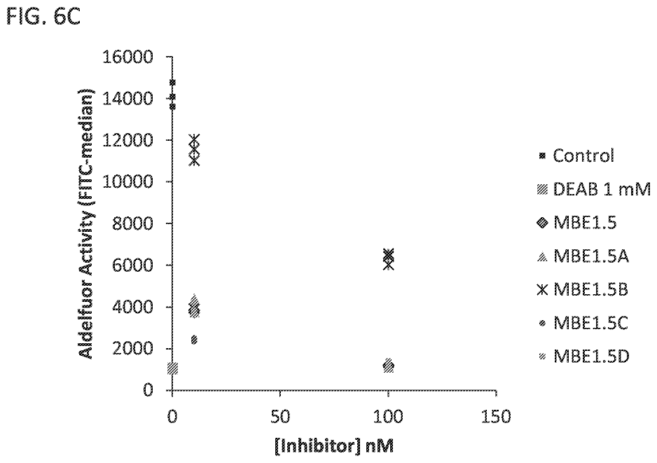

FIG. 6A is a bar graph of percentage of ALDEFLUOR™-positive cells in the presence of various compounds described herein, and shows the percentage of SUM159-M1a-Aldh1a3 cells that are above background fluorescence levels, as detected by flow cytometry after incubation using the standard ALDEFLUOR™ protocol described herein with compounds at a concentration of 100 nM. Gating for background fluorescence was performed using 1 millimolar DEAB as a negative control. FIG. 6A demonstrates that MBE1-5, MBE1 and MBE1-6 are high-affinity compounds for the inhibition of ALDH1a3 activity.

FIG. 6B is a line graph of percentage of ALDEFLUOR™-positive cells in the presence of varying concentrations of MBE1 or MBE1.5, and shows the percentage of SUM 159-M1a-Aldh 1a3 cells that are above background fluorescence levels, as detected by flow cytometry after incubation according to the standard ALDEFLUOR™ protocol described herein combined with a dose titration of MBE1 or MBE1-5. The [inh-min]threshold was set at the lower bound of two standard deviations of control samples, while the IC₅₀ threshold was set at 50% of the average of control samples. FIG. 6B demonstrates that MBE1 and MBE1.5 show IC50 values in the 8-10 nanomolar range with inhibitory activity detected at concentrations as low as 2 nanomolar.

An ALDEFLUOR™ assay was also used to assess the relative activity of several compounds described herein against SUM159-M1a-Aldh1a3 cells. The activity of several compounds (Compound Nos. 1-17) in the assay at a concentration of 100 nM is reported in Table 2.

TABLE 2

| | Percent of Cells | |
|---|---|---|
| | Positive for | Inhi-bitory |
| | ALDEFLUOR ™ at | Activity at 100 nM |
| Cmpd. No. | 100 nM | (%) |
| 1/MBE1* | 0.73 | 99 |
| 2/MBE1.2 | 70 | 8 |
| 3/MBE1.3 | 34 | 55 |
| 4/MBE1.5 | 0.03 | 100 |
| 5/MBE1.5A | N.A. | N.A. |
| 6/MBE1.5B | N.A. | N.A. |
| 7/MBE1.5C | N.A. | N.A. |
| 8/MBE1.5D | N.A. | N.A. |
| 9/MBE1.6 | 13 | 83 |
| 10/MBE2 | 42 | 45 |
| 11/MBE3.1 | 76 | 0.60 |
| 12/MBE3.2 | 74 | 3.3 |
| 13/MBE3.3 | 45 | 42 |
| 14/MBE3.4 | 68 | 11 |
| 15/MBE3.5 | 63 | 18 |
| 16/MBE3.6 | 55 | 29 |
| 17MBE3.8 | 75 | 2 |

*The expression “1/MBE1” means that the compound may be identified herein as Compound No. 1 or Compound No. MBE1. Other similar expressions should be interpreted similarly.

FIG. 6C is a graph of ALDEFLUOR™ activity versus concentration, and shows the ALDEFLUOR™ activity of several compounds described herein against SUM 159-M1a-Aldh 1a3 cells at concentrations of 10 nM and 100 nM. In comparison to control (DMSO) or DEAB-treated cells, MBE 1.5C (Compound No. 7) is nearly twice as potent as MBE 1.5 (Compound No. 4), MBE 1.5A (Compound No. 5) or MBE 1.5D (Compound No. 8) at a concentration of 10 nM.

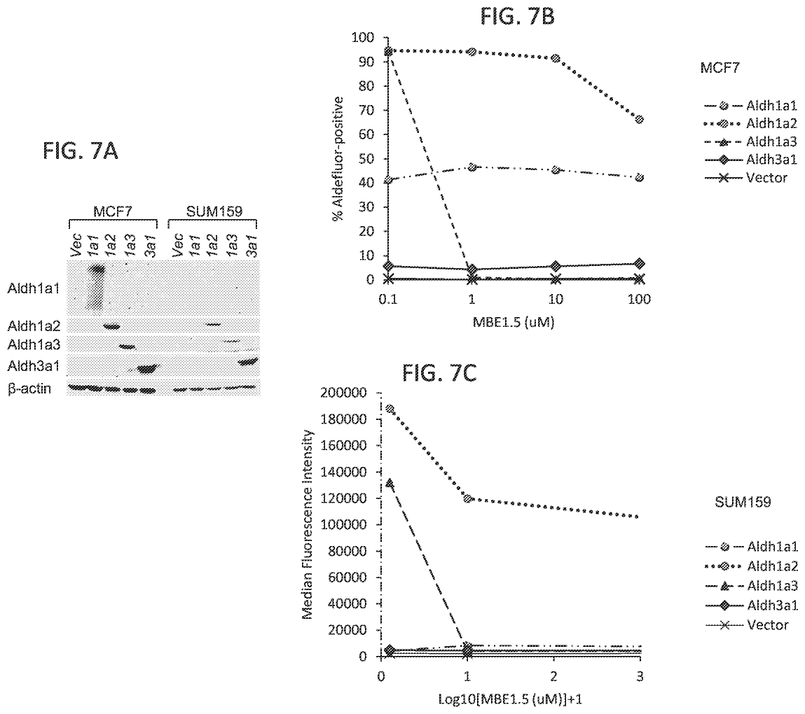

ALDH isoforms 1a1, 1a2, 1a3 and 3a1 were expressed in MCF7 or SUM159 cells, which were subsequently used in an ALDEFLUOR™ assay. FIG. 7A is a Western blot, and shows the expression of each ALDH isoform in the indicated cells. FIG. 7B is a line graph of percentage of ALDEFLUOR™-positive MCF7 cells expressing the indicated ALDH isoform versus the log of MBE 1.5 concentration, and shows that MBE 1.5 specifically inhibits ALDH1a3 at concentrations below 10 μM. FIG. 7C is a line graph of percentage of ALDEFLUOR™-positive SUM159 cells expressing the indicated ALDH isoform versus the log of MBE 1.5 concentration, and shows that MBE 1.5 specifically inhibits ALDH1a3 at concentrations below 10 μM.

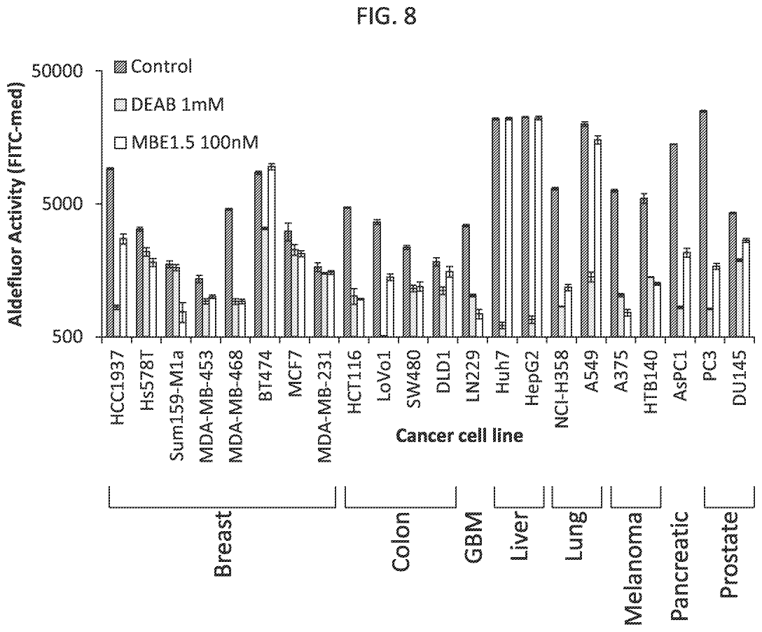

FIG. 8 is a bar graph of ALDEFLUOR™-positive cells in a variety of cancer types in the presence of 1 mM DEAB (a pan-ALDH inhibitor) or 100 nM MBE1.5 (a specific ALDH1a3 inhibitor), and shows that the majority of human cancer cell lines show ALDH1a3 activity. A notable exception is liver cancer, where it is expected that a large ALDEFLUOR™-positive population exists, and is driven by ALDH1a1.

### Biological Example 5B. Aldh1a3 Enzyme Inhibition Assay

Recombinant protein extraction: pET-Aldh1a3 transformed BL21-DE3 cultures induced at 20° C. for 19h with 0.3 mM IPTG rocking. Cultures were spun at 3500 g for 10 min, supernatants were poured off and allowed to drain fully. Cells were resuspended in 10 mM HEPES pH 7.4, 10 mM KCl. Cells were freeze-thawed in liquid nitrogen and then a 37° C. water bath for 10 cycles followed by ultrasonication at 50% amplitude, 3 sec on, 9 sec off for 10 cycles at 4° C. Cell extracts were spun at 16000×g for 5 minutes.

Reaction performed at 20° C. in reaction buffer (10 mM HEPES pH 7.4, 10 mM KCl, 0.1 M Resazurin, 1 mg/mL BSA, 200 uM NAD+, diaphorase and aldehyde substrate). Recombinant enzyme and inhibitor added immediately before assay. Reaction rate measured by resorufin fluorescence.

The IC₅₀ values of selected tested compounds are shown in Table 3 below.

TABLE 3
IC₅₀ values* for inhibition of hALDH1a3
and mALDH1a3 of selected compounds

| Cmpd. No. | hAldh1a3 IC₅₀ | mAldh1a3 IC₅₀ |
|---|---|---|
| 1 | B | B |
| 2 | E | E |
| 3 | D | D |
| 4 | A | B |
| 5 | A | C |
| 6 | D | D |
| 7 | A | B |
| 8 | A | B |
| 9 | A | C |
| 10 | D | D |
| 11 | E | E |
| 12 | E | E |
| 13 | E | D |
| 14 | E | E |
| 15 | E | E |
| 16 | E | E |
| 17 | E | E |
| 18 | B | D |
| 19 | C | D |
| 20 | D | E |
| 21 | E | E |
| 22 | E | E |
| 23 | E | D |
| 24 | E | E |
| 26 | E | E |
| 27 | E | E |
| 28 | E | E |
| 29 | E | E |
| 30 | E | D |
| 33 | B | B |
| 34 | E | E |
| 35 | C | A |
| 37 | D | E |
| 38 | C | D |
| 39 | C | D |
| 40 | D | C |
| 42 | D | D |
| 43 | C | D |
| 45 | C | C |
| 46 | D | D |
| 47 | B | A |
| 48 | C | C |
| 49 | E | E |
| 50 | B | D |
| 52 | C | B |
| 53 | A | A |
| 54 | C | E |
| 55 | C | C |
| 56 | B | C |
| 59 | A | A |
| 60 | B | C |
| 61 | B | D |
| 62 | D | E |
| 63 | B | C |
| 64 | B | C |
| 68 | D | E |
| 69 | E | E |
| 57 | E | E |
| 85 | E | E |
| 70 | A | C |
| 71 | C | D |
| 72 | C | C |
| 73 | D | E |
| 76 | E | E |
| 77 | B | C |
| 78 | A | C |
| 79 | C | C |
| 80 | A | C |
| 81 | A | A |
| 83 | B | D |
| 84 | C | C |
| 86 | E | D |
| 87 | C | A |
| 88 | E | E |
| 89 | E | D |
| 90 | E | E |
| 91 | E | D |
| 92 | B | B |
| 93 | C | C |
| 94 | D | E |
| 95 | E | E |
| 96 | E | E |
| 97 | D | E |
| 98 | D | D |
| 99 | C | C |
| 100 | E | E |
| 101 | D | D |
| 103 | E | E |
| 104 | E | E |
| 105 | E | E |
| 106 | E | E |
| 108 | D | C |
| 109 | B | A |
| 111 | E | E |
| 112 | C | C |
| 113 | E | E |
| 114 | E | E |
| 115 | E | E |
| 116 | C | D |
| 118 | E | E |
| 119 | E | E |
| 120 | E | E |
| 122 | E | E |
| 123 | E | E |
| 125 | C | E |
| 126 | C | A |
| 127 | D | E |
| 132 | C | C |
| 134 | C | C |
| 135 | E | E |
| 136 | D | E |
| 137 | E | E |
| 31 | E | E |
| 32 | E | E |
| 41 | E | E |
| 51 | E | E |
| 58 | E | E |
| 102 | E | E |

*The IC₅₀ values are reported herein according to the Activity Level: A <100 nM; B: 100 nM-250 nM; C: 250 nM-1 uM (micromolar); D: 1 uM-5 uM; E: >5 uM.

The enzyme inhibition data correlates well with the results obtained from the ALDEFLUOR™ assay as described herein.

### Biological Example 6. In Vivo Xenografts Studies

### Mouse Models and Zenografts

All mice were originally ordered from the Jackson Laboratory (Bar Harbor, Me.) and breeding was conducted in a specific pathogen-free (SPF) barrier facility. Toxicity experiments were performed on 8-12-week old male and female C57Bl6 mice. MBE1 was dissolved at 50 mg/mL in DMSO followed by a 1:2 dilution into Kolliphor EL, followed by a 1:5 dilution into PBS to yield a 5 mg/ml MBE1 solution, (10% DMSO, 10% Kolliphor EL, 80% PBS). This was administered via IP injection at ascending doses from 12.5 to 200 mg/kg body mass in a set of 3 mice at 24h intervals. Body condition score, food uptake, fecal/urine production, behavior and body weight were measured as toxicity readouts. Five mice were then treated every 3 days with 25 mg/kg MBE1 for 18 days. Neither experiment showed any indication of either acute or chronic toxicity.

All xenograft experiments were conducted on 8-week old female mice (athymic Nu/Nu, or NOD/SCID Gamma). Xenograft experiments were conducted using 125,000 SUM 159-M1a cells in 100 μL PBS for tail vein or intracardiac injection. Mice were randomized following injection. Bioluminescent imaging (BLI) was conducted using the IVIS 200 system and retroorbital luciferin injection. For drug treatment, MBE1 was given to mice via IP injection in conjunction with paclitaxel (5 mg/mL in 10% ethanol, 10% Kolliphor EL, 80% PBS) at the time intervals and dosages indicated in FIG. 9A (tail vein injection) and FIG. 10A (intracardiac injection). The results were shown in FIGS. 9B and 10B, which demonstrate that MBE1 is an effective therapeutic to treat established metastatic disease, and establishes that MBE1 and the compounds disclosed herein can be used to effectively inhibit ALDH1a3 and its downstream effects in vivo.

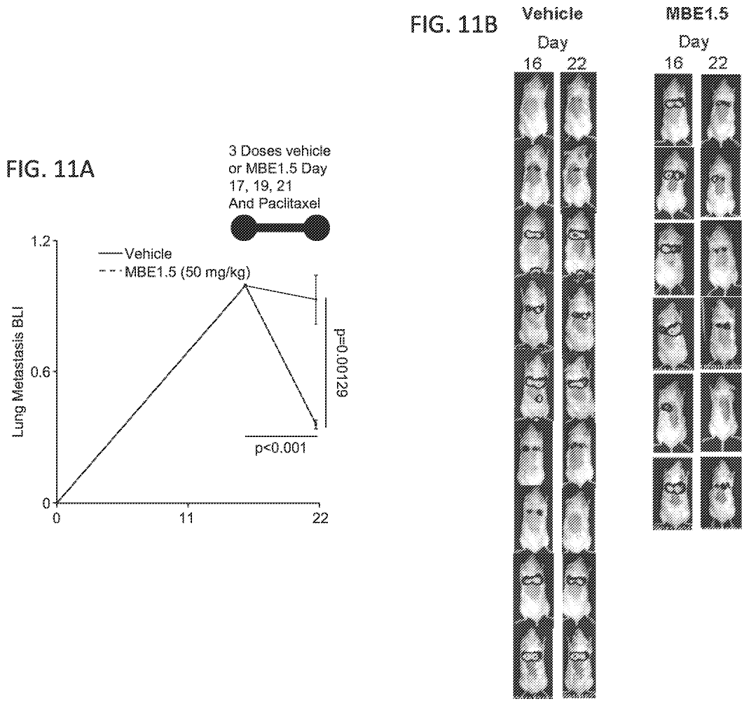

In another xenograft experiment, mice were injected with SUM 159-M1-p44 cells via tail-vein injection, as described above, and randomized following injection. Metastatic burden was imaged on day 16 via intravital imaging. Mice were then treated with paclitaxel (25 mg/kg) and either vehicle or MBE1.5 (50 mg/kg) on days 17, 19 and 21. Lung metastatic burden was then imaged on day 22. Signals were normalized to day 16. As can be seen from FIGS. 11A and 11B, three doses of 50 mg/kg MBE1.5 in combination with 25 mg/kg paclitaxel, administered on days 17, 19 and 21 caused regression of established metastatic disease in a mouse xenograft model.

In yet another xenograft experiment, mice were injected with MDA-MB-468 cells via mammary fat pad, and were randomized following injection. Mice were treated with MBE1.5 (25 mg/kg daily, n=6) or vehicle (n=12), and paclitaxel (12.5 mg/kg every other day) for 12 days. Primary tumor measurements were taken by caliper between each treatment group.

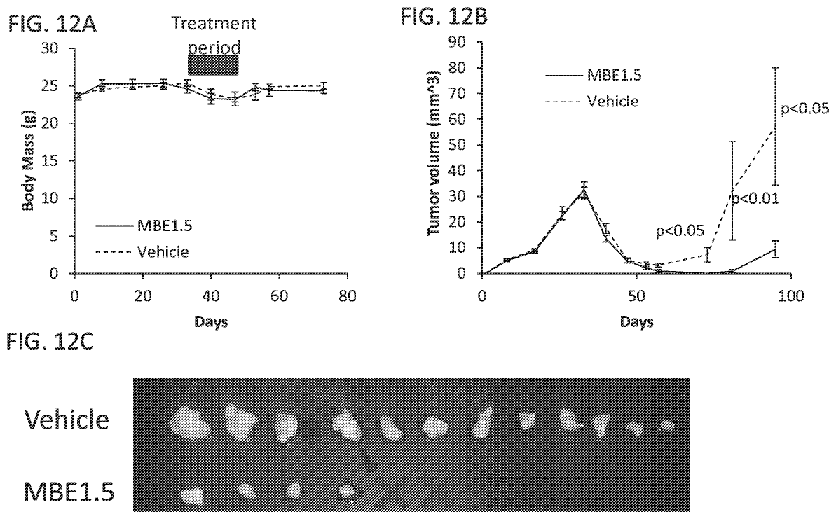

As shown in FIGS. 12A-12C, there was no gross toxicity associated with MBE1.5 treatment in this experiment and 12-day treatment with MBE1.5 caused regression of primary breast tumors. The tumors of two mice in the MBE1.5 treatment group were completely eliminated by the treatment.

In another xenograft experiment, mice were injected with Sum-159-M1a-Aldh 1a3 cells via tail-vein injection, and were randomized following injection. Mice were treated with MBE1.5 (25 mg/kg daily) or vehicle, and paclitaxel (12.5 mg/kg every other day) for twelve days. Lung metastasis progression was tracked by intravital bioluminescence.

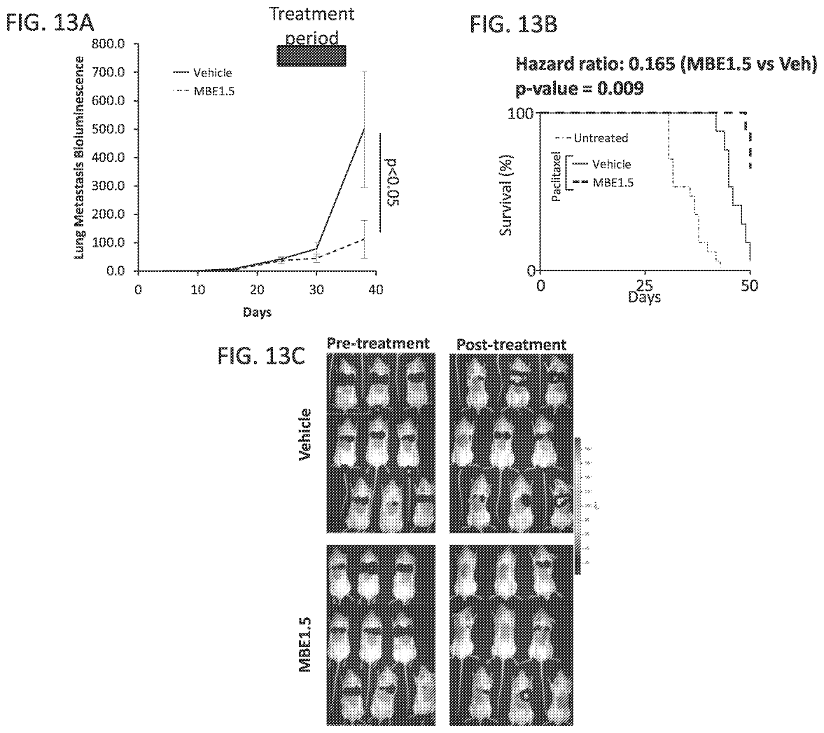

As shown in FIGS. 13A-13C, 12-day treatment with MBE1.5 extended survival in mice with late-stage established breast cancer lung metastasis.

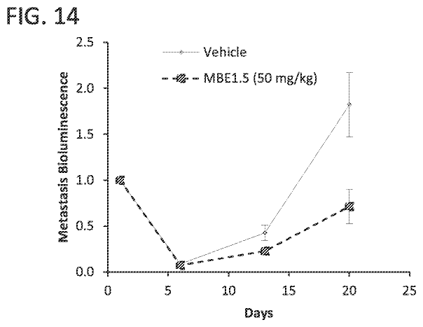

In another xenograft experiment, mice were injected HCT116 cells via left ventricle injection and monitored with BLI on a weekly basis during the experiment. MBE1.5 or vehicle control were injected subcutaneously every day following injection at 50 mg/kg. At day 7 following injection, paclitaxel was dosed at 25 mg/kg every three days for a total of 6 injections. N=10 mice per group. The results were shown in FIG. 14.

As can be seen from FIG. 14, the combination treatment of MBE1.5 and paclitaxel slows colorectal cancer metastasis.

### Biological Example 7. Pharmacokinetic Profiling of Test Compounds

Three male CD-1/C57BL/6 mice per group were dosed with compound MBE1 (alternatively referred to herein as Compound No. 1) or MBE1.5 (alternatively referred to herein as Compound No. 4) at 10 mg/kg for oral gavage or 1 mg/kg for intravenous dosing. Cage side observations were performed and no adverse reactions to either MBE1 or MBE1.5 treatment were observed from 0 hours to 24 hours post-dosing. Blood was drawn from subjects and plasma was extracted at 6 timepoints. Plasma concentrations of each compound was profiled by LC-MS and absolute concentrations were derived by comparison to a reference standard. Linear regression was performed to calculate oral bioavailability of 88% and a plasma half-life of 1.79 hours for IV bolus dosing and 1.37 hours from PO oral gavage dosing for compound MBE1. For compound MBE1-5, oral bioavailability of 62.5% was calculated with a plasma half-life of 1.22 hours for IV bolus dosing and 1.70 hours from PO oral gavage dosing.

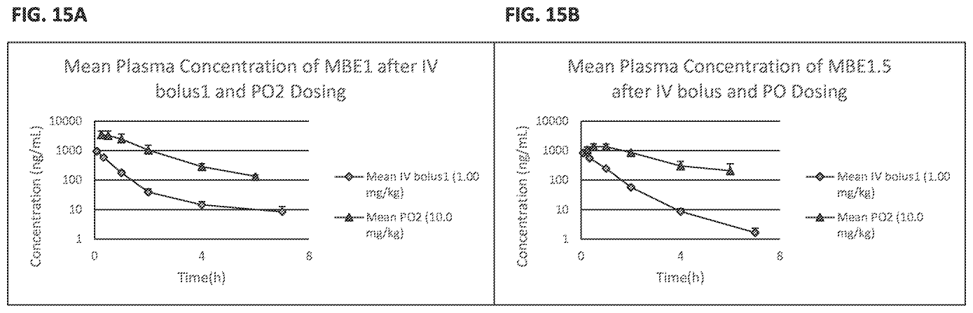

FIG. 15A and FIG. 15B demonstrate plasma concentrations of MBE1 at each bioanalysis time point demonstrating sufficient stability to therapeutically inhibit Aldh1a3 for in vivo models following treatment at once or twice per day.

### Biological Example 8. Mechanism of Aldh1a3 in Controlling Fatty Acid Metabolism

HEK293T cells were grown to full confluence and media was exchanged with complete DMEM media supplemented with 10% dialyzed fetal bovine serum. Three replicate plates per group were treated with 10 uM MBE1.5 or control DMSO for 1 hour followed by organic extraction with 40:40:20 methanol:acetonitrile:water w/0.5% formic acid (ice cold). Plates were immediately transferred to ice and incubated for 5 minutes then 50 ul 15% NH4HCO3 was added. Cells lysates were scraped and centrifuged at 15000 RCF. Supernatants were then analyzed via LC-MS to quantify various metabolites using standard metabolomic protocols.

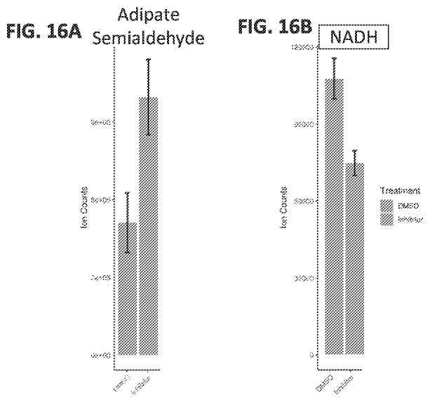

FIG. 16A and FIG. 16B demonstrate increases in adipate semialdehyde (a medium chain fatty acid involved in lipid catabolism) upon MBE1.5 treatment with concomitant depletion of NADH demonstrating reduced fatty acid metabolism.

### Biological Example 9. Pharmacologic Treatment Studies

C57/Bl6 mice homozygous for the spontaneous mutation (Lepr^{db}, referred to as db/db) that develop severe obesity and Type 2 Diabetes within 12 weeks were raised through standard husbandry practice and we separated into two groups followed by acclimatization for 9 days in single housing.

Daily food intake and body mass were monitored each day and baseline levels of plasma insulin and glucose were monitored to confirm identical group characteristics.

Compound MBE1 was dissolved to 200 mg/mL in DMSO followed by suspension into a 0.5% methycellulose, 0.5% Tween-80 solution. Mice were treated via oral gavage once per day to achieve 40 mg per kg body mass MBE1 or were treated with vehicle control. Mouse body mass and food consumption were monitored daily over 14 days.

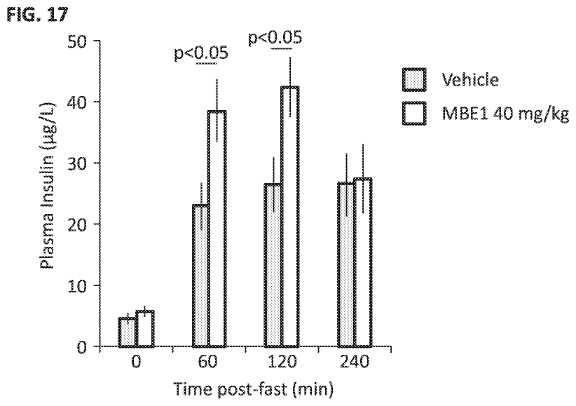

At Day 14, food was removed from mouse cages for 16 hours, and then food was reintroduced. Plasma insulin levels were assayed by ELISA at baseline and at 1 hour, 2 hour and 4 hours following provision of chow and ad libitum feeding. FIG. 17 is a graph of plasma insulin in the control group vs MBE1 treated group following fast and refeed challenge demonstrating that treatment of a subject with Type 2 Diabetes with an Aldh1a3 inhibitory compound effectively improve insulin secretion in a Type 2 Diabetes model.

### Biological Example 10 Enzyme Activity Assay Measuring ALDH Activities of Pancreatic Cells from Diabetic Mice

The ALDEFLUOR™ assay assesses the ability of cells to oxidize bodipy-aminoacetaldehyde (BAAA) to bodipy-aminoacetate (BAA) to form a non-cell membrane permeable product. This activity can be used to sort live cells and thereby discriminate between ALDH activity levels within heterogenous populations.

In the ALDEFLUOR™ assay used in this example, 14 month-old C57/BL6 high fat diet-induced severe obese/diabetic mice and 3 month-old lean C57/B16 mice were euthanized and pancreatic cells were extracted with 1 mg/mL Collagenase 1 digestion in PBS for 1 hour at 37 C. Single cell suspensions were obtained by filtration through 40 μm cell sieves. Cells were washed by two sequential centrifugations and resuspension in PBS.

Cells were counted, centrifuged and resuspended at 1,000,000 cells/mL in ALDEFLUOR™ buffer (Stemcell Technologies). ALDEFLUOR™ substrate (Stemcell Technologies, 1:200) and test compound (MBE 1.5 at 10 μM or 1 mM DEAB were added to cell suspension and incubated at 37° C. for 45 minutes with vortexing every 15 minutes. Cells were centrifuged and resuspended in ALDEFLUOR™ buffer with DAPI at 5 μg/mL. Samples were analyzed with the BD LSR2 flow cytometry platform.

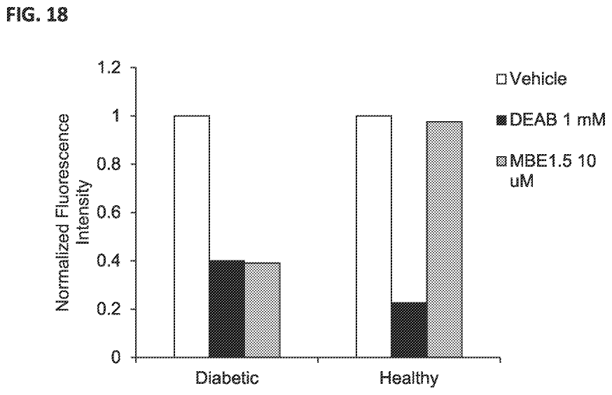

FIG. 18 is a bar graph of percentage of ALDEFLUOR™-positive cells in the presence of various compounds described herein, and shows the percentage of pancreatic islet cells that are above background fluorescence levels, as detected by flow cytometry after incubation using the standard ALDEFLUOR™ protocol described herein with compounds at a concentration of 10 μM. Gating for background fluorescence was performed using 1 millimolar DEAB as a negative control. FIG. 18 demonstrates that MBE1.5 is high-affinity compound for the inhibition of ALDH1a3 activity that is expressed only in diabetic pancreas extracts and not healthy pancreas extracts.

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A compound of Formula I, or a pharmaceutically acceptable salt thereof: 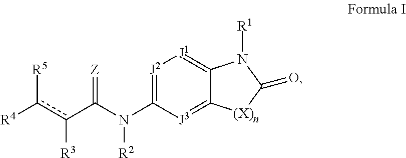
wherein:
X at each occurrence is independently selected from O, NR¹⁰, and CR²⁰R²¹, provided that at most one X is selected from O and NR¹⁰;
n is 1, 2, 3, or 4;
J¹, J², and J³ are each independently selected from CR²² or N, preferably, at least one of J¹, J², and J³ is not N;
R¹ and R² are each independently hydrogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), or a nitrogen protecting group;
R³ and R⁴ are joined to form an optionally substituted aryl, an optionally substituted heteroaryl, an optionally substituted carbocyclic (e.g., C₃₋₈ carbocyclic), or an optionally substituted heterocyclic ring (e.g., 3-8 membered heterocyclic ring);
Z is O, and R⁵ is hydrogen, NR¹¹R¹², —CR²³R²⁴R²⁵, or —OR³⁰;
or Z is O, and R³, R⁴ and R⁵ are joined to form an optionally substituted bicyclic or polycyclic ring system, wherein the ring system is an aryl, heteroaryl, carbocyclic, or heterocyclic ring system;
or R⁵ and Z are joined to form an optionally substituted aryl, an optionally substituted heteroaryl, an optionally substituted carbocyclic (e.g., C₃₋₈ carbocyclic), or an optionally substituted heterocyclic ring (e.g., 3-8 membered heterocyclic ring); and
“ [symbol] ” in Formula I indicates the bond is an aromatic bond, a double bond or a single bond as valance permits, and when a single bond, the two carbons forming the bond can be optionally further substituted as valance permits;
wherein:
R¹⁰ at each occurrence is independently hydrogen, a nitrogen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, or an optionally substituted 3-8 membered heterocyclic ring;
R²⁰ and R²¹ at each occurrence are each independently hydrogen, halogen, —OR³¹, —NR¹³R¹⁴, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl; or
R¹⁰ and one of R²⁰ and R²¹ are joined to form a bond, an optionally substituted 4-8 membered heterocyclic ring or an optionally substituted 5 or 6 membered heteroaryl ring, wherein the other of R²⁰ and R²¹ is defined above;
R²⁰ and R²¹ together with the carbon they are both attached to form —C(O)—, an optionally substituted C₃₋₈ carbocyclic ring, or an optionally substituted 3-8 membered heterocyclic ring; or
one of R²⁰ and R²¹ in one CR²⁰R²¹ is joined with one of R²⁰ and R²¹ in a different CR²⁰R²¹ to form a bond, an optionally substituted C₃₋₈ carbocyclic ring, an optionally substituted 3-8 membered heterocyclic ring, wherein the others of R²⁰ and R²¹ are defined above;
R²² at each occurrence is independently hydrogen, halogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), —CN, —S(O)-alkyl, —S(O)₂-alkyl, or —OR³¹;
one of R¹¹ and R¹² is hydrogen or a nitrogen protecting group, and the other of R¹¹ and R¹² is hydrogen, a nitrogen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl;
one of R²³, R²⁴, and R²⁵ is hydrogen, halogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, an optionally substituted 5-10 membered heteroaryl, —OR³¹, or —NR¹³R¹⁴, and the other two of R²³, R²⁴, and R²⁵ are independently selected from hydrogen, fluorine, or methyl, preferably, —CR²³R²⁴R²⁵ is not —CH₃;
R³⁰ is hydrogen, an oxygen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, or an optionally substituted 3-8 membered heterocyclic ring; and
wherein:
each of R¹³ and R¹⁴ at each occurrence is independently hydrogen, a nitrogen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl; or R¹³ and R¹⁴ are joined to form a 3-8 membered optionally substituted heterocyclic or a 5-10 membered optionally substituted heteroaryl; and
R³¹ at each occurrence is hydrogen, an oxygen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl.

2. The compound of claim 1, or a pharmaceutically acceptable salt thereof, characterized as having Formula I-1 or I-2: 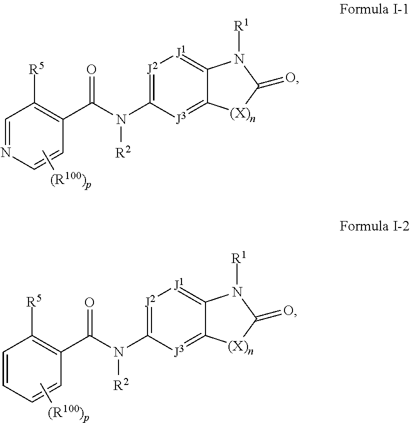
wherein:
R¹⁰⁰ at each occurrence is independently selected from halogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), —CN, or —OR³¹, wherein R³¹ is defined in claim 1; and
p is 0, 1, 2, or 3, preferably, p is 0 or 1.

3. The compound of claim 2, or a pharmaceutically acceptable salt thereof, characterized as having Formula I-1-A or Formula I-2-A: 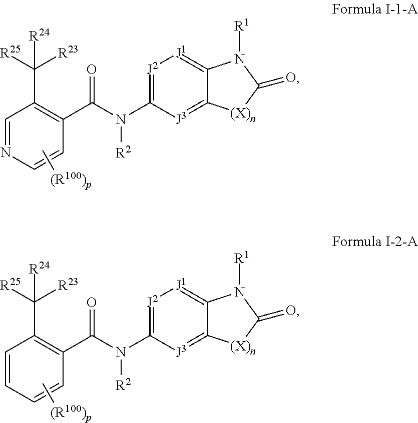
wherein:
R²³ is hydrogen or fluorine;
R²⁴ is hydrogen or fluorine;
R²⁵ is hydrogen; fluorine; C₁₋₄ alkyl optionally substituted with 1-3 fluorines and/or a C₃₋₆ cycloalkyl; a C₁₋₄ alkoxy optionally substituted with 1-3 fluorines and/or a C₃₋₆ cycloalkyl; a C₃₋₆cycloalkoxy optionally substituted with 1-3 substituents independently selected from fluorine and methyl; a C₃₋₄ cycloalkyl optionally substituted with 1-3 substituents independently selected from fluorine and methyl; or a 3-6 membered heterocyclic ring optionally substituted with 1-3 substituents independently selected from fluorine and methyl; and
at least one of R²³, R²⁴, and R²⁵ is not hydrogen.

4. The compound of claim 3, or a pharmaceutically acceptable salt thereof, characterized as having Formula I-1-A1, Formula I-1-A2, Formula I-1-A3, Formula I-2-A1, Formula I-2 A2; Formula I-2-A3: 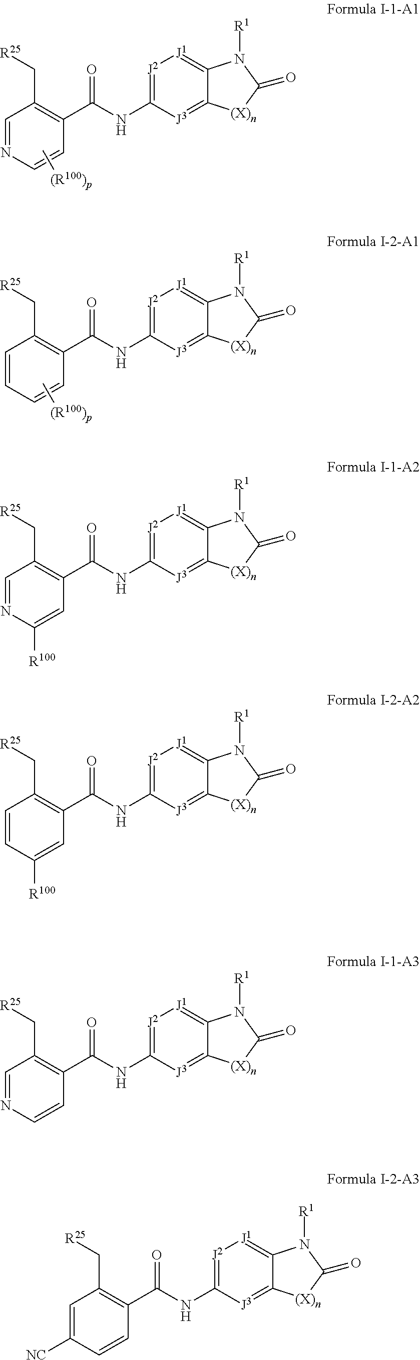
wherein:
R²⁵ is C₁₋₄ alkyl optionally substituted with 1-3 fluorines and/or a C₃₋₆ cycloalkyl, preferably, methyl, ethyl, n-propyl, isopropyl, or —CF₃; or a C₃₋₆ cycloalkyl optionally substituted with 1-3 substituents independently selected from fluorine and methyl, preferably, cyclopropyl or cyclobutyl; for example, R²⁵ is methyl, ethyl, n-propyl, isopropyl, difluoromethyl, trifluoromethyl, —CH₂—CF₃, —CH₂-cyclopropyl, cyclopropyl or cyclobutyl.

5. The compound of claim 2, or a pharmaceutically acceptable salt thereof, characterized as having Formula I-1-B, I-1-C, I-2-B, or I-2-C: 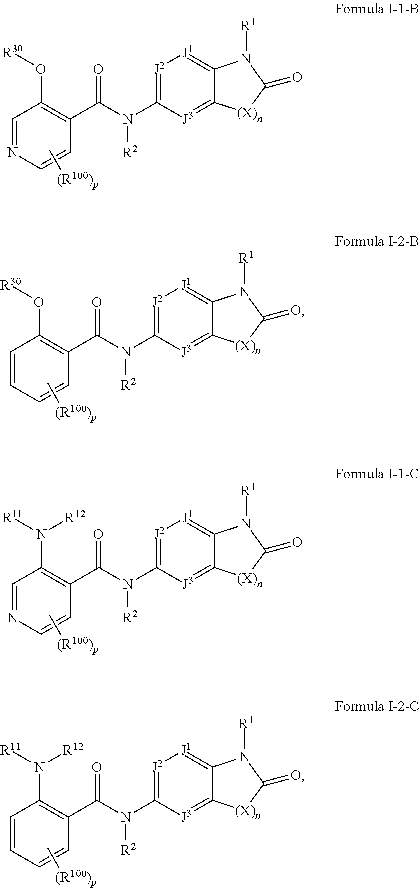
wherein:
R³⁰ is hydrogen; C₁₋₄ alkyl optionally substituted with 1-3 fluorines or a C₃₋₆ cycloalkyl, preferably, methyl, ethyl, n-propyl, isopropyl, difluoromethyl, trifluoromethyl, —CH₂—CF₃, or —CH₂-cyclopropyl; a C₃₋₆ cycloalkyl optionally substituted with 1-3 substituents independently selected from fluorine and methyl, preferably, cyclopropyl or cyclobutyl; or a 3-6 membered heterocyclic ring optionally substituted with 1-3 substituents independently selected from fluorine and methyl, preferably, 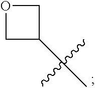 [SMILES: *C1COC1]
wherein one of R¹¹ and R¹² is hydrogen or a nitrogen protecting group, and the other of R¹¹ and R¹² is hydrogen, a nitrogen protecting group, C₁₋₆ alkyl optionally substituted with 1-3 fluorines or a C₃₋₆ cycloalkyl, preferably, methyl, ethyl, n-propyl, isopropyl, difluoromethyl, trifluoromethyl, —CH₂—CF₃, or —CH₂-cyclopropyl; a C₃₋₆ cycloalkyl optionally substituted with 1-3 substituents independently selected from fluorine and methyl, preferably, cyclopropyl or cyclobutyl; or a 3-6 membered heterocyclic ring optionally substituted with 1-3 substituents independently selected from fluorine and methyl, preferably,

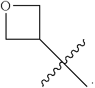 [SMILES: *C1COC1]

6. The compound of claim 2, or a pharmaceutically acceptable salt thereof, characterized as having Formula I-1-B1, Formula I-1-B2, Formula I-2-B1, Formula I-2-B2: 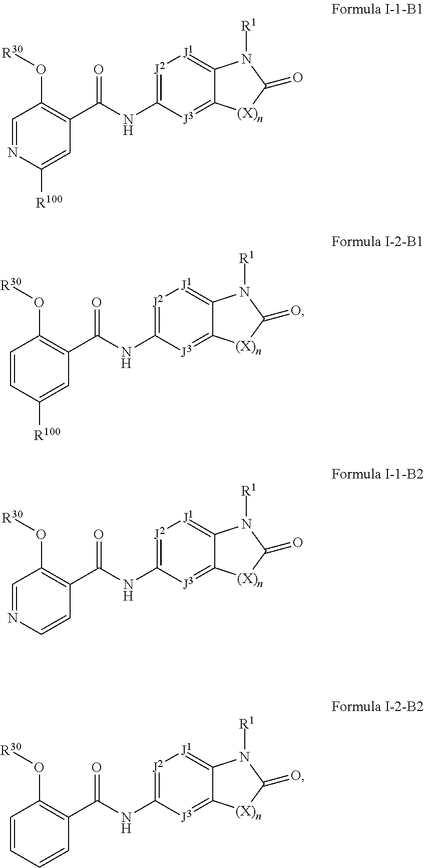
wherein R³⁰ is hydrogen, methyl, ethyl, n-propyl, isopropyl, difluoromethyl, trifluoromethyl, —CH₂—CF₃, —CH₂-cyclopropyl, cyclopropyl or cyclobutyl.

7. The compound of any one of claims 2-6, or a pharmaceutically acceptable salt thereof, wherein R¹⁰⁰ at each occurrence is independently selected from F; Cl; C₁₋₄ alkyl optionally substituted with 1-3 fluorines, preferably, methyl, ethyl, n-propyl, isopropyl, or —CF₃; a C₁₋₄ alkoxy optionally substituted with 1-3 fluorines, preferably, methoxy, ethoxy, n-propoxy, isopropoxy, or —OCF₃; a C₃₋₆ cycloalkyl optionally substituted with 1-3 substituents independently selected from fluorine and methyl, preferably, cyclopropyl or cyclobutyl; and —CN.

8. The compound of any one of claims 2-6, or a pharmaceutically acceptable salt thereof, wherein p is 1, and R¹⁰⁰ is F, Cl, methyl, ethyl, n-propyl, isopropyl, —CF₃, methoxy, ethoxy, n-propoxy, isopropoxy, —OCF₃, cyclopropyl, or —CN.

9. The compound of claim 1, or a pharmaceutically acceptable salt thereof, wherein
Z is O;
R³ and R⁴ are joined to form an optionally substituted phenyl, an optionally substituted 5 or 6-membered heteroaryl, e.g., having one or two ring nitrogen atoms, an optionally substituted C₄₋₇ cycloalkyl group (preferably cyclopentyl or cyclohexyl), or an optionally substituted 4 to 7-membered (preferably 6-membered) heterocyclic ring having one or two ring heteroatoms; and
R⁵ is —O—R³⁰ or —CR²³R²⁴R²⁵.

10. The compound of claim 9, or a pharmaceutically acceptable salt thereof, wherein R³ and R⁴ are joined to form a phenyl optionally substituted with one or two substituents independently selected from F; Cl; C₁₋₄ alkyl optionally substituted with 1-3 fluorines, preferably, methyl, ethyl, n-propyl, isopropyl, or —CF₃; a C₁₋₄ alkoxy optionally substituted with 1-3 fluorines, preferably, methoxy, ethoxy, n-propoxy, isopropoxy, or —OCF₃; a C₃₋₆ cycloalkoy optionally substituted with 1-3 substituents independently selected from fluorine and methyl; a C₃₋₆ cycloalkyl optionally substituted with 1-3 substituents independently selected from fluorine and methyl, preferably, cyclopropyl or cyclobutyl; and —CN.

11. The compound of claim 9, or a pharmaceutically acceptable salt thereof, wherein R³ and R⁴ are joined to form a 5 or 6-membered heteroaryl, preferably, pyrazole, imidazole, oxazole, thiazole, isoxazole, isothiazole, pyridyl, pyrimidinyl, pyridazinyl, or pyrazinyl, which is optionally substituted with one or two (preferably one) substituents independently selected from F; Cl; C₁₋₄ alkyl optionally substituted with 1-3 fluorines, preferably, methyl, ethyl, n-propyl, isopropyl, or —CF₃; a C₁₋₄ alkoxy optionally substituted with 1-3 fluorines, preferably, methoxy, ethoxy, n-propoxy, isopropoxy, or —OCF₃; a C₃₋₆ cycloalkoy optionally substituted with 1-3 substituents independently selected from fluorine and methyl; a C₃₋₆ cycloalkyl optionally substituted with 1-3 substituents independently selected from fluorine and methyl, preferably, cyclopropyl or cyclobutyl; and —CN.

12. The compound of claim 9, or a pharmaceutically acceptable salt thereof, wherein R³ and R⁴ are joined to form a 5 or 6-membered saturated ring system optionally containing one or two (preferably one) ring heteroatoms selected from O or N, which is optionally substituted with one or two substituents independently selected from F and C₁₋₄ alkyl, wherein the C₁₋₄ alkyl is optionally substituted with 1-3 fluorines.

13. The compound of any one of claims 1-12, or a pharmaceutically acceptable salt thereof wherein R⁵ is ethyl, n-propyl, isopropyl, n-butyl, isobutyl, sec-butyl, —CH₂—CHF₂, —CH₂—CF₃, —CF₃, —CH₂-cyclopropyl, —CH₂-cyclobutyl, —CH₂—O—CH₃, —CH₂—O—C₂H₅, —CH₂—O-n-propyl, —CH₂—O-isopropyl, —C₂H₄-cyclopropyl, —C₂H₄-cyclobutyl, methoxy, ethoxy, n-propoxy, isopropoxy, n-butoxy, isobutoxy, sec-butoxy, —O—CH₂—CF₃, —O—CF₃, —O—CH₂-cyclopropyl, —O—CH₂-cyclobutyl, —O—C₂H₄-cyclopropyl, or —O—C₂H₄-cyclobutyl.

14. The compound of any one of claims 1-13, or a pharmaceutically acceptable salt thereof, wherein J¹ is N.

15. The compound of any one of claims 1-13, or a pharmaceutically acceptable salt thereof, wherein J¹ is CH.

16. The compound of any one of claims 1-15, or a pharmaceutically acceptable salt thereof, wherein J² is N.

17. The compound of any one of claims 1-15, or a pharmaceutically acceptable salt thereof, wherein J² is CR²².

18. The compound of claim 17, or a pharmaceutically acceptable salt thereof, wherein J² is CR²² and R²² is hydrogen, F, Cl, CN, or methyl.

19. The compound of any one of claims 1-18, or a pharmaceutically acceptable salt thereof, wherein J³ is N.

20. The compound of any one of claims 1-18, or a pharmaceutically acceptable salt thereof, wherein J³ is CH.

21. The compound of any one of claims 1-20, or a pharmaceutically acceptable salt thereof, wherein R¹ is hydrogen.

22. The compound of any one of claims 1-21, or a pharmaceutically acceptable salt thereof, wherein n is 1.

23. The compound of any one of claims 1-21, or a pharmaceutically acceptable salt thereof, wherein n is 2.

24. The compound of any one of claims 1-21, or a pharmaceutically acceptable salt thereof, wherein n is 3.

25. The compound of any one of claims 1-24, or a pharmaceutically acceptable salt thereof wherein at least one instance of X is CR²⁰R²¹, wherein R²⁰ and R²¹ are independently hydrogen or C₁₋₄ alkyl, or R²⁰ and R²¹, together with the carbon they are both attached to, form a C₃₋₆ cycloalkyl (preferably, cyclopropyl, cyclobutyl, or cyclopentyl) or an oxetanyl ring.

26. The compound of any one of claims 1-25, or a pharmaceutically acceptable salt thereof, wherein one instance of X is O.

27. The compound of any one of claims 1-25, or a pharmaceutically acceptable salt thereof, wherein one instance of X is NR¹⁰, wherein R¹⁰ is hydrogen or C₁₋₄ alkyl.

28. The compound of any one of claims 1-21, or a pharmaceutically acceptable salt thereof, wherein the 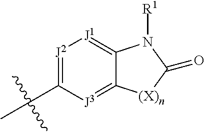
is selected from the following: 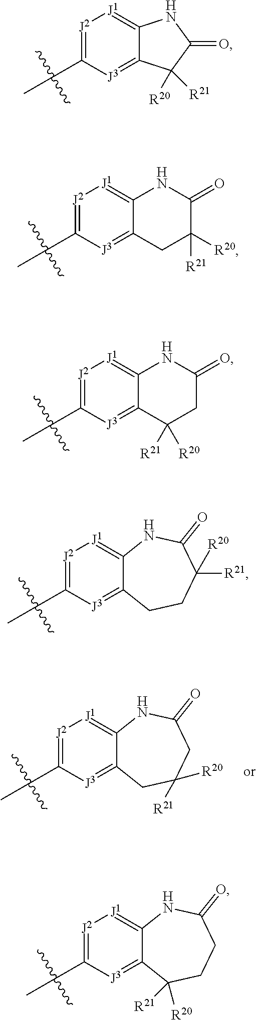
wherein R²⁰ and R²¹ are independently hydrogen or C₁₋₄ alkyl (e.g., methyl, ethyl, etc.), or R²⁰ and R²¹, together with the carbon they are both attached to, form a cyclopropyl, cyclobutyl, cyclopentyl, or an oxetanyl ring.

29. The compound of any one of claims 1-21, or a pharmaceutically acceptable salt thereof; wherein the 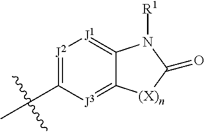
is selected from the following: 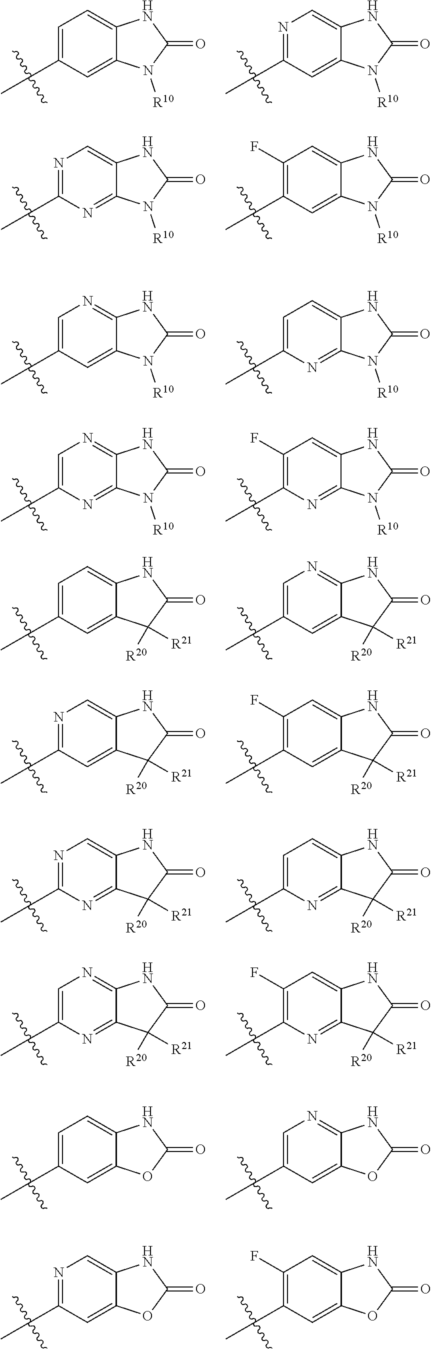 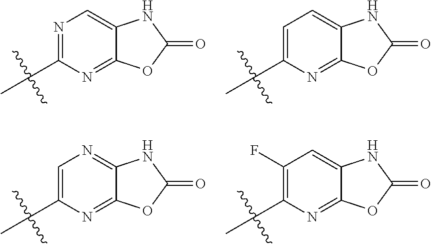
wherein:
R¹⁰ is independently hydrogen or C₁₋₄ alkyl (e.g., methyl, ethyl, etc.);
R²⁰ and R²¹ are independently hydrogen or C₁₋₄ alkyl (e.g., methyl, ethyl, etc.), or R²⁰ and R²¹, together with the carbon they are both attached to, form a cyclopropyl, cyclobutyl, cyclopentyl, or an oxetanyl ring.

30. The compound of any one of claims 1-21, or a pharmaceutically acceptable salt thereof, wherein the 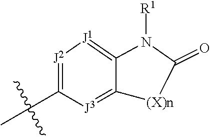
is selected from the following: 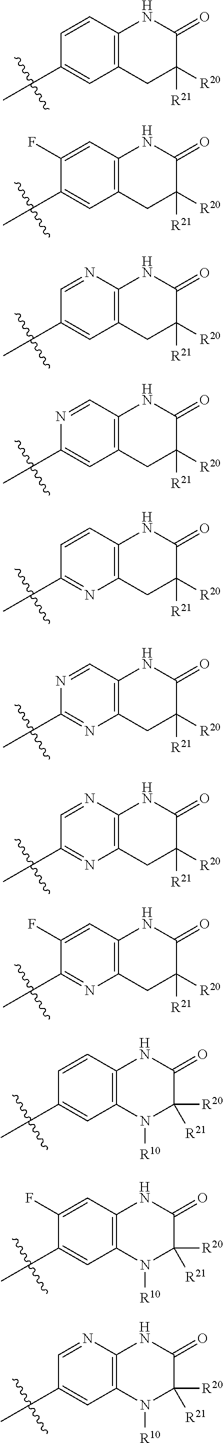 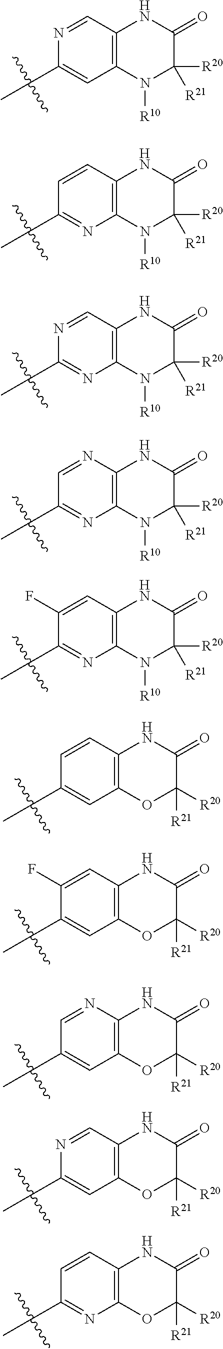 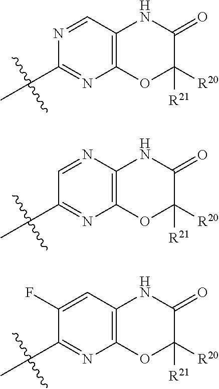
wherein:
R¹⁰ is independently hydrogen or C₁₋₄ alkyl (e.g., methyl, ethyl, etc.);
R²⁰ and R²¹ are independently hydrogen or C₁₋₄ alkyl (e.g., methyl, ethyl, etc.), or R²⁰ and R²¹, together with the carbon they are both attached to, form a cyclopropyl, cyclobutyl, cyclopentyl, or an oxetanyl ring.

31. The compound of any one of claims 1-21, or a pharmaceutically acceptable salt thereof, wherein the 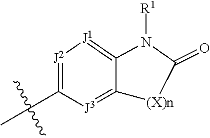
is selected from the following: 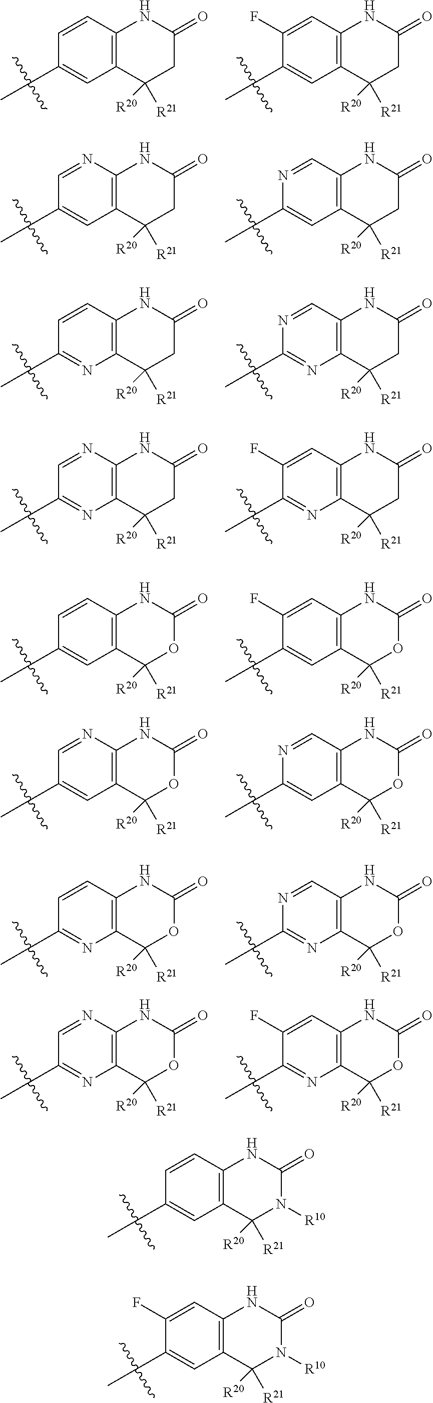 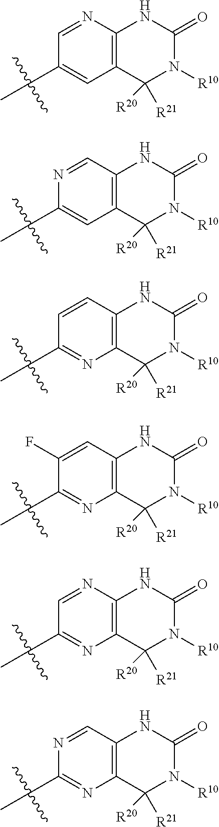
wherein:
R¹⁰ is independently hydrogen or C₁₋₄ alkyl (e.g., methyl, ethyl, etc.);
R²⁰ and R²¹ are independently hydrogen or C₁₋₄ alkyl (e.g., methyl, ethyl, etc.), or R²⁰ and R²¹, together with the carbon they are both attached to, form a cyclopropyl, cyclobutyl, cyclopentyl, or an oxetanyl ring.

32. The compound of any one of claims 1-21, or a pharmaceutically acceptable salt thereof wherein the 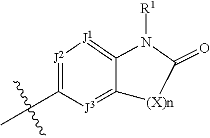
is selected from the following: 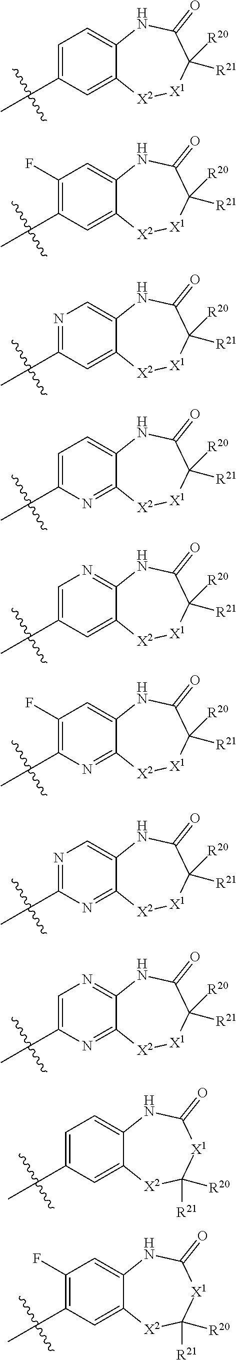 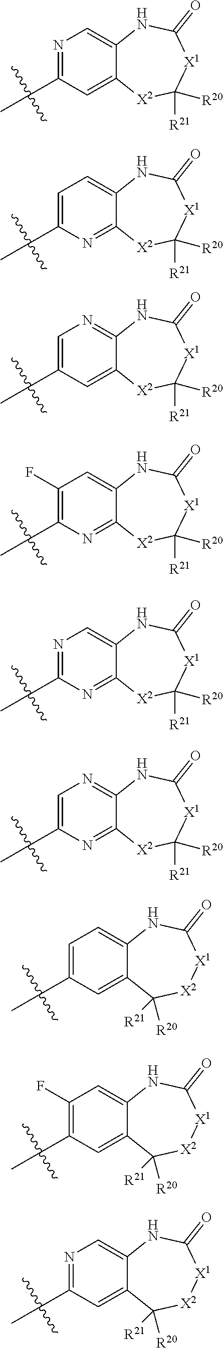 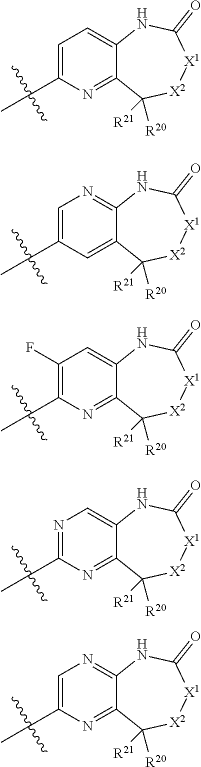
wherein:
X¹ and X² are independently O, NR¹⁰, or CH₂, provided that at least one of X¹ and X² is CH₂;
R¹⁰ is hydrogen or C₁₋₄ alkyl (e.g., methyl, ethyl, etc.);
R²⁰ and R²¹ are independently hydrogen or C₁₋₄ alkyl (e.g., methyl, ethyl, etc.), or R²⁰ and R²¹, together with the carbon they are both attached to, form a cyclopropyl, cyclobutyl, cyclopentyl, or an oxetanyl ring.

33. The compound of any one of claims 1-21, or a pharmaceutically acceptable salt thereof, wherein:
(X)ₙ in the formula includes 1-3 CR²⁰R²¹ units, wherein for at least one CR²⁰R²¹ unit, R²⁰ and R²¹ are both methyl;
one of R²⁰ and R²¹ is methyl, and the other of R²⁰ and R²¹ is ethyl or methoxy; or
R²⁰ and R²¹, together with the carbon they are both attached to, form a cyclopropyl, cyclobutyl, or an oxetanyl ring.

34. The compound of any one of claims 1-21, or a pharmaceutically acceptable salt thereof wherein the 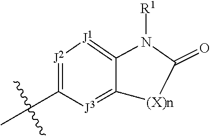
in Formula I is selected from the following:

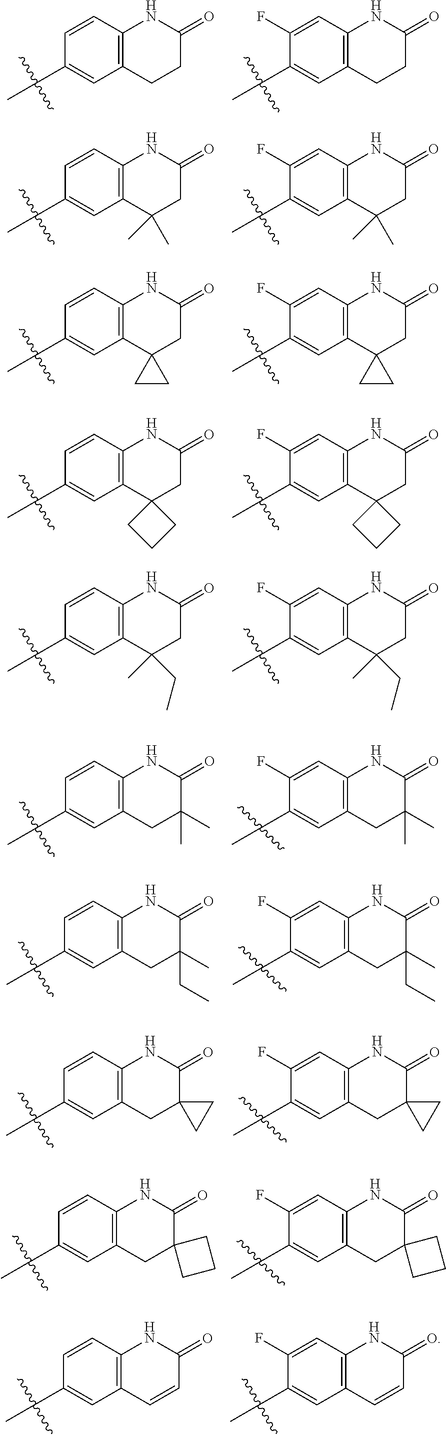

35. The compound of any one of claims 1-21, or a pharmaceutically acceptable salt thereof wherein the 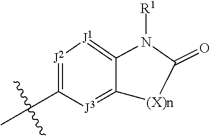
in Formula I is selected from the following:

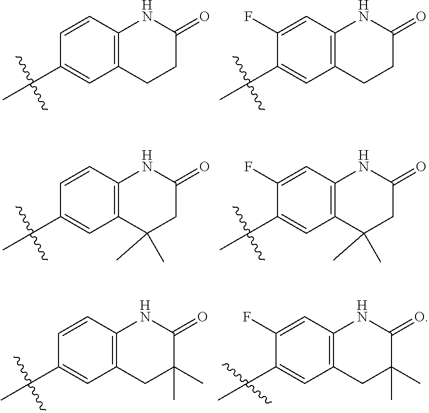

36. The compound of any one of claims 1-21, or a pharmaceutically acceptable salt thereof, wherein the 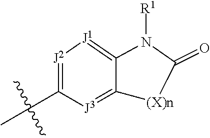
in Formula I is selected from the following:

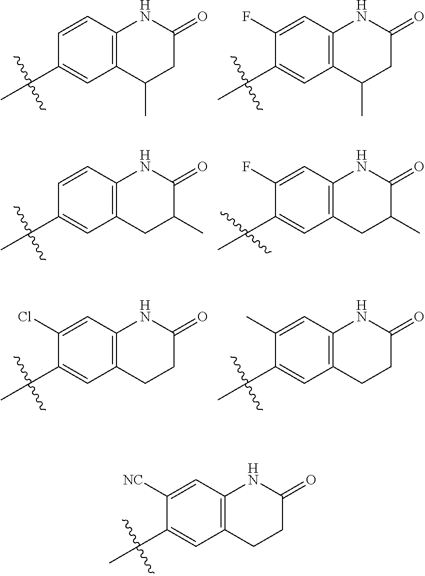

37. A compound of Formula II, or a pharmaceutically acceptable salt thereof: 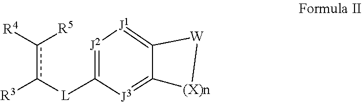
wherein:
W is —N(R¹)—C(O)—, —N(R¹)—S(O)—, or —N(R¹)—S(O)₂—;
L is —(CR^{A1}R^{B1})ₜ1-Q¹-Q²-Q³-(CR^{A2}R^{B2})ₜ₂ wherein:
Q¹ and Q³ are independently null, O or NR²;
Q² is null, —C(O)—, —C(═Z)—, —S(O)—, or —S(O)₂—;
t1 is 0, 1, 2, or 3;
t2 is 0, 1, 2, or 3; and
R^{A1}, R^{B1}, R^{A2}, and R^{B2} at each occurrence are independently hydrogen, C₁₋₄ alkyl (e.g., methyl), or fluorine, or
two adjacent CR^{A1}R^{B1} or two adjacent CR^{A2}R^{B2} can form —C(R^{A1})═C(R^{B1})—, —C(R^{A2})═C(R^{B2})—, or 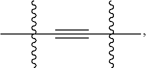
wherein R^{A1}, R^{B1}, R^{A2}, and R^{B2} at each occurrence are independently hydrogen, C₁₋₄ alkyl (e.g., methyl), or fluorine;
X at each occurrence is independently selected from O, NR¹⁰, and CR²⁰R²¹, provided that at most one X is selected from O and NR¹⁰;
n is 1, 2, 3, or 4;
J¹, J², and J³ are each independently selected from CR²² or N, preferably, at least one of J¹, J², and J³ is not N;
R¹ and R² at each occurrence are each independently hydrogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), or a nitrogen protecting group;
R³ and R⁴ are joined to form an optionally substituted aryl, an optionally substituted heteroaryl, an optionally substituted carbocyclic (e.g., C₃₋₈ carbocyclic), or an optionally substituted heterocyclic ring (e.g., 3-8 membered heterocyclic ring);
R⁵ is hydrogen, —NR¹¹R¹², —CR²³R²⁴R²⁵, or —OR³⁰;
R³, R⁴ and R⁵ are joined to form an optionally substituted bicyclic or polycyclic ring system, wherein the ring system is an aryl, heteroaryl, carbocyclic, or heterocyclic ring system;
or when Q² is —C(═Z)—, R⁵ and Z are joined to form an optionally substituted aryl, an optionally substituted heteroaryl, an optionally substituted carbocyclic (e.g., C₃₋₈ carbocyclic), or an optionally substituted heterocyclic ring (e.g., 3-8 membered heterocyclic ring);
“ [symbol] ” in Formula II indicates the bond is an aromatic bond, a double bond or a single bond as valance permits, and when a single bond, the two carbons forming the bond can be optionally further substituted as valance permits;
wherein:
R¹⁰ at each occurrence is independently hydrogen, a nitrogen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, or an optionally substituted 3-8 membered heterocyclic ring;
R²⁰ and R²¹ at each occurrence are each independently hydrogen, halogen, —OR³¹, —NR¹³R¹⁴, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl; or
R¹⁰ and one of R²⁰ and R²¹ are joined to form a bond, an optionally substituted 4-8 membered heterocyclic ring or an optionally substituted 5 or 6 membered heteroaryl ring, wherein the other of R²⁰ and R²¹ is defined above;
R²⁰ and R²¹ together with the carbon they are both attached to form —C(O)—, an optionally substituted C₃₋₈ carbocyclic ring, or an optionally substituted 3-8 membered heterocyclic ring; or
one of R²⁰ and R²¹ in one CR²⁰R²¹ is joined with one of R²⁰ and R²¹ in a different CR²⁰R²¹ to form a bond, an optionally substituted C₃₋₈ carbocyclic ring, an optionally substituted 3-8 membered heterocyclic ring, wherein the others of R²⁰ and R²¹ are defined above;
R²² at each occurrence is independently hydrogen, halogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), —CN, —S(O)-alkyl, —S(O)₂-alkyl, or —OR³¹;
one of R¹¹ and R¹² is hydrogen or a nitrogen protecting group, and the other of R¹¹ and R¹² is hydrogen, a nitrogen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl;
one of R²³, R²⁴, and R²⁵ is hydrogen, halogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, an optionally substituted 5-10 membered heteroaryl, —OR³¹, or NR¹³R¹⁴, and the other two of R²³, es, and R²⁵ are independently selected from hydrogen, fluorine, or methyl, preferably, —CR²³R²⁴R²⁵ is not —CH₃;
R³⁰ is hydrogen, an oxygen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃-$ carbocyclic ring, or an optionally substituted 3-8 membered heterocyclic ring; and
wherein:
each of R¹³ and R¹⁴ at each occurrence is independently hydrogen, a nitrogen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl; or R¹³ and R¹⁴ are joined to form a 3-8 membered optionally substituted heterocyclic or a 5-10 membered optionally substituted heteroaryl; and
R³¹ at each occurrence is hydrogen, an oxygen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl.

38. The compound of claim 37, or a pharmaceutically acceptable salt thereof, wherein W is —NH—C(O)— or NH—S(O)₂—.

39. The compound of claim 37 or 38, or a pharmaceutically acceptable salt thereof, wherein L is —(CR^{A1}R^{B1})ₜ1—N(R²)—, wherein t1 is 1 or 2.

40. The compound of claim 37 or 38, or a pharmaceutically acceptable salt thereof, wherein L is —(CR^{A1}R^{B1})ₜ1—, wherein t1 is 1 or 2.

41. The compound of claim 37 or 38, or a pharmaceutically acceptable salt thereof, wherein L is —(CR^{A1}R^{B1})^{t}1—N(R²)—C(O)—, wherein t1 is 1 or 2.

42. The compound of claim 37 or 38, or a pharmaceutically acceptable salt thereof, wherein L is —N(R²)—C(O)—(CR^{A2}R^{B2})ₜ₂—, wherein t2 is 1 or 2.

43. The compound of claim 37 or 38, or a pharmaceutically acceptable salt thereof, wherein L is (CR^{A1}R^{B1})ₜ1—N(R²)—C(O)—(CR^{A2}R^{B2})ₜ₂—, wherein t1 and t2 are independently 0, 1 or 2.

44. A compound of Formula III, or a pharmaceutically acceptable salt thereof, 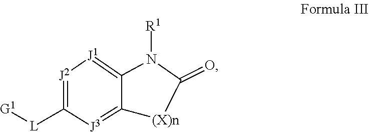
wherein:
X at each occurrence is independently selected from O, NR¹⁰, and CR²⁰R²¹, provided that at most one X is selected from O and NR¹⁰;
n is 1, 2, 3, or 4;
J¹, J², and J³ are each independently selected from CR²² or N, preferably, at least one of J¹, J², and J³ is not N;
R¹ is hydrogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), or a nitrogen protecting group;
L is NH, O, or selected from: 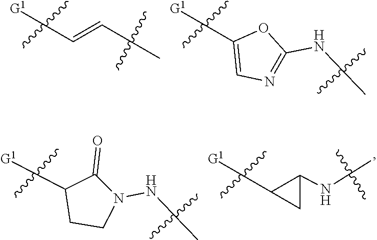
G¹ is an optionally substituted phenyl, optionally substituted heteroaryl (e.g., 5- or 6-membered heteroaryl, or 8-10 membered bicyclic heteroaryl), or an optionally substituted heterocyclyl,
wherein:
R¹⁰ at each occurrence is independently hydrogen, a nitrogen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, or an optionally substituted 3-8 membered heterocyclic ring;
R²⁰ and R²¹ at each occurrence are each independently hydrogen, halogen, —OR³¹, —NR¹³R¹⁴, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl; or
R¹⁰ and one of R²⁰ and R²¹ are joined to form a bond, an optionally substituted 4-8 membered heterocyclic ring or an optionally substituted 5 or 6 membered heteroaryl ring, wherein the other of R²⁰ and R²¹ is defined above;
R²⁰ and R²¹ together with the carbon they are both attached to form —C(O)—, an optionally substituted C₃₋₈ carbocyclic ring, or an optionally substituted 3-8 membered heterocyclic ring; or
one of R²⁰ and R²¹ in one CR²⁰R²¹ is joined with one of R²⁰ and R²¹ in a different CR²⁰R²¹ to form a bond, an optionally substituted C₃₋₈ carbocyclic ring, an optionally substituted 3-8 membered heterocyclic ring, wherein the others of R²⁰ and R²¹ are defined above;
R²² at each occurrence is independently hydrogen, halogen, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆alkynyl), —CN, —S(O)-alkyl (e.g., —S(O)—C₁₋₆alkyl), —S(O)₂-alkyl (e.g., —S(O)₂—C₁₋₆ alkyl), or —OR³¹;
wherein:
each of R¹³ and R¹⁴ at each occurrence is independently hydrogen, a nitrogen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl; or R¹³ and R¹⁴ are joined to form a 3-8 membered optionally substituted heterocyclic or a 5-10 membered optionally substituted heteroaryl; and
R³¹ at each occurrence is hydrogen, an oxygen protecting group, an optionally substituted alkyl (e.g., optionally substituted C₁₋₆ alkyl), an optionally substituted alkenyl (e.g., optionally substituted C₂₋₆ alkenyl), an optionally substituted alkynyl (e.g., optionally substituted C₂₋₆ alkynyl), an optionally substituted C₃₋₈ carbocyclic ring, an optionally substituted 3-8 membered heterocyclic ring, an optionally substituted phenyl, or an optionally substituted 5-10 membered heteroaryl.

45. The compound of claim 44, or a pharmaceutically acceptable salt thereof, characterized as having a Formula III-1 or III-2:

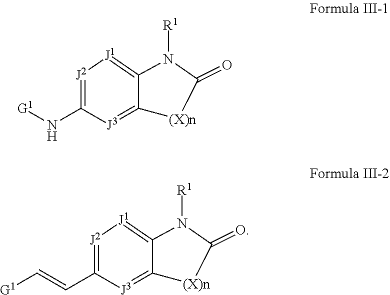

46. The compound of claim 44 or 45, or a pharmaceutically acceptable salt thereof, wherein the 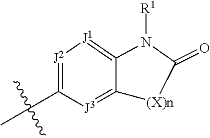
in Formula III is selected from the following:

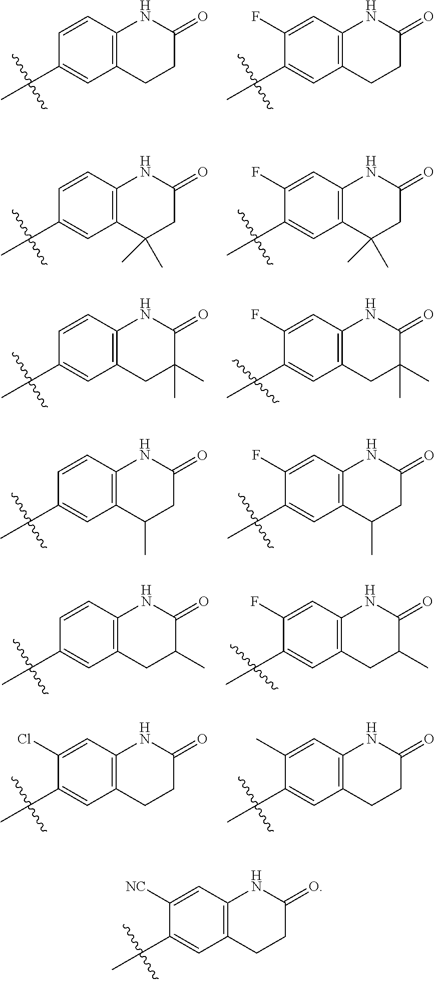

47. The compound of any one of claims 44-46, or a pharmaceutically acceptable salt thereof, wherein G¹ is selected from: 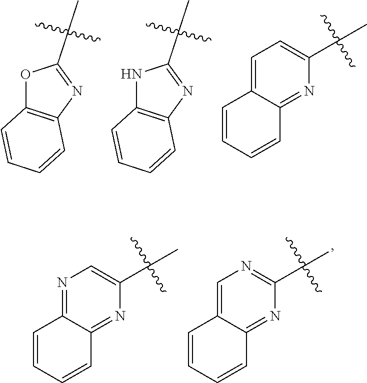
wherein each of which is optionally substituted, for example, with one or two substituents independently selected from Cl, methyl, and hydroxyl.

48. The compound of any one of claims 44-46, or a pharmaceutically acceptable salt thereof, wherein G¹ is selected from:

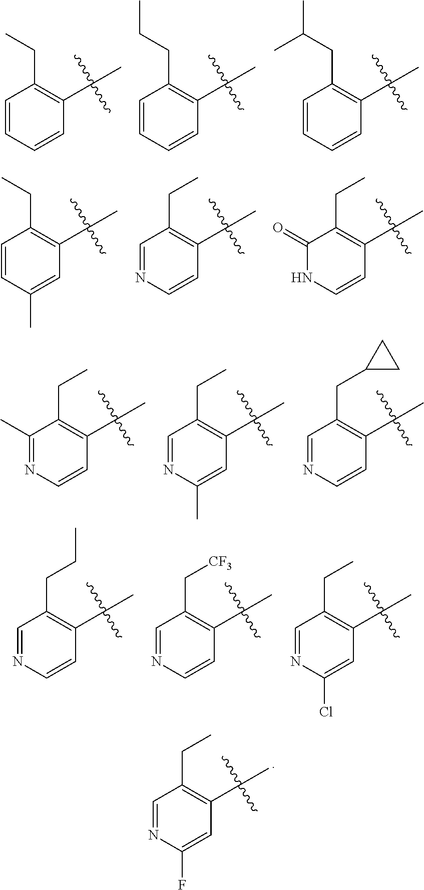

49. A compound selected from any of Compound Nos. 1-138, or a pharmaceutically acceptable salt thereof.

50. A pharmaceutical composition comprising the compound of any one of claims 1-49, or a pharmaceutically acceptable salt thereof, and optionally a pharmaceutically acceptable excipient or carrier.

51. A method of treating cancer in a subject in need thereof, the method comprising administering to the subject a therapeutically effective amount of the compound of any one of claims 1-49 or a pharmaceutical salt thereof, or the pharmaceutical composition of claim 50.

52. A method of treating metastatic cancer or chemoresistant cancer in a subject in need thereof, the method comprising administering to the subject a therapeutically effective amount of the compound of any one of claims 1-49 or a pharmaceutical salt thereof, or the pharmaceutical composition of claim 50.

53. A method of treating or preventing metastasis of a cancer in a subject in need thereof, the method comprising administering to the subject an effective amount of the compound of any one of claims 1-49 or a pharmaceutical salt thereof, or the pharmaceutical composition of claim 50.

54. A method of sensitizing cancer for chemotherapy in a subject in need thereof, the method comprising administering to the subject an effective amount of the compound of any one of claims 1-49 or a pharmaceutical salt thereof, or the pharmaceutical composition of claim 50.

55. The method of any one of claims 51-54, further comprising administering to the subject an effective amount of a second anti-cancer therapy, such as a chemotherapeutic agent or a therapeutic antibody.

56. The method of any one of claims 51-55, wherein the cancer is a breast cancer, colorectal cancer, kidney cancer, ovarian cancer, gastric cancer, thyroid cancer, testicular cancer, cervical cancer, nasopharyngeal cancer, esophageal cancer, bile duct cancer, lung cancer, pancreatic cancer, prostate cancer, bone cancer, blood cancer, brain cancer, liver cancer, mesothelioma, melanoma, and/or sarcoma.

57. A method of treating or preventing type 2 diabetes in a subject in need thereof, the method comprising administering to the subject an effective amount of the compound of any one of claims 1-49 or a pharmaceutical salt thereof, or the pharmaceutical composition of claim 50.

58. A method of treating or preventing a metabolic disease in a subject in need thereof, the method comprising administering to the subject an effective amount of the compound of any one of claims 1-49 or a pharmaceutical salt thereof; or the pharmaceutical composition of claim 50.

59. A method of inhibiting an aldehyde dehydrogenase in a subject in need thereof, comprising administering to the subject an effective amount of the compound of any one of claims 1-49 or a pharmaceutical salt thereof, or the pharmaceutical composition of claim 50.

60. A method of treating a disease or disorder associated with aldehyde dehydrogenase, preferably, a disease or disorder associated with aldehyde dehydrogenase isoform 1a3 (ALDH1a3) in a subject in need thereof, comprising administering to the subject an effective amount of the compound of any one of claims 1-49 or a pharmaceutical salt thereof, or the pharmaceutical composition of claim 50.

61. The method of claim 60, wherein the disease or disorder is a proliferative disease or disorder or a metabolic disease or disorder.

62. A method of treating an endothelial cell or smooth muscle cell disease or disorder, such as pulmonary arterial hypertension or neointimal hyperplasia in a subject in need thereof, comprising administering to the subject an effective amount of the compound of any one of claims 1-49 or a pharmaceutical salt thereof, or the pharmaceutical composition of claim 50.
